(19) United States
(12) Patent Application Publication
(10) Pub. No.: US 2025/0043269 A1
(43) Pub. Date: Feb. 6, 2025

(54) Precise Genome Editing Using Retrons

(71) Applicants: The J. David Gladstone Institutes, a testamentary trust established under the Will of J. David Glad, San Francisco, CA (US); The Regents of the University of California, Oakland, CA (US)

(72) Inventors: Santiago C. Lopez, Oakland, CA (US); Seth Shipman, San Francisco, CA (US)

(21) Appl. No.: 18/707,490

(22) PCT Filed: Nov. 3, 2022
(86) PCT No.: PCT/US2022/079220

§ 371 Date: May 3, 2024

Related U.S. Application Data
(60) Provisional application No. 63/275,287, filed on Nov. 3, 2021.

(51) Int. Cl. C12N 15/10; C12N 9/12; C12N 9/22; C12N 15/11

(52) U.S. Cl. CPC C12N 15/102; C12N 9/1276; C12N 9/22; C12N 15/111; C12N 2310/20; C12Y 207/07049

(57) ABSTRACT

Described herein are compositions and methods that provide precise editing of cells, including mammalian cells and human cells. The compositions and methods utilize retrons as repair donors, retron reverse transcriptases to make those retron repair donors, retron-encoded guide RNAs, and CRISPR nucleases.

### CROSS-REFERENCE TO RELATED APPLICATIONS

This application claims the benefit of priority to U.S. Provisional Ser. No. 63/275,287, filed Nov. 3, 2021, which is incorporated by reference as if fully set forth herein.

### INCORPORATION BY REFERENCE OF SEQUENCE LISTING

A Sequence Listing is provided herewith as an xml file. “2283595.xml” created on Nov. 2, 2022 and having a size of 114,688 bytes. The content of the xml file is incorporated by reference herein in its entirety.

### TECHNICAL FIELD

The present disclosure generally relates to systems, methods and compositions used for precise genome editing, including nucleic acid insertions, replacements, and deletions at targeted and precise genome sites, wherein said systems, methods, and compositions are based on novel and/or engineered retrons.

### BACKGROUND

Exogenous DNA can be introduced into cells as a template to edit the cell's genome. However, the delivery of in vitro-produced DNA to target cells can be inefficient, and low abundance of template DNA reduces the rate of precise editing. A potential tool to produce template DNA inside cells is a retron, a bacterial retroelement thought to be involved in phage defense. The ssDNA generated by retrons has been used for genome engineering in two contexts: for bacterial genomic editing, with the λ Red Beta recombinase (Farzadfard et al. Science 346, 1256272, (2014)); and for yeast genomic editing with a homology-directed repair (HDR) template for Cas9 editing (Sharon et al. Cell 175, 544-557.e516, (2018)). However, such efforts in bacteria and yeast suffered from lower-than-expected efficiency and context-restriction. Such problems may stem from elements in the endogenous form of the retron such as (1) a branched retron structure with a phosphodiester bond linking the 5′ end of the ssDNA to a 2′ hydroxyl of the retron msr RNA, (2) invariant retron flanking regions that may be required for retron reverse transcription, but are not part of the repair template, (3) limited total retron length, and (4) a native poly T stretch in retrons that functions as a terminator for Pol III transcription.

### SUMMARY

Described herein are compositions, systems, and methods that provide precise editing of cells, tissues, organs, and organisms, including precise editing in mammalian and human cells, tissues, and organs. The compositions, systems, and methods, in various embodiments, are capable of precise editing under in vitro conditions. In other embodiments, the compositions, systems, and methods are capable of precise editing under n vivo conditions. In still other embodiments, the compositions, systems, and methods are capable of precise editing under ex vivo conditions. The compositions and methods utilize programmable nucleases (e.g., CRISPR nucleases, proteins containing zinc finger domains (ZFP), or proteins containing TALE domains (e.g., TALEN)) combined with retrons as repair donors, and in certain embodiments, further combined with a retron reverse transcriptase to carry out the synthesis of the retron repair donors (i.e., by way of the RT-catalyzed conversion of the ncRNA molecule to its cognate msDNA (multicopy single-stranded DNA and which includes the single stranded DNA product of reverse transcription, i.e., the RT-DNA). The specific retron constructs (e.g., engineered retron DNA, retron ncRNAs, and retron msDNA) and retron reverse transcriptases described herein are particularly useful for targeted genome cutting and precise genomic modification (e.g., precise editing) of mammalian cells, tissues, and organs, including human cells, tissues, and organs.

In various aspects, the present specification describes nucleic acid molecules encoding the recombinant retrons and/or recombinant retron components (e.g., a recombinant ncRNA and/or a recombinant retron RT). In still other aspects, the present disclosure provides genome editing systems comprising recombinant retron components (e.g., recombinant ncRNAs, recombinant msDNAs (including the RT-DNAs), and/or recombinant retron RTs), programmable nucleases (e.g., programmable nucleases, such as CRISPR-Cas proteins, ZFPs, and TALENS), and guide RNAs (in the case where RNA-guide nucleases are used in said genome editing systems). In further aspects, the disclosure provides nucleic acid molecules encoding the herein described genome editing systems and said components thereof, as well as polypeptides making up the components of said genome editing systems. In yet another aspect, the disclosure provides vectors for transferring and/or expressing said genome editing systems, e.g., under in vitro, ex vivo, and in vivo conditions. In still other aspects, the disclosure provides cell-delivery compositions and methods, including compositions for passive and/or active transport to cells (e.g., plasmids), delivery by virus-based recombinant vectors (e.g., AAV and/or lentivirus vectors), delivery by non-virus-based systems (e.g., liposomes and LNPs), and delivery by virus-like particles. Depending on the delivery system employed, the retron precision editing systems described herein may be delivered in the form of DNA (e.g., plasmids or DNA-based virus vectors). RNA (e.g., ncRNA and mRNA delivered by LNPs), a mixture of DNA and RNA, protein (e.g., virus-like particles), and ribonucleoprotein (RNP) complexes. Any suitable combinations of approaches for delivering the components of the herein disclosed retron precision editing systems may be employed. In one embodiment, each of the components of the retron precision editing systems described herein is delivered by an all-RNA system, e.g., the delivery of one or more RNA molecules (e.g., mRNA and/or ncRNA) by one or more LNPs, wherein the one or more RNA molecules form the ncRNA and guide RNA (as needed) and/or are translated into the polypeptide components (e.g., the RT and a programmable nuclease). In yet another aspect, the disclosure provides methods for genome editing by introducing a retron precision editing system described herein into a cell (e.g., under in vitro, in vivo, or ex vivo conditions) comprising a target edit site, thereby resulting in an edit at the target edit. In other aspects, the disclosure provides formulations comprising any of the aforementioned components for delivery to cells and/or tissues, including in vitro, in vivo, and ex vivo delivery, recombinant cells and/or tissues modified by the recombinant retron precision editing systems and methods described herein, and methods of modifying cells by conducting genome editing and related DNA donor-dependent methods, such as recombineering, or cell recording, using the herein disclosed retron precision editing systems. The disclosure also provides methods of making the recombinant retrons, retron precision editing systems and components thereof (including ncRNAs, RT-DNAs, and retron RTs), vectors, compositions and formulations described herein, as well as to pharmaceutical compositions and kits for modifying cells under in vitro, in vivo, and ex vivo conditions that comprise the herein disclosed genome editing and/or modification systems.

One embodiment provides an engineered retron ncRNA comprising: an msr region, an msd region having an msd stem and msd loop, and an a1/a2 duplex region, wherein a1/a2 duplex region comprises at least 7 nucleotide base pairs, wherein the a1/a2 duplex further comprises a guide RNA, and wherein the msd loop comprises a repair template. In one embodiment, the engineered retron ncRNA of claim 1, wherein the msd stem is between 12 and 30 nucleotide base pairs in length. In another embodiment, the msd loop is between 5-14 nucleotides in length or alternately is at least 12 nucleotides in length and optionally may comprise the repair template. In one embodiment, the a1/a2 duplex is modified by increasing its length by at least 15 nucleotides, or by at least 16 nucleotides, or by at least 17 nucleotides, or by at least 18 nucleotides, or by at least 19 nucleotides, or by at least 20 nucleotides, or by at least 22 nucleotides, or by at least 25 nucleotides, or by at least 30 nucleotides. In one embodiment, the guide RNA binds to a target genomic DNA. In another embodiment, the guide RNA binds to a target genomic DNA in a bacterial, yeast, or mammalian cell. In one embodiment, the guide RNA is fused to the end of either strand of the a1/a2 duplex. In one embodiment, the mammalian cell is a human cell. In one embodiment, the repair template binds to a target genomic DNA. In one embodiment, the repair template binds to a target genomic DNA in a bacterial, yeast, or mammalian cell. In another embodiment, the repair template binds to a target genomic DNA having at least one allele with a mutation or polymorphism. In one embodiment, the repair template comprises one or more non-complementary nucleotides compared to the target genomic DNA. In one embodiment, the repair template comprises two or more, or three or more non-complementary nucleotides compared to the target genomic DNA. In one embodiment, the non-complementary nucleotides are ‘repair’ nucleotides that can substitute for mutant, variant, or polymorphism nucleotides in the target genomic DNA. In one embodiment, the msd stem is at least 12 nucleotides in length. In another embodiment, the msd stem is 30 or fewer nucleotides in length.

One embodiment provides for a composition comprising a carrier and the engineered retron ncRNA described herein.

Another embodiment provides a method comprising administering the engineered retron ncRNA described herein, or the composition described herein to a subject or to cell(s) from the subject. In one embodiment, wherein the subject has, or is suspected of having or developing a disease or condition. In one embodiment, the disease or condition is cystic fibrosis, thalassemia, sickle cell anemia. Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

One embodiment provides for an expression cassette comprising a nucleotide sequence encoding the engineered ncRNA described herein, and optionally a nucleotide sequence encoding a retron reverse transcriptase. In one embodiment, the nucleotide sequence encoding the engineered ncRNA further comprises a first promoter, wherein the first promoter is optionally an RNA polymerase III promoter. In one embodiment, the first promoter is a 7SK, U6, or H1 RNA polymerase III promoter. In one embodiment, the first promoter is an RNA polymerase II promoter. In one embodiment the nucleotide sequence encoding the retron reverse transcriptase further comprises a second promoter. In one embodiment, the second promoter is the same or different as the first promoter.

One embodiment provides a vector comprising the expression cassette described herein.

Another embodiment provides a composition comprising a carrier and the expression cassette described herein or the vector described herein.

One embodiment provides a method comprising administering the expression cassette described herein or the vector described herein, or the composition of described herein to a subject or to cell(s) from the subject. In one embodiment, the subject has, or is suspected of having or developing a disease or condition. In one embodiment, the disease or condition is cystic fibrosis, thalassemia, sickle cell anemia. Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

Another embodiment provides a gene editing system comprising: one or more vectors comprising one or more nucleotide sequences encoding an engineered retron ncRNA described herein, a retron reverse transcriptase, and a Cas nuclease. In one embodiment, the retron reverse transcriptase and Cas nuclease are encoded as a fusion protein. In one embodiment, the nucleotide sequence encoding the fusion protein comprising the retron reverse transcriptase and the Cas nuclease further comprises a ribosomal skipping sequence. In one embodiment, the skipping sequence comprises DxExNPGP (SEQ ID NO: 9), and each x is independently an amino acid. In one embodiment, the skipping sequence comprises one of the following sequences:

T2A
(SEQ ID NO: 10))
(GSG) EGRGSLL TCGDVEENPGP
P2A
(SEQ ID NO: 11)
(GSG) ATNFSLLKQAGDVEENPGP
E2A
(SEQ ID NO: 12)
(GSG) QCTNYALLKLAGDVESNPGP
F2A
(SEQ ID NO: 13)
(GSG) VKQTLNFDLLKLAGDVESNPGP

In another embodiment, the one or more vectors comprise one or more promoters. In one embodiment, the guide RNA of the ncRNA binds to a target genomic DNA. In one embodiment, the guide RNA of the ncRNA binds to a target genomic DNA in a bacterial, yeast, or mammalian cell. In another embodiment, the guide RNA of the ncRNA binds to a target genomic DNA in a mammalian cell. In one embodiment, the mammalian cell is a human cell. In another embodiment, the repair template of the ncRNA binds to a target genomic DNA. In one embodiment, the repair template of the ncRNA binds to a target genomic DNA in a bacterial, yeast, or mammalian cell. In one embodiment, the repair template of the ncRNA binds to a target genomic DNA having at least one allele with a mutation or polymorphism. In one embodiment, the repair template of the ncRNA comprises one or more non-complementary nucleotides compared to the target genomic DNA. In another embodiment, the repair template of the ncRNA comprises two or more, or three or more non-complementary nucleotides compared to the target genomic DNA. In one embodiment, the non-complementary nucleotides are ‘repair’ nucleotides that can substitute for mutant, variant, or polymorphism nucleotides in the target genomic DNA. In another embodiment, at least one promoter is an RNA polymerase III promoter. In one embodiment, the RNA polymerase III promoter is a 7SK, U6, or H1 RNA polymerase III promoter. In one embodiment, at least one promoter is an RNA polymerase II promoter. Another embodiment provides a first vector encoding the ncRNA and a second vector encoding the retron reverse transcriptase and Cas nuclease.

One embodiment provides a carrier and the gene editing system described herein.

Another embodiment provides a method comprising administering the gene editing system described herein, or the composition described herein to a subject or to cell(s) from the subject. In one embodiment, the subject has, or is suspected of having or developing a disease or condition. In one embodiment, the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease. Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

One embodiment provides a method of genetically editing one or more cells, comprising:
1. transfecting a population of cells with the expression cassette of described herein, or the gene editing system described herein to generate a population of transfected cells; and
2. selecting one or more cells from the population of transfected cells as genetically edited cells.
   In one embodiment, selecting one or more cells comprises generating colonies from individual transfected cells to provide isogenic individual colonies and selecting one or more precisely edited cells from at least one isogenic colony. One embodiment further comprises sequencing one or more genomic target sites in cells from one or more isogenic individual colonies to confirm that the genomic target sites in at least one of the isogenic individual colonies are precisely edited, thereby generating precisely edited cells. Another embodiment further comprises administering a population of the precisely edited cells to a subject. In one embodiment, the subject has, or is suspected of having or developing a disease or condition. In one embodiment, the disease or condition is cystic fibrosis, thalassemia, sickle cell anemia. Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

In one aspect, described herein are engineered retron ncRNAs that include (1) a guide RNA linked or inserted into an al or a2 complementary region of the ncRNA; and (2) a repair template inserted into the ncRNA msd-encoding loop. Also described herein are nucleic acid constructs encoding the engineered ncRNAs, recombinant msDNAs formed from the ncRNA by reverse transcription (including the RT-DNA), and compositions comprising one or more retron components, including recombinant ncRNAs, msDNAs (including the RT-DNAs), and retron RT, and/or nucleic acid molecules encoding same. Also described herein are compositions and methods of administering such engineered retron components, including engineered retron ncRNAs, to a cell, tissue, or organ of a subject, e.g., by in vitro, in vivo, or ex vivo delivery methods, are also described herein.

Expression cassettes and expression systems are also described herein. For example, described herein are expression systems that include at least one expression cassette or expression vector having a promoter operably linked to a DNA segment encoding a retron reverse transcriptase, a DNA segment encoding a programmable nuclease (e.g., a Cas nuclease), or a DNA segment encoding both a retron reverse transcriptase and a Cas nuclease. The expression systems can also include an expression cassette or expression vector that includes a promoter operably linked to a DNA segment encoding a retron ncRNA. The retron can be an engineered retron ncRNA that includes (1) a guide RNA linked or inserted into an a1 or a2 complementary region of the ncRNA; and (2) a repair template inserted into the ncRNA msd-encoding loop. The components described above, including a programmable nuclease (e.g., a Cas9 nuclease), retron RT, an engineered retron ncRNA, and a guide RNA and be configured in any suitable arrangement on one or multiple different expression cassettes. For instance, the programmable nuclease and the retron RT can be encoded as a single fusion protein on an expression cassette. In such configurations, the fusion protein may comprise the nuclease at the amino terminal end. In other configurations, the fusion protein may comprise the retron RT at the amino terminal end. In other embodiments, the retron ncRNA may be encoded from its own expression vector, or encoded on the same expression cassette as the programmable nuclease and retron RT. In still other embodiments, the guide RNA may be encoded on its own expression cassette, or on the same expression cassette as the ncRNA and/or the programmable nuclease and/or the retron RT. In addition, the ncRNA may be engineered to include the guide RNA linked or inserted into the ncRNA, e.g., into an al or a2 complementary region (i.e., the a1/a2 duplex). Compositions and methods of administering such expression cassette or expression vector to a cell or a subject are also described herein.

The expression cassettes and/or expression systems can be administered to a subject or to cells from a subject. Cells that modified to include precisely edited genomic loci can be administered to a subject. Such methods can treat, prevent, or reduce the onset of a disease or condition in the subject, or reduce one or more symptoms of said disease or condition in the subject.

For example, the methods can genetically edit one or more cells by comprising: (a) transfecting a population of cells with any of the expression cassettes or the expression systems described herein to generate a population of transfected cells; and (b) selecting one or more cells from the population of transfected cells as genetically edited cells. To select one or more cells from the population of transfected cells, colonies can be generated from the transected cells to provide isogenic individual colonies and one or more precisely edited cells can be identified and select from at least one isogenic colony. Precisely edited cells can be identified by sequencing one or more genomic target sites in cells from one or more isogenic individual colonies to confirm that the genomic target sites in at least one of the isogenic individual colonies are precisely edited. The methods can also include administering a population of the precisely edited cells to a subject.

The engineered retron ncRNAs, expression cassettes, and expression systems (as well as compositions thereof) can be used to treat a subject having a disease or condition, or a subject suspected of having or developing a disease or condition. For example, the disease or condition can be a genetic disease or condition.

### DESCRIPTION OF THE FIGURES

FIG. 1A-1M illustrate structural modifications to retron ncRNA that affect RT-DNA production. FIG. 1A shows schematic diagrams illustrating at the top the conversion of the ncRNA to RT-DNA, and at the bottom a schematic of the Eco1 retron operon. FIG. 1B shows a gel illustrating endogenous RT-DNA production from Eco1 in BL21-AI wild-type cells (wt) and in BL21-AI cells with a knockout of the retron operon (KO), as analyzed by polyacrylamide electrophoresis (PAGE). FIG. 1C is a schematic diagram of the RT-DNA and the plasmid that expresses the ncRNA. The positions of primers in the RT-DNA and the plasmid for qPCR analysis of RT-DNA production. As illustrated, the primer pair will amplify a fragment by using both the RT-DNA and the msd portion of the plasmid as a template. However, the other primer pair will only amplify a fragment by using the plasmid as a template. FIG. 1D graphically illustrates enrichment of the RT-DNA/plasmid template over the plasmid alone (+), relative to the uninduced condition (−), as measured by qPCR. Circles represent each of three biological replicates. FIG. 1E is a schematic of the variant library construction and analysis. FIG. 1F graphically illustrates the relative RT-DNA abundance of each stem length variant as a percent of wild type RT-DNA abundance. Circles represent each of three biological replicates. Wild type length abundance is shown along with a dashed line at 100%. FIG. 1G graphically illustrates the relative RT-DNA abundance of each loop length variant as a percent of the value of five base loops. Circles represent each of three biological replicates, each of which is the average of five loops at that length with differing base content. A dashed line is shown at 100%. FIG. 1H is a schematic illustrating the a1 and a2 regions of the retron ncRNA. FIG. 1I is a schematic illustrating linking of a1/a2 region variants to a barcode in the msd loop for sequencing. FIG. 1J, graphically illustrates the relative RT-DNA abundance of each a1/a2 length variant as a percent of wild type. Circles represent each of three biological replicates. Wild type length is shown along with a dashed line at 100%. FIG. 1K is a schematic diagram illustrating methods for sequencing RT-DNA variants in the library. The methods involved tailing purified RT-DNA with a string of polynucleotides using a template-independent polymerase (TdT), and then generating a complementary strand via an adapter-containing, inverse anchored primer. Finally, a second adapter was ligated to this double-stranded DNA and the tagged RT-DNA was indexed and analyzed by multiplexed sequencing. FIG. 1L shows PAGE analysis illustrating the addition of nucleotides to the 3′ end of a single-stranded DNA, as controlled by reaction time. FIG. 1M graphically illustrates RT-DNA abundance in the a1/a2 length library, using a TdT-based sequencing.

FIG. 2A-2I illustrates RT-DNA production in eukaryotic cells. FIG. 2A shows a schematic of the retron cassette for expression in yeast, with qPCR primers indicated. FIG. 2B illustrates enrichment of the Eco1 RT-DNA/plasmid template over the plasmid alone as detected by qPCR in yeast, with each construct shown relative to uninduced expression. Circles show each of three biological replicates, with the wt a1/a2 length and the extended a1/a2. FIG. 2C illustrates enrichment of the Eco2 RT-DNA/plasmid template over the plasmid alone as detected by qPCR in yeast in yeast, using methods like those described in FIG. 2B. FIG. 2D shows a schematic of expression of retrons in mammalian cells, as detected by qPCR (primers indicated). FIG. 2E illustrates enrichment of the Eco1 RT-DNA/plasmid template over the plasmid alone as detected by qPCR in HEK293T cells, using methods like those described in FIG. 2B. FIG. 2F illustrates enrichment of the Eco2 RT-DNA/plasmid template over the plasmid alone as detected by qPCR in HEK293T cells, using methods like those described in FIG. 2B. FIG. 2G shows a gel of Eco1 and Eco2 RT-DNA isolated from yeast and subjected to PAGE analysis. The ladder is shown at a different exposure to the left of the gel image. FIG. 2H illustrates enrichment of Eco1 RT-DNA/plasmid template when uninduced compared to a dead RT construct. Closed circles show each of three biological replicates, with dead RT version and live RT. FIG. 2I Illustrates enrichment of Eco1 RT-DNA/plasmid template in HEK293T cells, using methods like those described in FIG. 2B.

FIG. 3A-3U illustrate improvements extend to applications in genome editing. FIG. 3A shows a schematic of an RT-DNA template for recombineering. The retron ncRNA was modified in the msd region to include a long loop that contains homology to a bacterial genomic locus but has one or more nucleotide modifications (repair nucleotides; asterisks). FIG. 3B graphically illustrates fold enrichment of the Eco1-based recombineering RT-DNA/plasmid template over the plasmid alone in E. coli, as detected by qPCR, with each construct shown relative to uninduced. Circles show each of three biological replicates, with wild type a1/a2 length and extended a1/a2. FIG. 3C shows PAGE gel illustrating purified RT-DNA from wild type (a1/a2 length: 12 bp) and extended (a1/a2 length: 22 bp) recombineering constructs. FIG. 3D graphically illustrates the percent of cells precisely edited, as quantified by multiplexed sequencing, for the wt and extended recombineering constructs. FIG. 3E shows a schematic of a hybrid RT-DNA that includes a guide RNA (gRNA) for genome editing in yeast. FIG. 3F graphically illustrates the percent of colonies edited based on phenotype) at 24 and 48 hours. Circles show each of three biological replicates, with wt (a1/a2 length: 12 bp) and extended a1/a2 (two extended versions, v1 and v2: a1/a2 length: 27 bp). Induction conditions are shown below the graph for the RT and Cas9. FIG. 3G shows examples of images from each condition plotted in FIG. 3F, at 24 h. Induction conditions are shown above each image. FIG. 3H graphically illustrates the quantity of precise editing of the ADE2 locus in yeast as detected by Illumina sequencing, and as plotted as in FIG. 3F. FIG. 3I graphically illustrates the percent of E. coli cells that were precisely edited at one locus, as quantified by multiplexed sequencing, for the wt (black) and extended (green) recombineering construct. FIG. 3J graphically illustrates the percent of E. coli cells that were precisely edited at one locus, as quantified by multiplexed sequencing, for the wt and extended recombineering construct. FIG. 3K graphically illustrates the percent of E. coli cells that were precisely edited at one locus, as quantified by multiplexed sequencing, for the wt and extended recombineering construct. FIG. 3L graphically illustrates the percent of yeast cells with precise edits as quantified by multiplexed sequencing, for the wild type and extended recombineering constructs targeting TRP2 E64X. FIG. 3M graphically illustrates the percent of yeast cells with precise edits as quantified by multiplexed sequencing, for the wild type and extended recombineering constructs targeting FAA1 P233X. FIG. 3N graphically illustrates the percent of yeast cells with precise edits as quantified by multiplexed sequencing, for the wild type and extended recombineering constructs targeting CAN1 G444X. FIG. 3O graphically illustrates the percent of yeast cells with precise edits as quantified by multiplexed sequencing, for the wild type and extended recombineering constructs targeting LYP1 E27X. FIG. 3P graphically illustrates the percent of yeast cells with imprecise edits as quantified by multiplexed sequencing, for the wild type and extended recombineering constructs targeting TRP2. FIG. 3Q graphically illustrates the percent of yeast cells with imprecise edits as quantified by multiplexed sequencing, for the wild type and extended recombineering constructs targeting FAA1. FIG. 3R graphically illustrates the percent of yeast cells with precise edits as quantified by multiplexed sequencing, for the wild type and extended recombineering constructs targeting CAN1. FIG. 3S graphically illustrates the percent of yeast cells with precise edits as quantified by multiplexed sequencing, for the wild type and extended recombineering constructs targeting LYP1. FIG. 3T illustrates that different retrons mediate precise genome editing in yeast. Retron Eco1, Eco4 and Sen2 ncRNAs were engineered for genome editing as described in Example 1 and were co-expressed alongside the retron reverse transcriptases and SpCas9, with the goal of introducing a 2 bp mutation in the ADE2 gene. The precise edit rates after were estimated by deep sequencing of the ADE2 gene. FIG. 3U illustrates where the repair template can be inserted into the msd stem—insertions can be into the P4a, P4b, or P4c region of the retron msd as shown in FIG. 3U.

FIG. 4A-4P illustrate precise editing by retrons can be used in human cells. FIG. 4A graphically illustrates the percent of precise edits for different single-promoter constructs designed to edit the ADE2 locus in yeast (S. cerevisiae). The arrangement of proteins is indicated below, and the fusion linkers are described in Example 1. Circles represent data for each of three biological replicates. FIG. 4B shows a schematic diagram of the elements for editing in human cells. At the top are the integrated protein cassettes that were used to generate the data in FIGS. 4C-4H. The diagram at the bottom illustrates features of the plasmid for transient transfection of site-specific ncRNA/gRNA. FIG. 4C graphically illustrates the percent of precise editing at the AAVS1 locus in HEK293T cells as determined by Illumina sequencing. Proteins present during editing are shown below the graph. Circles represent each of three biological replicates. FIG. 4D graphically illustrates the percent of precise editing at the EMX1 locus in HEK293T cells as determined by Illumina sequencing. Proteins present during editing are shown below the graph. Circles represent each of three biological replicates. FIG. 4E graphically illustrates the percent of precise editing at the FANCF locus in HEK293T cells as determined by Illumina sequencing. Proteins present during editing are shown below the graph. Circles represent each of three biological replicates. FIG. 4F graphically illustrates the percent of precise editing at the HEK3 locus in HEK293T cells as determined by Illumina sequencing. Proteins present during editing are shown below the graph. Circles represent each of three biological replicates. FIG. 4G graphically illustrates the percent of precise editing at the HEK4 locus in HEK293T cells as determined by Illumina sequencing. Proteins present during editing are shown below the graph. Circles represent each of three biological replicates. FIG. 4H graphically illustrates the percent of precise editing at the RNF2 locus in HEK293T cells as determined by Illumina sequencing. Proteins present during editing are shown below the graph. Circles represent each of three biological replicates. FIG. 4I graphically illustrates that percent of ADE2 loci in yeast with imprecise edits or with sequencing errors at 24 and 48 hours. Closed circles show each of three biological replicates, with wt a1/a2 length and extended a1/a2 (two extended versions, v1 and v2). Induction conditions are shown below the graph for the RT and Cas9. FIG. 4J illustrates breakdown of the data shown in FIG. 4I by type of edit/error. FIG. 4K graphically illustrates the percent of cells with imprecisely edited (indels) at the AAVS1 locus, as quantified by multiplexed sequencing, when using an ncRNA/gRNA plasmid and either Cas9 alone or Cas9 and Eco1 reverse transcriptase. Individual circles represent each of three biological replicates. FIG. 4L graphically illustrates the percent of cells with imprecisely edited (indels) at the EMX1 locus, as quantified by multiplexed sequencing, when using an ncRNA/gRNA plasmid and either Cas9 alone or Cas9 and Eco1 reverse transcriptase. Individual circles represent each of three biological replicates. FIG. 4M graphically illustrates the percent of cells with imprecisely edited (indels) at the FANCF locus, as quantified by multiplexed sequencing, when using an ncRNA/gRNA plasmid and either Cas9 alone or Cas9 and Eco1 reverse transcriptase. Individual circles represent each of three biological replicates. FIG. 4N graphically illustrates the percent of cells with imprecisely edited (indels) at the HEK3 locus, as quantified by multiplexed sequencing, when using an ncRNA/gRNA plasmid and either Cas9 alone or Cas9 and Eco1 reverse transcriptase. Individual circles represent each of three biological replicates. FIG. 4O graphically illustrates the percent of cells with imprecisely edited (indels) at the HEK4 locus, as quantified by multiplexed sequencing, when using an ncRNA/gRNA plasmid and either Cas9 alone or Cas9 and Eco1 reverse transcriptase. Individual circles represent each of three biological replicates. FIG. 4P graphically illustrates the percent of cells with imprecisely edited (indels) at the RNF2 locus, as quantified by multiplexed sequencing, when using an ncRNA/gRNA plasmid and either Cas9 alone or Cas9 and Eco1 reverse transcriptase. Individual circles represent each of three biological replicates.

FIG. 5 is a schematic illustration of retron-mediated genomic DNA editing. At the left is shown the retron system that can be used to generate DNA in cells via a reverse transcriptase. The reverse transcriptase partially reverse transcribes the retron non-coding RNA (ncRNA) into DNA. For editing, the ncRNA is altered in two ways: (1) to harbor a CRISPR sgRNA for genome targeting; and (2) to provide a msd encode the desired edited sequence (e.g., a nucleotide or two that is different from the genomic DNA sequence (asterisk), flanked by regions of homology to the target site, which allows the RT-DNA to serve as a DNA repair template. Upon nuclease-mediated targeting of the genome, the RT-DNA repairs the genomic target site. As shown herein, the editing is inserted precisely.

### DETAILED DESCRIPTION

Described herein are compositions and methods that enable precise editing of human cells. The compositions and methods involve use of a CRISPR nuclease for targeted genome cutting, and a retron-reverse transcriptase construct to generate a repair donor inside the cell. Prior to this invention, workers have been able to edit bacterial and yeast cells, but not mammalian cells. The compositions and methods described herein provide modifications that enable use in mammalian (human) cells.

### Retrons

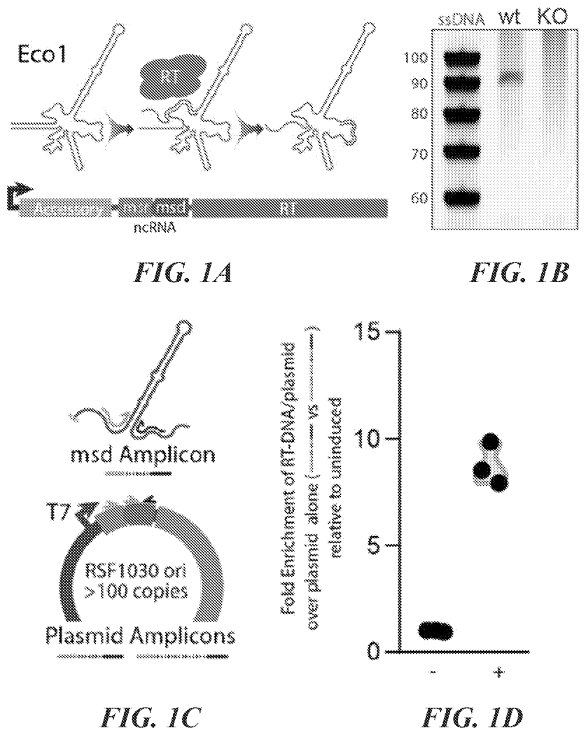

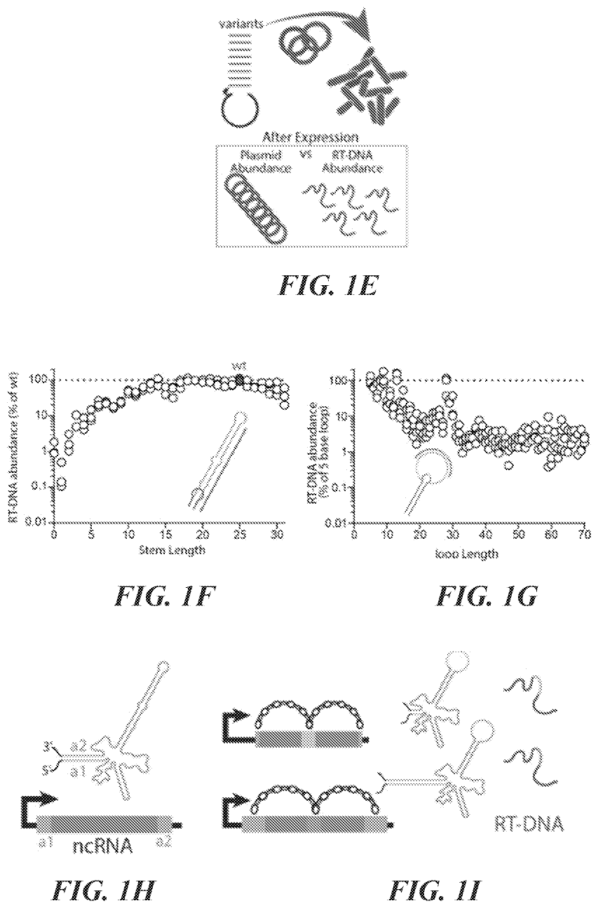

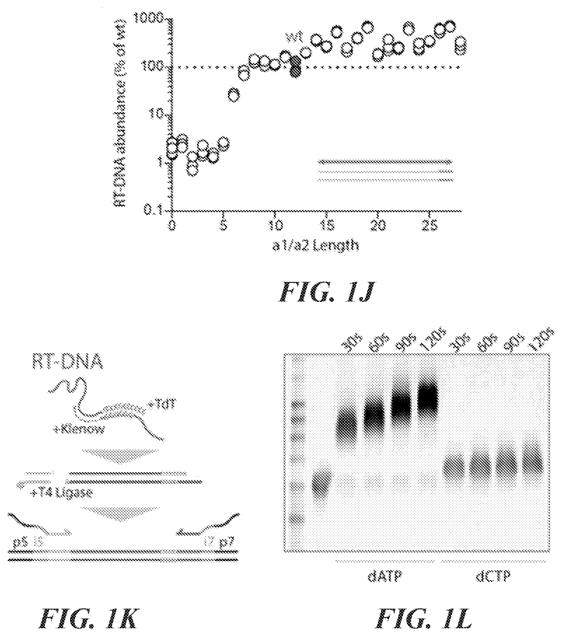

Retrons are defined by their unique ability to produce an unusual satellite DNA known as msDNA (multicopy single-stranded DNA). A typical retron operon consists of a gene encoding a retron reverse transcriptase (RT) (encoded by the ret gene) and a region encoding a non-coding RNA (ncRNA), which includes two contiguous and inverted non-coding sequences referred to as the msr and msd. The ncRNA serves both as a primer site (i.e., the msr region) for binding of the retron RT and template for the reverse transcriptase (i.e., the msd region), and a gene encoding an accessory protein (FIG. 1A).

The ret gene and the non-coding RNA (including the misr and msd) are transcribed as a single RNA transcript, processed to separate the ret region and the ncRNA region as separate transcripts. The ncRNA then becomes folded into a specific secondary structure. The 5′ and 3′ ends of ncRNA are referred to generally as the a1 and a2 complementary regions and can hybridize to one another to form a stem or duplex region referred to as the “a1/a2 stem” or the “a1/a2 duplex” of the ncRNA.

The retron RT, once translated, binds the ncRNA downstream from the msd locus (without being bound by theory, the binding may involve the a1/a2 duplex) and initiates reverse transcription of the msd region as a template sequence, thereby generating a single strand DNA reverse transcriptase product (i.e., the RT-DNA, with a characteristic hairpin structure, which in wild type retrons varies in length from about 48 to 163 bases). The RT-DNA, as part of the priming event, is covalently attached to a 2′OH group present in a conserved branching guanosine residue. Reverse transcription halts before reaching the msr locus. It is thought that cellular RNase H degrades the template RNA during reverse transcription. The result is the formation of a chimeric molecule of RNA (the remaining portions of the ncRNA not removed by processing) and DNA (the single stranded RT-DNA product covalently attached to the ncRNA), which is referred to as “msDNA.” See FIG. 1A.

A large number of retrons have been identified and can be modified or engineered as described herein. One of the first described retrons found in E. coli is called Eco1 (previously called Ec86). In BL21 E. coli cells, this retron is present and active, producing reverse transcriptase DNA that can be detected at the population level. The wild type Eco1 retron can be eliminated from BL21 E. coli cells by removing the retron operon from the genome (FIG. 1B). In the absence of this native operon, the ncRNA and reverse transcriptase can be expressed from a plasmid lacking the accessory protein. Since the accessory protein is a core component of the phage-defense conferred by retrons, this reduced system would reduce phage defense capacity, yet cells with ncRNA-reverse transcriptase encoding plasmids continue to produce abundant reverse transcribed DNA. The accessory protein coding region is not included in the engineered retrons.

An example of an Eco1 wild-type retron non-coding RNA (ncRNA) sequence is shown below as SEQ ID NO: 1.

```
    1 ATGCGCACCC TTAGCGAGAG GTTTATCATT AAGGTCAACC
  41 TCTGGATGTT GTTTCGGCAT CCTGCATTGA ATCTGAGTTA
  81 CTGTCTGTTT TCCTTGTTGG AACGGAGAGC ATCGCCTGAT
121 GCTCTCCGAG CCAACCAGGA AACCCGTTAT TTCTGACGTA
161 AGGGTGCGCA
```

An example of an Eco1 human-codon optimized reverse transcriptase (RT) sequence that can be used is shown below as SEQ ID NO: 2.

| | |
|---|---|
| 1 | ATGAAATCTG CAGAGTATCT GAATACGTTC CGCCTTAGGA |
| 41 | ATTTGGGCCT CCCCGTGATG AACAATCTCC ACGATATGAG |
| 81 | CAAGGCGACT CGAATATCCG TGGAAACGCT GAGACTGCTC |
| 121 | ATCTATACAG CAGACTTTCG GTACAGGATC TACACGGTCG |
| 161 | AAAAGAAGGG GCCTGAGAAA CGCATGCGAA CAATTTATCA |
| 201 | ACCTAGCCGA GAGCTCAAGG CGTTGCAGGG CTGGGTTCTT |
| 241 | CGAAACATCC TTGACAAACT CTCATCATCA CCCTTTAGTA |
| 281 | TTGGGTTTGA AAAGCACCAA AGCATCCTTA ACAACGCGAC |
| 321 | GCCACACATA GGTGCCAATT TCATATTGAA CATCGACTTG |
| 361 | GAGGATTTTT TTCCGAGCCT CACAGCCAAT AAAGTGTTCG |
| 401 | GTGTTTTTCA CAGTCTTGGG TACAATCGCC TTATTAGTTC |
| 411 | CGTTCTTACC AAGATTTGTT GTTACAAGAA TCTCTTGCCC |
| 481 | CAGGGAGCAC CCAGCAGTCC GAAATTGGCG AATTTGATTT |
| 521 | GTTCCAAGCT CGATTATCGA ATACAAGGGT ACGCGGGCAG |
| 561 | CCGGGGACTC ATCTATACCC GCTACGCAGA CGATCTTACG |
| 601 | CTGTCTGCCC AATCAATGAA GAAGGTCGTA AAGGCGCGGG |
| 641 | ATTTCTTGTT TTCTATCATC CCGTCCGAGG GCTTGGTAAT |
| 681 | TAATTCCAAA AAGACTTGTA TCTCAGGACC ACGATCTCAG |
| 721 | CGAAAAGTGA CAGGACTCGT CATTTCTCAA GAAAAAGTCG |
| 761 | GTATAGGGAG AGAGAAGTAT AAGGAAATCC GCGCGAAGAT |
| 801 | CCACCACATA TTCTGTGGCA AGAGCAGCGA GATAGAACAC |
| 841 | GTCCGAGGCT GGTTGTCCTT CATACTGAGC GTGGACTCAA |
| 881 | AAAGCCACCG CCGGTTGATC ACCTATATTT CAAAACTGGA |
| 921 | AAAGAAATAT GGAAAGAACC CACTCAACAA AGCTAAAACA |
| 961 | TAG |

An example of an Eco2 human-codon optimized reverse transcriptase (RT) sequence is shown below as SEQ ID NO. 3.

| | |
|---|---|
| 1 | ATGACAAAAA CTTCAAAGCT GGATGCGCTG CGGGCGGCTA |
| 41 | CTAGTAGGGA AGATTTGGCG AAGATTCTCG ACATAAAGTT |
| 81 | GGTGTTTCTG ACAAACGTGT TGTACCGCAT AGGATCCGAC |
| 121 | AACCAGTATA CGCAATTCAC AATACCCAAA AAGGGTAAAG |
| 161 | GTGTCCGCAC CATCAGCGCA CCAACGGACC GACTTAAGGA |
| 201 | TATACAGAGG AGGATTTGTG ATCTTCTTAG TGACTGTAGG |
| 241 | GATGAAATCT TTGCGATTAG GAAGATCTCT AATAATTACT |
| 281 | CATTCGGCTT CGAAAGAGGA AAATCAATTA TACTCAATGC |
| 321 | TTACAAGCAT CGAGGGAAGC AAATTATATT GAACATCGAC |
| 361 | CTTAAGGACT TCTTTGAGAG CTTTAACTTT GGGAGAGTCC |
| 401 | GGGGGTACTT TCTCTCCAAC CAGGACTTCT TGTTGAACCC |
| 441 | AGTTGTGGCA ACAACGTTGG CGAAGGCCGC CTGCTACAAC |
| 481 | GGGACTCTGC CTCAGGGGTC CCCATGTTCC CCTATTATAA |
| 521 | GTAACCTTAT CTGTAACATT ATGGACATGC GGCTCGCAAA |
| 561 | GCTCGCCAAG AAGTACGGCT GCACTTATAG TCGATATGCG |
| 601 | GATGACATTA CGATCAGCAC CAATAAAAAT ACCTTCCCGT |
| 641 | TGGAGATGGC GACTGTGCAG CCTGAAGGGG TTGTGCTGGG |
| 681 | CAAAGTGCTC GTAAAGGAGA TTGAAAATTC AGGTTTCGAG |
| 721 | ATTAACGATT CTAAGACTAG ATTGACCTAC AAAACAAGTA |
| 761 | GGCAAGAAGT CACCGGGCTG ACGGTTAATC GGATTGTAAA |
| 801 | CATTGATCGG TGCTACTACA AAAAGACGAG GGCGCTGGCT |
| 841 | CACGCATTGT ATCGGACAGG AGAATATAAG GTCCCAGACG |
| 881 | AGAACGGTGT TCTGGTATCT GGAGGGCTTG ACAAGTTGGA |
| 921 | GGGTATGTTT GGGTTTATCG ACCAGGTGGA TAAATTCAAC |
| 961 | AACATTAAAA AAAAGTTGAA TAAGCAACCC GACAGATATG |
| 1001 | TTCTGACAAA TGCCACTTTG CACGGATTTA AGCTCAAATT |
| 1041 | GAACGCCAGG GAGAAAGCCT ATAGCAAATT CATCTACTAC |
| 1081 | AAATTCTTCC ACGGTAATAC TTGTCCCACG ATCATAACAG |
| 1121 | AGGGTAAGAC GGATAGGATT TACCTTAAAG CTGCCCTCCA |
| 1161 | TAGCCTCGAG ACAAGTTATC CTGAACTGTT TCGGGAGAAA |
| 1201 | ACAGATAGTA AGAAGAAGGA GATAAATCTG AATATTTTTA |
| 1241 | AAAGCAATGA GAAGACCAAG TATTTCCTGG ATCTCAGCGG |
| 1281 | CGGCACAGCA GACCTCAAGA AATTCGTGGA ACGCTACAAA |
| 1321 | AATAACTACG CTTCCTATTA CGGCAGCGTA CCGAAACAAC |
| 1361 | CGGTGATAAT GGTGCTTGAT AACGACACAG GCCCGTCAGA |
| 1401 | CCTGTTGAAC TTTTTGAGAA ACAAAGTTAA GAGTTGTCCA |
| 1441 | GATGATGTAA CAGAAATGCG CAAGATGAAG TACATACATG |
| 1481 | TGTTTTACAA TCTGTACATA GTTCTGACTC CCCTGTCTCC |
| 1521 | ATCTGGAGAG CAAACGTCTA TGGAGGACCT CTTTCCTAAA |
| 1561 | GATATATTGG ACATTAAGAT AGATGGCAAG AAATTCAATA |
| 1601 | AAAACAATGA CGGTGACTCC AAAACAGAGT ATGGGAAGCA |
| 1001 | CATATTCTCA ATGCGCGTTG TACGAGATAA AAAGAGGAAG |
| 1001 | ATAGATTTCA AGGCATTTTG CTGTATCTTC GATGCTATTA |
| 1001 | AGGATATTAA AGAACATTAC AAACTGATGT TGAATTCCTA |
| 1001 | G |

An example of an Eco1 wild-type retron reverse transcriptase sequence is shown below as SEQ ID NO: 4.

```
    1 KSAEYLNTFR LRNLGLPVMN NLHDMSKATR ISVETLRLLI
  41 YTADFRYRIY TVEKKGPEKR MRTIYQPSRE LKALQGWVLR
  81 NILDKLSSSP FSIGFEKHQS ILNNATPHIG ANFILNIDLE
121 DFFPSLTANK VEGVFHSLGY NRLISSVLTK ICCYKNLLPQ
161 GAPSSPKLAN LICSKLDYRI QGYAGSRGLI YTRYADDLTL
201 SAQSMKKVVK ARDFLFSIIP SEGLVINSKK TCISGPRSQR
241 KVTGLVISQE KVGIGREKYK EIRAKIHHIF CGKSSEIEHV
281 RGWLSFILSV DSKSHRRLIT YISKLEKKYG KNPLNKAKT
```

An example of an Eco2 wild-type retron reverse transcriptase sequence is shown below as SEQ ID NO: 5.

| | |
|---|---|
| 1 | CACGCATGTA GGCAGATTTG TTGGTTGTGA ATCGCAACCA |
| 41 | GTGGCCTTAA TGGCAGGAGG AATCGCCTCC CTAAAATCCT |
| 81 | TGATTCAGAG CTATACGGCA GGTGTGCTGT GCGAAGGAGT |
| 121 | GCCTGCATGC GT |

An example of a sequence for an Eco4 retron reverse transcriptase is shown below as SEQ ID NO: 6.

```
    1 MSIDIETTLQ KAYPDFDVLL KSRPATHYKV YKIPKRTIGY
  41 RIIAQPTPRV KAIQRDIIEI LKQHTHIHDA ATAYVDGKNI
  81 LDNAKIHQSS VYLLKLDLVN FFNKITPELL FKALARQKVD
121 ISDTNKNLLK QFCFWNRTKR KNGALVLSVG APSSPFISNI
161 VMSSFDEEIS SFCKENKISY SRYADDLTFS TNERDVLGLA
201 HQKVKTTLIR FFGTRIIINN NKIVYSSKAH NRHVTGVTLT
241 NNNKLSLGRE RKRYITSLVF KFKEGKLSNV DINHLRGLIG
281 FAYNIEPAFI ERLEKKYGES TIKSIKKYSE GG
```

An example of a sequence for a Sen2 retron reverse transcriptase is shown below as SEQ ID NO: 7.

```
    1 MDILQHISDL LLTKKSEIIS FSLTAPYRYK IYKIAKRNSD
  41 KKRTIAHPSK ELKFIQREIT EYLTDKLPVH ECAFAYKKGS
  81 SIKTNAQVHL HTKYLLKMDF ENFFPSITPR LFFSKLRLAN
121 IDLTADDKVL LENILFFKSK RNSNLRLSIG APSSPLISNF
161 VMYFWDIEVQ EICSKIGVNY TRYADDLTFS TNNKDVLFDI
201 PDMLENVLPK YSLGRIRINH EKTVFSSKGH NRHVTGITLT
241 NDNKLSIGRE RKRKISAMIH HFINGKLSTD ECNKLVGLLA
281 FAKNIEPSFY KSMVIKYGSD NIYKLQKQKD K
```

This section is provided as a retron overview, other types of retrons are described throughout the application and can be engineered as described herein.

In addition, any of the retrons described in Mestre et al., Systematic Prediction of Genes Functionally Associated with Bacterial Retrons and Classification of The Encoded Tripartite Systems, Nucleic Acids Research Volume 48, Issue 22, 16 Dec. 2020, Pages 12632-12647” (incorporated herein by reference) may be used as a starting point by which to introduce the modifications described herein to result in the engineered retrons ncRNAs, msDNAs, and RT-DNAs described herein. These retron sequences are provided as follows in Table A:

TABLE A
Retron sequences that may be modified as described herein

| NCBI Accession^{a} | Species/strain |
|---|---|
| fig\|670897.3.peg.2382 | Escherichia coli 2362-75 |
| WP_000111473.1 | Escherichia coli |
| fig\|286156.4.peg.5031 | Photorhabdus australis |
| fig\|171439.3.peg.1995 | Photorhabdus luminescens |
| | subsp. luminescens |
| fig\|1004151.3.peg 110 | Photorhabdus khanji NC19 |
| fig\|1736225.3.peg.2969 | Erwinia sp. Leaf53 |
| fig\|1897730.3.peg.2912 | Citrobacter sp. CFSAN044567 |
| fig\|286156.4.peg.5031 | Aeromonas australiensis |
| fig\|1460083.3.peg.4429 | Serratia liquefaciens FK01 |
| fig\|585.10.peg.2369 | Proteus vulgaris |
| WP_140315795.1 | Vibrio parahaemolyticus |
| fig\|670.147.peg.3463 | Vibrio parahaemolyticus |
| fig\|1516159.4.peg.4737 | Vibrio coralliirubri |
| fig\|190893.12.peg.246 | Vibrio coralliilyticus |
| fig\|643674.5.peg.820 | Paenalcaligenes hominis |
| fig\|1122619.3.peg.2381 | Oligella ureolytica DSM 18253 |
| fig\|29489.5.peg.3423 | Aeromonas enteropelogenes |
| fig\|1899355.18.peg.3566 | Oceanospirillaceae bacterium |
| fig\|49186.3.peg.4362 | Marinobacterium stanieri |
| fig\|672.375.peg.4377 | Vibrio vulnificus |
| fig\|584.202.peg.1668 | Proteus mirabilis |
| fig\|394935.10.peg.4407 | Chromobacterium haemolyticum |
| fig\|1196083.117.peg.637 | Snodgrassella alvi |
| fig\|1196083.120.peg.2046 | Snodgrassella alvi |
| fig\|1196083.114.peg.825 | Snodgrassella alvi |
| fig\|550.250.peg.2975 | Enterobacter cloacae |
| fig\|680.27.peg.793 | Vibrio campbellii |
| fig\|1348393.3.peg.352 | Pseudoalteromonas sp. H105 |
| fig\|644.85.peg.4392 | Aeromonas hydrophila |
| fig\|1234128.4.peg.4777 | Vibrio parahaemolyticus SNUVpS-1 |
| fig\|69219.6.peg.2213 | Enterobacter cloacae subsp. dissolvens |
| fig\|208224.13.peg.2962 | Enterobacter kobei |
| fig\|672.332.peg.2758 | Vibrio vulnificus |
| fig\|1777131.3.peg.2267 | Chromobacterium sp. F49 |
| fig\|945550.3.peg.1167 | Vibrio sinaloensis DSM 21326 |
| fig\|648.75.peg.922 | Aeromonas caviae |
| fig\|1238221.3.peg.2053 | Vibrio parahaemolyticus VPTS-2009 |
| fig\|56192.3.peg.3860 | Photobacterium iliopiscarium |
| fig\|1806667.7.peg 3169 | Marinomonas gallaica |
| fig\|272773.3.peg.1019 | Salinivibrio costicola subsp. alcaliphilus |
| WP_073265166.1 | Pseudomonas punonensis |
| fig\|1946584.3.peg.2789 | Halomonas sp. UBA3074 |
| fig\|2030880.3.peg.665 | SAR86 cluster bacterium |
| fig\|80854.14.peg.530 | Moritella viscosa |
| fig\|1902503.3.peg.1072 | Marinomonas sp. QM202 |
| fig\|1122212.3.peg.1985 | Marinospirillum minutulum DSM 6287 |
| fig\|40576.4.peg.4387 | Xenorhabdus bovienii |
| fig\|287094.3.peg.78 | Alteromonas addita |
| fig\|1805633.3.peg.1469 | Acinetobacter sp. SFA |
| fig\|1945927.3.peg.1017 | Acinetobacter sp. UBA1497 |
| fig\|202956.9.peg.1680 | Acinetobacter towneri |
| fig\|1811612.3.peg.155 | Moraxellaceae bacterium |
| | REDSEA-S32_B1 |
| fig\|573.14330.peg.438 | Klebsiella pneumoniae |
| fig\|470.1294.peg.971 | Acinetobacter baumannii |
| fig\|762966.3.peg.2452 | Parasutterella excrementihominis |
| | YIT 11859 |
| fig\|470.3514.peg.1550 | Acinetobacter baumannii |
| fig\|470.2538.peg.3022 | Acinetobacter baumannii |
| fig\|48296.130.peg.276 | Acinetobacter pittii |
| fig\|663.91.peg.4688 | Vibrio alginolyticus |
| fig\|296199.3.peg.4813 | Vibrio gigantis |
| fig\|1367490.3.peg.3583 | Aliivibrio fischeri ETJB5C |
| fig\|326537.3.peg.3698 | Colwellia polaris |
| fig\|1175631.4.peg.4191 | Pectobacterium wasabiae CFBP 3304 |
| WP_001403504.1 | Escherichia coli |
| fig\|549.21.peg.1734 | Pantoea agglomerans |
| fig\|140100.3.peg.2972 | Vibrio cholerae |
| fig\|693 153.4.peg.1176 | Vibrio atlanticus |
| fig\|1238430.3.peg.1911 | Vibrio nigripulchritudo AM115 |
| fig\|1123036.3.peg.144 | Psychromonas arctica DSM 14288 |
| fig\|173990.3.peg.3319 | Rheinheimera pacifica |
| fig\|1869214.4.peg.3809 | Rheinheimera sp. |
| fig\|1898113.7.peg.1514 | Idiomarinaceae bacterium |
| fig\|29484.39.peg.1876 | Yersinia frederiksenii |
| fig\|1761793.3.peg.274 | Marinobacter sp. DSM 26671 |
| fig\|587.48.peg.2666 | Providencia rettgeri |
| fig\|573.4147.peg.1684 | Klebsiella pneumoniae |
| fig\|1263833.3.peg.2872 | Serratia marcescens VGH107 |
| fig\|1690502.3.peg.467 | Pantoea sp. CFSAN033090 |
| fig\|1029989.3.peg.5037 | Salmonella enterica subsp. enterica |
| | serovar Agona str. 0292 |
| fig\|211759.3.peg.770 | Serratia marcescens |
| fig\|29483.5.peg.2283 | Yersinia aldovae |
| fig\|1268238.3.peg.3466 | Escherichia coli O5:K4(L):H4 |
| | str. ATCC 23502 |
| fig\|548.121.peg.2368 | Klebsiella aerogenes |
| fig\|196024.6.peg.1825 | Aeromonas dhakensis |
| fig\|386429.3.peg.3784 | Pseudoalteromonas sp. BSi20495 |
| fig\|666.2089.peg.3167 | Vibrio cholerae |
| WP_159353404.1 | Vibrio cholerae |
| fig\|670.362.peg.2186 | Vibrio parahaemolyticus |
| fig\|615.398.peg.1671 | Serratia marcescens |
| fig\|571.188.peg.5401 | Klebsiella oxytoca |
| fig\|1389422.3.peg.2794 | Klebsiella pneumoniae LAU-KP1 |
| fig\|1082704.3.peg.1242 | Lonsdalea britannica |
| fig\|1686379.3.peg.3365 | Citrobacter sp. MGH104 |
| fig\|83655.55.peg.221 | Leclercia adecarboxy lata |
| fig\|550.532.peg.617 | Enterobacter cloacae |
| fig\|349965.6.peg.153 | Yersinia intermedia ATCC 29909 |
| fig\|1947028.3.peg.31 | Pantoea sp. UBA2708 |
| fig\|29484.34.peg.3725 | Yersinia frederiksenii |
| fig\|314608.4.peg.222 | Shewanella benthica KT99 |
| fig\|585.16.peg.3620 | Proteus vulgaris |
| fig\|1117313.3.peg.4128 | Pseudoalteromonas arctica A 37-1-2 |
| fig\|1236543.3.peg.1328 | Shewanella putrefaciens |
| | JCM 20190 = NBRC 3908 |
| fig\|550.520.peg.1818 | Enterobacter cloacae |
| fig\|592316.4.peg.43 | Pantoea sp. At-9b |
| fig\|1903177.3.peg.4556 | Vibrio sp. I0N.261.45.E1 |
| fig\|1435069.3.peg.925 | Vibrio tritonius |
| fig\|666.3258.peg.1211 | Vibrio cholerae |
| fig\|1579504.3.peg.1822 | Shewanella sp. ECSMB14102 |
| fig\|727.548.peg.1576 | Haemophilus influenzae |
| EIJ70524.1 | Haemophilus parahaemolyticus HK385 |
| fig\|1121935.3.peg.14 | Hahella ganghwensis DSM 17046 |
| fig\|400668.8.peg.2509 | Marinomonas sp. MWYL1 |
| fig\|1777491.3.peg.1212 | Alteromonas sp. Mac1 |
| fig\|2013797.3.peg.1728 | Gammaproteobacteria bacterium HGW- |
| | Gammaproteobacteria-15 |
| fig\|1008297.7.peg.4158 | Salmonella enterica subsp. enterica |
| | serovar Typhimurium str. 798 |
| EDM6246721.1 | Salmonella enterica subsp. enterica |
| | serovar Typhimurium |
| fig\|421.19.peg.3278 | Methylomonas methanica |
| fig\|758.17.peg.102 | Rodentibacter pneumotropicus |
| fig\|726.60.peg.864 | Haemophilus haemolyticus |
| fig\|1035188.3.peg.348 | Haemophilus pittmaniae HK 85 |
| fig\|670.79.peg.3738 | Vibrio parahaemolyticus |
| fig\|1481663.12.peg.913 | Vibrio metoecus |
| fig\|1123402.3.peg.611 | Thorsellia anophelis DSM 18579 |
| fig\|668.83.peg.3088 | Aliivibrio fischeri |
| fig\|290110.6.peg.2319 | Xenorhabdus budapestensis |
| fig\|568766.10.peg.822 | Dickeya sp. NCPPB 3274 |
| fig\|470.4268.peg.2217 | Acinetobacter baumannii |
| fig\|1977881.3.peg.1569 | Acinetobacter sp. ANC 4470 |
| fig\|548.171.peg.2395 | Klebsiella acrogenes |
| fig\|584.105.peg.1823 | Proteus mirabilis |
| fig\|1275975.3.peg.1756 | Salmonella enterica subsp. enterica |
| | serovar Newport str. Henan 3 |
| fig\|615.474.peg.3994 | Serratia marcescens |
| fig\|61647.13.peg.3699 | Pluralibacter gergoviae |
| fig\|549.22.peg.222 | Pantoca agglomerans |
| fig\|991944.3.peg.3216 | Vibrio cholerae HE-25 |
| WP_001022871.1 | Vibrio cholerae |
| fig\|1638949.3.peg.1051 | Vibrio sp. ECSMB14106 |
| fig\|73010.3.peg.2815 | Aeromonas encheleia |
| fig\|1444141.3.peg 3893 | Escherichia coli 3-373-03_S3_C1 |
| fig\|232.5.peg.1080 | Alteromonas sp. |
| fig\|1175295.3.peg.21 | Pseudoalteromonas sp. PAMC 22718 |
| fig\|265726.7.peg.3430 | Photobacterium halotolerans |
| WP_009585554.1 | Acinetobacter |
| fig\|2004649.3.peg.1632 | Acinetobacter sp. WCHA29 |
| fig\|1324350.3.peg.2817 | Acinetobacter equi |
| fig\|2048003.3.peg.1682 | Alteromonas flava |
| fig\|571.171.peg.5963 | Klebsiella oxytoca |
| fig\|573.4060.peg.3574 | Klebsiella pneumoniae |
| fig\|1173850.3.peg.2995 | Salmonella enterica subsp. enterica |
| | serovar Indiana str. ATCC 51959 |
| fig\|1123516.3.peg.1267 | Hydrogenovibrio halophilus DSM 15072 |
| fig\|1981674.3.peg.1814 | Pseudomonas sp. R.9(2017) |
| fig\|1947311.3.peg.2053 | Pseudomonas sp. UBA2684 |
| fig\|1198309.3.peg.4291 | Pseudomonas fluorescens ICMP 11288 |
| fig\|715451.3.peg.1743 | Alteromonas naphthalenivorans |
| fig\|316.285.peg.730 | Pseudomonas stutzeri |
| fig\|1190606.3.peg.313 | Enterovibrio calviensis IF-211 |
| WP_009176189.1 | |
| WP_097050713.1 | Thalassospira xiamenensis |
| fig\|1208323.3.peg.893 | Celeribacter baekdonensis B30 |
| KZK95863.1 | Pseudovibrio sp. Ad46 |
| fig\|101571.310.peg.3956 | Burkholderia ubonensis |
| fig\|1882791.3.peg.1790 | Burkholderia sp. CF099 |
| fig\|1736536.3.peg.4809 | Variovorax sp. Root434 |
| PIG30812.1 | Janthinobacterium sp. 35 |
| fig\|1798244.3.peg.1046 | Gallionellales bacterium GWA2_55_18 |
| fig\|1131551.3.peg.1124 | Methylotenera sp. IP/1 |
| fig\|1843082.3.peg.1574 | Macromonas sp. BK-30 |
| fig\|279058.16.peg.4721 | Collimonas arenae |
| fig\|1548123.6.peg.1144 | Pusillimonas sp. T2 |
| fig\|380394.4.peg.276 | Acidithiobacillus ferrooxidans ATCC 53993 |
| WP_080292858.1 | |
| fig\|101571.162.peg.3605 | Burkholderia ubonensis |
| fig\|1382803.3.peg.22 | Chromobacterium amazonense |
| fig\|930.4.peg.3851 | Acidithiobacillus thiooxidans |
| fig\|1261658.3.peg.1787 | Bibersteinia trebalosi Y31 |
| fig\|1679001.3.peg.631 | Pasteurellaceae bacterium NI1060 |
| fig\|1334187.3.peg.653 | Haemophilus influenzae KR494 |
| fig\|1581107.3.peg.1286 | Neisseria sp. HMSC15G01 |
| fig\|486.24.peg.152 | Neisseria lactamica |
| fig\|1953412.3.peg.1956 | bacterium UBP10_UBA1160 |
| WP_090322045.1 | Nitrosomonas oligotropha |
| fig\|2013740.3.peg.1400 | Deltaproteobacteria bacterium |
| | HGW-Deltaproteobacteria-13 |
| fig\|1907413.3.peg.3170 | Rhizobium sp. RU33A |
| fig\|1817963.3.peg.856 | Roseomonas deserti |
| fig\|2035448.3.peg 1752 | Rhizobium sp. C5 |
| WP_014077019.1 | |
| fig\|1648404.4.peg.2797 | Erythrobacter atlanticus |
| fig\|359.11.peg.6331 | Agrobacterium rhizogenes |
| fig\|887144.4.peg.573 | Rhizobium taibaishanense |
| fig\|1116389.3.peg.333 | Devosia insulae DS-56 |
| fig\|121719.10.peg.3421 | Pannonibacter phragmitetus |
| fig\|34002.6.peg.3570 | Paracoccus alcaliphilus |
| fig\|1940281.4.peg 1560 | Hoeflea sp. |
| fig\|1040981.5.peg.1561 | Mesorhizobium ciceri WSM4083 |
| fig\|410764.3.peg.807 | Rhizobium multihospitium |
| fig\|1825934.3.peg.3111 | Rhizobium anhuiense |
| fig\|1952824.3.peg.3061 | Rhodobiaceae bacterium UBA3976 |
| fig\|1871086.3.peg.2153 | Brevundimonas sp. |
| fig\|588932.9.peg.647 | Brevundimonas naejangsanensis |
| fig\|1951751.3.peg.1538 | Erythrobacteraceae bacterium UBA1460 |
| fig\|1843368.3.peg 904 | Sphingobium sp. RAC03 |
| fig\|155892.10.peg.3219 | Caulobacter vibrioides |
| fig\|43057.4.peg.4537 | Rhodobacter azotoformans |
| fig\|1514904.3.peg.974 | Ahrensia marina |
| fig\|1338034.3.peg.722 | Vibrio parahaemolyticus |
| | O1:Kuk str. FDA_R31 |
| fig\|150340.18.peg.1837 | Vibrio antiquarius |
| fig\|196024.5.peg.3821 | Aeromonas dhakensis |
| fig\|244366.32.peg.1886 | Klebsiella variicola |
| fig\|180957.35.peg.1654 | Pectobacterium brasiliense |
| fig\|55601.149.peg.665 | Vibrio anguillarum |
| fig\|121723.5.peg.2901 | Photobacterium sp. SKA34 |
| fig\|584.170.peg.837 | Proteus mirabilis |
| fig\|40324.136.peg.3276 | Stenotrophomonas maltophilia |
| fig\|1122188.5.peg.411 | Lysobacter spongiicola DSM 21749 |
| fig\|2032566.3.peg.2826 | Xanthomonadaceae bacterium NML93-0792 |
| fig\|287.1731.peg.2578 | Pseudomonas aeruginosa |
| fig\|251702.3.peg.1529 | Pseudomonas syringae pv. antirrhini |
| fig\|1960829.3.peg.5912 | Pseudomonas sp. MF6394 |
| fig\|76759.17.peg.5093 | Pseudomonas monteilii |
| fig\|1981678.3.peg.5241 | Pseudomonas sp. R45(2017) |
| fig\|1699620.3.peg.3028 | Pseudomonas sp. RIT-PI-r |
| fig\|191391.4.peg.2140 | Pseudomonas salomonii |
| fig\|1844093.4.peg 7190 | Pseudomonas sp. 22 E 5 |
| fig\|287.1744.peg.1414 | Pseudomonas aeruginosa |
| fig\|287.1987.peg.910 | Pseudomonas aeruginosa |
| fig\|287.4372.peg.4481 | Pseudomonas aeruginosa |
| fig\|1856685.4.peg.2159 | Pseudomonas sp. TCU-HL1 |
| fig\|1718920.3.peg.3357 | Pseudomonas sp. ICMP 8385 |
| fig\|1781066.3.peg.2816 | Duganella sp. HH101 |
| fig\|95485.5.peg.60 | Burkholderia stabilis |
| fig\|1572871.6.peg.588 | Janthinobacterium sp. BJB304 |
| WP_034208069.1 | Burkholderia cepacia |
| WP_074283015.1 | Burkholderia sp. GAS332 |
| fig\|1168169.3.peg.2570 | Methylomonas sp. 11b |
| fig\|1899355.16.peg.1328 | Oceanospirillaceae bacterium |
| WP_093197597.1 | Variovorax sp. YR750 |
| fig\|1660091.3.peg.1650 | Bordetella sp. SCN 67-23 |
| fig\|134375.17.peg.4387 | Achromobacter sp. |
| fig\|426114.10.peg.1990 | Thiomonas arsenitoxy dans |
| fig\|1947551.3.peg.1903 | Stenotrophomonas sp. UBA2302 |
| fig\|1914330.4.peg.2242 | Salinisphaera sp. |
| fig\|1947037.3.peg.890 | Pantoea sp. UBA5707 |
| WP_094422719.1 | Kosakonia cowanii |
| WP_079496884.1 | |
| WP_088126255.1 | Enterobacter kobei |
| WP_049614309.1 | Yersinia |
| WP_048263135.1 | Pectobacterium peruviense |
| WP_040197602.1 | Klebsiella pneumoniae |
| fig\|669.34.peg.1586 | Vibrio harveyi |
| fig\|672.219.peg.1032 | Vibrio vulnificus |
| fig\|670.1028.peg.1775 | Vibrio parahaemolyticus |
| WP_065207673.1 | Photobacterium phosphoreum |
| fig\|1869214.3.peg.2231 | Rheinheimera sp. |
| WP_029795910.1 | Vibrio parahaemolyticus |
| fig\|1191302.3.peg.1081 | Vibrio crassostreae 9ZC77 |
| fig\|668.70.peg.1192 | Aliivibrio fischeri |
| fig\|28229.4.peg.4229 | Colwellia psychrerythraea |
| fig\|1855726.3.peg.270 | Burkholderia sp. KK1 |
| fig\|1674888.3.peg.829 | Burkholderiales bacterium Beta_02 |
| fig\|687412.4.peg.1108 | Pseudorhodobacter aquimaris |
| WP_092465129.1 | Donghicola eburneus |
| fig\|1120653.3.peg.5479 | Ensifer sp. LC384 |
| fig\|121719.5.peg.401 | Pannonibacter phragmitetus |
| fig\|1798804.3.peg.1597 | Rhizobium sp. 58 |
| fig\|1946675.3.peg 3089 | Kordiimonas sp. UBA4487 |
| fig\|36861.5.peg.1400 | Thiobacillus denitrificans |
| fig\|1115835.3.peg.1003 | Methylotenera versatilis 79 |
| fig\|1797188.3.peg.1508 | Acidobacteria bacterium |
| | RIFCSPLOWO2_12_FULL_60_22 |
| fig\|57320.3.peg.123 | Pseudodesulfovibrio profundus |
| fig\|1267534.3.peg.1238 | Acidobacteriaceae bacterium KBS 89 |
| fig\|1951344.3.peg.527 | Acidobacteriaceae bacterium UBA1307 |
| WP_006226461.1 | Achromobacter marplatensis |
| fig\|1503054.4.peg.5764 | Burkholderia stagnalis |
| WP_006159686.1 | Cupriavidus basilensis |
| WP_090191767.1 | unclassified Duganella |
| fig\|539.8.peg.1698 | Eikenella corrodens |
| fig\|1946925.3.peg.2129 | Micavibrio sp. UBA5701 |
| WP_047031309.1 | Hocflea sp. IMCC20628 |
| fig\|1946134.3.peg.1092 | Brevundimonas sp. UBA6547 |
| WP_093914930.1 | Sulfitobacter marinus |
| fig\|1862950.3.peg.1234 | Rhizobiales bacterium NRL2 |
| fig\|1166078.4.peg.1483 | Aurcimonas phyllosphaerae |
| fig\|709015.3.peg.734 | Pontibacter actiniarum DSM 19842 |
| WP_092160028.1 | Desulfovibrio ferrireducens |
| fig\|2026749.3.peg.3364 | Ignavibacteriae bacterium |
| WP_033771991.1 | Pantoca agglomerans |
| WP_097097099.1 | unclassified Enterobacteriaceae |
| | (miscellaneous) |
| fig\|1444151.3.peg.2733 | Escherichia coli 2-177-06_S3_C2 |
| WP_137545672.1 | Escherichia coli |
| fig\|573.14856.peg.3852 | Klebsiella pneumoniae |
| WP_072021595.1 | Serratia marcescens |
| fig\|29571.3.peg.478 | Halomonas subglaciescola |
| WP_004534676.1 | |
| WP_095622523.1 | Halomonas sp. WRN001 |
| fig\|376427.4.peg.3223 | Halomonas gudaonensis |
| fig\|862908.3.peg.745 | Halobacteriovorax marinus SJ |
| SCJ40239.1 | uncultured Clostridium sp. |
| fig\|717962.3.peg.287 | Coprococcus catus GD/7 |
| WP_014642259.1 | Halobacillus halophilus |
| fig\|2009042.3.peg.2106 | Pseudomonas sp. Irchel 3H7 |
| fig\|1981718.3.peg.4346 | Pseudomonas sp. B39(2017) |
| fig\|665135.13.peg.1401 | Pseudomonas sp. In5 |
| fig\|1949067.3.peg.5629 | Pseudomonas sp. PICF141 |
| WP_007948552.1 | Pseudomonas sp. GM21 |
| SFB61662.1 | Delftia tsuruhatensis |
| WP_011615687.1 | |
| WP_014778098.1 | |
| fig\|1429083.4.peg.2612 | Pseudomonas hussainii |
| WP_095024014.1 | Pseudomonas |
| WP_090203690.1 | Pseudomonas asplenii |
| fig\|564423.8.peg.1646 | Pseudomonas tolaasii NCPPB 2192 |
| WP_078802277.1 | Pseudomonas fluorescens |
| WP_090453229.1 | Pseudomonas |
| fig\|1306420.5.peg.1032 | Burkholderia pseudomallei MSHR5848 |
| fig\|1357270.3.peg.1923 | Pseudomonas syringae UB246 |
| fig\|2018067.3.peg.2950 | Pseudomonas sp. FDAARGOS_380 |
| fig\|317.311.peg.3241 | Pseudomonas syringae |
| fig\|287.2309.peg.126 | Pseudomonas aeruginosa |
| WP_039522442.1 | Pectobacterium brasiliense |
| WP_080861357.1 | Klebsiella pneumoniae |
| WP_014542745.1 | Erwinia sp. Ejp617 |
| OSL25696.3 | Escherichia coli TA255 |
| fig\|1125693.3.peg.761 | Proteus mirabilis WGLW4 |
| KMK80587.1 | Pectobacterium atrosepticum ICMP 1526 |
| fig\|550.717.peg.2037 | Enterobacter cloacae |
| ACS86154.1 | Dickeya paradisiaca Ech703 |
| WP_050122514.1 | Yersinia frederiksenii |
| WP_081334048.1 | Alteromonas macleodii |
| WP_055016254.1 | Pseudoalteromonas sp. P1-13-1a |
| fig\|56799.5.peg.478 | Colwellia sp. |
| fig\|666.3375.peg.2486 | Vibrio cholerae |
| PIW62005.1 | Shewanella sp. |
| | CG12_big_fil_rev_8_21_14_0_65_47_15 |
| OCA54994.1 | Photorhabdus namnaonensis |
| CNK75559.1 | Yersinia frederiksenii |
| WP_024248662.1 | Escherichia |
| WP_088618141.1 | Methylovulum psychrotolerans |
| WP_051669880.1 | |
| PCJ98666.1 | Alteromonadaceae bacterium |
| WP_081919471.1 | Acidithiobacillus ferrivorans |
| WP_055769167.1 | Stenotrophomonas |
| WP_039422954.1 | Xanthomonas vesicatoria |
| WP_078568253.1 | Xanthomonas campestris |
| WP_093486747.1 | unclassified Pseudoxanthomonas |
| WP_077445058.1 | Rhodanobacter sp. C05 |
| WP_092576562.1 | Achromobacter sp. NFACC18-2 |
| fig\|1330528.3.peg.2198 | Escherichia coli NCCP 15656 |
| fig\|83655.67.peg.2965 | Leclercia adecarboxylata |
| fig\|573.10044.peg.2850 | Klebsiella pneumoniae |
| WP_071888955.1 | Enterobacterales |
| fig\|1799789.3.peg.4357 | Paraglaciecola hydrolytica |
| fig\|2024839.8.peg.1563 | Marinovum sp. |
| fig\|1381081.7.peg.1167 | Vibrio panuliri |
| fig\|670.908.peg.3444 | Vibrio parahaemolyticus |
| fig\|626887.3.peg.2431 | Marinobacter nanhaiticus D15-8W |
| fig\|1913989.101.peg.1616 | Gammaproteobacteria bacterium |
| fig\|262489.9.peg.2938 | delta proteobacterium MLMS-1 |
| fig\|2035207.3.peg.545 | Janthinobacterium sp. 67 |
| fig\|28095.13.peg.1040 | Burkholderia gladioli |
| fig\|941449.3.peg.1262 | Desulfovibrio sp. X2 |
| fig\|1768806.3.peg.778 | Rhodospirillaceae bacterium CCH5-H10 |
| WP_083634830.1 | Desulfovibrio sp. DV |
| fig\|1231.4.peg.574 | Nitrosospira multiformis |
| fig\|604089.3.peg.1142 | Flavobacterium sinopsychrotolerans |
| fig\|357523.3.peg.1851 | Flavobacterium sp. 11 |
| fig\|1423323.5.peg.321 | Flavobacterium sp. AED |
| fig\|178356.3.peg.502 | Flavobacterium xinjiangense |
| fig\|1946545.3.peg.3457 | Flavobacterium sp. UBA4120 |
| fig\|150146.3.peg.2822 | Flavobacterium gillisiae |
| fig\|229203.4.peg.1981 | Flavobacterium degerlachei |
| fig\|280093.5.peg.432 | Flavobacterium granuli |
| fig\|728056.4.peg.1154 | Flavobacterium oncorhynchi |
| fig\|143224.8.peg.2343 | Zobellia uliginosa |
| fig\|1225176.3.peg.4300 | Cecembia lonarensis LW9 |
| fig\|1434700.3.peg.581 | Moheibacter sediminis |
| fig\|996.47.peg.468 | Flavobacterium columnare |
| fig\|172045.56.peg.2231 | Elizabethkingia miricola |
| fig\|2024823.3.peg.2086 | Altibacter sp. |
| fig\|2026728.18.peg.4090 | Crocinitomicaceae bacterium |
| fig\|980584.3.peg.2930 | Aquimarina agarivorans |
| fig\|1946744.3.peg.1682 | Leeuwenhoekiella sp. UBA1003 |
| fig\|1046627.3.peg.2526 | Bizionia argentinensis JUB59 |
| fig\|906888.15.peg.37 | Nonlabens ulvanivorans |
| fig\|407022.4.peg.2865 | Olivibacter domesticus |
| fig\|1500282.3.peg.3713 | Chry scobacterium sp. CF365 |
| WP_084550290.1 | Chryseobacterium scophthalmum |
| fig\|190304.8.peg.741 | Fusobacterium nucleatum subsp. |
| | nucleatum ATCC 25586 |
| fig\|1352.1731.peg.603 | Enterococcus faecium |
| fig\|1428.658.peg.666 | Bacillus thuringiensis |
| fig\|1497681.3.peg.3095 | Listeria newyorkensis |
| fig\|1396.1440.peg.4237 | Bacillus cereus |
| fig\|1917876.3.peg.2997 | Blautia sp. Marseille-P3087 |
| fig\|1952168.3.peg.215 | Lachnospiraceae bacterium UBA7480 |
| fig\|1907659.3.peg.1085 | Blantia sp. Marseille-P3201T |
| fig\|1265309.16.peg.461 | Epibacterium mobile F1926 |
| fig\|853.163.peg.215 | Faecalibacterium prausnitzii |
| fig\|1264.5.peg.4 | Ruminococcus albus |
| fig\|1500289.3.peg.4469 | Chryseobacterium sp. OV705 |
| fig\|1197728.3.peg.2386 | Prevotella conceptionensis 9403948 |
| fig\|1947486.3.peg.2515 | Sphingobacterium sp. UBA1897 |
| fig\|529.12.peg.1303 | Ochrobactrum anthropi |
| fig\|1523429.3.peg.2936 | Rhizobium sp. AAP116 |
| fig\|1761878.3.peg.469 | Paenibacillus sp. cl6col |
| fig\|1462996.4.peg.2634 | Paemibacillus yonginensis |
| fig\|582475.4.peg.4724 | Lysinibacillus xylanilyticus |
| fig\|1773.7915.peg.7638 | Mycobacterium tuberculosis |
| fig\|360310.3.peg.4853 | Bacillus sp. CDB3 |
| fig\|1396.515.peg.2936 | Bacillus cereus |
| fig\|662367.4.peg.242 | Spirosoma endophyticum |
| fig\|1895719.3.peg.2950 | Bacteroidales bacterium 45-6 |
| fig\|906888.9.peg.926 | Nonlabens ulvanivorans |
| fig\|694433.3.peg.2346 | Saprospira grandis DSM 2844 |
| fig\|1167006.5.peg.2941 | Desulfocapsa sulfexigens DSM 10523 |
| fig\|649724.3.peg.304 | Clostridium sp. ATCC BAA-442 |
| fig\|1505.32.peg.2959 | Paeniclostridium sordellii |
| fig\|1953142.3.peg.1858 | Bacteroidetes bacterium UBA1947 |
| fig\|2029590.3.peg.2754 | Mucilaginibacter sp. MD40 |
| fig\|29581.33.peg.2300 | Janthinobacterium lividum |
| fig\|40324.292.peg.236 | Stenotrophomonas maltophilia |
| fig\|1403329.3.peg.287 | Listeria monocytogenes Lm25180 |
| fig\|1121865.3.peg.1262 | Enterococcus columbae |
| | DSM 7374 = ATCC 51263 |
| fig\|1120746.3.peg 3113 | bacterium MS4 |
| fig\|1952299.3.peg.221 | Ruminococcaceae bacterium UBA2656 |
| fig\|1965604.3.peg.686 | Anacromassilibacillus sp. An250 |
| fig\|1673717.3.peg.805 | Anaeromassilibacillus senegalensis |
| WP_116884683.1 | Victivallis vadensis |
| fig\|1948697.3.peg.196 | Lentisphaeria bacterium UBA4640 |
| fig\|1232460.3.peg.46 | Clostridiales bacterium VE202-28 |
| WP_007864340.1 | Clostridiales |
| WP_055649738.1 | Hungatella hathewayi |
| fig\|1226325.3.peg.2005 | Clostridium sp. KLE 1755 |
| fig\|1432052.10.peg.3166 | Eisenbergiella tayi |
| fig\|208479.8.peg.4376 | Enterocloster bolteae |
| fig\|1298920.3.peg.1959 | [Desulfotomaculum] guttoideum DSM 4024 |
| fig\|1776047.3.peg.4241 | Clostridium sp. C105KSO15 |
| fig\|1946596.3.peg.2399 | Hungatella sp. UBA4396 |
| fig\|1946603.3.peg.924 | Hungatella sp. UBA7603 |
| fig\|1410651.3.peg.407 | [Clostridium] aerotolerans DSM 5434 |
| fig\|1697784.3.peg.9617 | Clostridia bacterium UC5.1-1D4 |
| fig\|1745713.3.peg.3865 | Bariatricus massiliensis |
| fig\|180332.3.peg.1515 | Robinsoniella peoriensis |
| WP_072851604.1 | Lactonifactor longoviformis |
| WP_003507561.1 | Clostridiales |
| fig\|1111728.3.peg.587 | Budvicia aquatica DSM 5075 = ATCC 35567 |
| fig\|1122977.4.peg.2473 | Pragia fontium DSM 5563 = ATCC 49100 |
| fig\|1950915.3.peg.189 | Clostridiales bacterium UBA644 |
| fig\|1950927.3.peg.912 | Clostridiales bacterium UBA7187 |
| ERK60856.1 | Oscillibacter sp. KLE 1728 |
| WP_009260579.1 | Flavonifractor plautii |
| fig\|1235797.3.peg.2409 | Oscillibacter sp. 1-3 |
| fig\|1520815.3.peg.1262 | Ruminococcaceae bacterium D5 |
| fig\|1855302.3.peg.1138 | Pseudobutyrivibrio sp. JW11 |
| fig\|43305.5.peg.3631 | Butyrivibrio proteoclasticus |
| fig\|411463.15.peg.1791 | Eubacterium ventriosum ATCC 27560 |
| fig\|1235792.3.peg.3837 | Lachnospiraceae bacterium M18-1 |
| fig\|97139.3.peg.669 | Schaedlerella arabinosiphila |
| fig\|1291051.3.peg.1165 | Mediterraneibacter glycyrrhizinilyticus |
| | JCM 13369 |
| fig\|1532.6.peg.4793 | Blautia coccoides |
| fig\|1121114.4.peg.5478 | Blantia producta ATCC 27340 = DSM 2950 |
| fig\|1262776.3.peg.1908 | Clostridium sp. CAG: 149 |
| fig\|1262792.3.peg.1164 | Clostridium sp. CAG:299 |
| fig\|1262995.3.peg.2852 | Firmicutes bacterium CAG:646 |
| fig\|537007.17.peg.3146 | Blautia hansenii DSM 20583 |
| fig\|1965569.3.peg 1928 | Lachnoclostridium sp. An169 |
| fig\|1952411.3.peg.2018 | Ruminococcaceae bacterium UBA6353 |
| fig\|1965578.3.peg.1947 | Pseudoflavonifractor sp. An187 |
| WP_001775049.1 | Escherichia coli |
| WP_012602583.1 | |
| WP_015962464.1 | Enterobacteriaceae bacterium strain FGI 57 |
| fig\|1005999.3.peg.3342 | Leminorella grimontil ATCC |
| | 33999 = DSM 5078 |
| fig\|1378073.3.peg.795 | Enterobacter sp. CC120223-11 |
| fig\|911023.3.peg.138 | Yokenella regensburgei ATCC 49455 |
| fig\|1834193.3.peg.4113 | Enterococcus sp. 9E7_DIV0242 |
| fig\|1649188.10.peg.406 | Listeria goaensis |
| fig\|1430899.3.peg 278 | Listeria fleischmannii 1991 |
| fig\|1211844.4.peg.748 | Candidatus Stoquefichus massiliensis AP9 |
| fig\|1658109.3.peg.34 | Candidatus Stoquefichus sp. SB1 |
| fig\|1262793.3.peg.950 | Clostridium sp. CAG:302 |
| fig\|1262908.3.peg.1120 | Mycoplasma sp. CAG:956 |
| fig\|1674844.3.peg.242 | Clostridiales bacterium Firm_06 |
| fig\|1410672.3.peg.2823 | Ruminococcus flavefaciens ND2009 |
| fig\|1947424.3.peg.1718 | Ruminococcus sp. UBA4310 |
| fig\|1265.9.peg.2602 | Ruminococcus flavefaciens |
| fig\|1336236.3.peg 1817 | Ruminococcus flavefaciens ATCC 19208 |
| CDC65895.1 | Ruminococcus sp. CAG:57 |
| WP_092946213.1 | Ruminococcaceae bacterium YRB3002 |
| fig\|1307.1644.peg 1532 | Streptococcus suis |
| WP_050516365.1 | Escherichia coli |
| WP_097505494.1 | Escherichia coli |
| fig\|573.15585.peg.2343 | Klebsiella pneumoniae |
| WP_023581669.1 | Proteus hauseri |
| WP_079656969.1 | Serratia marcescens |
| WP_090085157.1 | Phytobacter sp. SCO41 |
| fig\|573.15584.peg.1543 | Klebsiella pneumoniae |
| WP_023330997.1 | Enterobacter cloacae complex |
| fig\|72407.673. peg.2552 | Klebsiella pneumoniae subsp. pneumoniae |
| CNM01182.1 | Yersinia pseudotuberculosis |
| CNG88012.1 | Yersinia enterocolitica |
| fig\|1925763.3.peg.649 | Marinobacter salexigens |
| PKW24121.1 | Marinobacter sp. LV10R510-8 |
| WP_045597342.1 | Vibrio vulnificus |
| WP_098972386.1 | Aeromonas sp. CU5 |
| WP_005172873.1 | Yersinia enterocolitica |
| WP_052979504.1 | Enterobacteriaceae |
| WP_083069261.1 | Pantoea vagans |
| WP_053911905.1 | Pseudoalteromonas sp. SW0106-04 |
| fig\|1916082.18.peg.39 | Alteromonadaceae bacterium |
| WP_046555216.1 | Arsukibacterium sp. MJ3 |
| KPW01986.1 | Pseudoalteromonas sp. P1-8 |
| WP_094277737.1 | Oceanimonas baumannii |
| fig\|1414654.3.peg.2005 | Oceanisphaera psychrotolerans |
| WP_008133621.1 | unclassified Pseudoalteromonas |
| KQA22543.1 | Vibrio metoecus |
| WP_000284440.1 | Vibrio cholerae |
| WP_011261677.1 | Aliivibrio fischeri |
| KEE40622.1 | |
| WP_012982829.1 | |
| ALL66139.1 | Paraburkholderia caribensis MBA4 |
| WP_093223969.1 | Pseudomonas vancouverensis |
| fig\|2015553.3.peg.2940 | Pseudomonas sp. PGPPP1 |
| WP_096082869.1 | Pseudomonas aeruginosa |
| ONM67687.1 | Pseudomonas aeruginosa |
| fig\|316.213.peg.2906 | Pseudomonas stutzeri |
| WP_078734267.1 | Pseudomonas fluorescens |
| WP_079384669.1 | Pseudomonas aeruginosa |
| WP_095948157.1 | Variovorax boronicumulans |
| WP_011625020.1 | Shewanella sp. MR-7 |
| WP_100292553.1 | Aeromonas cavernicola |
| WP_055021484.1 | Pseudoalteromonas sp. P1-26 |
| PHS01491.1 | Oceanobacter sp. |
| fig\|2024618.3.peg.1141 | Acinetobacter sp. BS1 |
| WP_114139108.1 | Klebsiella pneumoniae |
| WP_077749737.1 | Pseudomonas sp. FSL W5-0299 |
| WP_078451378.1 | Pseudomonas aeruginosa |
| WP_007245785.1 | Pseudomonas syringae group |
| fig\|316.280.peg.1454 | Pseudomonas stutzeri |
| WP_086822222.1 | Pseudomonas aeruginosa |
| WP_073268605.1 | Pseudomonas punonensis |
| WP_095280108.1 | Lelliottia jeotgali |
| WP_095715328.1 | Citrobacter sp. TSA-1 |
| WP_050111525.1 | Yersinia |
| WP_013724211.1 | Aeromonas veronii |
| WP_021140819.1 | Aeromonas salmonicida |
| fig\|1094342.5.peg.1611 | Alcanivorax xenomutans |
| fig\|1932666.4.peg.1886 | Haliea sp. |
| WP_087148323.1 | Crenothrix polyspora |
| WP_064022638.1 | Methylomonas sp. DH-1 |
| PIY64876.1 | Shewanella sp. |
| | CG_4_10_14_0_8_um_filter_42_13 |
| WP_006710190.1 | Vibrio ichthyoenteri |
| WP_045040928.1 | Photobacterium iliopiscarium |
| WP_054543201.1 | Vibrio splendidus |
| WP_080540293.1 | Vibrio vulnificus |
| fig\|2032624.3.peg.2540 | Halomonas sp. WN018 |
| KJT50308.1 | Salmonella enterica subsp. enterica |
| | serovar Heidelberg str. RI-11-014588 |
| WP_005761319.1 | |
| ODQ05744.1 | Shigella sp. FC130 |
| KKW01006.1 | Candidatus Saccharibacteria bacterium |
| | GW2011_GWC2_48_9 |
| KMZI2260.1 | Candidatus Burkholderia humilis |
| SFQ04394.1 | Ralstonia sp. NFACC01 |
| WP_025373922.1 | Advenella mimigardefordensis |
| WP_093341200.1 | Variovorax sp. PDC80 |
| WP_091453700.1 | Giesbergeria anulus |
| SAY51889.1 | Neisseria weaveri |
| WP_065255232.1 | Moraxella lacunata |
| WP_049330876.1 | Neisseria |
| fig\|1196095.197.peg.151 | Gilliamella apicola |
| WP_072956843.1 | Vibrio gazogenes |
| fig\|857087.3.peg.3286 | Methylomonas methanica MC09 |
| fig\|1952222.3.peg.1307 | Methylococcaceae bacterium UBA3127 |
| WP_039486261.1 | Vibrio sinaloensis |
| WP_065545234.1 | Vibrio scophthalmi |
| WP_033094845.1 | Colwellia psychrerythraea |
| WP_057552475.1 | Vibrio cholerae |
| WP_004726393.1 | Vibrio furnissii |
| fig\|2020862.3.peg.1934 | Halobacteriovorax sp. |
| fig\|624.1260.peg.1437 | Shigella sonnei |
| WP_011516221.1 | Burkholderiales |
| fig\|1947370.3.peg.1923 | Pusillimonas sp. UBA4517 |
| WP_038400955.1 | Yersinia pseudotuberculosis |
| fig\|1951903.3.peg.117 | Halieaceae bacterium UBA3099 |
| WP_024914507.1 | Chania multitudinisentens |
| WP_042893228.1 | Enterobacteriaceae |
| WP_038238211.1 | Xenorhabdus szentirmaii |
| EXI65661.1 | Candidatus Accumulibacter sp. SK-12 |
| WP_016452106.1 | Delftia |
| WP_013517170.1 | Alicycliphilus denitrificans |
| OXC73828.1 | Caballeronia sordidicola |
| AIO65205.1 | Burkholderia oklahomensis |
| WP_013234866.1 | Herbaspirillum seropedicae |
| WP_082884385.1 | Piscirickettsiaceae bacterium NZ-RLO1 |
| fig\|2006849.4.peg.371 | Xanthomonadales bacterium |
| WP_074262787.1 | Paraburkholderia phenazinium |
| WP_009906786.1 | Burkholderia thailandensis |
| WP_022524328.1 | |
| WP_081817450.1 | Halomonas sp. HL-48 |
| WP_020312233.1 | Pseudomonas syringae |
| KPY75916.1 | Pseudomonas amygdali pv. tabaci |
| fig\|1793966.3.peg.180 | Pseudomonas fluvialis |
| fig\|1891229.16.peg.2033 | Pseudomonadales bacterium |
| WP_099454886.1 | Pseudomonas putida |
| WP_092400423.1 | Pseudomonas sp. NFACC39-1 |
| WP_012315430.1 | Pseudomonas putida |
| WP_020799819.1 | Pseudomonas sp. G5(2012) |
| WP_004574016.1 | |
| fig\|1435425.3.peg.787 | Pseudomonas sp. QTF5 |
| WP_045490543.1 | Pseudomonas sp. StFLB209 |
| WP_011506503.1 | Chromohalobacter salexigens |
| fig\|1609967.3.peg 3047 | Halomonas sp. HG01 |
| fig\|1492738.3.peg.2698 | Flavobacterium seoulense |
| WP_092849245.1 | Algibacter pectinivorans |
| WP_025835957.1 | Bacteroides |
| fig\|2025877.3.peg.668 | Parabacteroides sp. AT13 |
| fig\|246787.6.peg.2081 | Bacteroides cellulosilyticus |
| fig\|1339287.3.peg.1113 | Bacteroides fragilis str. 3986 T(B)9 |
| fig\|1946017.3.peg.1516 | Alistipes sp. UBA940 |
| WP_038655380.1 | Mucinivorans hirudinis |
| WP_093669272.1 | Tenacibaculum sp. MAR_2009_124 |
| WP_073241067.1 | Flavobacterium flevense |
| WP_096193803.1 | Cytophagales bacterium TFI 002 |
| WP_076357635.1 | |
| WP_073238193.1 | Pedobacter caeni |
| WP_076451370.1 | |
| WP_091906542.1 | Porphyromonadaceae bacterium KH3R.12 |
| WP_051365712.1 | Flavobacterium saliperosum |
| fig\|1938609.3.peg.1765 | Flavobacterium sp. LM4 |
| SDJ72221.1 | Flavobacterium noncentrifugens |
| fig\|1985174.3.peg.2584 | Chitinophagaceae bacterium IBVUCB2 |
| WP_092737749.1 | Riemerella columbipharyngis |
| fig\|192149.3.peg.42 | Muricauda sp. |
| fig\|418630.3.peg.1685 | Rhodobacter megalophilus |
| fig\|1915314.3.peg.3469 | Thioclava sp. DLFJ5-1 |
| fig\|2030815.3.peg.2725 | Marinosulfonomonas sp. |
| fig\|2035451.3.peg.4632 | Rhizobium sp. L18 |
| WP_043872258.1 | Celeribacter indicus |
| WP_055683826.1 | Jannaschia rubra |
| fig\|1947537.3.peg.498 | Sphingopyxis sp. UBA6198 |
| WP_069065961.1 | Sphingobium sp. RAC03 |
| WP_084280100.1 | Novosphingobium sp. B1 |
| fig\|1895845.3.peg.487 | Sphingobium sp. 66-54 |
| GAK73419.1 | Agrobacterium rubi TR3 = NBRC 13261 |
| WP_090966398.1 | Aureimonas phyllosphaerae |
| WP_091860144.1 | Bosea robiniae |
| WP_085092006.1 | Azospirillum oryzae |
| fig\|1528100.4.peg.28 | Methylomagnum ishizawai |
| fig\|32057.3.peg.9515 | Calothrix sp. PCC 7103 |
| fig\|103690.10.peg.3571 | Nostoc sp. PCC 7120 = FACHB-418 |
| fig\|1137095.11.peg.15 | Scytonema sp. HK-05 |
| CDZ48826.1 | Neorhizobium galegae bv. officinalis |
| WP_072340070.1 | Devosia enhydra |
| OYR18277.1 | Ochrobactrum thiophenivorans |
| WP_093509439.1 | Sphingopyxis sp. YR583 |
| WP_081799025.1 | Novosphingobium resinovorum |
| PIY55545.1 | Zetaproteobacteria bacterium |
| | CG_4_10_14_0_8_um_filter_49_80 |
| SDT44912.1 | Bradyrhizobium canariense |
| WP_096350346.1 | |
| WP_074962594.1 | Jannaschia rubra |
| WP_038724888.1 | Burkholderia pseudomallei |
| WP_012217410.1 | Burkholderia multivorans |
| WP_100428762.1 | Janthinobacterium sp. 67 |
| WP_082161008.1 | Candidatus Competibacter denitrificans |
| AFL73219.1 | Thiocystis violascens DSM 198 |
| WP_014427842.1 | |
| fig\|364030.3.peg.3554 | Thiomonas delicata |
| KGW20495.1 | Burkholderia pseudomallei MSHR2451 |
| SFE83076.1 | Variovorax sp. OK212 |
| WP_013028226.1 | Sideroxydans lithotrophicus |
| WP_080311424.1 | Burkholderia pseudomallei |
| fig\|337.13.peg.3872 | Burkholderia glumae |
| WP_082643860.1 | Pseudomonas |
| CKH90039.1 | Pseudomonas aeruginosa |
| WP_083287254.1 | unclassified Janthinobacterium |
| WP_122648546.1 | Burkholderia pseudomallei |
| WP_082706753.1 | unclassified Pseudomonas |
| WP_080936076.1 | Klebsiella pneumoniae |
| WP_000746343.1 | Enterobacteriaceae |
| EMX54653.1 | Escherichia coli MP020980.2 |
| WP_053270700.1 | Escherichia coli |
| fig\|1736224.3.peg.3731 | Serratia sp. Leaf51 |
| fig\|1175299.4.peg.709 | Dickeya zeae ZJU1202 |
| WP_001461245.1 | Enterobacteriaceae |
| fig\|617145.3.peg.3535 | Vibrio splendidus IF-157 |
| fig\|1440054.3.peg.3851 | Vibrio sp. OY15 |
| fig\|617135.3.peg.594 | Aliivibrio fischeri ZF-211 |
| WP_023267764.1 | Shewanella decolorationis |
| fig\|1481663.36.peg.3628 | Vibrio metoecus |
| fig\|670.893.peg.2716 | Vibrio parahaemolyticus |
| fig\|680.33.peg.5391 | Vibrio campbellii |
| fig\|298386.8.peg.4344 | Photobacterium profundum SS9 |
| fig\|663.73.peg.714 | Vibrio alginolyticus |
| fig\|1333511.3.peg.3208 | Pseudoalteromonas haloplanktis TAB23 |
| WP_064574154.1 | Hafnia paralvei |
| WP_064645509.1 | Obesumbacterium proteus |
| fig\|630.105.peg.4248 | Yersinia enterocolitica |
| fig\|400673.7.peg.1969 | Legionella pneumophila str. Corby |
| WP_092678546.1 | Rosenbergiella nectarea |
| WP_069476513.1 | Raoultella ornithinolytica |
| fig\|1267535.3.peg.2394 | Bryobacterales bacterium KBS 96 |
| WP_000446053.1 | Acinetobacter baumannii |
| fig\|1948587.3.peg.786 | Gammaproteobacteria bacterium UBA1902 |
| WP_014949305.1 | Alteromonas macleodii |
| fig\|1797397.3.peg.2386 | Bdellovibrionales bacterium |
| | RIFOXYC1_FULL_54_43 |
| fig\|1386968.3.peg.847 | Francisella tularensis subsp. |
| | novicida PA10-7858 |
| WP_074900850.1 | |
| fig\|1975705.3.peg.898 | Psychrobacter sp. FDAARGOS_221 |
| WP_066184577.1 | Arcobacter |
| fig\|1780380.4.peg.4010 | Eubacteriaceae bacterium CHKCI004 |
| fig\|556261.3.peg.2546 | Clostridium sp. D5 |
| fig\|1193534.6.peg.2375 | uncultured Flavonifractor sp. |
| fig\|1042163.3.peg.3771 | Brevibacillus laterosporus LMG 15441 |
| WP_062492190.1 | Paenibacillus sp. 32O-W |
| WP_081674606.1 | Lactobacillus harbinensis |
| WP_050781686.1 | Lactobacillus coryniformis |
| WP_021109137.1 | Enterococcus faecium |
| WP_046309803.1 | Staphylococcus |
| CBL03706.1 | Gordonibacter pamelacae 7-10-1-b |
| WP_090944285.1 | Pelosinus propionicos |
| WP_077305443.1 | Clostridium beijerinckii |
| fig\|410072.5.peg.40 | Coprococcus comes |
| WP_011669870.1 | Leptospira borgpetersenii |
| WP_015565235.1 | Faecalibacterium prausnitzij |
| CUO23478.1 | Faecalibacterium prausnitzii |
| WP_085748688.1 | Rhizobacter gummiphilus |
| WP_093270014.1 | Psychrobacillus sp. OK032 |
| SHE86352.1 | Atopostipes suicloacalis DSM 15692 |
| WP_000346292.1 | unclassified Streptococcus |
| WP_080465410.1 | Lactobacillus plantarum |
| WP_080662531.1 | Lactobacillus brevis |
| WP_093131554.1 | Salinibacillus kushneri |
| WP_093336905.1 | Salibacterium halotolerans |
| fig\|1974627.3.peg.386 | Candidatus Levybacteria bacterium |
| | CG_4_9_14_0_2_um_filter_35_21 |
| fig\|1802603.3.peg.453 | Candidatus Woykebacteria bacterium |
| | RIFCSPHIGHO2_12_FULL_45_10 |
| fig\|392734.5.peg.3006 | Terriglobus roseus |
| AGL61879.1 | Candidatus Saccharimonas aalborgensis |
| fig\|319224.16.peg.2726 | Shewanella putrefaciens CN-32 |
| fig\|1720343.3.peg.1263 | Pseudoalteromonas sp. |
| | 1_2015MBL_MicDiv |
| fig\|1136158.3.peg.3691 | Vibrio cyclitrophicus 1F97 |
| fig\|666.3017.peg.1000 | Vibrio cholerae |
| fig\|1909458.3.peg.2277 | Salinivibrio sp. ML198 |
| fig\|1638949.3.peg.831 | Vibrio sp. ECSMB14106 |
| fig\|493915.3.peg.158 | Pseudoalteromonas sp. NJ631 |
| BAC94535.1 | Vibrio vulnificus YJ016 |
| fig\|1191313.3.peg.1135 | Vibrio splendidus 1S-124 |
| fig\|670.1244.peg.3807 | Vibrio parahaemolyticus |
| fig\|1659714.3.peg.4264 | Citrobacter braakii |
| fig\|1192730.4.peg.1976 | Salmonella enterica subsp. enterica |
| | serovar Kintambo |
| fig\|550.1216.peg.4296 | Enterobacter cloacae |
| WP_072269713.1 | Serratia |
| WP_053898075.1 | Escherichia coli |
| fig\|624.1264.peg.1635 | Shigella sonnei |
| fig\|1181777.3.peg.78 | Escherichia coli KTE233 |
| fig\|1802256.3.peg.310 | Sulfurimonas sp. RIFOXYB12_FULL_35_9 |
| PHR73342.1 | Arcobacter sp. |
| fig\|2014260.3.peg.3813 | bacterium (Candidatus Blackallbacteria) |
| | CG13_big_fil_rev_8_21_14_2_50_49_14 |
| WP_042497590.1 | Vibrio maritimus |
| WP_063522799.1 | Vibrio sp. H100D65 |
| WP_004186757.1 | Enterobacteriaceae |
| WP_040122746.1 | Vibrio |
| WP_086046550.1 | Vibrio harveyi group |
| WP_063849005.1 | Enterobacter cloacae |
| WP_023486614.1 | Enterobacteriaceae |
| WP_070992278.1 | Pseudoalteromonas byunsanensis |
| fig\|1005665.3.peg.2532 | Kosakonia oryzendophytica |
| fig\|1219066.3.peg.3636 | Vibrio parahaemolyticus NBRC 12711 |
| fig\|1225184.4.peg.1222 | Pantoea sp. A4 |
| fig\|675814.3.peg.1256 | Vibrio coralliilyticus ATCC BAA-450 |
| SFR59865.1 | Pseudobutyrivibrio sp. NOR37 |
| fig\|853.16.peg.1112 | Faecalibacterium pransnitzii |
| fig\|1965572.3.peg.1423 | Pseudoflavonifractor sp. An176 |
| fig\|588581.3.peg.3589 | Ruminiclostridium papyrosolvens DSM 2782 |
| fig\|1396.1409.peg.4169 | Bacillus cereus |
| fig\|1428.538.peg.4047 | Bacillus thuringiensis |
| fig\|1465.16.peg.946 | Brevibacillus laterosporus |
| WP_087385137.1 | |
| AIF42417.1 | Virgibacillus sp. SK37 |
| WP_076543941.1 | Halanaerobium kushneri |
| fig\|1121093.3.peg.3089 | Bacillus panaciterrae DSM 19096 |
| fig\|29367.3.peg.2029 | Clostridium puniceum |
| WP_089719707.1 | Halanaerobium congolense |
| fig\|307249.3.peg.3585 | uncultured Sporomusa sp. |
| WP_072949666.1 | Ruminococcus flavefaciens |
| CCX81854.1 | Ruminococcus sp. CAG:108 |
| fig\|1491.669.peg.2217 | Clostridium botulinum |
| fig\|1872455.3.peg.401 | Alkaliphilus sp. |
| fig\|576117.5.peg.4005 | Celeribacter halophilus |
| fig\|1225647.3.peg.1829 | Phacobacter sp. 11ANDIMAR09 |
| fig\|1380380.4.peg.1574 | Ahrensia sp. 13_GOM-1096m |
| fig\|293.7.peg 2956 | Brevundimonas diminuta |
| WP_095437634.1 | Rhizobium sp. 11515TR |
| fig\|1912891.7.peg.702 | Sphingobium sp. |
| fig\|1736574.3.peg.4024 | Pseudoxanthomonas sp. Root630 |
| fig\|227946.13.peg.4105 | Xanthomonas translucens pv. poae |
| fig\|1761791.3.peg.4793 | Lysobacter sp. yr284 |
| fig\|1560195.5.peg.485 | Janthinobacterium sp. BJB301 |
| fig\|1503054.43.peg.6257 | Burkholderia stagnalis |
| fig\|1207504.10.peg.4279 | Burkholderia pseudomultivorans |
| WP_092172515.1 | unclassified Pseudomonas |
| WP_074815429.1 | Pseudomonas syringae |
| fig\|150146.3.peg.3162 | Flavobacterium gillisiae |
| fig\|76832.8.peg.3775 | Myroides odoratimimus |
| fig\|1202724.3.peg.994 | Flavobacterium akiainvivens |
| fig\|1805473.3.peg.3678 | Chryseobacterium timonianum |
| fig\|253.33.peg.3826 | Chryseobacterium indologenes |
| WP_076561634.1 | Chryseobacterium indoltheticum |
| fig\|2024823.3.peg.95 | Altibacter sp. |
| fig\|1250278.4.peg 3462 | Salegentibacter sp. Hel_I_6 |
| fig\|1797342.3.peg.689 | Bacteroidetes bacterium GWF2_33_38 |
| WP_084184261.1 | Chryscobacterium ureilyticum |
| fig\|1948560.3.peg.3003 | Deltaproteobacteria bacterium UBA6106 |
| fig\|1392.364.peg.2564 | Bacillus anthracis |
| fig\|872970.3.peg.1713 | Amphibacillus marinus |
| fig\|1385514.3.peg.313 | Pontibacillus yanchengensis Y32 |
| fig\|76853.4.peg.2614 | Solibacillus silvestris |
| fig\|1423774.3.peg.1262 | Lactobacillus nantensis DSM 16982 |
| fig\|1410670.3.peg.2844 | Ruminococcus flavefaciens MA2007 |
| fig\|169435.7.peg.1348 | Anaerotruncus colihominis |
| fig\|1946597.3.peg.2104 | Hungatella sp. UBA4568 |
| fig\|1948087.3.peg.796 | Firmicutes bacterium UBA6113 |
| fig\|642492.3.peg.2638 | Cellulosilyticum lentocellum DSM 5427 |
| fig\|1950841.3.peg.2383 | Clostridiales bacterium UBA2436 |
| fig\|555512.3.peg.1251 | Salipiger marinus |
| fig\|383381.3.peg.2538 | Erythrobacter sp. JL475 |
| WP_081629462.1 | |
| fig\|1736258.3.peg.3392 | Methylobacterium sp. Leaf112 |
| fig\|1950192.3.peg.426 | Anaerolineales bacterium UBA2232 |
| fig\|170623.6.peg.4661 | Azotobacter beijerinckii |
| fig\|170623.7.peg.704 | Azotobacter beijerinckii |
| fig\|1981099.3.peg.513 | Niveispirillum lacus |
| fig\|1250539.3.peg.3491 | Pelagibaca abyssi |
| fig\|1947582.3.peg.2979 | Sulfitobacter sp. UBA1132 |
| fig\|1909294.17.peg.3456 | Rhizobiales bacterium |
| fig\|1735583.3.peg.1657 | Pseudovibrio sp. W64 |
| fig\|670.1220.peg.4688 | Vibrio parahaemolyticus |
| fig\|1004786.3.peg.925 | Alteromonas mediterranea DE1 |
| fig\|2013797.3.peg.2109 | Gammaproteobacteria bacterium HGW- |
| | Gammaproteobacteria-15 |
| fig\|1948580.3.peg.3400 | Gammaproteobacteria bacterium UBA1012 |
| fig\|1714300.3.peg.306 | Marinobacterium profundum |
| fig\|1961547.3.peg.1371 | Desulfobulbaceae bacterium UBA2273 |
| fig\|441162.10.peg.6621 | Burkholderia oklahomensis C6786 |
| fig\|615.307.peg.4666 | Serratia marcescens |
| fig\|631.3.peg.1883 | Yersinia intermedia |
| fig\|1763535.3.peg.1547 | Hydrogenophaga crassostreae |
| fig\|43263.5.peg.2702 | Pseudomonas alcaligenes |
| fig\|244366.46.peg.3595 | Klebsiella variicola |
| fig\|1224150.8.peg.3856 | Dickeya paradisiaca NCPPB 2511 |
| fig\|61645.10.peg.2019 | Enterobacter asburiae |
| fig\|1948706.3.peg.2225 | Opitutae bacterium UBA1333 |
| fig\|2026771.13.peg.1697 | Opitutae bacterium |
| fig\|2026771.11.peg.1955 | Opitutae bacterium |
| fig\|2026772.5.peg.424 | Opitutales bacterium |
| fig\|2026801.20.peg.1798 | Verrucomicrobiales bacterium |
| fig\|2026801.14.peg.1176 | Verrucomicrobiales bacterium |
| fig\|1951369.3.peg.1157 | Akkermansiaceae bacterium UBA6946 |
| fig\|1977087.12.peg.1918 | Proteobacteria bacterium |
| fig\|2026779.14.peg.4171 | Planctomycetaceae bacterium |
| fig\|2026779.28.peg.3264 | Planctomycetaceae bacterium |
| fig\|2026779.30.peg.3181 | Planctomycetaceae bacterium |
| fig\|2026779.29.peg.2310 | Planctomycetaceae bacterium |
| fig\|1797235.3.peg 3 | Acinetobacter sp. RIFCSPHIGHO2_12_41_5 |
| fig\|316.284.peg.937 | Pseudomonas stutzeri |
| fig\|296.11.peg.442 | Pseudomonas fragi |
| fig\|1981714.3.peg.993 | Pseudomonas sp. B5(2017) |
| fig\|50340.44.peg.6020 | Pseudomonas fuscovaginae |
| fig\|1761897.3.peg.509 | Pseudomonas sp. ok272 |
| fig\|1402514.3.peg.154 | Pseudomonas aeruginosa BWHPSA014 |
| fig\|1938440.3.peg.5997 | Pseudomonas sp. T |
| fig\|1566250.3.peg 959 | Pseudomonas sp. NFACC02 |
| fig\|316.357.peg.479 | Pseudomonas stutzeri |
| fig\|287.4433.peg.2945 | Pseudomonas aeruginosa |
| fig\|1970515.3.peg.709 | Hydrogenophilales bacterium 12-61-10 |
| fig\|95486.85.peg.1748 | Burkholderia cenocepacia |
| fig\|292.61.peg.8104 | Burkholderia cepacia |
| fig\|1408450.3.peg.3766 | Methylobacter tundripaludum 21/22 |
| fig\|157910.3.peg.5727 | Paraburkholderia tuberum |
| fig\|279058.16.peg.4239 | Collimonas arenae |
| fig\|1537272.3.peg.1916 | Janthinobacterium sp. HH100 |
| fig\|1218081.3.peg.1751 | Paraburkholderia kururiensis subsp. |
| | thiooxydans NBRC 107107 |
| fig\|573.14059.peg.3113 | Klebsiella pneumoniae |
| fig\|40324.192.peg.51 | Stenotrophomonas maltophilia |
| fig\|1219041.3.peg.4613 | Sphingomonas azotifigens NBRC 15497 |
| fig\|1561196.3.peg.560 | Burkholderia sp. E7m39 |
| fig\|1882750.3.peg.1035 | Burkholderia sp. GAS332 |
| fig\|1736266.3.peg.1145 | Duganella sp. Leaf126 |
| fig\|2015350.3.peg.1640 | Burkholderia sp. AU18528 |
| fig\|58133.4.peg.815 | Nitrosospira sp. NpAV |
| fig\|1691980.3.peg.1912 | Rhodocyclaceae bacterium Paddy-1 |
| fig\|305.393.peg.1023 | Ralstonia solanacearum |
| fig\|56449.3.peg.3604 | Xanthomonas bromi |
| fig\|1281282.5.peg.1894. | Xanthomonas campestris pv. |
| | campestris str. CN14 |
| fig\|40324.334.peg 1103 | Stenotrophomonas maltophilia |
| fig\|1349793.3.peg.2529 | Hydrogenophaga taeniospiralis |
| | NBRC 102512 |
| fig\|1842727.3.peg.1491 | Rhodoferax koreense |
| fig\|1619952.3.peg.5158 | Burkholderiaceae bacterium 16 |
| fig\|1970380.3.peg.1914 | Halothiobacillus sp. 14-55-98 |
| fig\|2015568.3.peg.2963 | Burkholderiales bacterium PBB6 |
| fig\|1752215.3.peg.2312 | Gammaproteobacteria bacterium |
| | Ga0077554 |
| fig\|1706231.5.peg 3125 | Janthinobacterium sp. CG23_2 |
| fig\|2013716.3.peg.2169 | Betaproteobacteria bacterium |
| | HGW-Betaproteobacteria-4 |
| fig\|1946997.3.peg.3049 | Nitrospira sp. UBA7655 |
| fig\|765913.3.peg.2527 | Thiorhodococcus drewsii AZ1 |
| fig\|1743159.3.peg.1891 | Polynucleobacter yangtzensis |
| fig\|1597955.3.peg.3923 | Limnohabitans sp. DM1 |
| fig\|1184267.3.peg.1626 | Bdellovibrio exovorus JSS |
| fig\|101571.190.peg.3007 | Burkholderia ubonensis |
| fig\|123899.5.peg.1710 | Bordetella trematum |
| fig\|463035.3.peg.3900 | Bordetella genomosp. 12 |
| fig\|1395608.4.peg.211 | Bordetella genomosp. 5 |
| fig\|1947379.3.peg.2784 | Rhodoferax sp. UBA5149 |
| WP_074294985.1 | Paraburkholderia phenazinium |
| fig\|1324617.3.peg.820 | Paraburkholderia aspalathi |
| fig\|80868.3.peg.3458 | Acidovorax cattleyae |
| fig\|1388764.3.peg.1840 | Pseudogulbenkiania ferrooxidans EGD-HP2 |
| fig\|251747.15.peg.4695 | Chromobacterium subtsugae |
| fig\|670.1020.peg.382 | Vibrio parahaemolyticus |
| fig\|1055803.3.peg.1434 | Pseudoalteromonas sp. TB51 |
| fig\|1201036.3.peg.177 | Pseudochrobactrum sp. AO18b |
| fig\|1220581.4.peg.1434 | Agrobacterium rhizogenes NBRC 13257 |
| fig\|398.6.peg.6695 | Rhizobium tropici |
| fig\|931866.6.peg.8184 | Bradyrhizobium ottawaense |
| fig\|142585.3.peg.1658 | Bradyrhizobium sp. C9 |
| fig\|1082933.13.peg.1537 | Mesorhizobium amorphac CCNWGS0123 |
| fig\|1768789.3.peg.791 | Methylobacterium sp. CCH7-A2 |
| fig\|1381123.3.peg.3819 | Alishoeflea sp. 2WW |
| fig\|1297570.3.peg.1970 | Mesorhizobium sp. STM 4661 |
| fig\|935546.3.peg.3816 | Mesorhizobium loti NZP2037 |
| fig\|1128253.3.peg.1960 | Bradyrhizobium japonicum CCBAU 15354 |
| fig\|1444315.4.peg.3983 | Lysobacter capsici AZ78 |
| fig\|1185327.3.peg.1608 | Xanthomonas axonopodis pv. manihotis |
| | str. Xam668 |
| fig\|1881043.3.peg.2597 | Pseudoxanthomonas sp. GM95 |
| ALN84423.1 | Lysobacter capsici |
| fig\|56460.15.peg.1977 | Xanthomonas vesicatoria |
| fig\|1317116.6.peg.2759 | Oceanicola sp. 22II-s10i |
| fig\|564137.3.peg.4320 | Roseicitreum antarcticum |
| fig\|1952800.3.peg.3583 | Rhodobacteraceae bacterium UBA2553 |
| fig\|218673.12.peg.3041 | Sulfitobacter dubius |
| fig\|1912092.3.peg.2119 | Nioella sediminis |
| fig\|1736558.3.peg.5006 | Ensifer sp. Root558 |
| fig\|91360.5.peg.3717 | Desulforhopalus singaporensis |
| fig\|1948756.3.peg 2576 | Spirochaetia bacterium UBA2205 |
| fig\|1855322.3.peg.103 | Bradyrhizobium sp. Rc3b |
| fig\|1437360.11.peg.2429 | Bradyrhizobium erythrophlei |
| fig\|1871052.3.peg.1026 | Afipia sp. |
| fig\|1038860.3.peg.8756 | Bradyrhizobium elkanii WSM2783 |
| fig\|1898112.54.peg.3758 | Rhodospirillaceae bacterium |
| fig\|1660129.3.peg.4854 | Phenylobacterium sp. SCN 70-31 |
| fig\|1482074.3.peg.4109 | Hartmannibacter diazotrophicus |
| fig\|1970306.3.peg.552 | Acidocella sp. 35-58-6 |
| fig\|1686310.5.peg.1409 | Bartonella apis |
| fig\|1798192.3.peg.1953 | Thalassospira sp. KO164 |
| fig\|1235461.17.peg.11 | Sinorhizobium meliloti GR4 |
| fig\|442.12.peg 222 | Gluconobacter oxydans |
| fig\|1938607.3.peg.1954 | Sphingomonas sp. LM7 |
| fig\|1231624.3.peg.39 | Asaia bogorensis NBRC 16594 |
| fig\|1121271.3.peg.4112 | Gemmobacter nectariphilus DSM 15620 |
| fig\|33059.16.peg.1690 | Acidithiobacillus caldus |
| fig\|502025.10.peg.925 | Haliangium ochraceum DSM 14365 |
| fig\|1734406.3.peg.691 | Alphaproteobacteria bacterium BRH_c36 |
| fig\|1979207.3.peg.4304 | Parvularcula sp. |
| fig\|1953057.3.peg.74 | Parvularculaceae bacterium UBA4496 |
| fig\|858423.3.peg.10004 | Bradyrhizobium arachidis |
| fig\|267128.3.peg.2015 | Sphingopyxis granuli |
| fig\|582667.3.peg.5553 | Methylobacterium pseudosasicola |
| fig\|1187852.3.peg.2712 | Methylobacterium tarhaniae |
| fig\|582675.3.peg.1247 | Methylobacterium gossipiicola |
| fig\|1951640.3.peg.515 | Deferribacteraceae bacterium UBA6799 |
| fig\|1948417.4.peg.1606 | Alphaproteobacteria bacterium UBA6187 |
| fig\|45074.5.peg.981 | Legionella santicrucis |
| fig\|1434232.4.peg.2927 | Magnetofaba australis IT-1 |
| fig\|1945950.3.peg.3568 | Acinetobacter sp. UBA6526 |
| fig\|106654.22.peg.994 | Acinetobacter nosocomialis |
| fig\|1977883.3.peg.3023 | Acinetobacter sp. ANC 3903 |
| fig\|1945948.3.peg.700 | Acinetobacter sp. UBA5984 |
| fig\|1226327.3.peg.2796 | Acinetobacter kookii |
| fig\|1879049.4.peg.5949 | Acinetobacter sp. WCHAc010034 |
| fig\|1945955.3.peg.1951 | Acinetobacter sp. UBA7614 |
| fig\|1675530.3.peg.2149 | Acinetobacter genomosp. 33YU |
| fig\|1310638.3.peg.1006 | Acinetobacter baumannii 1437282 |
| fig\|1400001.4.peg.34 | Necropsobacter massiliensis |
| fig\|1132496.5.peg.136 | Pasteurella multocida subsp. |
| | multocida str. HN06 |
| fig\|1908263.4.peg.2604 | Rodentibacter trehalosifermentans |
| fig\|375432.4.peg.200 | Haemophilus influenzae R3021 |
| fig\|400668.8.peg.3776 | Marinomonas sp. MWYL1 |
| fig\|1913989.193.peg.841 | Gammaproteobacteria bacterium |
| fig\|856793.5.peg.1975 | Micavibrio aeruginosavorus ARL-13 |
| SBW23286.1 | Citrobacter europaeus |
| fig\|1736225.3.peg.985 | Erwinia sp. Leaf53 |
| fig\|29486.12.peg.818 | Yersinia ruckeri |
| fig\|914128.3.peg.2502 | Serratia symbiotica str. Tucson |
| fig\|1796497.3.peg.952 | Grimontia celer |
| fig\|1095649.3.peg.3298 | Vibrio cholerae O1 str. EM-1676A |
| fig\|137584.4.peg.1627 | Thalassomonas viridans |
| fig\|173990.3.peg.1773 | Rheinheimera pacifica |
| fig\|1720343.3.peg.3189 | Pseudoalteromonas sp. 1_2015MBL_MicDiv |
| fig\|1202962.4.peg.1481 | Moritella marina ATCC 15381 |
| fig\|669.50.peg.2993 | Vibrio harveyi |
| fig\|691.32.peg.1517 | Vibrio natriegens |
| fig\|156578.3.peg.2521 | Alteromonadales bacterium TW-7 |
| fig\|661.14.peg.380 | Photobacterium angustum |
| fig\|654.94.peg.1733 | Aeromonas veronii |
| fig\|703.9.peg.319 | Plesiomonas shigelloides |
| fig\|589873.36.peg.1971 | Alteromonas australica |
| fig\|28107.3.peg.3571 | Pseudoalteromonas espejiana |
| fig\|1547444.3.peg.4264 | Pseudoalteromonas sp. PLSV |
| fig\|629266.7.peg.847 | Pseudomonas syringae pv. |
| | actinidiae str. M302091 |
| fig\|251722.19.peg.4059 | Pseudomonas amygdali pv. aesculi |
| fig\|587851.4.peg.1470 | Pseudomonas chlororaphis |
| | subsp. aureofaciens |
| fig\|1265490.3.peg.2330 | Pseudomonas sp. URMO17WK12:I8 |
| fig\|316.101.peg.3534 | Pseudomonas stutzeri |
| fig\|1916993.3.peg.4917 | Pseudomonas putida |
| fig\|1628833.3.peg.2448 | Pseudomonas sp. ES3-33 |
| fig\|1283291.4.peg.1991 | Pseudomonas sp. URMO17WK12:I11 |
| fig\|83963.5.peg.3885 | Pseudomonas syringae pv. papulans |
| fig\|1206777.3.peg.4334 | Pseudomonas sp. Lz4W |
| fig\|113268.3.peg.3785 | Bathymodiolus platifrons |
| | methanotrophic gill symbiont |
| fig\|1131284.3.peg.1562 | zeta proteobacterium SCGC AB-137-C09 |
| fig\|2026807.7.peg.2258 | Zetaproteobacteria bacterium |
| fig\|281689.4.peg.2060 | Desulfuromonas acetoxidans DSM 684 |
| fig\|1188231.4.peg.1200 | Mariprofundus ferrooxydans M34 |
| fig\|1367489.3.peg.682 | Aliivibrio fischeri SA1G |
| fig\|1873135.3.peg.4249 | Shewanella sp. SACH |
| fig\|663.73.peg.2465 | Vibrio alginolyticus |
| fig\|1588629.3.peg.1134 | Aeromonas sp. L_1B5_3 |
| fig\|1121922.3.peg.3454 | Glaciecola pallidula DSM |
| | 14239 = ACAM 615 |
| fig\|351745.9.peg.2506 | Shewanella sp. W3-18-1 |
| fig\|29497.20.peg.3798 | Vibrio splendidus |
| fig\|1367486.3.peg 187 | Aliivibrio fischeri CB37 |
| fig\|511062.4.peg.1890 | Oceanimonas sp. GK1 |
| fig\|654.12.peg.188 | Aeromonas veronii |
| fig\|29497.21.peg.4482 | Vibrio splendidus |
| fig\|1659713.3.peg.560 | Enterobacter bugandensis |
| fig\|1124991.3.peg.3617 | Morganella morganii subsp. morganii KT |
| fig\|104623.3.peg.1381 | Serratia sp. ATCC 39006 |
| fig\|1256989.3.peg.902 | Providencia alcalifaciens R90-1475 |
| fig\|1125694.3.peg.1143 | Proteus mirabilis WGLW6 |
| fig\|574096.6.peg.2693 | Pantoea allif |
| fig\|1095774.3.peg.2623 | Pantoca ananatis PA13 |
| fig\|869692.4.peg.2910 | Escherichia coli 3003 |
| WP_140159440.1 | Escherichia coli |
| fig\|550.437.peg.1444 | Enterobacter cloacae |
| fig\|573.13605.peg.2600 | Klebsiella pneumoniae |
| fig\|550.285.peg.3783 | Enterobacter cloacae |
| fig\|1265672.3.peg.3869 | Salmonella enterica subsp. enterica |
| | serovar Agona str. 70.E.05 |
| fig\|573.10028.peg.542 | Klebsiella pneumoniae |
| fig\|749537.3.peg.218 | Escherichia coli MS 115-1 |
| ANK06786.1 | Escherichia coli O25b:H4 |
| fig\|670.880.peg.975 | Vibrio parahaemolyticus |
| fig\|1192730.4.peg.3 | Salmonella enterica subsp. enterica |
| | serovar Kintambo |
| fig\|1224144.4.peg.4030 | Dickeya sp. CSL RW240 |
| fig\|568766.10.peg.2937 | Dickeya sp. NCPPB 3274 |
| fig\|1076549.3.peg.4260 | Pantoea rodasii |
| fig\|548.102.peg.3401 | Klebsiella acrogenes |
| fig\|630.90.peg.1795 | Yersinia enterocolitica |
| fig\|79883.5.peg.266 | Bacillus horikoshii |
| fig\|180861.3.peg.3762 | Bacillus thuringiensis serovar sumiyoshiensis |
| fig\|1390.157.peg.339 | Bacillus amyloliquefaciens |
| fig\|293386.15.peg.304 | Bacillus stratosphericus |
| fig\|1053181.3.peg 3820 | Bacillus cereus BAG2X1-3 |
| fig\|1884375.3.peg.681 | Paemibacillus sp. PDC88 |
| fig\|334735.5.peg.923 | Sporosarcina korcensis |
| fig\|79884.3.peg.1120 | Bacillus pseudalcaliphilus |
| fig\|1628206.3.peg.4802 | Bacillus sp. LK2 |
| fig\|1396.1605.peg.6235 | Bacillus cereus |
| fig\|182710.3.peg.317 | Oceanobacillus iheyensis |
| fig\|860.10.peg.486 | Fusobacterium periodonticum |
| fig\|1855308.3.peg.1467 | Trichococcus ilyis |
| fig\|931626.3.peg.151 | Acetobacterium woodii DSM 1030 |
| fig\|1965575.3.peg 2547 | Lachnoclostridium sp. An181 |
| fig\|1352.2757.peg.71 | Enterococcus faecium |
| fig\|1299895.3.peg.900 | Listeria monocytogenes CFSAN002349 |
| fig\|53346.29.peg.1591 | Enterococcus mundtii |
| fig\|1649188.10.peg.1545 | Listeria goaensis |
| fig\|158847.6.peg.432 | Megamonas hypermegale |
| fig\|1121289.3.peg.2775 | Clostridiisalibacter paucivorans DSM 22131 |
| fig\|1950885.3.peg.858 | Clostridiales bacterium UBA4693 |
| fig\|1965576.3.peg.1978 | Pseudoflavonifractor sp. An184 |
| fig\|1952416.3.peg.1629 | Ruminococcaceae bacterium UBA642 |
| fig\|1262803.3.peg.8 | Clostridium sp. CAG:413 |
| fig\|28037.216.peg.60 | Streptococcus mitis |
| fig\|1074052.3.peg.33 | Streptococcus sobrinus TCI-9 |
| fig\|1304.207.peg.1536 | Streptococcus salivarius |
| fig\|1154859.3.peg.955 | Streptococcus agalactiae LMG 14609 |
| fig\|1080071.3.peg.332 | Streptococcus orisasini |
| fig\|1139219.3.peg.2194 | Enterococcus dispar ATCC 51266 |
| fig\|1834176.3.peg.811 | Enterococcus sp. 3G1_DIV0629 |
| fig\|1622.15.peg.947 | Lactobacillus murinus |
| fig\|565651.6.peg.1942 | Enterococcus faecalis ARO1/DG |
| fig\|1473546.3.peg.703 | Lysinibacillus sp. BF-4 |
| fig\|37734.13.peg.137 | Enterococcus casseliflavus |
| fig\|492670.92.peg.623 | Bacillus velezensis |
| fig\|1639.1907.peg.2641 | Listeria monocytogenes |
| fig\|1123489.3.peg.170 | Veillonella magna DSM 19857 |
| fig\|1280687.3.peg.1880 | Butyrivibrio fibrisolvens YRB2005 |
| fig\|1262889.3.peg.680 | Eubacterium sp. CAG:38 |
| fig\|1235800.3.peg.2226 | Lachnospiraceae bacterium 10-1 |
| fig\|1897035.3.peg.445 | Firmicutes bacterium CAG:552_39_19 |
| fig\|199.588.peg.774 | Campylobacter concisus |
| fig\|1111133.4.peg.219 | Peptoniphilus sp. BV3AC2 |
| fig\|936589.3.peg.875 | Veillonella sp. AS16 |
| WP_070600378.1 | |
| fig\|1896998.3.peg.1750 | Coprococcus sp. CAG: 131-related_45_246 |
| fig\|41170.3.peg.3013 | Exiguobacterium acetylicum |
| fig\|59620.44.peg.897 | uncultured Clostridium sp. |
| fig\|1262843.3.peg.313 | Clostridium sp. CAG:813 |
| fig\|1262834.3.peg.1287 | Clostridium sp. CAG:715 |
| fig\|1256219.3.peg.760 | Lactobacillus paracasei subsp. |
| | paracasei Lpp230 |
| fig\|115778.31.peg 1994 | Leuconostoc gelidum subsp. gasicomitatum |
| fig\|29385.174.peg.531 | Staphylococcus saprophyticus |
| fig\|1295.21.peg.75 | Staphylococcus schleiferi |
| fig\|148814.13.peg.1360 | Lactobacillus kunkeei |
| fig\|1282.1242.peg.673 | Staphylococcus epidermidis |
| fig\|1581078.3.peg.1186 | Staphylococcus sp. HMSC10C03 |
| fig\|1891097.3.peg.280 | Macrococcus goetzii |
| WP_080703103.1 | |
| fig\|1214184.3.peg.1129 | Streptococcus suis 22083 |
| fig\|1154771.3.peg.209 | Streptococcus agalactiae FSL C1-487 |
| fig\|1415765.3.peg.1578 | Streptococcus mitis 21/39 |
| fig\|1581074.3.peg.720 | Granulicatella sp. HMSC31F03 |
| fig\|1349.233.peg.712 | Streptococcus uberis |
| fig\|1946281.3.peg.392 | Catabacter sp. UBA5893 |
| fig\|1328309.5.peg.1889 | Lactobacillus plantarum IPLA88 |
| fig\|1214190.3.peg.2034 | Streptococcus suis YS17 |
| fig\|29385.135.peg.2098 | Staphylococcus saprophyticus |
| fig\|1715184.3.peg.1265 | Aerococcus sp. HMSC035B07 |
| fig\|1881068.3.peg.2940 | Sphingomonas sp. OV641 |
| fig\|1522072.3.peg.3829 | Sphingobium sp. ba1 |
| fig\|1802172.3.peg.237 | Sphingopyxis sp. |
| | RIFCSPHIGHO2_12_FULL_65_19 |
| fig\|1128204.3.peg.2189 | Bradyrhizobium elkanji CCBAU 43297 |
| fig\|1708715.5.peg.4517 | Ensifer aridi |
| fig\|195105.3.peg.2062 | Haematobacter massiliensis |
| fig\|1283312.3.peg.4182 | Sphingomonas wittichii DC-6 |
| fig\|1120654.4.peg.406 | Ensifer sp. LC499 |
| fig\|529.36.peg.3144 | Ochrobactrum anthropi |
| fig\|1194716.3.peg.4774 | Sinorhizobium meliloti AK75 |
| fig\|1660088.4.peg.2967 | Agrobacterium sp. SCN 61-19 |
| fig\|1951259.3.peg.2515 | Sphingomonadales bacterium UBA6174 |
| fig\|1912891.5.peg.2102 | Sphingobium sp. |
| fig\|1670800.3.peg.1844 | Mesorhizobium oceanicum |
| fig\|2032658.3.peg.157 | Alphaproteobacteria bacterium WMHbin7 |
| fig\|1819565.5.peg.2208 | Flavimaricola marinus |
| fig\|1245469.3.peg.1160 | Bradyrhizobium oligotrophicum S58 |
| fig\|1615890.4.peg.173 | Bradyrhizobium sp. LTSP849 |
| fig\|56454.3.peg.3464 | Xanthomonas hortonmm |
| fig\|40324.384.peg.1060 | Stenotrophomonas maltophilia |
| fig\|1801972.3.peg.1832 | Planctomycetes bacterium |
| | RBG_19FT_COMBO_48_8 |
| fig\|1978765.3.peg.3488 | Nitrospira sp. ST-bin5 |
| fig\|2009322.3.peg.2770 | Leptolyngbya ohadii IS1 |
| fig\|1325564.3.peg.3733 | Nitrospira japonica |
| fig\|43662.9.peg.1688 | Pseudoalteromonas piscicida |
| fig\|670.134.peg.4439 | Vibrio parahaemolyticus |
| fig\|998520.3.peg.3325 | Pseudoalteromonas agarivorans |
| fig\|1723759.3.peg.401 | Pseudoalteromonas sp. P1-26 |
| fig\|672.133.peg.585 | Vibrio vulnificus |
| fig\|1324960.19.peg.585 | Aeromonas salmonicida subsp. |
| | pectinolytica 34mel |
| fig\|196024.16.peg.3965 | Aeromonas dhakensis |
| fig\|654.27.peg.4266 | Aeromonas veronii |
| fig\|1802253.3.peg.1045 | Sulfurimonas sp. RIFCSPLOWO2_12_36_12 |
| fig\|636.16.peg.3905 | Edwardsiella tarda |
| fig\|1124958.3.peg.5012 | Salmonella enterica subsp. enterica |
| | serovar Muenster str. 0315 |
| fig\|573.10007.peg.225 | Klebsiella pneumoniae |
| fig\|1946737.3.peg.4002 | Leclercia sp. UBA1284 |
| fig\|1398203.3.peg.3712 | Xenorhabdus bovienii str. kraussei Quebec |
| fig\|615.247.peg.2151 | Serratia marcescens |
| fig\|52441.3.peg.3752 | Nitrosomonas aestuarii |
| fig\|1951948.3.peg.242 | Hyphomonadaceae bacterium UBA2389 |
| fig\|165186.29.peg.27 | uncultured Ruminococcus sp. |
| fig\|2013842.3.peg.1881 | Synergistetes bacterium HGW-Synergistetes-1 |
| fig\|411484.7.peg.436 | Clostridium sp. SS2/1 |
| fig\|460384.4.peg.447 | Enterocloster lavalensis |
| fig\|1761781.3.peg.2961 | Clostridium sp. DSM 8431 |
| fig\|1451.25.peg.614 | Paenibacillus amylolyticus |
| fig\|1776378.3.peg.2009 | Paenibacillus phocaensis |
| fig\|1866315.3.peg.2122 | Bacillus sp. N35-10-4 |
| fig\|1034836.4.peg.4077 | Bacillus amyloliquefaciens XH7 |
| fig\|1397.14.peg.5097 | Bacillus circulans |
| fig\|1497681.5.peg.772 | Listeria newyorkensis |
| fig\|1053224.3.peg.4333 | Bacillus cereus VD021 |
| fig\|1374.4.peg.2798 | Planococcus kocunii |
| fig\|458233.11.peg.419 | Macrococcus caseolyticus JCSC5402 |
| fig\|417368.6.peg.944 | Enterococcus thailandicus |
| fig\|1353.16.peg.736 | Enterococcus gallinarum |
| fig\|1639.1307.peg.2578 | Listeria monocytogenes |
| fig\|1649188.4.peg.450 | Listeria goaensis |
| fig\|333990.5.peg.1279 | Carobacterium sp. AT7 |
| fig\|1121085.3.peg.4805 | Bacillus aidingensis DSM 18341 |
| fig\|659243.6.peg.1163 | Bacillus siamensis |
| fig\|1965645.3.peg.1428 | Alistipes sp. An54 |
| fig\|1950664.3.peg.363 | Bacteroidales bacterium UBA5918 |
| fig\|681398.3.peg.1596 | Paludibacter jiangxiensis |
| fig\|1947481.3.peg.1596 | Sphingobacterium sp. UBA1498 |
| fig\|1946424.3.peg.2345 | Dysgonomonas sp. UBA4861 |
| fig\|188932.3.peg.968 | Pedobacter cryoconitis |
| fig\|505249.7.peg.1802 | Arcobacter marinus |
| fig\|1802259.3.peg.374 | Sulfurimonas sp. RIFOXYD12_FULL_33_39 |
| fig\|1872629.13.peg.663 | Arcobacter sp. |
| fig\|497650.4.peg.949 | Sulfurovum sp. enrichment culture clone C5 |
| fig\|1981711.3.peg.707 | Pseudomonas sp. B8(2017) |
| fig\|287.926.peg.3808 | Pseudomonas aeruginosa |
| fig\|157782.3.peg.183 | Pseudomonas parafulva |
| fig\|1225174.5.peg.576 | Pseudomonas mendocina S5.2 |
| fig\|237610.8.peg.4301 | Pseudomonas psychrotolerans |
| fig\|1116369.3.peg.182 | Hoeflea sp. 108 |
| WP_080858354.1 | |
| fig\|1679460.3.peg.2715 | Marinibacterium profundimaris |
| fig\|1811547.3.peg.510 | Maritimibacter sp. REDSEA-S28_B5 |
| fig\|93684.8.peg.518 | Roseivivax halotolerans |
| EMZ69714.1 | Escherichia coli 174900 |
| fig\|103796.87.peg.3165 | Pseudomonas syringae pv. actinidiae |
| WP_078828851.1 | Pantoea ananatis |
| fig\|2018067.3.peg.1734 | Pseudomonas sp. FDAARGOS_380 |
| fig\|294.255.peg.5151 | Pseudomonas fluorescens |
| fig\|287.4271.peg.5445 | Pseudomonas aeruginosa |
| fig\|46677.3.peg.3237 | Pseudomonas agarici |
| fig\|83964.10.peg.849 | Pseudomonas coronafaciens pv. porri |
| fig\|1932113.4.peg.2793 | Pseudomonas sp. PA1(2017) |
| fig\|1712677.3.peg.189 | Pseudomonas sp. 2822-15 |
| fig\|1479235.3.peg.2741 | Halomonas sp. HL-48 |
| fig\|227946.12.peg.3247 | Xanthomonas translucens pv. poae |
| fig\|40324.220.peg.2801 | Stenotrophomonas maltophilia |
| fig\|227946.13.peg.35 | Xanthomonas translucens pv. poae |
| fig\|487909.15.peg.4212 | Xanthomonas translucens pv. undulosa |
| fig\|40324.145.peg.2120 | Stenotrophomonas maltophilia |
| fig\|1182783.3.peg.8 | Xanthomonas campestris JX |
| fig\|1736581.3.peg.4144 | Lysobacter sp. Root667 |
| fig\|470.4256.peg.2128 | Acinetobacter baumanmii |
| fig\|1804984.3.peg.4735 | Burkholderia sp. OLGA172 |
| fig\|1882792.3.peg.5959 | Burkholderia sp. CF145 |
| fig\|1458357.5.peg.7849 | Caballeronia jiangsuensis |
| fig\|674703.3.peg.3992 | Rhodoplanes sp. Z2-YC6860 |
| fig\|1230476.3.peg.595 | Bradyrhizobium sp. DFCI-1 |
| fig\|1752222.3.peg.1730 | Rhizobiales bacterium Ga0077525 |
| fig\|1948848.3.peg 320 | Patescibacteria group bacterium UBA6220 |
| fig\|1860092.3.peg.3966 | Alphaproteobacteria bacterium |
| | MedPE-SWcel |
| fig\|398.7.peg.3301 | Rhizobium tropici |
| fig\|418630.3.peg.960 | Rhodobacter megalophilus |
| fig\|56.40.peg.5712 | Sorangium cellulosum |
| fig\|1660160.3.peg.2510 | Acidobacteria bacterium SCN 69-37 |
| fig\|1661042.3.peg.2224 | Pseudomonas sp. NBRC 111127 |
| fig\|1712678.3.peg.4198 | Pseudomonas sp. 2822-17 |
| fig\|1736561.3.peg.128 | Pseudomonas sp. Root562 |
| fig\|76760.8.peg.1730 | Pseudomonas rhodesiae |
| fig\|1295133.4.peg.7170 | Pseudomonas putida JCM 18798 |
| fig\|1718917.3.peg.3132 | Pseudomonas sp. ICMP 460 |
| fig\|237306.3.peg.591 | Pseudomonas syringae pv. persicae |
| fig\|1079060.3.peg.1479 | Pseudomonas savastanoi pv. |
| | phaseolicola 1644R |
| fig\|1981714.3.peg.1068 | Pseudomonas sp. B5(2017) |
| fig\|1419583.3.peg.4516 | Pseudomonas mandelli PD30 |
| fig\|1718918.3.peg.4166 | Pseudomonas sp. ICMP 561 |
| fig\|64988.7.peg.76 | Alcanivorax jadensis |
| fig\|1961564.3.peg.685 | Desulfovibrionaceae bacterium UBA5546 |
| fig\|2004648.3.peg.1747 | Acinetobacter sp. WCHA39 |
| fig\|1080187.3.peg.399 | Cupriavidus sp. UYPR2.512 |
| fig\|76114.8.peg.258 | Aromatoleum aromaticum EbN1 |
| fig\|196367.9.peg.6286 | Caballeronia sordidicola |
| fig\|1217418.3.peg.694 | Cupriavidus sp. HPC(L) |
| fig\|1752216.3.peg.4007 | Nitrosomonadales bacterium Ga0074132 |
| fig\|1249621.3.peg.3614 | Cupriavidus sp. HMR-1 |
| fig\|179879.8.peg.6514 | Burkholderia anthina |
| fig\|1246301.3.peg.4482 | Variovorax paradoxus B4 |
| WP_092746164.1 | Acidovorax valerianellae |
| fig\|536.30.peg.857 | Chromobacterium violaceum |
| fig\|1961112.3.peg.115 | Planctomycetes bacterium UTPLA1 |
| figj44574.5.peg.4575 | Nitrosomonas communis |
| fig\|265901.4.peg.190 | Photobacterium sp. J15 |
| fig\|80852.21.peg.1289 | Aliivibrio wodanis |
| fig\|1136159.3.peg.2534 | Vibrio cyclitrophicus 1F111 |
| fig\|24.6.peg.4539 | Shewanella putrefaciens |
| fig\|888433.3.peg.1974 | Pseudoalteromonas sp. GutCa3 |
| fig\|196024.6.peg.3651 | Aeromonas dhakensis |
| fig\|1352943.3.peg.5028 | Vibrio harveyi E385 |
| WP_088124663.1 | Vibrio cholerae |
| fig\|29497.15.peg.1220 | Vibrio splendidus |
| fig\|670.413.peg.1227 | Vibrio parahaemolyticus |
| fig\|584.91.peg.3337 | Proteus mirabilis |
| fig\|263819.5.peg.201 | Yersinia aleksiciae |
| fig\|1656094.3.peg.1449 | Alteromonas confluentis |
| fig\|634.5.peg.607 | Yersinia bercovieri |
| fig\|630.85.peg.4080 | Yersinia enterocolitica |
| fig\|1212491.3.peg.1847 | Legionella fallonii LLAP-10 |
| fig\|1498499.3.peg.2812 | Legionella norrlandica |
| fig\|1844092.4.peg.3143 | Pseudomonas sp. 8 R. 14 |
| fig\|1441629.3.peg.2119 | Pseudomonas cichorii JBC1 |
| WP_092369835.1 | Pseudomonas seleniipraecipitans |
| fig\|477228.3.peg.2040 | Pseudomonas stutzeri TS44 |
| fig\|317.249.peg.4299 | Pseudomonas syringae |
| fig\|1597.16.peg.2055 | Lactobacillus paracasei |
| fig\|1184720.6.peg.2708 | Rhizobium anhuiense |
| fig\|1566263.3.peg.185 | Rhizobium sp. NFR03 |
| fig\|1951216.3.peg.1622 | Rhizobiales bacterium UBA1909 |
| fig\|1219052.3.peg.3572 | Sphingomonas pruni NBRC 15498 |
| fig\|376620.8.peg.122 | Gluconobacter japonicus |
| fig\|1736587.3.peg.2638 | Devosia sp. Root685 |
| WP_011269850.1 | Xanthomonas campestris |
| fig\|1195246.3.peg.467 | Alishewanella agri BL06 |
| fig\|1931276.3.peg.1294 | Haliangium sp. UPWRP_2 |
| fig\|1931204.4.peg.12 | Confluentimicrobium sp. |
| fig\|2052957.3.peg.3497 | Pseudorhodobacter sp. MZDSW-24AT |
| fig\|1952825.3.peg.2772 | Rhodobiaceae bacterium UBA4205 |
| fig\|1189622.3.peg.1716 | Pseudomonas amygdali pv. tabaci str. 6605 |
| fig\|294.173.peg.2741 | Pseudomonas fluorescens |
| fig\|294.122.peg.3307 | Pseudomonas fluorescens |
| fig\|1198456.3.peg.4053 | Pseudomonas guguanensis |
| fig\|1855380.3.peg.1951 | Pseudomonas sp. Z003-0.4C(8344-21) |
| fig\|1144330.3.peg.3879 | Pseudomonas sp. GM48 |
| fig\|86265.3.peg.2799 | Pseudomonas thivervalensis |
| fig\|1881035.3.peg.3817 | Mitsuaria sp. PDC51 |
| fig\|511.8.peg.774 | Alcaligenes faecalis |
| fig\|1095552.3.peg.2955 | Methylobacter luteus IMV-B-3098 |
| fig\|1690268.3.peg.1172 | Acidovorax sp. SD340 |
| fig\|871652.3.peg.1451 | Poseidonocella sedimentorum |
| fig\|1946868.3.peg.175 | Methylophaga sp. UBA1490 |
| fig\|1924940.3.peg.1147 | Mameliella sp. |
| fig\|1912891.7.peg.5370 | Sphingobium sp. |
| fig\|1236503.3.peg.1539 | Acetobacter persici JCM 25330 |
| fig\|1745182.3.peg.1942 | Paracoccus sp. MKU1 |
| fig\|1112.5.peg.2875 | Porphyrobacter neustonensis |
| WP_051585410.1 | Sphingomonas paucimobilis |
| fig\|1082931.4.peg.3584. | Pelagibacterium halotolerans B2 |
| fig\|1907665.3.peg 5475 | Agrobacterium sp. DSM 25558 |
| fig\|1841652.4.peg.3782 | Agrobacterium sp. 13-626 |
| fig\|1736312.3.peg.3441 | Rhizobium sp. Leaf262 |
| fig\|1768770.3.peg.4687 | Caulobacter sp. CCH5-E12 |
| fig\|355591.9.peg.1867 | Marinobacter vinifirmus |
| fig\|1869214.4.peg.1848 | Rheinheimera sp. |
| fig\|1946470.3.peg.3546 | Erythrobacter sp. UBA2510 |
| fig\|1860090.3.peg.231 | Roseobacter sp. MedPE-SWde |
| fig\|2020902.8.peg.1814 | Ponticaulis sp. |
| fig\|940286.3.peg.3612 | Komagataeibacter oboediens 174Bp2 |
| fig\|1736380.3.peg.1842 | Rhizobium sp. Leaf453 |
| fig\|665126.3.peg.2283 | Prosthecomicrobium hirschii |
| fig\|2029410.3.peg.1956 | Mesorhizobium sp. WSM4311 |
| WP_003169203.1 | Brevundimonas diminuta |
| fig\|1884373.3.peg.3317 | Mesorhizobium sp. YR577 |
| fig\|989436.3.peg.3203 | Pseudovibrio sp. Ad5 |
| fig\|1736359.3.peg.3976 | Rhizobium sp. Leaf386 |
| fig\|104102.12.peg.3797 | Acetobacter tropicalis |
| fig\|1500305.3.peg.4736 | Rhizobium sp. OK665 |
| fig\|1842535.30.peg.6 | Blastomonas sp. RAC04 |
| fig\|70775.16.peg.91 | Pseudomonas plecoglossicida |
| fig\|287.4262.peg.2063 | Pseudomonas aeruginosa |
| WP_017702484.1 | Pseudomonas syringae |
| fig\|1357292.3.peg.4700 | Pseudomonas syringae pv. pisi str. PP1 |
| fig\|287.2436.peg.3554 | Pseudomonas aeruginosa |
| fig\|76758.3.peg.4722 | Pseudomonas orientalis |
| fig\|1904755.3.peg.3469 | Pseudomonas sp. 43NM1 |
| fig\|47879.37.peg.693 | Pseudomonas corrugata |
| fig\|1771311.3.peg.1935 | Pseudomonas sp. ATCC PTA-122608 |
| fig\|2008975.3.peg.1976 | Pseudomonas sp. Irchel 3E13 |
| fig\|1259798.3.peg.1121 | Pseudomonas sp. LAMO17WK12:12 |
| fig\|1736487.3.peg.2103 | Noviherbaspirillum sp. Root189 |
| fig\|1706231.5.peg.1557 | Janthinobacterium sp. CG23_2 |
| fig\|1804984.3.peg.4700 | Burkholderia sp. OLGA172 |
| fig\|54067.3.peg.2925 | Xylophilus ampelinus |
| fig\|40324.357.peg.1426 | Stenotrophomonas maltophilia |
| fig\|1967657.4.peg.913 | Salmonella enterica subsp. enterica |
| | serovar Telelkebir |
| fig\|573.14330.peg.816 | Klebsiella pneumoniae |
| fig\|615.357.peg.16 | Serratia marcescens |
| fig\|1122616.3.peg.231 | Oceanospirillum beijerinckii DSM 7166 |
| fig\|314276.4.peg.1389 | Idiomarina baltica OS145 |
| fig\|1038921.4.peg.2790 | Pseudomonas chlororaphis subsp. |
| | aureofaciens 30-84 |
| fig\|292.72.peg.3280 | Burkholderia cepacia |
| fig\|1899355.18.peg.947 | Oceanospirillaceae bacterium |
| fig\|2015356.3.peg.5401 | Burkholderia sp. AU33647 |
| fig\|206665.3.peg.1731 | Desulfonauticus submarinos |
| fig\|1987165.3.peg.2564 | Sphingobium sp. GW456-12-10-14-TSB1 |
| fig\|1283312.3.peg.2182 | Sphingomonas wittichii DC-6 |
| fig\|1223566.3.peg.1810 | Bradyrhizobium sp. CCGE-LA001 |
| fig\|76761.16.peg.744 | Pseudomonas veronii |
| PIY00499 1 | Hydrogenophilales bacterium |
| | CG_4_10_14_3_um_filter_58_23 |
| fig\|305.94.peg.4778 | Ralstonia solanacearum |
| fig\|1758178.5.peg.2546 | Celeribacter ethanolicus |
| fig\|1354263.4.peg.2524 | Hafnia paralvei ATCC 29927 |
| fig\|1125979.3.peg.1941 | Rhizobium sp. PDO1-076 |
| fig\|1338032.3.peg.3393 | Vibrio parahaemolyticus O1:K33 |
| | str. CDC_K4557 |
| fig\|1898112.54.peg.3344 | Rhodospirillaceae bacterium |
| fig\|1432558.3.peg.4265 | Klebsiella pneumoniae ISC21 |
| fig\|333962.3.peg.2767 | Providencia heimbachae |
| fig\|60552.10.peg.2414 | Burkholderia vietnamiensis |
| WP_011808964.1 | Verminephrobacter eiseniae |
| fig\|1844107.4.peg.2966 | Pseudomonas sp. 58 R 12 |
| fig\|1952916.3.peg.906 | Synergistaceae bacterium UBA5549 |
| fig\|458817.8.peg.542 | Shewanella halifaxensis HAW-EB4 |
| fig\|1674859.3.peg.1291 | Spirochaetales bacterium Spiro_03 |
| fig\|1121434.3.peg.22 | Halodesulfovibrio aestuarii DSM 10141 |
| fig\|1262899.3.peg.286 | Fusobacterium sp. CAG:439 |
| fig\|57320.3.peg.1084 | Pseudodesulfovibrio profundus |
| fig\|1736444.3.peg.3753 | Acinetobacter sp. Root1280 |
| fig\|1310670.3.peg.2122 | Acinetobacter sp. 907131 |
| fig\|505345.6.peg.150 | Gallibacterium genomosp. 3 |
| fig\|670.887.peg.4391 | Vibrio parahaemolyticus |
| fig\|196024.16.peg.2750 | Aeromonas dhakensis |
| fig\|663.48.peg.132] | Vibrio alginolyticus |
| fig\|28141.133.peg.4717 | Cronobacter sakazakii |
| fig\|1117315.3.peg.3 | Pseudoalteromonas haloplanktis ATCC 14393 |
| fig\|1917164.4.peg.2739 | Shewanella sp. UCD-KL21 |
| fig\|2006083.3.peg.3222 | Photobacterium sp. CECT 9192 |
| fig\|584.227.peg.2146 | Proteus mirabilis |
| fig\|1792834.4.peg.1793 | Marinicella sediminis |
| fig\|1333513.3.peg 3775 | Pseudoalteromonas haloplanktis TAE56 |
| fig\|1305826.3.peg.1246 | Streptomyces sp. Amel2xC10 |
| WP_048809063.1 | Microbacterium ginsengisoli |
| fig\|1987376.3.peg.4246 | Pseudonocardia sp. N23 |
| fig\|164115.3.peg.6832 | Streptomyces niveiscabiei |
| fig\|285676.33.peg.4994 | Micromonospora saelicesensis |
| SFF52649.1 | Streptomyces alni |
| fig\|1100822.3.peg.6408 | Streptomyces sp. AmelKG-E11A |
| WP_098467790.1 | |
| fig\|1190417.3.peg.2916 | Geodermatophilus telluris |
| SDS16714.1 | Agrococcus carbonis |
| fig\|692370.5.peg.1108 | Altererythrobacter dongtanensis |
| fig\|1736370.3.peg.1383 | Sphingomonas sp. Leaf412 |
| fig\|1759074.3.peg.2615 | Sphingopyxis sp. HIX |
| fig\|1120928.3.peg.922 | Acinetobacter tjernbergiae |
| | DSM 14971 = CIP 107465 |
| fig\|470.4268.peg.2032 | Acinetobacter baumannii |
| fig\|1217627.3.peg.995 | Acinetobacter baumannii NIPH 67 |
| fig\|28450.149.peg.5786 | Burkholderia pseudomallei |
| fig\|2032650.3.peg.3543 | Magnetococcales bacterium HCHbin5 |
| fig\|101571.169.peg.3909 | Burkholderia ubonensis |
| fig\|396597.7.peg.2128 | Burkholderia ambifaria MEX-5 |
| fig\|869212.3.peg.3514 | Tarneriella parva DSM 21527 |
| fig\|1196083.80.peg.1885 | Snodgrassella alvi |
| fig\|1304886.3.peg.1257 | Desulfotignum balticum DSM 7044 |
| fig\|555.16.peg.1362 | Pectobacterium carotovorum |
| | subsp. carotovorum |
| fig\|1421338.3.peg.151 | Enterobacter asburiae L1 |
| fig\|443144.3.peg.785 | Geobacter sp. M21 |
| fig\|1265503.3.peg.1271 | Colwellia piezophila ATCC BAA-637 |
| fig\|55601.100.peg.1601 | Vibrio anguillarum |
| fig\|299766.9.peg.4890 | Enterobacter hormaechei subsp. steigerwaltii |
| fig\|243231.5.peg.1360 | Geobacter sulfurreducens PCA |
| fig\|1263083.3.peg.558 | Klebsiella variicola CAG:634 |
| fig\|57706.9.peg.1106 | Citrobacter braakii |
| fig\|1619244.3.peg 1323 | Enterobacter bugandensis |
| SHO56340.1 | Vibrio quintilis |
| fig\|688.15.peg.906 | Aliivibrio logei |
| fig\|663.75.peg.4800 | Vibrio alginolyticus |
| fig\|1967612.3.peg.4508 | Salmonella enterica subsp. |
| | houtenae serovar 50:24, z23:- |
| fig\|82985.3.peg.3508 | Pragia fontium |
| fig\|55207.5.peg.1840 | Pectobacterium betavasculorum |
| fig\|582.25.peg.388 | Morganella morganii |
| fig\|1006598.5.peg.80 | Serratia plymuthica RVH1 |
| fig\|82977.3.peg.3268 | Buttiauxella agrestis |
| CRY53703.1 | Yersinia intermedia |
| fig\|595494.3.peg.706 | Tolumonas auensis DSM 9187 |
| fig\|1217694.3.peg 3300 | Acinetobacter sp. CIP 64.2 |
| fig\|470.2679.peg.715 | Acinetobacter baumannii |
| fig\|1879050.4.peg.2797 | Acinetobacter wuhouensis |
| fig\|2004650.3.peg.1818 | Acinetobacter chinensis |
| fig\|648.80.peg.1405 | Aeromonas caviae |
| fig\|1217722.3.peg.1866 | Pseudomonas sp. S13.1.2 |
| fig\|294.88.peg.4234 | Pseudomonas fluorescens |
| fig\|629262.5.peg.1917 | Pseudomonas syringae pv. |
| | japonica str. M301072 |
| WP_053932309.1 | Pseudomonas coronafaciens |
| fig\|1844101.3.peg.4702 | Pseudomonas sp. 31 R 17 |
| fig\|380021.13.peg.6149 | Pseudomonas protegens |
| fig\|287.3716.peg.2163 | Pseudomonas aeruginosa |
| fig\|287.3208.peg.1464 | Pseudomonas aeruginosa |
| fig\|1952221.3.peg.555 | Methylococcaceae bacterium UBA2780 |
| fig\|1869214.3.peg.3542 | Rheinheimera sp. |
| fig\|375286.7.peg.830 | Janthinobacterium sp. Marseille |
| fig\|536.26.peg.4616 | Chromobacterium violaceum |
| fig\|983548.3.peg.2977 | Dokdonia sp. 4H-3-7-5 |
| fig\|307480.5.peg.1780 | Chryscobacterium vrystaatense |
| fig\|1262921.3.peg.2213 | Prevotella sp. CAG:1185 |
| fig\|1965649.3.peg.4193 | Butyricimonas sp. An62 |
| fig\|1951558.3.peg.3731 | Chitinophagaceae bacterium UBA4411 |
| fig\|1950669.3.peg.2035 | Bacteroidales bacterium UBA6192 |
| fig\|1869230.3.peg.3025 | Chry seobacterium sp. CBo1 |
| fig\|1500294.3.peg.2814 | Chryscobacterium sp. YR485 |
| fig\|1756149.11.peg.2545 | Elizabethkingia bruuniana |
| fig\|1137281.3.peg.1436 | Xanthomarina gelatinilytica |
| fig\|1964365.5.peg.2525 | Sneathiella sp. |
| fig\|28450.428.peg.5049 | Burkholderia pseudomallei |
| fig\|1628751.3.peg.813 | Nostoc linckia z16 |
| fig\|60137.10.peg.1138 | Sulfitobacter pontiacus |
| fig\|1580596.3.peg.2701 | Phaeobacter piscinae |
| fig\|1041141.4.peg.4741 | Rhizobium leguminosarum |
| | bv. viciae 128C53 |
| WP_063290764.1 | unclassified Pseudovibrio |
| fig\|1816219.4.peg.1873 | Colwellia sp. PAMC 21821 |
| fig\|651.3.peg 13 | Aeromonas media |
| fig\|134375.17.peg.3222 | Achromobacter sp. |
| WP_011296194.1 | Cupriavidus pinatubonensis |
| fig\|1513890.4.peg.2322 | Pseudomonas chlororaphis subsp. piscium |
| fig\|294.193.peg.980 | Pseudomonas fluorescens |
| fig\|287.2516.peg.114 | Pseudomonas aeruginosa |
| fig\|2006083.3.peg.3219 | Photobacterium sp. CECT 9192 |
| fig\|458817.8.peg.538 | Shewanella halifaxensis HAW-EB4 |
| fig\|1073383.3.peg.1289 | Aeromonas veronii AMC34 |
| fig\|190893.14.peg.2110 | Vibrio coralliilyticus |
| fig\|663.144.peg.2659 | Vibrio alginolyticus |
| fig\|55601.106.peg.926 | Vibrio anguillarum |
| fig\|669.51.peg.5531 | Vibrio harveyi |
| fig\|1250059.5.peg.3511 | Tenacibaculum sp. MAR_2009_124 |
| fig\|906888.6.peg.2449 | Nonlabens ulvanivorans |
| WP_042276051.1 | Nonlabens sediminis |
| fig\|1953167.3.peg.1254 | Bacteroidetes bacterium UBA6221 |
| fig\|991.14.peg.629 | Flavobacterium hydatis |
| fig\|1121890.3.peg.5 | Flavobacterium frigidarium DSM 17623 |
| fig\|387094.4.peg.1115 | Flavobacterium hercynium |
| fig\|1946558.3.peg 2413 | Flavobacterium sp. UBA7665 |
| fig\|253.27.peg.2882 | Chryseobacterium indologenes |
| fig\|1685010.5.peg.4376 | Chryseobacterium glaciei |
| fig\|1500289.3.peg.4103 | Chryseobacterium sp. OV705 |
| fig\|1500298.3.peg.2823 | Chry seobacterium sp. YR561 |
| fig\|1797331.3.peg.2180 | Bacteroidetes bacterium GWE2_29_8 |
| fig\|1947498.3.peg.1366 | Sphingobacterium sp. UBA4616 |
| WP_074239321.1 | Chitinophaga niabensis |
| fig\|192149.7.peg.174 | Muricauda sp. |
| fig\|718222.3.peg.4924 | Bacillus cereus TIAC219 |
| fig\|1053210.3.peg.211 | Bacillus cereus HuB4-10 |
| fig\|2026089.3.peg.6077 | Paenibacillus sp. XY044 |
| fig\|1938610.3.peg.3678 | Flavobacterium sp. LM5 |
| fig\|1947482.3.peg.632 | Sphingobacterium sp. UBA1575 |
| fig\|1948844.3.peg.823 | Patescibacteria group bacterium UBA6130 |
| fig\|986.7.peg.83 | Flavobacterium johnsoniae |
| fig\|1950382.3.peg.461 | Bacteroidales bacterium UBA1181 |
| fig\|1947145.3.peg.376 | Prevotella sp. UBA3765 |
| fig\|1122989.3.peg.367 | Prevotella oris DSM 18711 = JCM 12252 |
| fig\|1896974.3.peg.2001 | Bacteroides sp. 43 108 |
| fig\|2014804.3.peg.4306 | Lewinellaceae bacterium SD302 |
| fig\|1428.517.peg.2380 | Bacillus thuringiensis |
| fig\|1428.574.peg.3351 | Bacillus thuringiensis |
| fig\|1428.590.peg.5685 | Bacillus thuringiensis |
| fig\|720554.3.peg.188 | Hungateiclostridium clariflavum DSM 19732 |
| fig\|1122203.4.peg.2283 | Marinococcus halotolerans DSM 16375 |
| fig\|1462525.3.peg 3489 | Thalassobacillus sp. TM-1 |
| fig\|1395513.3.peg.363 | Sporolactobacillus laevolacticus DSM 442 |
| fig\|1262834.3.peg.1229 | Clostridium sp. CAG:715 |
| SCI87282.1 | uncultured Roseburia sp. |
| fig\|1952116.3.peg.2349 | Lachnospiraceae bacterium UBA6480 |
| WP_069150959.1 | Lachnospiraceae |
| fig\|1265.10.peg.3100 | Ruminococcus flavefaciens |
| fig\|1120998.3.peg.2858 | Anaerovorax odorimutans DSM 5092 |
| WP_072702499.1 | Butyrivibrio hungatei |
| fig\|1232453.3.peg.2795 | Clostridiales bacterium VE202-21 |
| fig\|39485.11.peg.251 | Lachnospira eligens |
| WP_072832325.1 | |
| fig\|1509.24.peg.2487 | Clostridium sporogenes |
| SCI88558.1 | uncultured Clostridium sp. |
| fig\|1490.6.peg.2635 | Paraclostridium bifermentans |
| fig\|1947399.3.peg.1322 | Hungateiclostridiaceae bacterium UBA3548 |
| fig\|1953138.3.peg.796 | Bacteroidetes bacterium UBA1312 |
| fig\|1950875.3.peg.364 | Clostridiales bacterium UBA4139 |
| fig\|1396.1518.peg.4860 | Bacillus cereus |
| fig\|1305675.3.peg.2174 | Bacillus solimangrovi |
| fig\|1423.436.peg.4365 | Bacillus subtilis |
| fig\|361277.6.peg.1539 | Terribacillus saccharophilus |
| fig\|1392.356.peg.4724 | Bacillus anthracis |
| fig\|1053189.3.peg.4219 | Bacillus cereus BAG5X1-1 |
| WP_079442297.1 | Clostridium chromitreducens |
| fig\|1953262.3.peg.783 | Candidatus Omnitrophica bacterium |
| | UBA1562 |
| fig\|1797955.3.peg.2304 | Elusimicrobia bacterium |
| | RIFOXYA12_FULL_51_18 |
| fig\|1953111.3.peg.2436 | Acidobacteria bacterium UBA7540 |
| WP_099010551.1 | Escherichia coli |
| fig\|1005565.3.peg.1153 | Escherichia coli 3006 |
| fig\|158822.8.peg.1905 | Cedecea neteri |
| fig\|1444060.3.peg.4830 | Escherichia coli 4-203-08_S1_C1 |
| fig\|29484.22.peg.4571 | Yersinia frederiksenii |
| fig\|529823.3.peg.332 | Cellvibrio sp. OA-2007 |
| fig\|48296.218.peg.3142 | Acinetobacter pittii |
| fig\|550.518.peg.3445 | Enterobacter cloacae |
| fig\|204773.6.peg.4 | Herminiimonas arsenicoxy dans |
| fig\|670.190.peg.4348 | Vibrio parahaemolyticus |
| fig\|44577.7.peg.209 | Nitrosomonas ureae |
| fig\|1125747.3.peg.1 | Paraglaciecola agarilytica NO2 |
| fig\|1338034.3.peg.2437 | Vibrio parahaemolyticus 01:Kuk |
| | str. FDA_R31 |
| fig\|1952844.3.peg.2619 | Rhodocyclaccae bacterium UBA5533 |
| fig\|1288788.3.peg 2384 | Vibrio parahaemolyticus 3631 |
| fig\|644.31.peg.975 | Aeromonas hydrophila |
| fig\|498292.3.peg.28 | Flavobacterium swingsii |
| fig\|1948088.3.peg.4515 | Firmicutes bacterium UBA6132 |
| fig\|1408433.3.peg.3094 | Crocinitomix catalasitica ATCC 23190 |
| WP_074236572.1 | Chryscobacterium zeac |
| fig\|1127353.3.peg.1738 | Salmonella enterica subsp. enterica |
| | serovar Newport str. #11-4 |
| fig\|1881110.4.peg.120 | Pantoca sesami |
| fig\|34038.6.peg.27 | Rahnella aquatilis |
| fig\|630.95.peg.4256 | Yersinia enterocolitica |
| fig\|149387.11.peg.1139 | Salmonella enterica subsp. |
| | enterica serovar Brandenburg |
| fig\|1343738.3.peg.2232 | Vibrio cholerae 2012EL-1759 |
| fig\|1423.175.peg.4339 | Bacillus subtilis |
| fig\|2021695.3.peg.3399 | Bacillus sp. 7894-2 |
| fig\|189426.10.peg.597 | Paenibacillus odonfer |
| fig\|2020949.3.peg.856 | Romboutsia weinsteinii |
| fig\|1243664.3.peg.1004 | Bacillus massiliogorillae |
| fig\|1855345.3.peg.2971 | Bacillus sp. RRD69 |
| fig\|1946358.3.peg.2514 | Clostridium sp. UBA4108 |
| fig\|1520.90.peg.2502 | Clostridium beijerinckii |
| fig\|79672.3.peg.288 | Bacillus thuringiensis serovar medellin |
| fig\|189426.19.peg.3973 | Paenibacillus odorifer |
| fig\|1497.3.peg.4049 | Clostridium formicaceticum |
| fig\|169760.4.peg.4269 | Paenibacillus stellifer |
| WP_073588670.1 | Anaerocolumna xylanovorans |
| fig\|1950815.3.peg.1585 | Clostridiales bacterium UBA1341 |
| fig\|1897004.3.peg.2166 | Eubacterium sp. 45 250 |
| fig\|1946293.3.peg.290 | Catabacter sp. UBA7571 |
| fig\|1796620.3.peg.3489 | Acutalibacter muris |
| fig\|76857.53.peg.2245 | Fusobacterium nucleatum subsp. |
| | polymorphum |
| fig\|2013784.3.peg.1260 | Firmicutes bacterium HGW-Firmicutes-3 |
| fig\|79884.3.peg.1106 | Bacillus pseudalcaliphilus |
| fig\|135461.47.peg.1552 | Bacillus subtilis subsp. subtilis |
| WP_016122013.1 | Bacillus cereus group |
| fig\|1499688.3.peg 3214 | Bacillus sp. LF1 |
| fig\|1318.8.peg.1495 | Streptococcus parasanguinis |
| fig\|1381091.3.peg.1173 | Streptococcus equi subsp. |
| | zooepidemicus SzAM60 |
| fig\|1409369.3.peg.815 | Staphylococcus aureus AMMC6050 |
| fig\|1497681.5.peg.3014 | Listeria newyorkensis |
| fig\|1095727.3.peg.409 | Streptococcus sp. SK643 |
| fig\|1318.12.peg.313 | Streptococcus parasanguinis |
| fig\|1681184.3.peg.4899 | Lysinibacillus sp. ZYM-1 |
| fig\|561879.29.peg.3569 | Bacillus safensis |
| fig\|1884359.3.peg.2978 | Psychrobacillus sp. OK028 |
| fig\|29367.3.peg.1112 | Clostridium puniceum |
| fig\|225345.3.peg.1103 | Clostridium chromiireducens |
| fig\|1345695.10.peg.2438 | Clostridium saccharobutylicum DSM 13864 |
| fig\|119641.3.peg.784 | Clostridium uliginosum |
| fig\|1761781.3.peg.88 | Clostridium sp. DSM 8431 |
| fig\|1946346.3.peg.2999 | Clostridium sp. UBA1056 |
| fig\|1492.48.peg.3952 | Clostridium butyricum |
| fig\|1121302.3.peg.4511 | Clostridium cavendishii DSM 21758 |
| fig\|398512.4.peg.5301 | Pseudobacteroides cellulosolvens |
| | ATCC 35603 = DSM 2933 |
| fig\|1946357.3.peg.500 | Clostridium sp. UBA3947 |
| fig\|642492.3.peg.3338 | Cellulosilyticum lentocellum DSM 5427 |
| fig\|1946690.3.peg.1553 | Lachnoclostridium sp. UBA3320 |
| fig\|397290.3.peg.150 | Lachnospiraceae bacterium A2 |
| fig\|97138.3.peg.1713 | Clostridium sp. ASF356 |
| fig\|1965545.3.peg.499 | Tyzzerella sp. An114 |
| fig\|1047063.3.peg.240 | WSI bacterium JGI 0000059-K21 |
| fig\|1192034.3.peg.1508 | Chondromyces apiculatus DSM 436 |
| AAA88323.1 | Myxococcus xanthus |
| fig\|33.8.peg.8196 | Myxococcus fulvus |
| fig\|215803.3.peg.1485 | Enhygromy xa salina |
| fig\|1406225.3.peg.2150 | Archangium violaceum Cb vi76 |
| fig\|1952931.3.peg.5137 | Verrucomicrobia subdivision |
| | 3 bacterium UBA6082 |
| fig\|1972460.3.peg.279 | Anaerolineaceae bacterium 4572_78 |
| fig\|1950201.3.peg.3297 | Anaerolineales bacterium UBA2796 |
| WP_015247705.1 | |
| fig\|1799658.3.peg.2777 | Planctomycetaceae bacterium |
| | SCGC AG-212-F19 |
| fig\|214688.26.peg.6659 | Gemmata obscuriglobus UQM 2246 |
| fig\|2023130.3.peg.4250 | Rhodopirellula sp. MGV |
| fig\|52.7.peg.9046 | Chondromyces crocatus |
| fig\|448385.16.peg.2914 | Sorangium cellulosum So ce56 |
| fig\|888845.4.peg.14202 | Minicystis rosea |
| fig\|1752210.3.peg.1275 | Deltaproteobacteria bacterium Ga0077539 |
| fig\|1391654.3.peg.3562 | Labilithrix luteola |
| fig\|1752218.3.peg.3670 | Planctomycetaceae bacterium Ga0077529 |
| WP_006981058.1 | Chthoniobacter flavus |
| fig\|1952939.3.peg.2584 | Verrucomicrobiaceae bacterium UBA1938 |
| fig\|2024858.3.peg.3711 | Sandaracinus sp. |
| WP_009096166.1 | Rhodopirellula sp. SWK7 |
| fig\|595453.3.peg.1506 | Rhodopirellula sp. SM50 |
| fig\|1263868.3.peg.4100 | Rhodopirellula curopaca SH398 |
| fig\|167547.3.peg.303 | Prochlorococcus marinus str. MIT 9311 |
| fig\|1499501.3.peg.459 | Prochlorococcus sp. SS52 |
| fig\|1905359.3.peg.4335 | marine bacterium AO1-C |
| WP_002700020.1 | Microscilla marina |
| fig\|1913989.145.peg.263 | Gammaproteobacteria bacterium |
| WP_073154989.1 | Seinonella peptonophila |
| fig\|46223.3.peg.3648 | Thermoflavimicrobium dichotomicum |
| fig\|1329796.3.peg.1947 | Risungbinella massiliensis |
| fig\|1123252.3.peg.3225 | Shimazuella kribbensis DSM 45090 |
| fig\|714067.3.peg.3719 | Kroppenstedtia eburnea |
| fig\|1341151.3.peg.628 | Laceyella sacchari 1-1 |
| fig\|2026763.3.peg.3089 | Myxococcales bacterium |
| fig\|1797895.3.peg.2518 | Deltaproteobacteria bacterium |
| | RIFOXYA12_FULL_58_15 |
| fig\|373672.4.peg.4082 | Chryseobacterium gambrini |
| fig\|1416778.5.peg.4374 | Cluyseobacterium arachidis |
| fig\|1603293.4.peg.829 | Flavobacterium sp. 316 |
| CCB70859.1 | Flavobacterium branchiophilum FL-15 |
| fig\|1986952.3.peg.951 | Sphingobacteriaceae bacterium |
| | GW460-11-11-14-LB5 |
| fig\|1476464.3.peg.4304 | Pedobacter xixiisoli |
| fig\|1761785.3.peg.3112 | Flavobacterium sp. ov086 |
| fig\|1664068.3.peg.3690 | bacterium 336/3 |
| fig\|880071.3.peg.3500 | Bernardetia litoralis DSM 6794 |
| fig\|1121902.3.peg.2906 | Eisenibacter elegans DSM 3317 |
| fig\|1509483.4.peg.1923 | Flectobacillus sp. BAB-3569 |
| fig\|1166018.3.peg 5312 | Fibrella aestuarina BUZ 2 |
| fig\|634771.3.peg.967 | Chitinophaga eiseniae |
| fig\|29529.3.peg.396 | Chitinophaga arvensicola |
| fig\|1891659.3.peg.6032 | Chitinophaga sp. CB10 |
| fig\|2033437.3.peg.3442 | Chitinophaga sp. MD30 |
| fig\|1004.4.peg.1677 | Chitinophaga sancti |
| fig\|1881041.3.peg.3698 | Chitinophaga sp. YR627 |
| fig\|1123078.3.peg.2010 | Runella zeae DSM 19591 |
| fig\|354355.3.peg.1846 | Niastella yeongjuensis |
| fig\|1951546.3.peg.1574 | Chitinophagaceae bacterium UBA1946 |
| fig\|1812911.3.peg.633 | Flavihumibacter sp. CACIAM 22H1 |
| fig\|477680.4.peg.4668 | Filimonas lacunae |
| fig\|221126.7.peg.3781 | Algibacter lectus |
| fig\|342954.4.peg.734 | Lacinutrix algicola |
| fig\|1871037.5.peg.2180 | Flavobacteriaceae bacterium |
| fig\|669041.3.peg.843 | Tenacibaculum dicentrarchi |
| WP_074538568.1 | Cellulophaga baltica |
| fig\|1248440.3.peg.1511 | Polaribacter franzmannii ATCC 700399 |
| fig\|1121007.3.peg 1574 | Aquimarina muelleri DSM 19832 |
| fig\|688867.3.peg.2332 | Ohtaekwangia koreensis |
| fig\|926565.3.peg.717 | Sporocytophaga myxococcoides DSM 11118 |
| fig\|1257021.3.peg.5265 | Flammeovirgaceae bacterium 311 |
| fig\|2044937.5.peg.2350 | candidate division KSB3 bacterium |
| fig\|1499966.3.peg.180 | Candidatus Moduliflexus flocculans |
| fig\|1948269.3.peg.1254 | Verrucomicrobia bacterium UBA6053 |
| fig\|694433.3.peg.3103 | Saprospira grandis DSM 2844 |
| fig\|2008677.3.peg 3337 | Mitsuaria noduli |
| fig\|946333.3.peg.3570 | Rhizobacter gummiphilus |
| fig\|1736433.3.peg.5559 | Rhizobacter sp. Root1221 |
| fig\|1500265.3.peg.5760 | Methylibium sp. YR605 |
| fig\|1121349.4.peg.2836 | Comamonas composti DSM 21721 |
| fig\|1082851.3.peg.91 | Comamonas serinivorans |
| fig\|1121480.5.peg.5468 | Pseudoduganella violaceinigra DSM 15887 |
| fig\|2045208.3.peg.1247 | Massilia violaceinigra |
| fig\|1736455.3.peg.3692 | Massilia sp. Root133 |
| fig\|34073.25.peg.8569 | Variovorax paradoxus |
| fig\|1884311.3.peg.7121 | Variovorax sp. OK202 |
| fig\|1123487.3.peg 1763 | Uliginosibacterium gangwonense DSM 18521 |
| fig\|2029111.3.peg.3032 | Comamonadaceae bacterium NML 120219 |
| fig\|1977087.20.peg.1226 | Proteobacteria bacterium |
| fig\|754436.4.peg.4454 | Photobacterium aphoticum |
| fig\|265726.7.peg.1038 | Photobacterium halotolerans |
| fig\|1121867.3.peg.59 | Enterovibrio calviensis DSM 14347 |
| fig\|1238431.3.peg.2655 | Vibrio nigripulchritudo BLFn1 |
| fig\|1384589.3.peg.2721 | [Erwinia] teleogrylli |
| fig\|1261127.3.peg.2947 | Citrobacter amalonaticus Y19 |
| fig\|349521.8.peg.3588 | Hahella chejuensis KCTC 2396 |
| fig\|525918.3.peg.1501 | Thiothrix caldifontis |
| fig\|1737490.4.peg.4974 | Agarilytica rhodophyticola |
| fig\|251229.3.peg.427 | Chroococcidiopsis thermalis PCC 7203 |
| fig\|1245923.3.peg.9587 | Scytonema millei VB511283 |
| fig\|1503470.5.peg.10896 | cyanobacterium TDX16 |
| fig\|2005460.3.peg.1118 | Chondrocystis sp. NIES-4102 |
| fig\|179408.3.peg.4679 | Oscillatoria nigro-viridis PCC 7112 |
| fig\|1612423.3.peg.5384 | Nostoc linckia z1 |
| fig\|63737.11.peg.472 | Nostoc punctiforme PCC 73102 |
| fig\|224013.5.peg.7163 | Nostoc piscinale CENA21 |
| fig\|1932621.3.peg 7363 | Nostoc sp. T09 |
| fig\|373994.3.peg.3383 | Rivularia sp. PCC 7116 |
| fig\|2005463.3.peg.257 | Calothrix sp. NIES-4105 |
| fig\|2005459.3.peg.7019 | Tolypothrix sp. NIES-4075 |
| fig\|184925.3.peg.2602 | Chlorogloeopsis fritschii PCC 9212 |
| fig\|454136.5.peg.3127 | Phormidium ambiguum IAM M-71 |
| fig\|203124.6.peg.2732 | Trichodesmium erythraeum IMS101 |
| fig\|2040638.3.peg.3067 | Tychonema bourrellyi FEM_GT703 |
| fig\|1880991.4.peg.2927 | Oscillatoriales cyanobacterium USR001 |
| fig\|1173028.3.peg.7115 | Oscillatoria sp. PCC 10802 |
| fig\|568701.4.peg.2073 | Moorea bouillonii PNG |
| fig\|927677.3.peg.4187 | Synechocystis sp. PCC 7509 |
| fig\|179408.3.peg.6267 | Oscillatoria nigro-viridis PCC 7112 |
| fig\|1710894.3.peg.2079 | Aphanizomenon flos-aquae LD13 |
| fig\|1947888.3.peg.4484 | Cyanobacteria bacterium UBA6047 |
| fig\|1705388.3.peg.1178 | Planktotbricoides sp. SR001 |
| fig\|454136.5.peg.4162 | Phormidium ambiguum IAM M-71 |
| fig\|2005458.3.peg.378 | Nostoc sp. NIES-4103 |
| fig\|1947874.3.peg.4590 | Cyanobacteria bacterium UBA1583 |
| fig\|1781255.3.peg.802 | Desertifilum sp. IPPAS B-1220 |
| fig\|1128427.4.peg.2346 | filamentous cyanobacterium ESFC-1 |
| fig\|1946321.3.peg.3928 | Chloracidobacterium sp. UBA7656 |
| fig\|118173.3.peg.1030 | Pseudanabaena sp. PCC 6802 |
| fig\|1922337.4.peg.4802 | Leptolyngbya sp. ‘hensonii’ |
| fig\|927668.3.peg.2766 | Pseudanabaena biceps PCC 7429 |
| fig\|1173020.3.peg.6191 | Chamaesiphon minutus PCC 6605 |
| fig\|329726.14.peg.4440 | Acaryochloris marina MBIC11017 |
| fig\|215803.3.peg.1649 | Enhygromyxa salina |
| fig\|1920190.3.peg.9548 | Archangium sp. Cb G35 |
| fig\|1961464.3.peg.5181 | Myxococcales bacterium UBA2376 |
| fig\|765913.3.peg.336 | Thiorhodococcus drewsil AZ1 |
| fig\|1396141.3.peg.2891 | Haloferula sp. BvORR071 |
| fig\|1961463.3.peg.5253 | Myxococcales bacterium UBA1671 |
| AAL40743.1 | Nannocystis exedens |
| fig\|54.3.peg.4123 | Nannocystis exedens |
| fig\|$3367.3.peg.3417 | Asanoa ferruginea |
| fig\|460265.11.peg.3882 | Methylobacterium nodulans ORS 2060 |
| fig\|298794.3.peg.462 | Methylobacterium variabile |
| fig\|190148.4.peg.3492 | Bradyrhizobium paxllaeri |
| fig\|1075417.3.peg.445 | Catalinimonas alkaloidigena |
| fig\|1429438.4.peg.7505 | Candidatus Entotheonella factor |
| fig\|1977087.12.peg.1756 | Proteobacteria bacterium |
| fig\|92487.3.peg.3972 | Thiothrix eikelboomii |
| fig\|1977087.20.peg.1473 | Proteobacteria bacterium |
| fig\|1123400.3.peg.3276 | Thiofilum flexile DSM 14609 |
| fig\|34062.8.peg.73 | Moraxella osloensis |
| fig\|1699623.3.peg.1502 | Psychrobacter sp. P11G3 |
| fig\|1123509.3.peg.848 | Zooshikella ganghwensis DSM 15267 |
| fig\|2026735.3.peg.2222 | Deltaproteobacteria bacterium |
| fig\|1977087.12.peg.2982 | Proteobacteria bacterium |
| fig\|2026763.4.peg.1195 | Myxococcales bacterium |
| fig\|1977087.12.peg.510 | Proteobacteria bacterium |
| fig\|1123508.3.peg.7252 | Zavarzinella formosa DSM 19928 |
| fig\|214688.26.peg 3091 | Gemmata obscuriglobus UQM 2246 |
| fig\|1908690.5.peg.1204 | Fimbriiglobus ruber |
| fig\|1805126.3.peg.4431 | Deltaproteobacteria bacterium |
| | CG2_30_63_29 |
| fig\|1882752.4.peg.1962 | Singulisphaera sp. GP187 |
| fig\|1636152.3.peg.5364 | Planctomyces sp. SH-PL62 |
| APR75442.1 | Minicystis rosea |
| fig\|54.3.peg.8798 | Nannocystis exedens |
| fig\|980254.4.peg.4083 | Roseimaritima ulvae |
| fig\|1856297.3.peg.3627 | Gammaproteobacteria bacterium 45_16_T64 |
| fig\|1219077.3.peg.1945 | Vibrio azureus NBRC 104587 |
| fig\|1334629.3.peg.167 | Myxococcus fulvus 124B02 |
| AAA25405.1 | Myxococcus xanthus |
| fig\|378806.16.peg.4444 | Stigmatella aurantiaca DW4/3-1 |
| WP_002615305.1 | Stigmatella aurantiaca |
| fig\|48.3.peg.757 | Archangium gephyra |
| fig\|448385.16.peg.2083 | Sorangium cellulosum So ce56 |
| fig\|52.7.peg.5100 | Chondromyces crocatus |
| fig\|1752210.3.peg.5621 | Deltaproteobacteria bacterium Ga0077539 |
| fig\|2024858.3.peg.6345 | Sandaracinus sp. |
| WP_012826728.1 | Haliangium ochraceum |
| fig\|927083.3.peg.3408 | Sandaracinus amylolyticus |
| WP_006977315.1 | Plesiocystis pacifica |
| fig\|1400863.5.peg.627 | Candidatus Competibacter |
| | denitrificans Run_A_D11 |
| fig\|1961463.3.peg.4303 | Myxococcales bacterium UBA1671 |
| fig\|1898731.3.peg.3099 | Curtobacterium sp. MCBA15_001 |
| fig\|1279028.3.peg.3374 | Curtobacterium sp. 314Chir4.1 |
| fig\|1898733.3.peg.2056 | Curtobacterium sp. MCBA15_004 |
| fig\|1795630.3.peg.3476 | Frondihabitans sp. PAMC 28766 |
| fig\|2033654.3.peg.3461 | Curtobacterium sp. ‘Ferrero’ |
| fig\|1736329.5.peg.1436 | Frondihabitans sp. Leaf304 |
| fig\|1736292.3.peg.1083 | Rathayibacter sp. Leaf185 |
| fig\|1736327.3.peg.206 | Rathayibacter sp. Leaf296 |
| fig\|1736311.3.peg.3668 | Curtobacterium sp. Leaf261 |
| fig\|1736308.3.peg.2333 | Frigoribacterium sp. Leaf254 |
| fig\|656366.8.peg.2905 | Arthrobacter alpinus |
| fig\|494023.3.peg.138 | Paeniglutamicibacter antarcticus |
| ASN40093.1 | Arthrobacter sp. 7749 |
| fig\|1494608.3.peg.465 | Arthrobacter sp. PAMC 25486 |
| fig\|656366.4.peg.2620 | Arthrobacter alpinus |
| fig\|656366.3.peg.1944 | Arthrobacter alpinus |
| fig\|1132441.3.peg.1888 | Arthrobacter sp. 35W |
| fig\|1704044.3.peg.520 | Arthrobacter sp. ERGS1:01 |
| fig\|1496689.3.peg.681 | Arthrobacter sp. L77 |
| fig\|1681197.3.peg.149 | Arthrobacter sp. RIT-PI-e |
| fig\|37921.12.peg.1481 | Arthrobacter agilis |
| fig\|1736303.3.peg.982 | Arthrobacter sp. Leaf234 |
| fig\|1312978.3.peg.1472 | Arthrobacter sp. H41 |
| fig\|1348338.3.peg.1472 | Leifsonia rubra CMS 76R |
| fig\|1452536.3.peg.1955 | Microbacterium sp. Cr-K20 |
| fig\|1736525.3.peg.446 | Leifsonia sp. Root4 |
| fig\|1529318.3.peg.434 | Cryobacterium sp. MLB-32 |
| fig\|1267973.3.peg.3479 | Arthrobacter sp. H5 |
| fig\|150121.3.peg.1900 | Agreia pratensis |
| fig\|123316.3.peg.955 | Agreia sp. VKM Ac-2052 |
| fig\|1052260.3.peg.3617 | Klenkia soli |
| fig\|1566299.3.peg.3962 | Klenkia marina |
| fig\|1736356.3.peg.3150 | Modestobacter sp. Leaf380 |
| fig\|1736354.3.peg.1787 | Geodermatophilus sp. Leaf369 |
| fig\|479431.6.peg.3115 | Nakamurella multipartita DSM 44233 |
| fig\|1090615.3.peg.2397 | Nakamurella panacisegetis |
| fig\|1306174.4.peg.4778 | Kineosporia aurantiaca JCM 3230 |
| fig\|546871.3.peg.1120 | Friedmanniella luteola |
| fig\|630515.4.peg.525 | Microlunatus soli |
| fig\|546874.3.peg.1181 | Friedmanniella sagamiharensis |
| BAK35674.1 | Microlunatus phosphovorus NM-1 |
| fig\|1380390.4.peg.72 | Solirubrobacterales bacterium URHD0059 |
| fig\|1283299.3.peg.2784 | Conexibacter woesei Iso977N |
| fig\|929712.3.peg.3165 | Patulibacter minatonensis DSM 18081 |
| fig\|1123262.3.peg.3125 | Solirubrobacter soli DSM 22325 |
| fig\|1861.4.peg.5240 | Geodermatophilus obscurus |
| fig\|137993.4.peg.1318 | Geodermatophilus africanus |
| fig\|1070870.3.peg.778 | Geodermatophilus nigrescens |
| fig\|1190417.3.peg.1785 | Geodermatophilus telluris |
| fig\|477641.3.peg.1023 | Modestobacter marinus |
| fig\|1798228.3.peg.3756 | Blastococcus sp. DSM 46838 |
| WP_091929708.1 | Blastococcus sp. DSM 46786 |
| SHH20361.1 | Jatrophihabitans endophyticus |
| fig\|1844.3.peg.1151 | Nocardioides luteus |
| fig\|748909.6.peg.1418 | Nocardioides alpinus |
| fig\|402596.3.peg.987 | Nocardioides exalbidus |
| fig\|1736322.3.peg.1963 | Nocardioides sp. Leaf285 |
| fig\|1445613.3.peg.3490 | Sciscionella sp. SE31 |
| fig\|543632.4.peg.9742 | Actinoplanes subtropicus |
| fig\|1036182.3.peg.2958 | Actinoplanes atraurantiacus |
| fig\|1246995.3.peg 737 | Actinoplanes friuliensis DSM 7358 |
| fig\|56427.3.peg.3052 | Couchioplanes caeruleus subsp. caeruleus |
| fig\|1710355.3.peg.2225 | Actinoplanes sp. TFC3 |
| fig\|649831.3.peg.2352 | Actinoplanes sp. N902-109 |
| fig\|35754.4.peg.6321 | Dactylosporangium aurantiacum |
| fig\|1881.4.peg.2703 | Micromonospora viridifaciens |
| fig\|47863.3.peg.3975 | Micromonospora globosa |
| fig\|285665.4.peg.2050 | Micromonospora coriariae |
| fig\|1192034.3.peg.4668 | Chondromyces apiculatus DSM 436 |
| fig\|1198133.3.peg.2442 | Myxococcus xanthus DZ2 |
| fig\|33.8.peg.521 | Myxococcus fulvus |
| fig\|394193.3.peg.7794 | Amycolatopsis saalfeldensis |
| fig\|369932.4.peg.5621 | Amycolatopsis niigatensis |
| fig\|1238180.3.peg.5340 | Amycolatopsis azurca DSM 43854 |
| fig\|589385.3.peg.7940 | Amycolatopsis xylanica |
| fig\|1068980.3.peg.1527 | Amycolatopsis nigrescens CSC17Ta-90 |
| fig\|1854586.3.peg.2100 | Amycolatopsis antarctica |
| fig\|587909.3.peg.3086 | Yahushiella deserti |
| fig\|2030.3.peg.3051 | Kibdelosporangium aridum |
| fig\|1382595.4.peg.3164 | Saccharopolyspora erythraea D |
| WP_013675061.1 | Pseudonocardia dioxanivorans |
| fig\|1660131.3.peg.2805 | Pseudonocardia sp. SCN 72-86 |
| fig\|366584.3.peg.4349 | Pseudonocardia oroxyli |
| fig\|1885031.4.peg.5241 | Pseudonocardia sp. Ae331_Ps2 |
| fig\|1690815.5.peg.5350 | Pseudonocardia sp. HH130630-07 |
| fig\|1123023.3.peg.3229 | Pseudonocardia acaciae DSM 45401 |
| fig\|1449976.3.peg.8114 | Kutzneria albida DSM 43870 |
| WP_007238159.1 | |
| fig\|1220583.3.peg.1582 | Gordonia aichiensis NBRC 108223 |
| GAB07179.1 | Gordonia amarae NBRC 15530 |
| fig\|1223545.3.peg.704 | Gordonia soli NBRC 108243 |
| fig\|1223540.3.peg.3237 | Gordonia desulfuricans NBRC 100010 |
| fig\|1112204.3.peg.4913 | Gordonia polyisoprenivorans VH2 |
| AFR49048.1 | Gordonia sp. KTR9 |
| fig\|402289.3.peg.1558 | Rhodococcus sp. HA99 |
| fig\|1077144.3.peg.224 | Dietzia alimentaria 72 |
| fig\|1344003.3.peg.1864 | Williamsia sterculiae |
| fig\|1463823.3.peg.3407 | Microbispora sp. NRRL B-24597 |
| fig\|1903117.3.peg.1566 | Williamsia sp. 1138 |
| fig\|1603258.4.peg.1828 | Williamsia herbipolensis |
| fig\|644548.3.peg.652 | Gordonia neofelifaecis NRRL B-59395 |
| fig\|1136941.3.peg.1310 | Gordonia phthalatica |
| fig\|1223542.3.peg.3439 | Gordonia malaquae NBRC 108250 |
| fig\|47312.10.peg.4232 | Tsukamurella pulmonis |
| fig\|57704.14.peg.421 | Tsukamurella tyrosinosolvens |
| fig\|521096.6.peg.2443 | Tsukamurella paurometabola DSM 20162 |
| fig\|1123241.3.peg.3642 | Nakamurella lactea DSM 19367 |
| fig\|1210073.4.peg.1031 | Nocardia salmonicida NBRC 100378 |
| fig\|1206740.4.peg.4617 | Nocardia thailandica NBRC 100428 |
| fig\|1210064.4.peg.2434 | Nocardia altamirensis NBRC 108246 |
| fig\|1123258.3.peg.1651 | Smaragdicoccus niigatensis |
| | DSM 44881 = NBRC 103563 |
| fig\|1443888.3.peg.2891 | Rhodococcus fascians 02-815 |
| fig\|1517936.4.peg.882 | Rhodococcus sp. CUA-806 |
| fig\|398843.6.peg.4214 | Rhodococcus kyotonensis |
| fig\|1813677.3.peg.4031 | Rhodococcus sp. EPR-157 |
| WP_008711873.1 | |
| fig\|1381122.3.peg.6103 | Rhodococcus erythropolis DN1 |
| fig\|1736210.3.peg.2766 | Rhodococcus sp. Leaf7 |
| fig\|1736300.3.peg.1279 | Rhodococcus sp. Leaf225 |
| fig\|1219012.3.peg.1705 | Rhodococcus corynebacterioides |
| | NBRC 14404 |
| fig\|1219023.3.peg.2791 | Rhodococcus rhodnii NBRC 100604 |
| fig\|616997.3.peg.2548 | Hoyosella altamirensis |
| fig\|1303689.4.peg.2934 | Rhodococcus koreensis |
| | JCM 10743 = NBRC 100607 |

^{a}Accession in Patric database (https://www.patricbrc.org/) or NCBI (https://www.ncbi.nlm.nih.gov/protein/)

### Engineered Retron Non-Coding RNAs

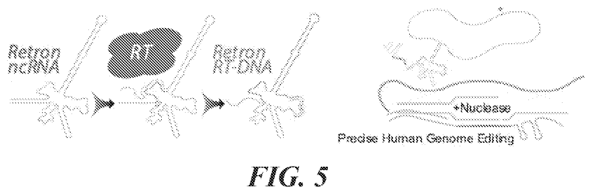

In various embodiments, the present disclosure provides engineered ncRNAs that are modified to include a guide RNA fused to the retron non-coding RNA (ncRNA). In various embodiments, the engineered retron ncRNA can be modified from its endogenous sequence (e.g., the endogenous ncRNA from retron sequences of Table A) in various ways, including but not limited to: (1) the ncRNA can be fused to a guide sequence (e.g., a CRISPR crRNA-tracrRNA), allowing the transcribed ncRNA to serve as a targeting molecule for a trans-expressed RNA-guided nuclease (e.g., a CRISPR nuclease); (2) the msd region (reverse transcribed region of the retron ncRNA) can be modified to contain a sequence that is reverse transcribed to provide DNA repair template; and (3) the a1/a2 duplex can be modified in length to facilitate increased production of the RT-DNA. A DNA repair template can comprise a single strand DNA product of reverse transcription which comprises a nucleotide sequence having a sequence modification (e.g., a desired one or more mutations, insertion, deletion, or inversion) that is flanked by regions of homology to a target genomic site. Such engineered retrons provide both the guide RNA (as part of the ncRNA) and the DNA repair template (encoded as part of the msd region, which is converted by the retron RT to a single strand RT-DNA which operates as the DNA repair template), thereby providing a vehicle to make the desired nucleotide changes at genomic sites (e.g., as shown in the embodiment of FIG. 5).

Sequences of the retron msr, msd, and/or reverse transcriptases used in the engineered retrons may be derived from a bacterial retron operon. Representative retrons are available such as those from gram-negative bacteria including, without limitation, myxobacteria retrons such as Myxococcus xanthus retrons (e.g., Mx65, Mx162) and Stigmatella aurantiaca retrons (e.g., Sa163); Escherichia coli retrons (e.g., Ec48, E67, Ec73, Ec78, EC83, EC86, EC107, and Ec107); Salmonella enterica; Vibrio cholerae retrons (e.g., Vc81. Vc95, Vc137); Vibrio parahaemolyticus (e.g., Vc96); and Nannocystis exedens retrons (e.g., Ne144), or those retrons available in Table A. Retron msr gene, msd gene, and ret gene nucleic acid sequences as well as retron reverse transcriptase protein sequences may be derived from any source, including those of Table A. Representative retron sequences, including msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and ABO79134; all of which sequences (as entered by the date of filing of this application) are herein incorporated by reference in their entireties. Any of these retron sequences or a variant thereof comprising a sequence can include variant nucleotides, added nucleotides, or fewer nucleotides.

### Guide Sequence Modifications

In various embodiments, the engineered ncRNA may comprise one or more guide sequences.

In certain embodiments, the guide RNA can be inserted into the a1/a2 complementarity region of the retron, which region of the ncRNA structure is where the 5′ and 3′ ends of the ncRNA fold back upon themselves (FIG. 1H).

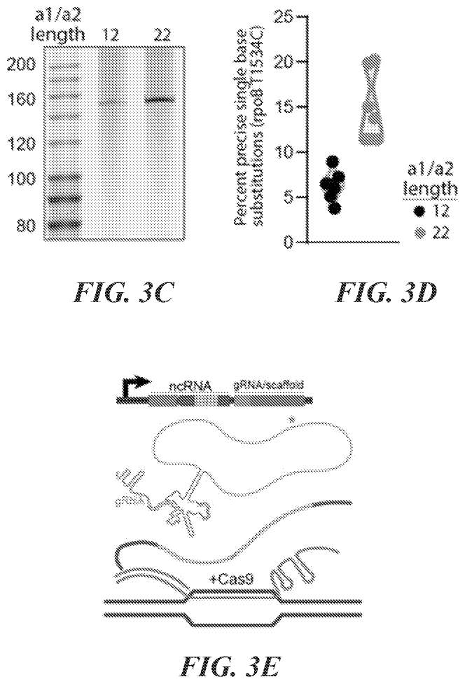

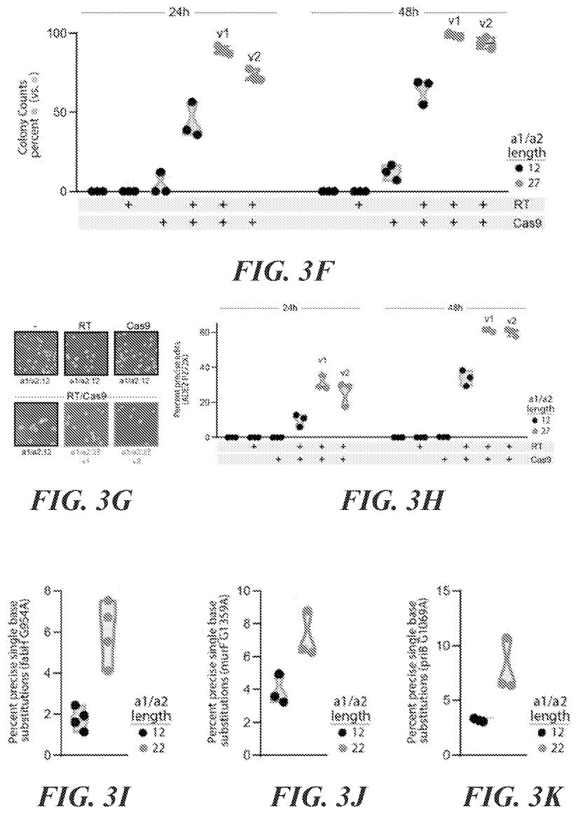

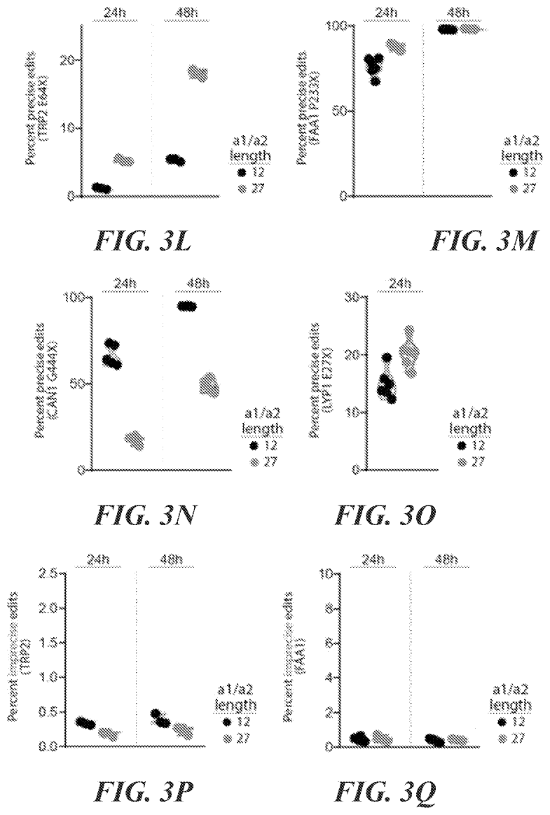

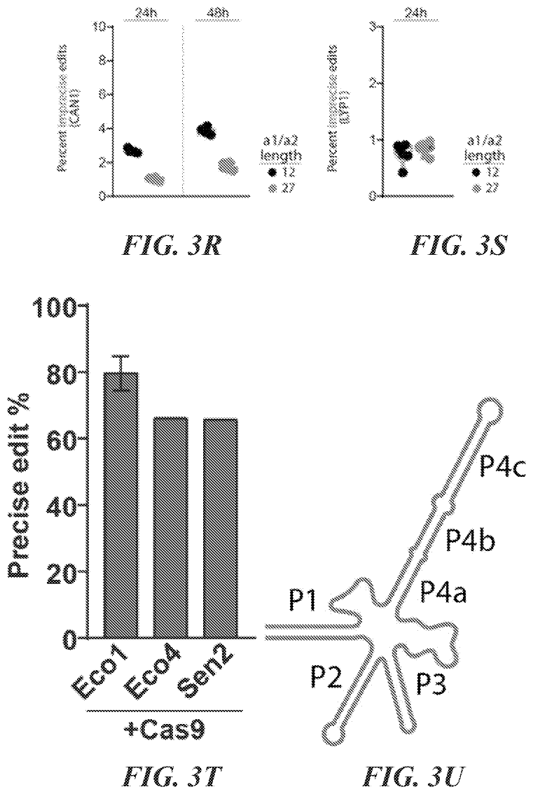

In one embodiment, the guide RNA can be coupled to the 3′ end of the ncRNA in the a1/a2 region. In another embodiment, the guide RNA can be coupled to the 5′ end of the ncRNA in the a1/a2 region. In various embodiments, a linker may separate the 3′ or 5′ retron end, as the case may be, and the guide DNA. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides in length. For instance, non-limiting embodiment wherein the gRNA is coupled to the 3′ end of the a1/a2 region is shown in FIG. 3E.

Experiments described herein show that reducing the length of a1/a2 complementarity below seven base pairs substantially impaired RT-DNA production (FIG. 1J). However, extending the a1/a2 length beyond the wild type a1/a2 length (about 13 nucleotides) resulted in increased production of RT-DNA relative to the wild type length (FIG. 1J). Importantly, this is the first modification to a retron ncRNA that has been shown to increase RT-DNA production.

The a1/a2 complementary region is lengthened to include the crRNA/gRNA relative to the corresponding region of a native retron. Such modifications can result in engineered retrons that provide enhanced production of ncRNA. In certain embodiments, the complementary region has a length at least 20, at least 21, at least 22, at least 23, at least 24, at least 25, at least 30, at least 35, at least 40, at least 45, at least 50, at least 55, at least 60, at least 65, or at least 70 nucleotides longer than the wild-type a1/a2 complementary region. For example, the self-complementary region may have a length ranging from 10 to 100 nucleotides longer than the native or wild-type complementary region, including any length within this range, such as 110, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, 50, 51, 52, 53, 54, 55, 56, 57, 58, 59, or 60 nucleotides longer. In certain embodiments, the a1/a2 complementary region has a length ranging from 20 to 50 nucleotides longer than the wild-type a1/a2 complementary region.

The guide RNA may include a nucleotide sequence that is complementary to a genomic target sequence (i.e., a “spacer” sequence), and thereby mediates binding of the RNA-guided nuclease to which it is complexed (e.g., a Cas9 nuclease-gRNA complex) by hybridization between the space sequence and a complementary strand of the genomic target site. For example, the gRNA can be designed with a sequence complementary to the sequence of a mutant genomic allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted allele may be a common genetic variant or a rare genetic variant. In certain embodiments, the gRNA is designed to selectively bind to an allele with single base-pair discrimination, for example, to allow binding of the nuclease-gRNA complex to a single nucleotide polymorphism (SNP) and modification of the SNP. In particular, the gRNA may be designed to target disease-relevant mutations of interest for the purpose of genome editing to remove the mutation from a gene.

The guide RNA can include a trans-activating crRNA (tracrRNA) scaffold recognized by a catalytically active RNA-guided nuclease (e.g., Cas9 nuclease). A guide RNA has the complementary sequence to the target DNA site, often referred to as a CRISPR RNA (crRNA), and a trans-activating crRNA (tracrRNA) scaffold that is recognized by a catalytically active Cas9 protein. The tracrRNA is made of up of a longer stretch of bases that are constant and provide the “stem loop” structure bound by the CRISPR nuclease. The crRNA can anneal to the tracrRNA through a direct repeat sequence to form a dual-guide RNA (dgRNA), or the crRNA-tracrRNA can be expressed as a single RNA transcript. When these RNA components hybridize they form a guide RNA which “programmably” targets CRISPR nucleases to DNA sequences depending on the complementarity of the crRNA and the presence of other DNA features (e.g. PAM sequences recognized by the nuclease). The guide RNA may be a single guide RNA comprising crRNA and tracrRNA sequences in a single RNA molecule, or the guide RNA may comprise two RNA molecules with crRNA and tracrRNA sequences residing in separate RNA molecules.

In certain embodiments, the gRNA is 5-50 nucleotides, 10-30 nucleotides, 15-25 nucleotides, 18-22 nucleotides, or 18-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. For example, as illustrated herein 20 base gRNAs can be useful for the human editing, whereas 18 base gRNAs were used in many experiments for editing yeast cells.

Examples of various CRISPR/Cas guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5): 1173-83; Wang et al., Cell. 2013 May 9:153(4):910-8; Auer et al., Genome Res. 2013 Oct. 31; Chen et al., Nucleic Acids Res. 2013 Nov. 1:41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al. Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9:3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23:56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entireties.

### Repair Template Modifications

In various other embodiments, the ncRNA may also be modified to include a nucleotide sequence that is reverse-transcribed to form the repair template. The repair template has a sequence that binds to a genomic DNA locus. Hence, in DNA form, the repair template sequence can be complementary to at least one chromosomal DNA strand. In some embodiments, the repair template is an HDR donor sequence which conducts repair a DNA break by way of the homology-dependent repair pathway.

However, the repair template has at least one nucleotide that is different from the complementary target sequence. In some cases, the repair template has at least two nucleotides, or at least three nucleotides, or at least four nucleotides, or at least five nucleotides, or more that are different from the complementary target sequence. These ‘different’ nucleotides are the repair nucleotides that can replace nucleotides or sequences (e.g., mutations) in the target chromosomal site.

The repair template segment of the ncRNA can have repair nucleotides that are adjacent to each other, or repair nucleotides that are separate from each other within the repair template segment. Such separations are warranted, for example, when the target chromosomal locus has two or more mutations that are not adjacent to each other.

The repair template must be sufficiently complementary for hybridization to the target sequence to mediate homologous recombination between the donor polynucleotide and genomic DNA at the target locus. For example, a homology arm may comprise a nucleotide sequence having at least about 80-100% sequence identity/complementarity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by the 5′ and 3′ arms of the repair template.

The corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm of the repair template) can be several hundred nucleotides. In some embodiments, the distance between a homology arm and the cleavage site is 200 nucleotides or less (e.g., at least 0, 10, 20, 30, 50, 75, 100, 125, 150, 175, and 200 nucleotides). In most cases, a smaller distance may give rise to a higher gene targeting rate. In a preferred embodiment, the repair template is substantially identical to the target genomic sequence, across its entire length except for the sequence changes to be introduced to a portion of the genome that encompasses both the specific cleavage site and the portions of the genomic target sequence to be altered.

A homology arm of the repair template can be of any length, e.g., 10 nucleotides or more, 15 nucleotides or more, 20 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another. However, in some instances the 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g., one may be 40% shorter or more, 50% shorter or more, sometimes 60% shorter or more, 70% shorter or more, 80% shorter or more, 90% shorter or more, or 95% shorter or more than the other homology arm.

The repair template segment of the ncRNA can therefore be of various lengths. In some cases, the repair template segment is at least 15 nucleotides, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 nucleotides in length.

The repair template is located within the ncRNA in the region that is reverse transcribed and that forms the msd stem (see FIGS. 3A, 3E, 3U; FIG. 5). For example, the repair template can be inserted into the P4a, P4b, or P4c region shown in FIG. 3U.

As shown herein, a minimal msd stem length is maintained in the msd to yield abundant RT-DNA. Although, the msd stem length can deviate by a small amount from the wild-type (wt) length of 25 base pairs, variants with stem lengths less than 12 and greater than 30 produced less than half as much RT-DNA compared to the wild type. Therefore, stem length of between 12 and 30 base pairs is preferred.

In some embodiments, the repair template is a donor DNA template that can be integrated into a host genome via HDR.

In certain embodiments, the repair template comprises or encodes a donor/template sequence, wherein the donor/template corrects/repairs/removes a mutation at the target genome site. For example, the mutation may be a mutated exon in a disease gene.

In certain embodiments, the repair template may encode or comprises a functional DNA element, such as a promoter, an enhancer, a protein binding sequence, a methylation site, or a homology region for assisting gene editing, etc.

By “donor DNA” or “donor DNA template” it is meant a single-stranded DNA to be inserted at a site cleaved by a programmable nuclease (e.g., a CRISPR/Cas effector protein or otherwise RNA-guided nuclease; a TALEN; a ZFN) (e.g., after dsDNA cleavage, after nicking a target DNA, after dual nicking a target DNA, and the like). The donor DNA template can contain sufficient homology to a genomic sequence at the target site, e.g., 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology.

Approximately 25, 50, 100, or 200 nucleotides, or more than 200 nucleotides, of sequence homology between a donor DNA template and a genomic sequence (or any integral value between 10 and 200 nucleotides, or more) can support homology-directed repair. Donor DNA template can be of any length, e.g., 50 nucleotides or more, 100 nucleotides or more, 250 nucleotides or more, 500 nucleotides or more, 1000 nucleotides or more, 5000 nucleotides or more, etc. A suitable donor DNA template can be from 50 nucleotides to 100 nucleotides, from 100 nucleotides to 500 nucleotides, from 500 nucleotides to 1000 nucleotides, from 1000 nucleotides to 5000 nucleotides, or from 5000 nucleotides to 10,000 nucleotides, or more than 10,000 nucleotides, in length.

As noted above, the donor DNA template comprises a first homology arm and a second homology arm. The first homology arm is at or near the 5′ end of the donor DNA; and comprises a nucleotide sequence that is at least partially complementary to a first nucleotide sequence in a target nucleic acid. The second homology arm is at or near the 3′ end of the donor DNA; and comprises a nucleotide sequence that is at least partially complementary to a second nucleotide sequence in the target nucleic acid. The first and second homology arms can each independently have a length of from about 10 nucleotides to 400 nucleotides; e.g., from 10 nucleotides (nt) to 15 nt, from 15 nt to 20 nt, from 20 nt to 25 nt, from 25 nt to 30 nt, from 30 nt to 35 nt, from 35 nt to 40 nt, from 40 nt to 45 nt, from 45 nt to 50 nt, from 50 nt to 75 nt, from 75 nt to 100 nt, from 100 nt to 125 nt, from 125 nt to 150 nt, from 150 nt to 175 nt, from 175 nt to 200 nt, from 200 nt to 225 nt, from 225 nt to 250 nt, from 250 nt to 275 nt, from 275 nt to 300 nt, from 325 nt to 350 nt, from 350 nt to 375 nt, or from 375 nt to 400 nt.

In certain embodiments, the donor DNA template is used for editing the target nucleotide sequence. In certain embodiments, the donor DNA template comprises one or more mutations to be introduced into the target polynucleotide. Examples of such mutations include substitutions, deletions, insertions, or a combination thereof. In certain embodiments, the mutation causes a shift in an open reading frame on the target polynucleotide. In certain embodiments, the donor polynucleotide alters a stop codon in the target polynucleotide. In certain embodiments, the donor polynucleotide corrects a premature stop codon. The correction can be achieved by deleting the stop codon, or by introducing one or more sequence changes to alter the stop codon to a codon. In certain embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment includes a fragment less than the entire copy of a gene but otherwise provides sufficient nucleotide sequence to restore the functionality of a wild type gene or non-coding regulatory sequence (e.g., sequences encoding long non-coding RNA).

In certain embodiments, the donor DNA template may be used to replace a single allele of a defective gene or defective fragment thereof. In another embodiment, the donor DNA template is used to replace both alleles of a defective gene or defective gene fragment. A “defective gene” or “defective gene fragment” is a gene or portion of a gene that when expressed, fails to generate a functioning protein or non-coding RNA with functionality of the corresponding wild-type gene.

In certain example embodiments, these defective genes may be associated with one or more disease phenotypes. In certain example embodiments, the defective gene or gene fragment is not replaced but the heterologous nucleic acid is used to insert donor polynucleotides that encode gene or gene fragments that compensate for or override defective gene expression such that cell phenotypes associated with defective gene expression are eliminated or changed to a different or desired cellular phenotype. This can be achieved by including the coding sequence of a therapeutic protein, such as a therapeutic antibody or functional fragment thereof, or a wild-type version of a defective protein associated with one or more disease phenotypes.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain embodiments, the donor DNA template manipulates a splicing site on the target polynucleotide. In certain embodiments, the donor DNA template disrupts a splicing site. The disruption may be achieved by inserting the polynucleotide to a splicing site and/or introducing one or more mutations to the splicing site. In certain embodiments, the donor polynucleotide may restore a splicing site. For example, the polynucleotide may comprise a splicing site sequence.

In certain embodiments, the donor DNA template to be inserted has a size from 10 bp to 50 kb in length, e.g., from 50 bp to ˜40 kb, from 100 bp to ˜30 kb, from 100 bp to ˜10 kb, from 100 bp to 300 bp, from 200 bp to 400 bp, from 300 bp to 500 bp, from 400 bp to 600 bp, from 500 bp to 700 bp, from 600 bp to 800 bp, from 700 bp to 900 bp, from 800 bp to 1000 bp, from 900 bp to 1100 bp, from 1000 bp to 1200 bp, from 1100 bp to 1300 bp, from 1200 bp to 1400 bp, from 1300 bp to 1500 bp, from 1400 bp to 1600 bp, from 1500 bp to 1700 bp, from 1600 bp to 1800 bp, from 1700 bp to 1900 bp, from 1800 bp to 2000 bp nucleotides in length.

In certain embodiments, the homologous arm on one or both ends of the sequence to be inserted is independently about 20 bp, 40 bp, 60 bp, 80 bp. 100 bp, 120 bp, or 150 bp.

The first homology arm and the second homology arm of the donor DNA flank a nucleotide sequence (“a nucleotide sequence of interest” or “an intervening nucleotide sequence”) that is to be introduced into a target nucleic acid. The nucleotide sequence of interest can comprise: i) a nucleotide sequence encoding a polypeptide of interest; ii) a nucleotide sequence encoding an exon of a gene; iii) a promoter sequence; iv) an enhancer sequence; v) a nucleotide sequence encoding a non-coding RNA; or vi) any combination of the foregoing.

The donor DNA can provide for gene correction, gene replacement, gene tagging, transgene insertion, nucleotide deletion, gene disruption, gene mutation, etc. For example, the donor DNA can be used to add, e.g., insert or replace, nucleic acid material to a target DNA (e.g. to “knock in” a nucleic acid that encodes a protein, an siRNA, an miRNA, etc.), to add a tag (e.g., 6×His, a fluorescent protein (e.g., a green fluorescent protein; a yellow fluorescent protein, etc.), hemagglutinin (HA). FLAG, etc.), to add a regulatory sequence to a gene (e.g. promoter, polyadenylation signal, internal ribosome entry sequence (IRES), 2A peptide, start codon, stop codon, splice signal, localization signal, enhancer, etc.), to modify a nucleic acid sequence (e.g., introduce a mutation), and the like. For example, the donor DNA can be used to modify DNA in a site-specific, i.e. “targeted”, way; for example gene knock-out, gene knock-in, gene editing, gene tagging, etc., as used in, for example, gene therapy, e.g. to treat a disease; or as an antiviral, antipathogenic, or anticancer therapeutic, the production of genetically modified organisms in agriculture, the large scale production of proteins by cells for therapeutic, diagnostic, or research purposes, the induction of pluripotent stem cells, biological research, the targeting of genes of pathogens for deletion or replacement, etc.

In some cases, the donor DNA comprises a nucleotide sequence encoding a polypeptide of interest. Polypeptides of interest include, e.g., a) functional versions of a polypeptide that comprises one or more amino acid substitutions, insertions, and/or deletions and that exhibits reduced function, e.g., where the reduced function is associated with or causes a pathological condition; b) fluorescent polypeptides; c) hormones; d) receptors for ligands; e) ion channels; f) neurotransmitters; g) and the like.

Non-limiting examples of polypeptides that can be encoded by a donor DNA include, e.g., ILIB (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJI1 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerasc family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINEI (serpin peptidase inhibitor, clade E (nexin, plasminogen activator inhibitor type 1), member 1), ALB (albumin), ADIPOQ (adiponectin, CIQ and collagen domain containing), APOB (apolipoprotein B (including Ag(x) antigen)). APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)). PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), vWF (von Willebrand factor), F2 (coagulation factor II (thrombin)), ICAM1 (intercellular adhesion molecule 1), TGFB1 (transforming growth factor, beta 1), NPPA (natriuretic peptide precursor A). IL10 (interleukin 10), EPO (ervthropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric Golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOXI (heme oxygenase (decycling) 1), APOC3 (apolipoprotein C-Ill), IL8 (interleukin 8), PROK (prokineticin 1), CBS (cystathionine-beta-synthase), NOS2 (nitric oxide synthasc 2, inducible), TLR4 (toll-like receptor 4), SELP (selectin P (granule membrane protein 140 kDa, antigen CD62)), ABCA 1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase). UTS2 (urotensin 2), THBD (thrombomodulin), F10 (coagulation factor X), CP (ceruloplasmin (ferroxidase)), TNFRSF11B (tumor necrosis factor receptor superfamily, member lib), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FNI (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface). APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRBI (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRSI (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDRITAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCRS (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIFIA (hypoxia inducible factor 1, alpha subunit (basic helix-loop-helix transcription factor)), CXCR4 (chemokine (C-X-C motif) receptor 4), PROC (protein C (inactivator of coagulation factors Va and Villa)), SCARB1 (scavenger receptor class B, member 1), CD79A (CD79a molecule, immunoglobulin-associated alpha), PLTP (phospholipid transfer protein), ADDI (adducin 1 (alpha)), FGG (fibrinogen gamma chain), SAA 1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomer ase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CAB INI (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SPI (Sp 1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBNI (fibrillin 1), CHKA (choline kinase alpha), BESTI (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit). TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IE17A (interleukin 17A), PTEN (phosphatase and tensin homolog), GSTM1 (glutathione S-transferase mu 1), DMD (dystrophin), GATA4 (GATA binding protein 4). F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member IB), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group ITA (platelets, synovial fluid)), B2M (beta-2-microglobulin), THBS1 (thrombospondin 1), GCG (glucagon). RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCHI (Notch homolog 1, translocation-associated (Drosophila)), UGTIA1 (UDP glucuronosyltransferase 1 family, polypeptide A1), IFNA1 (interferon, alpha 1), PPARD (peroxisome proliferator-activated receptor delta). SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTTI (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2). HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULTIA3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein). SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A 1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon). KCNE1 (potassium voltage-gated channel, Isk-related family, member 1), TFRC (transferrin receptor (p90, CD71)), COLlAl (collagen, type I, alpha 1), COLIA2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRFI (interferon regulatory factor 1), CFLAR (CASP8 and FADD-like apoptosis regulator), CALC A (calcitonin-related polypeptide alpha), EIF4E (eukaryotic translation initiation factor 4E), GSTPI (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PEC AMI (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-b-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXASI (thromboxane A synthase 1 (platelet)), CYP2J2 (cytochrome P450, family 2, subfamily J, polypeptide 2). TBXA2R (thromboxane A2 receptor), ADHIC (alcohol dehydrogenase IC (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylatc synthetase), SHC1 (SHC (Src homology 2 domain containing) transforming protein 1), LRP1 (low density lipoprotein receptor-related protein 1), SOCS3 (suppressor ofcytokine signaling 3), ADH1B (alcohol dehydrogenase IB (class 1), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fiagment of TgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor HI), NR112 (nuclear receptor subfamily 1, group 1, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLPIR (glucagon-like peptide 1 receptor), GHR (growth hormone receptor), GSR (glutathione reductase), NQO1 (NAD(P)H dehydrogenase, quinone 1), NR5A 1 (nuclear receptor subfamily 5, group A, member 1), GJB2 (gap junction protein, beta 2, 26 kDa). SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)). TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2). ADA (adenosine deaminase), JAK3 (Janus kinase 3), HSPA1A (heat shock 70 kDa protein 1A), FASN (fatty acid synthase), FGF1 (fibroblast growth factor 1 (acidic)), FII (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)). MYLK (myosin light chain kinase), BCF1E (butyrylcholinesterase), LIPE (lipase, hormone-sensitive), PRDX5 (peroxiredoxin 5), ADORA1 (adenosine A1 receptor), WRN (Weiner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CFIGA (chromogranin A (parathyroid secretory protein 1)), IAPP (islet amyloid polypeptide), RFIO (rhodopsin), ENPP1 (ectonucleotide pyrophosphatase/phosphodiesterase 1), PTF1LF1 (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha), F2RL3 (coagulation factor II (thrombin) receptor-like 3). CX3CL1 (chemokine (C-X3-C motif) ligand 1), BDKRB1 (bradykinin receptor B1). ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISFI (cytokine inducible SF12-containing protein), GAST (gastrin), MYOC (myocilin, trabecular mesh work inducible glucocorticoid response), ATPIA2 (ATPase, Na+/K+ transporting, alpha 2 polypeptide), NFl (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), FISF (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKGI (protein kinase, cGMP-dependent, type I), BMP2 (bone morphogenetic protein 2), CTNND1 (catenin (cadherin-associated protein), delta 1), CTF1 (cystathionase (cystathionine gamma-lyase)), CTSS (cathepsin S), VAV2 (vav 2 guanine nucleotide exchange factor). NPY2R (neuropeptide Y receptor Y2), IGFBP2 (insulin-like growth factor binding protein 2, 36 kDa), CD28 (CD28 molecule), GSTA 1 (glutathione S-transferase alpha 1), PPIA (peptidylprolyl isomerase A (cyclophilin A)), APOF1 (apolipoprotein FI (beta-2-glycoprotein I)), S100A8 (S100 calcium binding protein A8), IL11 (interleukin 11), ALOX15 (arachidonate 15-lipoxygenase), FBLN1 (fibulin 1), NR1F13 (nuclear receptor subfamily 1, group FI, member 3), SCD (stearoyl-CoA desaturase (delta-9-desaturase)), GIP (gastric inhibitory polypeptide), CF1 GB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), F1SD11B2 (hydroxy steroid (11-beta) dehydrogenase 2), CALCRL (calcitonin receptor-like), GALNT2 (UDP-N-acetyl-alpha-D-galactosaminc:polypeptide N-acetylgalactosaminyltransferase 2 (GaINAc-T2)), ANGPTL4 (angiopoictin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adeno virus E1B 19 kDa interacting protein 3), GCKR (glucokinase (hexokinase 4) regulator), S100A12 (S100 calcium binding protein A 12), PADI4 (peptidyl arginine deaminase, type IV), HSPA14 (heat shock 70 kDa protein 14), CXCR1 (chemokine (C-X-C motif) receptor 1), H19 (H19, imprinted matemally expressed transcript (non-protein coding)), KRTAP19-3 (keratin associated protein 19-3), insulin, RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha IC subunit), PRKAG2 (protein kinase, AMP-activated, gamma 2 non-catalytic subunit), CHAT (choline acetyltransferase), PTGDS (prostaglandin D2 synthase 21 kDa (brain)). NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11 A (tumor necrosis factor receptor superfamily, member 11a, NFKB activator), HSPA9 (heat shock 70 kDa protein 9 (mortalin)). CYSLTR1 (cysteinyl leukotriene receptor 1), MAT1A (methionine adenosyltransferase I, alpha), OPRL 1 (opiate receptor-like 1), IMPA1 (inositol(myo)-1(or 4)-monophosphatase 1), CLCN2 (chloride channel 2). DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)). ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa). EFNB2 (ephrin-B2), cystic fibrosis transmembrane conductance regulator (CFTR), GJB6 (gap junction protein, beta 6, 30 kDa), APOA2 (apolipoprotein A-II), AMPDI (adenosine monophosphate deaminase 1), DYSF (dysferlin, limb girdle muscular dystrophy 2B (autosomal recessive)), FDFT1 (famesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor). RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6). KHK (ketohexokinase (fructokinase)), SAT1 (spermidine/spermine NI-acetyltransferase 1), GGFI (gamma-glutamyl hydrolasc (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2 A, cGMP-stimulated). PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RFIOB (ras homolog gene family, member B), LY96 (lymphocyte antigen 96), FOXOl (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBARI (G protein-coupled bile acid receptor 1), PXK (PX domain containing serine/threonine kinase), IL33 (interleukin 33), TRIBI (tribbles homolog 1 (Drosophila)), PBX4 (pre-B-cell leukemia homeobox 4), NUPR1 (nuclear protein, transcriptional regulator, 1), 15-Sep(15 kDa selenoprotein), CILP2 (cartilage intermediate layer protein 2), TERC (telomerase RNA component), GGT2 (gamma-glutamyltransf erase 2), MT-COI (mitochondrially encoded cytochrome c oxidase I), UOX (urate oxidase, pseudogene), a CRISPR/Cas effector polypeptide, an enzymatically active CRISPR/Cas effector polypeptide (e.g., is capable of cleaving a target nucleic acid) and a CRISPR/Cas effector polypeptide that is not enzymatically active (e.g., does not cleave a target nucleic acid, but retains binding to the target nucleic acid). In some cases, the donor DNA encodes a wild-type version of any of the foregoing polypeptides; i.e., the donor DNA can encode a “normal” version that does not include a mutation(s) that results in reduced function, lack of function, or pathogenesis.

In some cases, the donor DNA comprises a nucleotide sequence encoding a fluorescent polypeptide. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPI, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, m PI urn (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, can be encoded.

In some cases, the donor DNA encodes an RNA, e.g., an siRNA, a microRNA, a short hairpin RNA (shRNA), an anti-sense RNA, a riboswitch, a ribozyme, an aptamer, a ribosomal RNA, a transfer RNA, and the like.

A donor DNA can include, in addition to a nucleotide sequence encoding one or more gene products (e.g., an RNA and/or a polypeptide), one or more transcriptional control elements, e.g., a promoter, an enhancer, and the like. In some cases, the transcriptional control element is inducible. In some cases, the promoter is reversible. In some cases, the transcriptional control element is constitutive. In some cases, the promoter is functional in a eukaryotic cell. In some cases, the promoter is a cell type-specific promoter. In some cases, the promoter is a tissue-specific promoter.

The nucleotide sequence of the donor DNA is typically not identical to the target nucleic acid (e.g., genomic sequence) that it replaces. Rather, the donor DNA may contain at least one or more single base changes, insertions, deletions, inversions or rearrangements with respect to the target nucleic acid (e.g., genomic sequence), so long as sufficient homology is present to support homology-directed repair (e.g., for gene correction, e.g., to convert a disease-causing base pair or a non-disease-causing base pair). In some cases, the donor DNA comprises a non-homologous sequence flanked by two regions of homology, such that homology-directed repair between the target DNA region and the two flanking sequences results in insertion of the non-homologous sequence at the target region. Donor DNA may also comprise a vector backbone containing sequences that are not homologous to the DNA region of interest (the target nucleic acid) and that are not intended for insertion into the DNA region of interest (the target nucleic acid). Generally, the homologous region(s) of a donor sequence will have at least 50% sequence identity to a target nucleic acid (e.g., a genomic sequence) with which recombination is desired. In certain cases, 60%, 70%, 80%, 90%, 95%, 98%, 99%, or 99.9% sequence identity is present. Any value between 1% and 100% sequence identity can be present, depending upon the length of the donor polynucleotide.

The donor DNA may comprise certain nucleotide sequence differences as compared to the target nucleic acid (e.g., genomic sequence), where such difference includes, e.g. restriction sites, nucleotide polymorphisms, selectable markers (e.g., drug resistance genes, fluorescent proteins, enzymes etc.), etc., which may be used to assess for successful insertion of the donor DNA at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence. In some cases, the donor DNA will include one or more nucleotide sequences to aid in localization of the donor to the nucleus of the recipient cell or to aid in the integration of the donor DNA into the target nucleic acid. For example, in some case, the donor DNA may comprise one or more nucleotide sequences encoding one or more nuclear localization signals and the like. In some cases, the donor DNA will include nucleotide sequences to recruit DNA repair enzymes to increase insertion efficiency. Fiuman enzymes involved in homology directed repair include MRN-CtIP. BLM-DNA2, Exol, ERCCI, Rad51, Rad52, Ligase 1, RoIQ, PARP1, Ligase 3, BRCA2, RecQ/BLM-ToroIIIa, RTEL, Roid, and Roth (Verma and Greenburg (2016) Genes Dev. 30 (10): 1138-1154). In some cases, the donor DNA is delivered as reconstituted chromatin (Cruz-Becerra and Kadonaga (2020) eLife 2020; 9:e55780 DOI: 10.7554/eLife.55780).

In some cases, the ends of the donor DNA are protected (e.g., from exonucleolytic degradation) by any convenient method and such methods are known to those of skill in the art. For example, one or more dideoxynucleotide residues can be added to the 3′ terminus of a linear molecule and/or self complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor DNA, additional lengths of sequence may be included outside of the regions of homology that can be degraded without impacting recombination.

Modifications to Length of the msd Stem Region of a ncRNA

In various embodiments, the specification relates to modifications of the length of the msd stem region that impact the amount of RT-DNA production relative to an unmodified retron. Without wishing to be bound by theory, it was surprisingly discovered that the msd stem length could tolerate some length adjustment, e.g., shortening or lengthening, relative to a wild type msd stem in the above ranges without significantly impacting production of RT-DNA and that the optimal length of the msd stem was 12 to 30 base pairs. See Example 2 for further discussion.

In various embodiments, the msd stem region can be modified so that it is less than 12 nucleotide base pairs. For example, the msd stem region can be 0, 1, 2, 3, 4, 5, 6, 7, 9, 10, or 11 nucleotide base pairs. In such embodiments where the msd stem region is shortened to below 12 nucleotide base pairs, the production of RT-DNA is reduced. e.g., a 30%, 35%, 40%, 45%, 50%, 55%, 60%, 65%, 70%, 75% or more reduction in RT-DNA production relative to wild type.

In various other embodiments, the msd stem region can be modified so that it is more than 30 base pairs. For example, the msd stem region can modified by increasing its length to 31, 32, 33, 34, 35, 36, 37, 38, 39, 40, 41, 42, 43, 44, 45, 46, 47, 48, 49, 50, or more nucleotide base pairs. In such embodiments where the msd stem region is longer than 30 nucleotide base pairs, the production of RT-DNA is reduced, e.g., a 30%, 35%, 40%, 45%, 50%, 55%, 60%, 65%, 70%, 75% or more reduction in RT-DNA production relative to wild type.

In various other embodiments, the msd stem region can be modified so that it is optimally between 12-30 base pairs. For example, the msd stem region can modified by adjusting its length to 12, 13, 14, 15, 16, 17, 18, 19, 20, 21, 22, 23, 24, 25, 26, 27, 28, 29, or nucleotide base pairs. In such embodiments where the msd stem region is between 12-30 base pairs, the production of RT-DNA is comparable to RT-DNA production relative to wild type.

Modifications to Length of the msd Loop Region of a ncRNA

In various other embodiments, the specification relates to modifications of the length of the msd loop region that result in modulation (e.g., increase or decrease) of the amount of RT-DNA production relative to an unmodified retron. In certain embodiments, the increased length of the msd loop can be due to the insertion or otherwise incorporation of a nucleotide sequence encoding a DNA donor template sequence. Without wishing to be bound by theory, it was surprising discovered that the msd loop region could tolerate significant lengthening beyond 5-14 nucleotides and even up to 70 or more nucleotides without a significant reduction in the production of RT-DNA. See Example 2 for further discussion.

Accordingly, in various embodiments, the ncRNA embodied herein may comprise an msd loop region having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84 85, 86, 87, 88, 89, 90, 91, 92, 93, 94, 95, 96, 97, 98, 99, 100, up to 150, up to 200, up to 250, up to 300, up to 350, up to 400, up to 450, up to 500, up to 550, up to 600, up to 650, up to 700, up to 750, up to 800, up to 850, up to 900, up to 950, up to 1000, up to 1050, up to 1100, up to 1150, up to 1200, up to 1250, up to 1300, up to 1350, up to 1400, up to 1450, up to 1500, up to 1550, up to 1600, up to 1650, up to 1700, up to 1750, up to 1800, up to 1850, up to 1900, up to 1950, up to 2000, up to 3000, up to 4000, up to 5000, or up to 10000 nucleotides.

Modifications to Length of a1/a2 Duplex of a ncRNA

As described in Example 2, the specification describes modified ncRNAs which contain an a1/a2 duplex having a modified length.

Turning first to Example 2, the impact of the length of the a1/a2 duplex on the production of RT-DNA was tested. The a1/a2 duplex is the region of an ncRNA structure where the 5′ and 3′ ends of the ncRNA comprise regions which are complementary to one another and fold back upon themselves to form a duplex region (e.g., see FIG. 1H). Without wishing to be bound by theory, the inventors hypothesized that the a1/a2 duplex plays a role in initiating reverse transcription (e.g. see FIG. 1H) and surprisingly discovered that reducing the length of the a1/a2 duplex to below 7 (seven) base pairs substantially impaired RT-DNA production (see e.g., FIG. 1J) relative to a retron with an unmodified a1/a2 duplex. These unexpected findings are consistent with a critical role of the a1/a2 duplex region in reverse transcription. It was further surprisingly discovered that when the a1/a2 duplex was lengthened beyond 7 bp, including up to about 30 bp or more, the production of RT-DNA substantially increased relative to the wild type length (see FIG. 1J). Importantly, it is believed that this is the first modification to a retron ncRNA that has been shown to increase RT-DNA production.

Accordingly, in other embodiments, the ncRNA embodied herein may comprise an a1/a2 duplex region having at least 7 nucleotide base pairs.

Accordingly, in still other embodiments, the ncRNA embodied herein may comprise an a1/a2 duplex region having at least 7 nucleotide base pairs, at least 8 nucleotide base pairs, at least 8 nucleotide base pairs, at least 8 nucleotide base pairs, at least 9 nucleotide base pairs, at least 10 nucleotide base pairs, at least 11 nucleotide base pairs, at least 12 nucleotide base pairs, at least 13 nucleotide base pairs, at least 14 nucleotide base pairs, at least 15 nucleotide base pairs, at least 16 nucleotide base pairs, at least 17 nucleotide base pairs, at least 18 nucleotide base pairs, at least 19 nucleotide base pairs, at least 20 nucleotide base pairs, at least 21 nucleotide base pairs, at least 22 nucleotide base pairs, at least 23 nucleotide base pairs, at least 24 nucleotide base pairs, at least 25 nucleotide base pairs, at least 26 nucleotide base pairs, at least 27 nucleotide base pairs, at least 28 nucleotide base pairs, at least 29 nucleotide base pairs, at least 30 nucleotide base pairs, at least 31 nucleotide base pairs, at least 32 nucleotide base pairs, at least 33 nucleotide base pairs, at least 34 nucleotide base pairs, at least 35 nucleotide base pairs, at least 36 nucleotide base pairs, at least 37 nucleotide base pairs, at least 38 nucleotide base pairs, at least 39 nucleotide base pairs, at least 40 nucleotide base pairs, at least 41 nucleotide base pairs, at least 42 nucleotide base pairs, at least 42 nucleotide base pairs, at least 43 nucleotide base pairs, at least 44 nucleotide base pairs, at least 45 nucleotide base pairs, at least 46 nucleotide base pairs, at least 47 nucleotide base pairs, at least 48 nucleotide base pairs, at least 49 nucleotide base pairs, but not more than 50, 100, 200, 300, 400, or 500 base pairs.

Expression of the ncRNA

In certain embodiments, the engineered ncRNA, which includes the guide RNA and the repair template, can be expressed from an expression cassette that includes a promoter operably liked to the ncRNA. The phrase “operably linked” or “under transcriptional control” as used herein means that the promoter is in the correct location and orientation in relation to a polynucleotide to control the initiation of transcription by RNA polymerase and expression of the ncRNA (or as described the reverse transcriptase and/or the Cas nuclease).

As illustrated herein, an inverted arrangement of the retron operon, with the ncRNA placed in the 3′ UTR of the reverse transcriptase transcript, can produce reverse transcribed msd DNA in bacteria, yeast, and mammalian cells. This is the first time that a single, unifying retron architecture has been shown to be compatible with all of these host systems, simplifying comparisons and portability across kingdoms.

The expression cassette can include any type of promoter to express the engineered ncRNA, which includes the guide RNA and the repair template. However, as illustrated herein, to provide precise editing in mammalian cells, including human cells, a change was made relative to the editing systems used in bacteria and yeast. This change involved use of RNA polymerase III (Pol III) promoters to express the ncRNA/gRNA in mammalian (e.g., human) cells.

RNA polymerase III (Pol III) is responsible for the synthesis of a large variety of small nuclear and cytoplasmic non-coding RNAs. Examples of PolIII promoters include the 7SK. U6 and H1 promoters. PolIII promoters can provide expression in a variety of cell types. PolIII promoters are typically compact, for example, providing expression from 5′-flanking sequences as short as 100 bp. In other cases, the PolIII promoter has more than 100 nucleotides. For example, the DNA elements for transcription of the H1 RNA gene are composed of the octamer, Staf transcription factor binding site, proximal sequence element (PSE) and TATA motifs.

An example of a sequence for a H1 promoter is shown below as SEQ ID NO: 8.

```
    1 AATTCGGAAC GCTGACGTCA TCAACCCGCT CCAAGGAATC
  41 GCGGGCCCAG TGTCACTAGG CGGGAACACC CAGCGCGCGT
  81 GCGCCCTGGC AGGAAGATGG CTGTGAGGGA CAGGGGAGTG
121 GCGCCCTGCA ATATTTGCAT GTCGCTATGT GTTCTGGGAA
161 ATCACCATAA ACGTGAAATG TCTTTGGATT TGGGAATCTT
201 ATAAGTTCTG TATGAGACCA C
```

Typically, transcription terminator/polyadenylation signals will also be present in the expression construct. PolIII terminates transcription at small PolyU stretch. In eukaryotes, a hairpin loop is not required, but may enhance termination efficiency in humans.

### Cas Nucleases and Retron Reverse Transcriptases

In various embodiments, the compositions, systems, and methods include use of two components, (1) a programmable nuclease (e.g., an RNA-guide CRISPR nuclease), and (2) a retron reverse transcriptase for synthesis of the msd DNA from the ncRNA. The programmable nuclease is targeted to a site in the genome by a guide RNA which can be fused or coupled to a retron non-coding RNA (ncRNA), which then generates a cut in the genome. This chromosomal break is then precisely repaired by the endogenous cellular machinery, using retron-derived reverse transcribed DNA (RT-DNA) as a repair template.

### Programmable Nucleases

In some cases, the programmable nuclease used for genome modification is a Cas nuclease. Any RNA-guided Cas nuclease capable of catalyzing site-directed cleavage of DNA to allow integration of donor polynucleotides can be used in genome editing, including CRISPR system type I, type II, or type III Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas1Od. CasF, CasG, CasH, Csy1, Csy2, Csy3, Csel (CasA), Csc2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquisl (NC_018721); Streptococcus thermophilus (YP_820832), Streptococcus mutans (WP_061046374, WP_024786433); Listena innocua (NP_472073); Listeria monocvtogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference in their entireties.

In another embodiment, the CRISPR nuclease from Prevotella and Francisella 1 (Cpf1) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Femandes et al. (2016) Postepy Biochem. 62(3):315-326; herein incorporated by reference.

C2c1 is another class II CRISPR/Cas system RNA-guided nuclease that may be used. C2c1, similarly to Cas9, depends on both a crRNA and tracrRNA for guidance to target sites. For a description of C2c1, see, e.g., Shmakov et al. (2015) Mol Cell. 60(3):385-397, Zhang et al. (2017) Front Plant Sci. 8:177; herein incorporated by reference.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided FokI nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

The reverse transcriptase is expressed in cells to synthesize the msd DNA from the ncRNA. As described above, the msd DNA includes the repair template within the msd loop. The retron reverse transcriptase can be expressed from the same expression cassette as the Cas nuclease, or the reverse transcriptase can be expressed from a different expression cassette than the Cas nuclease.

A variety of expression cassettes and/or expression vectors can be used to express the retron reverse transcriptase and the Cas nuclease.

Numerous vectors are available including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In addition to the CRISPR/Cas enzymes as programmable nucleases, the engineered retrons can also be used in combination with a programmable nuclease that does not use a guide RNA to recognize a target sequence, such as TALENs, ZFNs, and meganucleases.

For example, the subject engineered retron may encode or provide a msDNA that can serve as a donor or template sequence for HDR-mediated genome editing. Optionally, the RT of the engineered retron is fused to such sequence-specific nuclease, such that the msDNA, by way of being generated by the RT close to the site of HDR-mediated genome editing, can be more efficiently participate in the HDR-mediated genome editing.

In some embodiments, the non-CRISPR/Cas sequence-specific nuclease is or comprises a TALE Nuclease, a TALE nickase, Zinc Finger (ZF) Nuclease, ZF Nickase, meganuclease, or a combination thereof. In some embodiments, the non-CRISPR/Cas sequence-specific nuclease is or includes two, three, four, or more of an independently selected TALE Nuclease, TALE nickase, Zinc Finger (ZF) Nuclease, ZF Nickase, Meganuclease, restriction enzymes or a combination thereof. In some embodiments, the combination is or comprises a TALE Nuclease/a ZF Nuclease; a TALE Nickase/a ZF nickase.

In some embodiments, the non-CRISPR/Cas sequence-specific nuclease is or comprises a TALE Nuclease (Transcription Activator-Like Effector Nucleases (TALEN)). TALENs are restriction enzymes engineered to cut specific target DNA sequences. TALENs comprise a TAL effector (TALE) DNA-binding domain (which binds at or close to the target DNA), fused to a DNA cleavage domain which cuts target DNA. TALEs are engineered to bind to practically any desired DNA sequence. Thus in some embodiments, the TALEN comprises an N-terminal capping region, a DNA binding domain which may comprise at least one or more TALE monomers or half-monomers specifically ordered to target the genomic locus of interest, and a C-terminal capping region, wherein these three parts are arranged in a predetermined N-terminus to C-terminus orientation. Optionally, the TALEN includes at least one or more regulatory or functional protein domains.

In some embodiments, the TALE monomers or half monomers may be variant TALE monomers derived from natural or wild type TALE monomers but with altered amino acids at positions usually highly conserved in nature, and in particular have a combination of amino acids as RVDs that do not occur in nature, and which may recognize a nucleotide with a higher activity, specificity, and/or affinity than a naturally occurring RVD. The variants may include deletions, insertions and substitutions at the amino acid level, and transversions, transitions and inversions at the nucleic acid level at one or more locations. The variants may also include truncations.

In some embodiments, the TALE monomer/half monomer variants include homologous and functional derivatives of the parent molecules. In some embodiments, the variants are encoded by polynucleotides capable of hybridizing under high stringency conditions to the parent molecule-encoding wild-type nucleotide sequences.

In some embodiments, the DNA binding domain of the TALE has at least 5 of more TALE monomers and at least one or more half-monomers specifically ordered or arranged to target a genomic locus of interest. The construction and generation of TALEs or polypeptides of the invention may involve any of the methods known in the art.

Naturally occurring TALEs or “wild type TALEs” are nucleic acid binding proteins secreted by numerous species of proteobacteria. TALEs contain a nucleic acid binding domain composed of tandem repeats of highly conserved monomer polypeptides that are predominantly 33, 34 or 35 amino acids in length and that differ from each other mainly in amino acid positions 12 and 13. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain may comprise several repeats of TALE monomers and this may be represented as (X1-11-(X12X13)-X14-33 or 34 or 35)z, where z is optionally at least 5-40, such as 10-26.

The TALE monomers have a nucleotide binding affinity that is determined by the identity of the amino acids in its RVD. Polypeptide monomers with an RVD of NI preferentially bind to adenine (A), monomers with an RVD of NG preferentially bind to thymine (T), monomers with an RVD of HD preferentially bind to cytosine (C), monomers with an RVD of NN preferentially bind to both adenine (A) and guanine (G), monomers with an RVD of IG preferentially bind to T, monomers with an RVD of NS recognize all four base pairs and may bind to A, T, G or C. Thus, the number and order of the polypeptide monomer repeats in the nucleic acid binding domain of a TALE determines its nucleic acid target specificity. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In some embodiments, the TALE is a dTALE (or designerTALE), see Zhang et al., Nature Biotechnology 29:149-153 (2011), incorporated herein by reference.

In some embodiments, the TALE monomer comprises an RVD of HN or NH that preferentially binds to guanine, and the TALEs have high binding specificity for guanine containing target nucleic acid sequences. In come embodiments, polypeptide monomers having RVDs RN, NN, NK, SN, NH, KN, HN, NQ, HH, RG, KH, RH and SS preferentially bind to guanine. In some embodiments, polypeptide monomers having RVDs RN, NK, NQ, HH, KH, RH, SS and SN preferentially bind to guanine. In some embodiments, polypeptide monomers having RVDs HH, KH, NH, NK, NQ, RH, RN and SS preferentially bind to guanine. In some embodiments, the RVDs that have high binding specificity for guanine are RN, NH RH and KH. In some embodiments, polypeptide monomers having an RVD of NV preferentially bind to adenine and guanine as do monomers having the RVD HN. Monomers having an RVD of NC preferentially bind to adenine, guanine and cytosine, and monomers having an RVD of S (or S*), bind to adenine, guanine, cytosine and thymine with comparable affinity. In more embodiments, monomers having RVDs of H*, HA, KA, N*, NA, NC, NS, RA, and S* bind to adenine, guanine, cytosine and thymine with comparable affinity. Such polypeptide monomers allow for the generation of degenerative TALEs able to bind to a repertoire of related, but not identical, target nucleic acid sequences.

In certain embodiments, the TALE polypeptide has a nucleic acid binding domain containing polypeptide monomers arranged in a predetermined N-terminus to C-terminus order such that each polypeptide monomer binds to a nucleotide of a predetermined target nucleic acid sequence, and where at least one of the polypeptide monomers has an RVD of HN or NH and preferentially binds to guanine, an RVD of NV and preferentially binds to adenine and guanine, an RVD of NC and preferentially binds to adenine, guanine and cytosine or an RVD of S and binds to adenine, guanine, cytosine and thymine.

In some embodiments, each polypeptide monomer of the nucleic acid binding domain that binds to adenine has an RVD of NI, NN, NV, NC or S.

In certain embodiments, each polypeptide monomer of the nucleic acid binding domain that binds to guanine has an RVD of HN, NH, NN, NV, NC or S.

In certain embodiments, each polypeptide monomer of the nucleic acid binding domain that binds to cytosine has an RVD of HD, NC or S.

In some embodiments, each polypeptide monomer that binds to thymine has an RVD of NG or S.

In some embodiments, each polypeptide monomer of the nucleic acid binding domain that binds to adenine has an RVD of NI.

In certain embodiments, each polypeptide monomer of the nucleic acid binding domain that binds to guanine has an RVD of HN or NH.

In certain embodiments, each polypeptide monomer of the nucleic acid binding domain that binds to cytosine has an RVD of HD.

In some embodiments, each polypeptide monomer that binds to thymine has an RVD of NG.

In certain embodiments, the RVDs that have a specificity for adenine are NI, RI, KI, HI, and SI.

In certain embodiments, the RVDs that have a specificity for adenine are HN, SI and RI, most preferably the RVD for adenine specificity is SI.

In certain embodiments, the RVDs that have a specificity for thymine are NG, HG, RG and KG.

In certain embodiments, the RVDs that have a specificity for thymine are KG, HG and RG, most preferably the RVD for thymine specificity is KG or RG.

In certain embodiments, the RVDs that have a specificity for cytosine are HD, ND, KD, RD, HH, YG and SD.

In certain embodiments, the RVDs that have a specificity for cytosine are SD and RD.

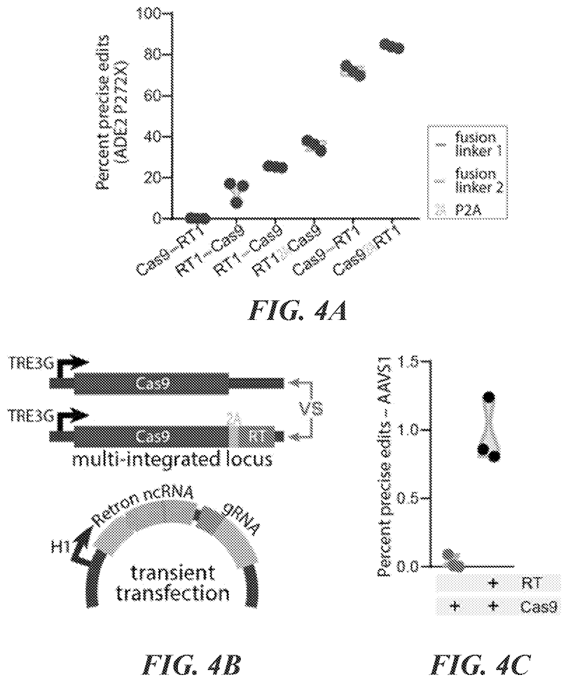

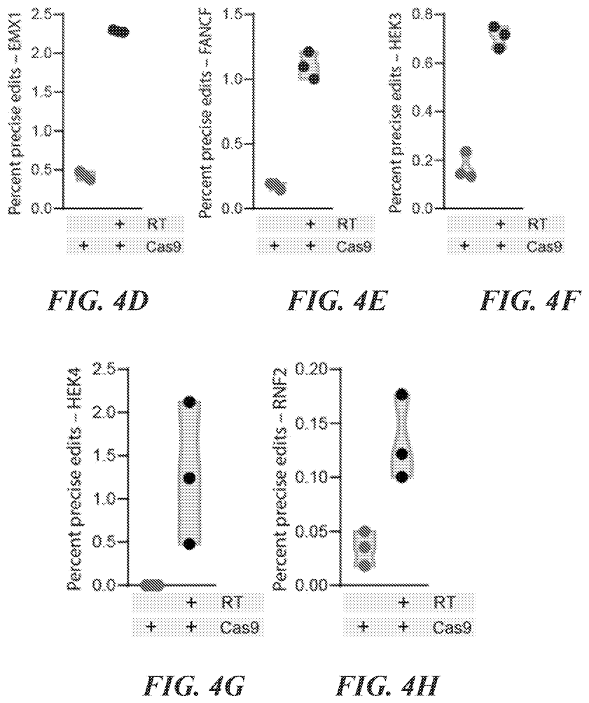

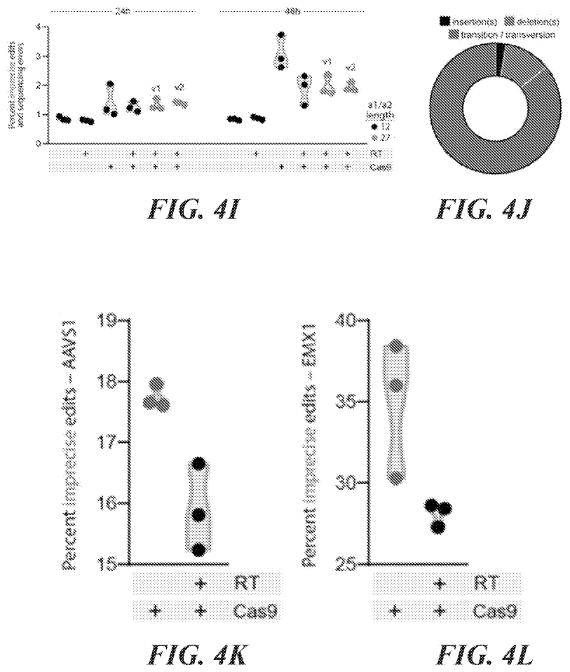

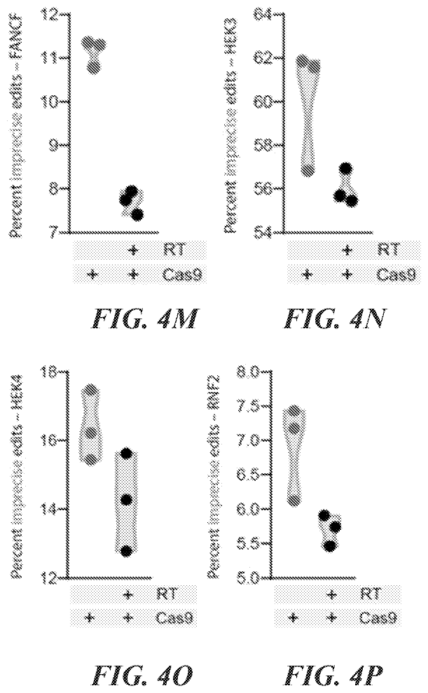

FIG. 4B of WO 2012/067428 provides representative RVDs and the nucleotides they target, the entire content of which is hereby incorporated herein by reference.

In certain embodiments, the variant TALE monomers may comprise any of the RVDs that exhibit specificity for a nucleotide as depicted in FIG. 4A of WO2012/067428. All such TALE monomers allow for the generation of degenerative TALEs able to bind to a repertoire of related, but not identical, target nucleic acid sequences.

In certain embodiments, the RVD SH may have a specificity for G, the RVD IS may have a specificity for A. and the RVD IG may have a specificity for T.

In certain embodiments, the RVD NT may bind to G and A. In certain embodiments, the RVD NP may bind to A, T and C. In certain embodiments, at least one selected RVD may be NI, HD, NG, NN, KN, RN, NH, NQ, SS, SN, NK, KH, RH, HH, KI, HI, RI, SI, KG, HG, RG, SD, ND, KD, RD, YG, HN, NV, NS, HA, S*, N*, KA, H*, RA, NA or NC.

The predetermined N-terminal to C-terminal order of the one or more polypeptide monomers of the nucleic acid or DNA binding domain determines the corresponding predetermined target nucleic acid sequence to which the TALE or polypeptides of the invention may bind.

As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8 of WO 2012/067428). Therefore, it follows that the length of the nucleic acid or DNA being targeted is equal to the number of full monomers plus two (see FIG. 44 of WO 2012/067428).

In certain embodiments, nucleic acid binding domains are engineered to contain 5, 6, 7, 8, 9, 10, 11, 12, 13, 14, 15, 16, 17, 18, 19, 20, 21, 22, 23, 24, 25, or more polypeptide monomers arranged in a N-terminal to C-terminal direction to bind to a predetermined 5, 6, 7, 8, 9, 10, 11, 12, 13, 14, 15, 16, 17, 18, 19, 20, 21, 22, 23, 24, 25 nucleotide length nucleic acid sequence.

In certain embodiments, nucleic acid binding domains are engineered to contain 5, 6, 7, 8, 9, 10, 11, 12, 13, 14, 15, 16, 17, 18, 19, 20, 21, 22, 23, 24, 25, 26 or more full length polypeptide monomers that are specifically ordered or arranged to target nucleic acid sequences of length 7, 8, 9, 10, 11, 12, 13, 14, 15, 16, 17, 18, 19, 20, 21, 22, 23, 24, 25, 26, 27 and 28 nucleotides, respectively. In certain embodiments, the polypeptide monomers are contiguous. In some embodiments, half-monomers may be used in the place of one or more monomers, particularly if they are present at the C-terminus of the TALE.

Polypeptide monomers are generally 33, 34 or 35 amino acids in length. With the exception of the RVD, the amino acid sequences of polypeptide monomers are highly conserved or as described herein, the amino acids in a polypeptide monomer, with the exception of the RVD, exhibit patterns that effect TALE activity, the identification of which may be used in preferred embodiments of the invention.

In certain embodiments, TALE polypeptide binding efficiency is increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE (including TALEs) polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. In certain embodiments, C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result. % homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Additional sequences for the conserved portions of polypeptide monomers and for N-terminal and C-terminal capping regions are included in the sequences with the following gene accession numbers: AAW59491.1, AAQ79773.2, YP_450163.1. YP_001912778.1, ZP_02242672.1, AAW59493.1, AAY54170.1, ZP_02245314.1, ZP_02243372.1, AAT46123.1, AAW59492.1, YP_451030.1, YP_001915105.1, ZP_02242534.1, AAW77510.1, ACD11364.1, ZP_02245056.1. ZP_02245055.1, ZP_02242539.1, ZP_02241531.1, ZP_02243779.1, AAN01357.1, ZP_02245177.1, ZP_02243366.1, ZP_02241530.1, AAS58130.3, ZP_02242537.1, YP_200918.1, YP_200770.1, YP_451187.1, YP_451156.1. AAS58127.2, YP_451027.1, UR_451025.1, AAA92974.1, UR_001913755.1, ABB70183.1, UR_451893.1, UR_450167.1, ABY60855.1, UR 200767.1, ZR_02245186.1, ZR_02242931.1, ZR_02242535.1, AAU54169.1, UR 450165.1, UR_001913452.1. AAS58129.3, ACM44927.1, ZR_02244836.1, AAT46125.1, UR_450161.1, ZR_02242546.1, AAT46122.1, UR_451897.1. AAF98343.1, UR_001913484.1, AAY54166.1, UR_001915093.1, UR_001913457.1. ZR_02242538.1, UR_200766.1, UR_453043.1, UR_001915089.1, UR_001912981.1, ZR_02242929.1, UR_001911730.1, UR_201654.1, UR_199877.1, ABB70129.1, UR_451696.1. UR_199876.1, AAS75145.1, AAT46124.1, UR_200914.1, UR 001915101.1, ZR_02242540.1, AAG02079.2, UR_451895.1, YP 451189.1, UR_200915.1, AAS46027.1, UR_001913759.1. UR_001912987.1, AAS58128.2, AAS46026.1, UR_201653.1, UR 202894.1, UR_001913480.1, ZR_02242666.1, R_001912775.1, ZR_02242662.1, AAS46025.1, AAC43587.1, BAA37119.1, NPJ544725.1, ABO77779.1, BAA37120.1, ACZ62652.1, BAF46271.1, ACZ62653.1. NPJ544793.1, ABO77780.1, ZR_02243740.1, ZR_02242930.1, AAB69865.1, AAY54168.1, ZR_02245191.1, UR_001915097.1. ZR 02241539.1, UR_451158.1, BAA37121.1, UR_001913182.1, UR_200903.1, ZR_02242528.1, ZR 06705357.1, ZR_06706392.1, AD148328.1, ZR_06731493.1, AD148327.1, ABO77782.1, ZR 06731656.1, NR_942641.1, AAY43360.1, ZR_06730254.1, ACN39605.1, UR_451894.1, UR_201652.1. UR_001965982.1, BAF46269.1, NPJ544708.1, ACN82432.1, ABO77781.1, P14727.2, BAF46272.1, AAY43359.1. BAF46270.1, NR_644743.1, ABG37631.1, AABOO675.1, YP 199878.1, ZR 02242536.1. CAA48680.1, ADM80412.1, AAA27592.1, ABG37632.1. ABP97430.1, ZR_06733167.1, AAY43358.1, 2KQ5_A, BAD42396.1, ABO27075.1, UR_002253357.1, UR_002252977.1, ABO27074.1, ABO27067.1, ABO27072.1, ABO27068.1, UR_003750492.1, ABO27073.1, NR_519936.1, ABO27071.1, ABO27070.1, and ABO27069.1, each of which is hereby incorporated by reference.

In certain embodiments, the programmable nuclease is a zinc finger nuclease (ZFN), such as an artificial zinc-finger nuclease having arrays of zinc-finger (ZF) modules to target new DNA-binding sites in a target sequence (e.g., target sequence or target site in the genome). Each zinc finger module in a ZF array targets three DNA bases. A customized array of individual zinc finger domains is assembled into a ZF protein (ZFP). The resulting ZFP can be linked to a functional domain such as a nuclease.

ZF nucleases (ZFN) may be used as alternative programmable nucleases for use in retron-based editing in place of RNA-guide nucleases. ZFN proteins have been extensively described in the art, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

In certain embodiments, the ZF-linked nuclease is a catalytic domain of the Type IIS restriction enzyme FokI (see Kim et al., PNAS U.S.A. 91:883-887, 1994; Kim et al., PNAS U.S.A. 93:1156-1160, 1996, both incorporated herein by reference).

In certain embodiments, the ZFN comprises paired ZFN heterodimers, resulting in increased cleavage specificity and/or decreased off-target activity. In this embodiment, each ZFN in the heterodimer targets different nucleotide sequences separated by a short spacer (see Doyon et al., Nat. Method 8:74-79, 2011, incorporated herein by reference).

In certain embodiments, the ZFN comprises a polynucleotide-binding domain (comprising multiple sequence-specific ZF modules) and a polynucleotide cleavage nickase domain.

In certain embodiments, the ZFs are engineered using libraries of two finger modules.

In certain embodiments, strings of two-finger units are used in ZFNs to improve DNA binding specificity from polyzinc finger peptides (see PNAS USA 98: 1437-1441, incorporated herein by reference).

In certain embodiments, the ZFN has more than 3 fingers. In certain embodiments, the ZFN has 4, 5, or 6 fingers. In certain embodiments, the ZF modules in the ZFN are separated by one or more linkers to improve specificity.

In certain embodiments, the ZF of the ZFN includes substitutions in the dimer interface of the cleavage domain that prevent homodimerization between ZFs but allow heterodimers to form.

In certain embodiments, the ZF of the ZFN has a design that retains activity while suppressing homodimerization.

In certain embodiments, the ZFN is any one of the ZF nucleases in Table 1 of Carroll et al., Genetics 188(4):773-782, 2011, incorporated herein by reference.

General principles and guidance for generating ZF, ZF arrays, and ZFN can be found in the art, such as the modular design (where the different modules can be rearranged and assembled into new combinations for new targets) of the ZF or ZF arrays in the ZFN as taught in Carroll et al., Nat. Protoc. 1: 1329-1341, 2006 (incorporated herein by reference); the new three-finger sets for engineered ZFs generated by using partially randomized libraries; profiling the DNA-binding specificities of engineered Cys2His2 zinc finger domains using a rapid cell-based method (see Nucleic Acids Res. 35: e81, incorporated by reference). ZFs for certain DNA triplets that work well in neighbor combination are described in Sander et al., 2011. Selection-free zinc-finger-nuclease engineering by context-dependent assembly (CoDA) is taught in Nat. Methods 8: 67-69). ToolGen describes the individual fingers in their collection that are best behaved in modular assembly (Kim et al., 2011). Preassembled zinc-finger arrays for rapid construction of ZFNs are taught in Nat. Methods 8:7.

Additional, non-limiting ZFs and AFNz that can be adapted for use in the instant invention include those described in WO2010/065123, WO2000/041566, WO2003/080809, WO2015/143046, WO2016/183298, WO2013/044008, WO2015/031619, WO2017/136049, WO2016/014794, WO2017/091512, WO1995/009233, WO2000/023464, WO2000/042219, WO2002/026960, WO2001/083793; U.S. Pat. Nos. 9,428,756, 9,145,565, 8,846,578, 8,524,874, 6,777,185, 6,599,692, 7,235,354, 6,503,717, 7,491,531, 7,943,553, 7,262,054, 8,680,021, 7,705,139, 7,273,923, 6,780,590, 6,785,613, 7,788,044, 7,177,766, 6,453,242, 6,794,136, 7,358,085, 8,383,766, 7,030,215, 7,013,219, 7,361,635, 7,939,327, 8,772,453, 9,163,245, 7,045,304, 8,313,925, 9,260,726, 6,689,558, 8,466,267, 7,253,273, 7,947,873, 9,388,426, 8,153,399, 8,569,253, 8,524,221, 7,951,925, 9,115,409, 8,772,008, 9,121,072, 9,624,498, 6,979,539, 9,491,934, 6,933,113, 9,567,609, 7,070,934, 9,624,509, 8,735,153, 9,567,573, 6,919,204, US2002-0081614, US2004-0203064, US2006-0166263, US2006-0292621, US2003-0134318, US2006-0294617, US2007-0287189, US2007-0065931, US2003-0105593, US2003-0108880, US2009-0305402, US2008-0209587, US2013-0123484, US2004-0091991, US2009-0305977, US2008-0233641, US2014-0287500, US2011-0287512, US2009-0258363, US20134-244332, US2007-0134796, US2010-0256221, US2005-0267061. US2012-0204282, US2012-0252122, US2010-0311124, US2016-0215298, US2008-0031109, US2014-0017214, US2015-0267205, US2004-0235002, US2004-0204345, US2015-0064789, US2006-0063231, US2011-0265198, US2017-0218349, all incorporated herein by reference.

Polynucleotides and vectors capable of expressing one or more of the programmable nucleases are also provided herein, which can be part of the vector system of the invention. The polynucleotides and vectors can be expressed in a cell, such as a eukaryotic cell, a mammalian cell, or a human cell. Suitable vectors, cells and expression systems are described in greater detail elsewhere herein, and can be suitable for use with the TALEs, the meganucleases, and the CRISPR-Cas nucleases.

In certain embodiments, the sequence-specific nuclease is a meganuclease.

Meganucleases are a class of sequence-specific endonucleases that recognize large DNA target sites (>12 bp). These proteins can cleave a unique chromosomal sequence without affecting overall genome integrity. Meganucleases create site specific DNA DSBs, and, in the presence of donor DNA, such as one present in the heterologous nucleic acid encompassed by or encoded by the engineered retron of the invention, promotes the integration of the donor DNA at the cleavage site through homologous recombination (HR).

In certain embodiments, the meganuclease is a homing endonuclease, which is a widespread class of proteins found in eukaryotes, bacteria and archaea.

In certain embodiments, the meganuclease is I-Scel, I-Cre-I, I-Dmol, or an engineered or a naturally occurring variant thereof. The hallmark of these proteins is a well conserved LAGLIDADG peptide motif, termed (do)decapeptide, found in one or two copies. Homing endonucleases with only one such motif, such as I-Crel or I-Ceul, function as homodimers. In contrast, larger proteins bearing two (do)decapeptide motifs, such as I-Scel, PI-Scel and I-Dmol are single chain proteins.

Additional homing nucleases are found at the website of homingendonuclease.net, which provides a database listing basic properties of known LAGLIDADG homing endonucleases. See also Taylor et al., Nucleic Acids Research 40 (W1): W110-W116, 2012 (all incorporated herein by reference).

In certain embodiments, specificity (or polynucleotide recognition) of the meganuclease is modified by altering the amino acids within the meganuclease, and/or by fusing other effector domains with the meganuclease.

In certain embodiments, the meganuclease is a megaTAL, which includes a DNA binding domain from a TALE.

In certain embodiments, any of the aforementioned programmable nucleases can be engineered to have nickase activity, wherein only one strand of a double stranded DNA target is cut.

Additional suitable natural and engineered programmable nucleases (non-RNA guided proteins) are described in WO2006/097853, WO2004/067736, WO2012/030747, WO2007/123636, WO2010/001189. WO2018/071565, WO2007/049095, WO2009/068937, WO2005/105989, WO2008/102198, WO2007/057781. WO2019/126558, WO2010/046786, US2010-0151556, US2014-0121115, US2011-0207199, US2012-0301456, US2013-0189759, US2011-0158974, US2010-0144012, US2014-0112904, US2013-0196320, US2010-0203031, US2010-0167357, US2012-0272348, US2012-0258537. US2011-0072527, US2013-0183282. US2014-0178942, US2012-0260356, US2013-0236946, US2010-0325745, US2011-0041194, US2014-0004608, US2011-0263028, US2011-0225664, US2013-0145487, US2013-0045539, US2012-0171191, US2015-0315557, US2014-0017731, US2011-0091441, US2014-0038239, US2010-0229252, US2009-0222937. US2010-0146651, US2013-0059387. US2011-0179507, US2013-0326644, US2006-0078552, US2004-0002092, US201240052582, US2009-0162937, US2010-0086533, US2009-0220476, U.S. Pat. Nos. 8,802,437, 7,842,489, 8,715,992, 8,426,177, 8,476,072, 9,365,864, 9,540,623, 9,273,296, 9,290,748, 8,163,514, 8,148,098, 8,143,016, 8,143,015, 8,133,697, 8,129,134, 8,124,369, 8,119,361, 7,897,372, 9,683,257, 10,287,626, 10,273,524, 10,000,746, 10,006,052, 7,919,605, 9,018,364, 10,407,672, 8,211,685, 9,365,864, 7,476,500, all incorporated herein by reference.

### Reverse Transcriptases

In certain embodiments, the nucleic acid comprising a retron reverse transcriptase sequence and/or a Cas nuclease sequence is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III.

Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

In one embodiment, an expression vector for expressing a retron reverse transcriptase and/or a Cas nuclease comprises a promoter “operably linked” to a polynucleotide encoding the msr gene, msd gene, and ret gene. The phrase “operably linked” or “under transcriptional control” as used herein means that the promoter is in the correct location and orientation in relation to a polynucleotide to control the initiation of transcription by RNA polymerase and expression of the retron reverse transcriptase and/or the Cas nuclease.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same.

Such sequences may include UTRs comprising an internal ribosome entry site (IRES). Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997) 22: 150-161. A multitude of IRES sequences are known and include sequences derived from a wide variety of viruses, such as from leader sequences of picomaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Nal. Acad. Sci. (2003) 100(25):15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like. A variety of nonviral IRES sequences will also find use herein, including, but not limited to IRES sequences from yeast, as well as the human angiotensin II type I receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17):7622-7635), vascular endothelial growth factor IRES (Baranick et al. (2008) Proc. Natl. Acod. Sci. U.S.A. 105(12):4733-4738, Stein et al. (1998) Mol. Cell. Biol. 18(6):3112-3119, Bert et al. (2006) RNA 12(6):1074-1083), and insulin-like growth factor 2 IRES (Pedersen et al. (2002) Biochem. J. 363(Pt 1):37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a retron reverse transcriptase and/or a Cas nuclease from an expression cassette.

In certain embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomvcin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (7-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129, Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Additional viral vectors which can be used for delivering the nucleic acid molecules of interest include those derived from the pox family of viruses, including vaccinia virus and avian poxvirus. By way of example, vaccinia virus recombinants expressing a nucleic acid molecule of interest (e.g., a retron reverse transcriptase and/or a Cas nuclease) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Chimeric alphavirus vectors comprised of sequences derived from Sindbis virus and Venezuelan equine encephalitis virus can also be used. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., a retron reverse transcriptase and/or a Cas nuclease) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of 17 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The Cas nuclease and the retron reverse transcriptase can be expressed as a single fusion mRNA. For example, the Cas nuclease and the retron reverse transcriptase can be translated as a long fusion protein with a cleavable linked between them. Alternatively, coding frame of the Cas nuclease and the retron reverse transcriptase can be same with a ribosomal skipping sequence between their coding regions. Hence, these two proteins can be expressed together as one long mRNA but during translation they become separated and can then perform their functions independently.

A ribosomal skipping sequence can thus be used between the coding region of the Cas9 nuclease and the reverse transcriptase. Ribosomal skipping sequences have peptidyl sequences of about 18-22 amino acids. Such ribosomal skipping sequences can induce the ribosome to skip translation of a polyprotein (or fusion protein), and they share the sequence DxExNPGP (SEQ ID NO: 9). Examples of ribosomal skipping sequences include those shown in the table below.

TABLE 1
Ribosomal Skipping Sequences

| Name | Sequence |
|---|---|
| T2A | (GSG) EGRGSLL TCGDVEENPGP (SEQ ID NO: 10) |
| P2A | (GSG) ATNFSLLKQAGDVEENPGP (SEQ ID NO: 11) |
| E2A | GSG) QCTNYALLKLAGDVESNPGP (SEQ ID NO: 12) |
| F2A | GSG) VKQTLNFDLLKLAGDVESNPGP (SEQ ID NO: 13) |

A polynucleotide encoding ribosomal skipping sequences can be used to allow production of multiple protein products (e.g., Cas9, retron reverse transcriptase) from a single vector. One or more ribosomal skipping sequences can be inserted between the coding sequences in the multicistronic construct. The ribosomal skipping sequences allow co-expressed proteins from the multicistronic construct to be produced at equimolar levels. See, e.g., Kim et al. (2011) PLoS One 6(4):e18556. Trichas et al. (2008) BMC Biol. 6:40, Provost et al. (2007) Genesis 45(10):625-629, Furler et al. (2001) Gene Ther. 8(11):864-873; herein incorporated by reference in their entireties.

Codon usage may be optimized to improve production of a Cas nuclease and/or retron reverse transcriptase in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

In some embodiments, the RT gene is a retron RT disclosed in Mestre et al, Nucleic Acids Research. Volume 48, Issue 22, 16 Dec. 2020, Pages 12632-12647; and Mestere et al., UG/Abi: “A Highly Diverse Family of Prokaryotic Reverse Transcriptases Associated With Defense Functions.” doi.org/10.1101/2021.12.02.470933 (incorporated herein by reference).

### Cell Delivery

### Vector Systems

In some embodiments, the engineered retrons, retron components, and retron editing systems are produced by a vector system comprising one or more vectors. In the vector system, the msr gene, the msd gene, and/or the ret gene may be provided by the same vector (i.e., cis arrangement of all such retron elements), wherein the vector comprises a promoter operably linked to the msr gene and/or the msd gene. In some embodiments, the promoter is further operably linked to the ret gene. In other embodiments, the vector further comprises a second promoter operably linked to the ret gene. Alternatively, the ret gene may be provided by a second vector that does not include the msr gene and/or the msd gene (i.e., trans arrangement of msr-msd and ret). In yet other embodiments, the msr gene, the msd gene, and the ret gene are each provided by different vectors (i.e., trans arrangement of all retron elements).

Numerous vectors are available for use in the vector or vector system, including but not limited to, linear polynucleotides, polynucleotides associated with ionic or amphiphilic compounds, plasmids, and viruses.

Examples of viral vectors include, but are not limited to, adenoviral vectors, adeno-associated virus (AAV) vectors, retroviral vectors, lentiviral vectors, and the like. An expression construct can be replicated in a living cell, or it can be made synthetically.

In some embodiments, the nucleic acid comprising an engineered retron sequence is under transcriptional control of a promoter. In some embodiments, the promoter is competent for initiating transcription of an operably linked coding sequence by a RNA polymerase I, II, or III.

Exemplary promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

Exemplary promoters for plant cell expression include the CaMV 35S promoter (Odell et al., 1985, Nature 313:810-812); the rice actin promoter (McElroy et al., 1990, Plant Cell 2:163-171); the ubiquitin promoter (Christensen et al., 1989, Plant Mol. Biol. 12:619-632; and Christensen et al., 1992, Plant Mol. Biol. 18:675-689); the pEMU promoter (Last et al., 1991, Theor. Appl. Genet. 81:581-588); and the MAS promoter (Velten et al., 1984, EMBO J. 3:2723-2730).

In additional embodiments, the retron-based vectors may also comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, FIt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

These and other promoters can be obtained from or incorporated into commercially available plasmids, using techniques well known in the art. See, e.g., Sambrook et al., supra.

In some embodiments, one or more enhancer elements is/are used in association with the promoter to increase expression levels of the constructs. Examples include the SV40 early gene enhancer, as described in Dijkema et al., EMBOJ (1985) 4:761, the enhancer/promoter derived from the long terminal repeat (LTR) of the Rous Sarcoma Virus, as described in Gorman et al., Proc. Natl. Acad. Sci. USA (1982b) 79:6777, and elements derived from human CMV, as described in Boshart er al., Cell (1985) 41:521, such as elements included in the CMV intron A sequence. All such sequences are incorporated herein by reference.

In one embodiment, an expression vector for expressing an engineered retron, including the msr gene, msd gene, and/or ret gene comprises a promoter operably linked to a polynucleotide encoding the msr gene, msd gene, and/or ret gene.

In some embodiments, the vector or vector system also comprises a transcription terminator/polyadenylation signal. Examples of such sequences include, but are not limited to, those derived from SV40, as described in Sambrook er al., supra, as well as a bovine growth hormone terminator sequence (see, e.g., U.S. Pat. No. 5,122,458).

Additionally, 5′-UTR sequences can be placed adjacent to the coding sequence to further enhance the expression. Such sequences may include UTRs comprising an internal ribosome entry site (IRES). Inclusion of an IRES permits the translation of one or more open reading frames from a vector. The IRES element attracts a eukaryotic ribosomal translation initiation complex and promotes translation initiation. See, e.g., Kaufian et al., Nuc. Acids Res. (1991) 19:4485-4490; Gurtu et al., Biochem. Biophys. Res. Comm. (1996) 229:295-298: Rees et al., BioTechniques (1996) 20:102-110; Kobayashi et al., BioTechniques (1996) 21:399-402; and Mosser et al., BioTechniques (199722 ISO-161)c. A multitude of IRES sequences are known and include sequences derived from a wide variety of viruses, such as from leader sequences of picomaviruses such as the encephalomyocarditis virus (EMCV) UTR (Jang et al., Virol. (1989) 63:1651-1660). the polio leader sequence, the hepatitis A virus leader, the hepatitis C virus IRES, human rhinovirus type 2 IRES (Dobrikova et al., Proc. Natl. Acad. Sci. (2003) 100(251:15125-151301)). an IRES element from the foot and mouth disease virus (Ramesh et al., Nucl. Acid Res. (1996) 24:2697-2700), a giardiavirus IRES (Garlapati et al., J Biol. Chem. (2004) 279(51):3389-33971) and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17): 7622-7635), vascular endothelial growth factor IRES (Baranick et al. (2008) Proc. Natl. Acad Sci. U.S.A. 105(12):4733-4738, Stein et al. (1998) Mol. Cell. Biol. 18(6):3112-3119, Bert et al. (2006) RNA 12(6): 1074-1083), and insulin-like growth factor 2 IRES (Pedersen et al. (2002) Biochem. J. 363(Pt 1):37-44).

These elements are commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express multiple bacteriophage recombination proteins for recombineering or an RNA-guided nuclease (e.g., Cas9) for HDR in combination with a retron reverse transcriptase from an expression cassette.

In some embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomycin, or tetracycline resistance), a lacZ gene (b-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, METIS, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (Yip), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct does not comprises a plasmid suitable for transforming a yeast cell.

In other embodiments, the expression construct comprises a virus or engineered construct derived from a viral genome. A number of viral based systems have been developed for gene transfer into mammalian cells. These include adenoviruses, retroviruses (g-retroviruses and lentiviruses), poxviruses, adeno-associated viruses, baculoviruses, and herpes simplex viruses (see e.g., Wamock et al. (2011) Methods Mol. Biol. 737:1-25; Walther et al. (2000) Drugs 60(2):249-271; and Lundstrom (2003) Trends Biotechnol. 21(3): 117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24): 2516-2527). Lentiviruses are a class of retroviruses that are particularly useful for delivering polynucleotides to mammalian cells because they are able to infect both dividing and nondividing cells (see e.g., Lois et al. (2002) Science 295:868-872; Durand et al. (2011) Viruses 3(2): 132-159; herein incorporated by reference).

A number of adenoviral vectors have also been described. Unlike retroviruses which integrate into the host genome, adenoviruses persist extrachromosomally thus minimizing the risks associated with insertional mutagenesis.

Additionally, various adeno-associated vims (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor LaboratoryPress); Carter. B. J. Current Opinion in Biotechnology (1992) 3:533-539, Muzyczka, N. Current Topics in Microbiol and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Other viral vectors include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

In some embodiments, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see. Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage 17 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

In other approaches to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification. T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao er al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2nd edition, 2007) and L. King The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al, Mol. Biotech. (1996) 5:209-221; and Hackland et al., Arch. Virol. (1994) 139:1-22.

### Delivery

In order to effect expression of engineered retron ncRNA, retron reverse transcriptase and/or Cas nuclease expression cassettes or vectors must be delivered into a cell. This delivery may be accomplished in vitro, as in laboratory procedures for transforming cells lines, or in vivo or ex vivo, as in the treatment of certain disease states.

Several methods for the transfer of expression constructs into cultured cells can be. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid comprising the engineered retron sequence may be positioned and expressed at different sites. In certain embodiments, the one or more expression cassettes comprising engineered retron ncRNA, retron reverse transcriptase and/or Cas nuclease may be stably integrated into the genome of the cell. This integration may be in the cognate location and orientation via homologous recombination (gene replacement) or it may be integrated in a random, non-specific location (gene augmentation). In yet further embodiments, the expression cassettes may be stably maintained into the cell as a separate, episomal segments of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression cassette(s) may simply consist of naked recombinant DNA or plasmids comprising the engineered retron. Transfer of the expression cassette(s) may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that expression cassette(s) interest may also be transferred in a similar manner in vivo and express engineered retron ncRNAs, retron reverse transcriptases and/or Cas nucleases.

In still another embodiment, naked DNA expression cassettes may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relics on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression cassettes may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposome may be complexed with a hemagglutinin virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091, Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising one or more engineered retron ncRNAs, retron reverse transcriptases and/or Cas nucleases may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration: these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

### Lipid Nanoparticles

The engineered retrons can be delivered by any known delivery system such as those described above. Non-limiting examples of delivery vehicles include lipid particles (e.g., Lipid nanoparticles (LNPs)), non-lipid nanoparticles, exosomes, liposomes, micelles, viral particles, Stable nucleic-acid-lipid particles (SNALPs), lipoplexes/polyplexes, DNA nanoclews, Gold nanoparticles, iTOP, Streptolysin O (SLO), multifunctional envelope-type nanodevice (MEND), lipid-coated mesoporous silica particles, inorganic nanoparticles, and polymeric delivery technology (e.g., polymer-based particles).

In some embodiments, the lipid delivery sytem includes lipid nanoparticles (LNP). In some embodiments the LNP are small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers. In some embodiments. LNPs may comprise a nucleic acid (e.g. engineered retron) into their interior space, into the inter membrane space, onto their exterior surface, or any combination thereof.

In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid. In some embodiments, an LNP further comprises a 5th lipid, besides any of the aforementioned lipid components. In some embodiments, the LNP encapsulates one or more elements of the active agent of the present disclosure. In some embodiments, an LNP further comprises a targeting moiety covalently or non-covalently bound to the outer surface of the LNP. In some embodiments, the targeting moiety is a targeting moiety that binds to, or otherwise facilitates uptake by, cells of a particular organ system.

In some embodiments, an LNP has a diameter of at least about 20 nm, 30 nm, nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 75-80 nm.

In some embodiments, the lipid nanoparticle compositions of the present disclosure are described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG lipid may be from about 0.1 mol-% to about 10 mol-%. In some embodiments, the mol-% of the PEG lipid may be from about 0.2 mol-% to about 5 mol-%. In some embodiments, the mol-% of the PEG lipid may be from about 0.5 mol-% to about 3 mol-%. In some embodiments, the mol-% of the PEG lipid may be from about 1 mol-% to about 2 mol-%. In some embodiments, the mol-% of the PEG lipid may be about 1.5 mol-%.

### i Ionizable Lipids

In some embodiments, an LNP disclosed herein comprises an ionizable lipid. In some embodiments, an LNP comprises two or more ionizable lipids.

In some embodiments, an ionizable lipid has a dimethylamine or an ethanolamine head. In some embodiments, an ionizable lipid has an alkyl tail. In some embodiments, a tail has one or more ester linkages, which may enhance biodegradability. In some embodiments, a tail is branched, such as with 3 or more branches. In some embodiments, a branched tail may enhance endosomal escape. In some embodiments, an ionizable lipid has a pKa between 6 and 7, which may be measured, for example, by TNS assay.

In some embodiments, an ionizable lipid has a structure of any of the formulas disclosed below, and all formulas disclosed in a reference publication and patent application publication cited below. In some embodiments, an ionizable lipid comprises a head group of any structure or formula disclosed below. In some embodiments, an ionizable lipid comprises a bridging moiety of any structure or formula disclosed below. In some embodiments, an ionizable lipid comprises any tail group, or combination of tail groups disclosed below. The present disclosure contemplates all permutations and combinations of head group, bridging moiety and tail group, or tail groups, disclosed herein.

In some embodiments, a head, tail, or structure of an ionizable lipid is described in US patent application US20170210697A1.

In some embodiments, a head, tail, or structure of an ionizable lipid is described in international patent application PCT/US2018/058555.

In some embodiments, a lipid is described in international patent applications WO2021077067, or WO2019152557, each of which is incorporated herein by reference in its entirety.

In some embodiments, an LNP described herein comprises a lipid, e.g., an ionizable lipid, disclosed in US 2019/0240354, which is incorporated herein by reference in its entirety.

In some embodiments, an LNP described herein comprises a lipid, e.g., an ionizable lipid, disclosed in US 2010/0130588, which is incorporated herein by reference in its entirety.

In some embodiments, the lipids disclosed in US 2021/0087135 can be used.

In some embodiments, the lipids disclosed in US 2021/0128488 can be used.

In some embodiments, an LNP described herein comprises a lipid, e.g., an ionizable lipid, disclosed in US 2020/0121809, which is incorporated herein by reference in its entirety.

In some embodiments, the lipids disclosed in US 2020/0121809 can be used.

In some embodiments, an LNP described herein comprises a lipid, e.g., an ionizable lipid, disclosed in US 2013/0108685, which is incorporated herein by reference in its entirety.

In some embodiments, an LNP described herein comprises a lipid, e.g., an ionizable lipid, disclosed in US 2013/0195920, which is incorporated herein by reference in its entirety.

In some embodiments, an LNP described herein comprises a lipid, e.g., an ionizable lipid, disclosed in US 2015/0005363, which is incorporated herein by reference in its entirety.

In some embodiments, an LNP described herein comprises a lipid, e.g., an ionizable lipid, disclosed in US 2014/0308304, which is incorporated herein by reference in its entirety.

In some embodiments, the lipids disclosed in US 2014/0308304 can be used.

In some embodiments, an LNP described herein comprises a lipid, e.g., an ionizable lipid, disclosed in US 2013/0053572, which is incorporated herein by reference in its entirety.

ii. Structural Lipids

In some embodiments, an LNP comprises a structural lipid. Structural lipids can be selected from the group consisting of, but are not limited to, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occurring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

iii. PEGylated Lipids

A PEGylated lipid is a lipid modified with polyethylene glycol. In some embodiments, the LNP comprises a compound of Formula I or a pharmaceutically acceptable salt thereof, as described herein above. In some embodiments, the LNP comprises a compound of Formula II or a pharmaceutically acceptable salt thereof, as described herein above.

In some embodiments, an LNP comprises an additional PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A 1, each of which is incorporated by reference herein in their entirety.

v. Phospholipids

In some embodiments, an LNP of the present disclosure comprises a phospholipid. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleovl-sn-glycero-3-phosphocho line (POPC), 1,2-di-O-octadecenyl-sn-glycero-3-phosphocholine (18:0 Diether PC). 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolaminc, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, an LNP includes DSPC. In certain embodiments, an LNP includes DOPE. In some embodiments, an LNP includes both DSPC and DOPE.

In some embodiments, a phospholipid tail may be modified in order to promote endosomal escape as described in U.S. 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

vi. Targeting Moieties

In some embodiments, the lipid nanoparticle further comprises a targeting moiety. The targeting moiety may be an antibody or a fragment thereof. The targeting moiety may be capable of binding to a target antigen.

In some embodiments, the pharmaceutical composition comprises a targeting moiety that is operably connected to a lipid nanoparticle. In some embodiments, the targeting moiety is capable of binding to a target antigen. In some embodiments, the target antigen is expressed in a target organ.

In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the lipid nanoparticles may be targeted when conjugated/attached/associated with a targeting moiety such as an antibody.

vii. Zwitterionic Amino Lipids

In some embodiments, an LNP comprises a zwitterionic lipid. In some embodiments, an LNP comprising a zwitterionic lipid does not comprise a phospholipid.

Zwitterionic amino lipids have been shown to be able to self-assemble into LNPs without phospholipids to load, stabilize, and release mRNAs intracellular as described in U.S. Patent Application 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destabilizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine α-amine may reduce immunogenicity as described in Walsh et al., Synthesis. Characterization and Evaluation of Ionizable Lysine-Based Lipids for siRNA Delivery, Bioconjug Chem. (2013).

viii. Additional Lipid Components

In some embodiments, the LNP compositions of the present disclosure further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15(4): 313-320.; Dillard, et al. PNAS 2021 Vol. 118 No. 52.).

ix. LNP Compositions

In some embodiments, a nanoparticle includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In another particular embodiment, the lipid component includes about 48.5 mol % ionizable lipid, about 10 mol % phospholipid, about 40 mol % structural lipid, and about 1.5 mol % of PEG lipid. In another particular embodiment, the lipid component includes about 48.5 mol % ionizable lipid, about 10 mol % phospholipid, about 39 mol % structural lipid, and about 2.5 mol % of PEG lipid. In some embodiments, the phospholipid may be DOPE or DSPC. In other embodiments, the PEG lipid may be PEG-DMG and/or the structural lipid may be cholesterol. The amount of active agent in a nanoparticle composition may depend on the size, composition, desired target and/or application, or other properties of the nanoparticle composition as well as on the properties of the active agent. For example, the amount of active agent useful in a nanoparticle composition may depend on the size, sequence, and other characteristics of the active agent. The relative amounts of active agent and other elements (e.g., lipids) in a nanoparticle composition may also vary. In some embodiments, the wt/wt ratio of the lipid component to an enzyme in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a enzyme in a nanoparticle composition may, for example, be measured using absorption spectroscopy (e.g., ultraviolet-visible spectroscopy).

In some embodiments, a nanoparticle composition comprising an active agent of the present disclosure is formulated to provide a specific E:P ratio. The E:P ratio of the composition refers to the molar ratio of nitrogen atoms in one or more lipids to the number of phosphate groups in an RNA active agent. In general, a lower E:P ratio is preferred. The one or more enzymes, lipids, and amounts thereof may be selected to provide an E:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the E:P ratio may be from about 2:1 to about 8:1. In other embodiments, the E:P ratio is from about 5:1 to about 8:1. For example, the E:P ratio may be about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

The mean size of a nanoparticle composition may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 am, from about 80 nm to about 90 am, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741. WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro: Lippincott, Williams & Wilkins, Baltimore, Md., 2006). Other different lipids or liposomal formulations including nanoparticles and methods of administration include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In some embodiments, the LNP encapsulates the engineered retron, e.g., an engineered nucleic acid construct, ncRNA, vector system, RNA molecule, and/or engineered nucleic acid-enzyme construct as described herein.

In some embodiments, the lipid nanoparticle comprises: one or more ionizable lipids; one or more structural lipids; one or more PEGylated lipids; and one or more phospholipids. In some embodiments, the one or more ionizable lipids is selected from the group consisting of those disclosed in Table X.

In some embodiments, the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone. In some embodiments, the one or more PEGylated lipids are selected from the group consisting of PEG-c-DOMG, PEG-DMG, PEG-DLPE, PEG-DMPE, PEG-DPPC, and PEG-DSPE.

In some embodiments, the one or more phospholipids are selected from the group consisting of 1,2-distearoyl-sn-glycero-3-phosphocholinc (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundccanoyl-sn-glycero-phosphocholine (DUPC), 1-palmitoyl-2-olcoyl-sn-glycero-3-phosphocho line (POPC), 1,2-di-O-octadecenyl-sn-glycero-3-phosphocholine (18:0 Diether PC), 1-oleoyl-2-cholesterylhemisuc cinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolaminc, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle comprises about 48.5 mol % ionizable lipid, about 10 mol % phospholipid, about 40 mol % structural lipid, and about 1.5 mol % of PEG lipid.

In some embodiments, the lipid nanoparticle comprises about 48.5 mol % ionizable lipid, about 10 mol % phospholipid, about 39 mol % structural lipid, and about 2.5 mol % of PEG lipid. In some embodiments, the LNP further comprises a targeting moiety operably connected to the LNP. In some embodiments, the LNP further comprises one or more additional components selected from the group consisting of DDAB, EPC, 14PA, 18BMP, DODAP, DOTAP, and C12-200.

In some embodiments, the engineered retron can be used for gene transfer, which may be performed under ex vivo or in vivo conditions. Ex vivo gene therapy refers to the isolation of cells from a subject, the delivery of a nucleic acid into cells in vitro, and the return of the modified cells back into the subject. This may involve the collection of a biological sample comprising cells from the subject. For example, blood can be obtained by venipuncture, and solid tissue samples can be obtained by surgical techniques according to methods well known in the art.

Usually, but not always, the subject who receives the cells (e.g., the recipient) is also the subject from whom the cells are harvested or obtained, which provides the advantage that the donated cells are autologous. However, cells can be obtained from another subject (e.g., a donor), a culture of cells from a donor, or from established cell culture lines. Accordingly, in some embodiments the cells are allogeneic to the recipient. Cells may be obtained from the same or a different species than the subject to be treated, but preferably are of the same species, and more preferably of the same immunological profile as the subject. Such cells can be obtained, for example, from a biological sample comprising cells from a close relative or matched donor, then transfected with nucleic acids (e.g., comprising an engineered retron), and administered to a subject in need of genome modification, for example, for treatment of a disease or condition.

In other embodiments, the engineered retron can be introduced in vivo (e.g., used in gene therapy) by physically delivering the engineered retron to a subject. Examples of physically introducing the engineered retron includes via injections, electroporation and transfection (e.g., calcium-mediated or liposome transfection, or the like).

### Cells

One aspect of the disclosure provides an isolated host cell that includes one or more of the compositions described herein, including, but not limited to, engineered retrons and/or retron components, engineered ncRNAs, engineered msDNA, engineered RT, nucleic acid molecules encoding the engineered retrons and/or retron components, and vector or vector systems encoding the engineered retrons and/or retron components, and any combinations thereof. In some embodiments, the host cell is a prokaryotic cell, an archaeal cell, or a eukaryotic host cell. In some embodiments, the eukaryotic host cell is a mammalian cell, such as a human cell, a non-human cell, or a non-human mammalian cell. In some embodiments, the host cell is an artificial cell or genetically modified cell. In some embodiments, the host cell is in vitro, such as a tissue culture cell. In some embodiments, the host cell is within a living host organism.

Cells that may contain any of the compositions described herein. The methods described herein are used to deliver recombinant retrons or components thereof into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

The present disclosure contemplates the use of any suitable host cell. For example, the cell host can be a mammalian cell. Mammalian cells of the present disclosure include human cells, primate cells (e.g., vero cells), rat cells (e.g., GH3 cells. OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells. Lncap (prostate cancer) cells, MCF-7 (breast cancer) cells, MDA-MB-438 (breast cancer) cells, PC3 (prostate cancer) cells, T47D (breast cancer) cells, THP-1 (acute mycloid leukemia) cells, U87 (glioblastoma) cells, SHSY5Y human neuroblastoma cells (cloned from a myeloma) and Saos-2 (bone cancer) cells. In some embodiments, the cells can be human embryonic kidney (HEK) cells (e.g., HEK 293 or HEK 293T cells). In some embodiments, the cells can be stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cells express stem cell markers and are capable of generating cells characteristic of all three germ layers (ectoderm, endoderm, mesoderm).

Some aspects of this disclosure provide cells comprising any of the compositions disclosed herein, including, but not limited to, engineered retrons and/or retron components, engineered ncRNAs, engineered msDNA, engineered RT, nucleic acid molecules encoding the engineered retrons and/or retron components, and vector or vector systems encoding the engineered retrons and/or retron components, and any combinations thereof. In some embodiments, a host cell is transiently or non-transiently transfected with one or more delivery systems described herein, including virus-based systems, virus-like particle systems, and non-virus-base delivery, including LNPs and liposomes. In some embodiments, a cell is transfected as it naturally occurs in a subject. In some embodiments, a cell that is transfected is taken from a subject, i.e., ex vivo transfection. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR. Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4. COS. COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3. EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KGI, KYOI, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more retron delivery systems described herein is used to establish a new cell line comprising one or more nucleic acid molecules encoding the recombinant retron-based gene editing systems described herein, or encoding at last a component of said systems (e.g., a recombinant ncRNA or a recombinant retron RT).

### Pharmaceutical Compositions

The engineered retron-based genome editing systems described herein, or one or more components thereof (e.g., engineered ncRNAs, engineered msDNA, engineered RT, nucleic acid molecules encoding the engineered retrons and/or retron components, guide RNAs, programmable nucleases) may be provided as pharmaceutical compositions. For example, one or more LNPs or other non-virus-based delivery system comprising one or more circular or linear RNA molecules encoding each of the components of the retron-based genome editing system may be formulated as a pharmaceutical composition for administering to a subject in need (e.g., a human in need of gene editing).

Formulations can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers an engineered retron as described herein.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Other aspects of the present disclosure relate to pharmaceutical compositions comprising any of the various components of the recombinant retron-based genome editing systems described herein, including, but not limited to, engineered retrons and/or retron components, engineered ncRNAs, engineered msDNA, engineered RT, nucleic acid molecules encoding the engineered retrons and/or retron components, programmable nucleases (e.g., RNA-guided nucleases), guide RNAs, and vector or vector systems encoding the engineered retrons and/or retron components, and any combinations thereof. The term“pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is“acceptable” in the sense of being compatible with the other ingredients of the formulation and not injurious to the tissue of the subject (e.g., physiologically compatible, sterile, physiologic pH, etc.).

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosscus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985. Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid. e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal or LNP, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxy)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016, and 4,921,757; each of which is incorporated herein by reference.

Further, the pharmaceutical composition can be provided as a pharmaceutical kit comprising (a) a container containing a recombinant retron-based genome editing system or one or more components thereof in lyophilized form and (b) a second container containing a pharmaceutically acceptable diluent (e.g., sterile water) for injection. The pharmaceutically acceptable diluent can be used for reconstitution or dilution of the lyophilized system of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Uses and Methods

The engineered retrons, components, and systems described herein can be used in a variety of applications, several non-limiting examples of which are described herein. In general, the engineered retron can be used in any suitable organism. In some embodiments, the organism is a eukaryote.

In some embodiments, the organism is an animal. In some embodiments, the animal is a fish, an amphibian, a reptile, a mammal, or a bird. In some embodiments, the animal is a farm animal or agriculture animal. Non-limiting examples of farm and agriculture animals include horses, goats, sheep, swine, cattle, llamas, alpacas, and birds, e.g., chickens, turkeys, ducks, and geese. In some embodiments, the animal is a non-human primate, e.g., baboons, capuchin monkeys, chimpanzees, lemurs, macaques, marmosets, tamarins, spider monkeys, squirrel monkeys, and vervet monkeys. In some embodiments, the animal is a pet. Non-limiting examples of pets include dogs, cats, horses, wolfs, rabbits, ferrets, gerbils, hamsters, chinchillas, fancy rats, guinea pigs, canaries, parakeets, and parrots.

In some embodiments, the organism is a plant. Plants that may be transfected with an engineered retron include monocots and dicots. Particular examples include, but arm not limited to, corn (maize), sorghum, wheat, sunflower, potato, cotton, rice, soybean, sugarbeet, sugarcane, tobacco, barley, and oilseed rape. Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers. Vegetables include, but are not limited to, crucifers, peppers, tomatoes, lettuce, green beans, lima beans, peas, and members of the genus Cucumis such as cucumber, cantaloupe, and musk melon. Ornamentals include, but are not limited to, azalea, hydrangea, hibiscus, roses, tulips, daffodils, petunias, carnation, poinsettia, and chrysanthemum.

In some embodiments, the engineered retrons, components, and systems described herein may be used for research tools, such as kits, functional genomics assays, and generating engineered cell lines and animal models for research and drug screening. The kit may comprise one or more reagents in addition to the engineered retron, such as a buffer, a control reagent, a control vector, a control RNA polynucleotide, a reagent for in vitro production of the polypeptide from DNA, and adaptors for sequencing. A buffer can be, for example, a stabilization buffer, a reconstituting buffer, a diluting buffer, a wash buffer, or a buffer for introducing a polypeptide and/or polynucleotide of the kit into a cell. In some instances, a kit can comprise one or more additional reagents specific for plants. One or more additional reagents for plants can include, for example, soil, nutrients, plants, seeds, spores, Agrobacterium, a T-DNA vector, and a pBINAR vector.

Exemplary but non-limiting uses may include the following.

### Production of Protein or RNA

In some embodiments, the single-stranded msDNA generated by the engineered retron of the invention can be used to produce a desired product of interest in cells.

In some embodiments, the retron is engineered with a heterologous sequence encoding a polypeptide of interest to allow production of the polypeptide from the retron msDNA generated in a cell. The polypeptide of interest may be any type of protein/peptide including, without limitation, an enzyme, an extracellular matrix protein, a receptor, transporter, ion channel, or other membrane protein, a hormone, a neuropeptide, an antibody, or a cytoskeletal protein, a functional fragment thereof, or a biologically active domain of interest. In some embodiments, the protein is a therapeutic protein, therapeutic antibody for use in treatment of a disease, or a template to fix a mutation or mutated exon in the genome.

Non-limiting examples of polypeptides of interest include: growth hormones, insulin-like growth factors (IGF-1), Fat-1, Phytase, xylanase, beta-glucanase, Lysozyme or lysostaphin, Histone deacetylase such as HDAC6, CD163, etc.

In other embodiments, the retron is engineered with a heterologous sequence encoding an RNA of interest to allow production of the RNA from the retron in a cell. The RNA of interest may be any type of RNA including, without limitation, a RNA interference (RNAi) nucleic acid or regulatory RNA such as, but not limited to, a microRNA (miRNA), a small interfering RNA (siRNA), a short hairpin RNA (shRNA), a small nuclear RNA (snRNA), a long non-coding RNA (lncRNA), an antisense nucleic acid, and the like.

### Gene Editing

In some embodiments, the retron is used for genome editing a desired site. A retron is engineered with a heterologous nucleic acid sequence encoding a donor polynucleotide suitable for use with nuclease genome editing system. The nuclease is designed to specifically target a location proximal to the desired edit (the nuclease should be designed such that it will not cut the target once the edit is properly installed). The nuclease (e.g., Cas or non-Cas) is linked to the retron, either by direct fusion to the RT or by fusion of the msDNA to the gRNA (only applicable for RNA-guided nucleases). A heterologous nucleic acid sequence is inserted into the retron msd. See for example FIG. 3, which shows a marker representing the edit.

In some embodiments, the heterologous nucleic acid sequence has 10-100 or more bp of homologous nucleic acid sequence to the genome on both sides of the desired edit. The desired edit (insertion, deletion, or mutation) is in between the homologous sequence.

In some embodiments, donor polynucleotides comprise a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′homology arm that hybridizes to a 3′ genomic target sequence. The homology arms are referred to herein as 5′ and 3′ (i.e., upstream and downstream) homology arms, which relate to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence.” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by the 5′ and 3′homology arms.

In some embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In some embodiments, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 15 nucleotides or more, 20 nucleotides or more, 50 nucleotides or more, 100 nucleotides or more, 250 nucleotides or more, 300 nucleotides or more, 350 nucleotides or more. 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another. However, in some instances the 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm. 2% shorter or less than the other homology arm, or only a few nucleotides less than the other homology arm. In other instances, the 5′ and 3′ homology arms are substantially different in length from one another, e.g., one may be 40% shorter or more. 50% shorter or more, sometimes 60% shorter or more. 70% shorter or more, 80% shorter or more, 90% shorter or more, or 95% shorter or more than the other homology arm.

The donor polynucleotide may be used in combination with an RNA-guided nuclease, which is targeted to a particular genomic sequence (i.e., genomic target sequence to be modified) by a guide RNA. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In some embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution. Such genetically modified cells can be used, for example, to alter phenotype, confer new properties, or produce disease models for drug screening.

In some embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx-2), Cas1O, Cas1Od, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16. CsaX, Csx3, Csx1, Csx15, Csf1, Csf2, Csf3, Csf4, and Cul966, and homologs or modified versions thereof.

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP_022552435, YP 002344900). Campylobacter coli (WP 060786116); Campylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC O 18721); Streptococcus thermophilus (YP 820832). Streptococcus mutans (WP 061046374, WP 024786433); Listeria innocua (NP 472073); Listeria monocytogenes (WP 061665472); Legionella pneunmophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacterid. 198(5): 797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The genomic target site will typically comprise a nucleotide sequence that is complementary to the gRNA and may further comprise a protospacer adjacent motif (PAM). In some embodiments, the target site comprises 20-30 base pairs in addition to a 3 or more base pair PAM. Typically, the first nucleotide of a PAM can be any nucleotide, while the two or more other nucleotides will depend on the specific Cas9 protein that is chosen. Exemplary PAM sequences are known to those of skill in the art and include, without limitation, NNG, NGN, NAG, and NGG, wherein N represents any nucleotide. In some embodiments, the allele targeted by a gRNA comprises a mutation that creates a PAM within the allele, wherein the PAM promotes binding of the Cas9-gRNA complex to the allele.

In some embodiments, the gRNA is 5-50 nucleotides, 10-30 nucleotides, 15-nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, or Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide oveiang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

C2c1 (Cas12b) is another class II CRISPR/Cas system RNA-guided nuclease that may be used. C2c1, similarly to Cas9, depends on both a crRNA and tracrRNA for guidance to target sites. See, e.g., Shmakov et al. (2015) Mol Cell. 60(3):385-397. Zhang et al. (2017) Front Plant Sci. 8:177; herein incorporated by reference.

In yet another embodiment, an engineered RNA-guided Fokl nuclease may be used. RNA-guided Fokl nucleases comprise fusions of inactive Cas9 (dCas9) and the Fokl endonuclease (FokI-dCas9), wherein the dCas9 portion confers guide RNA-dependent targeting on Fokl. For a description of engineered RNA-guided Fold nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

In other embodiments, any other Cas enzymes and variants described in other sections of the application (all incorporated herein) can be used similarly.

In some embodiments, the RNA-guided nuclease is provided in the form of a protein, optionally where the nuclease is complexed with a gRNA to form a ribonucleoprotein (RNP) complex. In some embodiments, the RNA-guided nuclease is provided by a nucleic acid encoding the RNA-guided nuclease, such as an RNA (e.g., messenger RNA) or DNA (expression vector). In some embodiments, the RNA-guided nuclease and the gRNA are both provided by vectors, such as the vectors and the vector system described in other parts of the application (all incorporated herein by reference). Both can be expressed by a single vector or separately on different vectors. The vectors encoding the RNA-guided nuclease and gRNA may be included in the vector system comprising the engineered retron msr gene, msd gene and ret gene sequences. In some embodiments, the RNA-guided nuclease is fused to the RT and/or the msDNA.

The RNP complex may be administered to a subject or delivered into a cell by methods known in the art, such as those described in U.S. Pat. No. 11,390,884, which is incorporated by reference herein in its entirety. In some embodiments, the endonuclease/gRNA ribonucleoprotein (RNP) complexes are delivered to cells by electroporation. Direct delivery of the RNP complex to a subject or cell eliminates the need for expression from nucleic acids (e.g., transfection of plasmids encoding Cas9 and gRNA). It also eliminates unwanted integration of DNA segments derived from nucleic acid delivery (e.g., transfection of plasmids encoding Cas9 and gRNA). An endonuclease/gRNA ribonucleoprotein (RNP) complex usually is formed prior to administration.

Codon usage may be optimized to further improve production of an RNA-guided nuclease and/or reverse transcriptase (RT) in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

In some embodiments, the engineered retron used for genome editing with nuclease genome editing systems can further include accessory or enhancer proteins for recombination. Examples of recombination enhancers can include nonhomologous end joining (NHEJ) inhibitors (e.g., inhibitor of DNA ligase IV, a KU inhibitor (e.g., KU70 or KU80), a DNA-PKc inhibitor, or an artemis inhibitor) and homologous directed repair (HDR) promoters, or both, that can enhance or improve more precise genome editing and/or the efficiency of homologous recombination. In some embodiments, the recombination accessory or enhancers can comprise C-terminal binding protein interacting protein (CtIP), cyclinB2, Rad family members (e.g. Rad50, Rad51, Rad52, etc).

CtIP is a transcription factor containing C2H2 zinc fingers that are involved in early steps of homologous recombination. Mammalian CtIP and its orthologs in other eukaryotes promote the resection of DNA double-strand breaks and are essential for meiotic recombination. HDR may be enhanced by using Cas9 nuclease associated (e.g. fused) to an N-terminal domain of CtIP, an approach that forces CtIP to the cleavage site and increases transgene integration by HDR. In some embodiments, an N-terminal fragment of CtIP, called HE for HDR enhancer, may be sufficient for HDR stimulation and requires the CtIP multimerization domain and CDK phosphorylation sites to be active. HDR stimulation by the Cas9-HE fusion depends on the guide RNA used, and therefore the guide RNA will be designed accordingly.

Using the gene editing system described herein, any target gene or sequence in a host cell can be edited or modified for a desired trait, including but not limited to: Myostatin (e.g., GDF8) to increase muscle growth, Pc POLLED to induce hairlessness; KISS1R to induce bore taint; Dead end protein (dnd) to induce sterility; Nano2 and DDX to induce sterility; CD163 to induce PRRSV resistance; RELA to induce ASFV resilience; CD18 to induce Mannheimia (Pasteurella) haemolytica resilience; NRAMP1 to induce tuberculosis resilience; Negative regulators of muscle mass (e.g., Myostatin) to increase muscle mass.

### Recombineering

Recombineering (recombination-mediated genetic engineering) can be used in modifying chromosomal as well as episomal replicons in cells, for example, to create gene replacements, gene knockouts, deletions, insertions, inversions, or point mutations. Recombineering can also be used to modify a plasmid or bacterial artificial chromosome (BAC), for example, to clone a gene or insert markers or tags.

The engineered retrons described herein can be used in recombineering applications to provide linear single-stranded or double-stranded DNA for recombination. Homologous recombination may be mediated by bacteriophage proteins such as RecE/RecT from Rac prophage or Redobd from bacteriophage lambda. The linear DNA should have sufficient homology at the 5′ and 3′ ends to a target DNA molecule present in a cell (e.g., plasmid, BAC, or chromosome) to allow recombination.

The linear double-stranded or single-stranded DNA molecule used in recombineering (i.e., donor polynucleotide) comprises a sequence having the intended edit to be inserted flanked by two homology arms that target the linear DNA molecule to a target site for homologous recombination. Homology arms for recombineering typically range in length from 13-300 nucleotides, or 20 to 200 nucleotides, including any length within this range such as 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nucleotides in length. In some embodiments, a homology arm is at least 15, at least 20, at least 30, at least 40, or at least 50 or more nucleotides in length. Homology arms ranging from 40-50 nucleotides in length generally have sufficient targeting efficiency for recombination; however, longer homology arms ranging from 150 to 200 bases or more may further improve targeting efficiency. In some embodiments, the 5′ homology arm and the 3′homology arm differ in length. For example, the linear DNA may have about 50 bases at the 5′ end and about 20 bases at the 3′ end with homology to the region to be targeted.

The bacteriophage homologous recombination proteins can be provided to a cell as proteins or by one or more vectors encoding the recombination proteins, such as the vector or vector system. In some embodiments, one or more vectors encoding the bacteriophage recombination proteins are included in the vector system comprising the engineered retron msr gene, msd gene, and/or ret gene sequences. Additionally, a number of bacterial strains containing prophage recombination systems are available for recombineering, including, without limitation, DY380, containing a defective 1 prophage with recombination proteins exo, bet, and gam; EL250, derived from DY380, which in addition to the recombination genes found in DY380, also contains a tightly controlled arabinose-inducible flpe gene (flpe mediates recombination between two identical frt sites); EL350, also derived from DY380, which in addition to the recombination genes found in DY380, also contains a tightly controlled arabinose-inducible ere gene (ere mediates recombination between two identical loxP sites; SW102, derived from DY380, which is designed for BAC recombineering using a galK positive/negative selection; SW105, derived from EL250, which can also be used for galK positive/negative selection, but like EL250, contain an ara-inducible Flpe gene; and SW106, derived from EL350, which can be used for galK positive/negative selection, but like EL350, contains an ara-inducible Cre gene. Recombineering can be carried out by transfecting bacterial cells of such strains with an engineered retron comprising a heterologous sequence encoding a linear DNA suitable for recombineering. For a discussion of recombineering systems and protocols, see, e.g., Sharan et al. (2009) Nat Protoc. 4(2): 206-223, Zhang et al. (1998) Nature Genetics 20: 123-128, Muyrers et al. (1999) Nucleic Acids Res. 27: 1555-1557, Yu et al. (2000) Proc. Natl. Acad. Sci U.S.A. 97 (11):5978-5983; herein incorporated by reference.

### Molecular Recording

In some embodiments, the heterologous sequence in the engineered retron construct comprises a synthetic CRISPR protospacer DNA sequence to allow molecular recording. The endogenous CRISPR Cas1-Cas2 system is normally utilized by bacteria and archaea to keep track of foreign DNA sequences originating from viral infections by storing short sequences (i.e., protospacers) that confer sequence-specific resistance to invading viral nucleic acids within genome-based arrays. These arrays not only preserve the spacer sequences but also record the order in which the sequences are acquired, generating a temporal record of acquisition events.

This system can be adapted to record arbitrary DNA sequences into a genomic CRISPR array in the form of “synthetic protospacers” that are introduced into cells using engineered retrons. Engineered retrons carrying the protospacer sequences can be used for integration of synthetic CRISPR protospacer sequences at a specific genomic locus by utilizing the CRISPR system Cas1-Cas2 complex. Molecular recording can be used to keep track of certain biological events by producing a stable genetic memory tracking code. See, e.g., Shipman el al. (2016) Science 353(6298): aafl 175 and International Patent Application Publication No. WO/2018/191525; herein incorporated by reference in their entireties.

In some embodiments, the CRISPR-Cas system is harnessed to record specific and arbitrary DNA sequences into a bacterial genome. The DNA sequences can be produced by an engineered retron within the cell. For example, the engineered retron can be used to produce the protospacers within the cell, which are inserted into a CRISPR array within the cell. The cell may be modified to include one or more engineered returns (or vector systems encoding them) that can produce one or more synthetic protospacers in the cell, wherein the synthetic protospacers are added to the CRISPR array. A record of defined sequences, recorded over many days, and in multiple modalities can be generated.

In some embodiments, the engineered retron comprises an msd protospacer nucleic acid region or an msr protospacer nucleic acid region. In the case of a msr protospacer nucleic acid region, the protospacer sequence is first incorporated into the msr RNA, which is reverse transcribed into protospacer DNA. Double stranded protospacer DNA is produced when two complementary protospacer DNA sequences having complementary sequences hybridize, or when a double-stranded structure (such as a hairpin) is formed in a single stranded protospacer DNA (e.g., a single msDNA can form an appropriate hairpin structure to provide the double stranded DNA protospacer).

In some embodiments, a single stranded DNA produced in vivo from a first engineered retron may be hybridized with a complementary single-stranded DNA produced in vivo from the same retron or a second engineered retron or may form a hairpin structure and then used as a protospacer sequence to be inserted into a CRISPR array as a spacer sequence. The engineered retron(s) should provide sufficient levels of the protospacer sequence within a cell for incorporation into the CRISPR array. The use of protospacers generated within the cell extends the in vivo molecular recording system from only capturing information known to a user, to capturing biological or environmental information that may be previously unknown to a user. For example, an msDNA protospacer sequence in an engineered retron construct may be driven by a promoter that is downstream of a sensor pathway for a biological phenomenon or environmental toxin. The capture and storage of the protospacer sequence in the CRISPR array records the event. If multiple msDNA protospacers are driven by different promoters, the activity of those promoters is recorded (along with anything that may be upstream of the promoters) as well as the relative order of promoter activity (based on the relative position of spacer sequences in the CRISPR array). At any point after the recording has taken place, the CRISPR array may be sequenced to determine whether a given biological or environmental event has taken place and the order of multiple events, given by the presence and relative position of msDNA-derived spacers in the CRISPR array.

In some embodiments, the synthetic protospacer further comprises an AAG PAM sequence at its 5′ end. Protospacers including the 5′ AAG PAM are acquired by the CRISPR array with greater efficiency than those that do not include a PAM sequence.

In some embodiments, Cas1 and Cas2 are provided by a vector that expresses the Cas1 and Cas2 at a level sufficient to allow the synthetic protospacer sequences produced by engineered retrons to be acquired by a CRISPR array in a cell. Such a vector system can be used to allow molecular recording in a cell that lacks endogenous Cas proteins.

### Therapeutic Applications

The engineered ncRNAs, reverse transcriptases, Cas nucleases, and the expression systems described herein and/or cells containing the engineered ncRNAs, reverse transcriptases, Cas nucleases, or expression systems can be administered to a subject. Such a subject may suffer from a disease or condition or be suspected of suffering from a disease or condition. Symptoms of the disease or condition can be reduced by such administration. In some cases, progression of the disease or condition can be prevented or reduced by such administration. In some cases, the subject may be asymptomatic but be genetically pre-disposed to developing disease or condition.

Hence, described herein are methods of administering one or more engineered ncRNAs, reverse transcriptases, Cas nucleases, and/or expression systems therefor and/or cells containing the engineered ncRNAs, reverse transcriptases, Cas nucleases, to a subject. The methods can provide prophylaxis, amelioration and/or therapy for a variety of diseases or conditions, including cystic fibrosis, thalassemia, sickle cell anemia. Huntington's disease, diabetes, Duchenne's Muscular Dystrophy, Tay-Sachs Disease, Marfan syndrome. Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

Also provided herein are methods of diagnosing, prognosing, treating, and/or preventing a disease, state, or condition in or of a subject, using the engineered retron of the invention.

Generally, the methods of diagnosing, prognosing, treating, and/or preventing a disease, state, or condition in or of a subject can include modifying a polynucleotide in a subject or cell thereof using a composition, system, or component thereof of the engineered retron as described herein, and/or include detecting a diseased or healthy polynucleotide in a subject or cell thereof using a composition, system, or component thereof of the engineered retron as described herein.

In some embodiments, the method of treatment or prevention can include using a composition, system, or component of the engineered retron to modify a polynucleotide of an infectious organism (e.g., bacterial or virus) within a subject or cell thereof.

In some embodiments, the method of treatment or prevention can include using a composition, system, or component of the engineered retron to modify a polynucleotide of an infectious organism or symbiotic organism within a subject.

In some embodiments, the composition, system, and components of the engineered retron can be used to develop models of diseases, states, or conditions.

In some embodiments, the composition, system, and components of the engineered retron can be used to detect a disease state or correction thereof, such as by a method of treatment or prevention described herein.

In some embodiments, the composition, system, and components of the engineered retron can be used to screen and select cells that can be used, for example, as treatments or preventions described herein.

In some embodiments, the composition, system, and components thereof can be used to develop biologically active agents that can be used to modify one or more biologic functions or activities in a subject or a cell thereof.

In general, the method can include delivering a composition, system, and/or component of the engineered retron to a subject or cell thereof, or to an infectious or symbiotic organism by a suitable delivery technique and/or composition. Once administered, the components can operate as described elsewhere herein to elicit a nucleic acid modification event. In some embodiments, the nucleic acid modification event can occur at the genomic, epigenomic, and/or transcriptomic level. DNA and/or RNA cleavage, gene activation, and/or gene deactivation can occur.

The composition, system, and components of the engineered retron as described elsewhere herein can be used to treat and/or prevent a disease, such as a genetic and/or epigenetic disease, in a subject; to treat and/or prevent genetic infectious diseases in a subject, such as bacterial infections, viral infections, fungal infections, parasite infections, and combinations thereof; to modify the composition or profile of a microbiome in a subject, which can in turn modify the health status of the subject; to modify cells ex vivo, which can then be administered to the subject whereby the modified cells can treat or prevent a disease or symptom thereof; or to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein of the engineered retron, and administering them to the subject.

Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component of the engineered retron, and comprising multiple Cas effectors.

Also provided is a method of treating a subject, e.g., a subject in need thereof, comprising inducing gene editing by transforming the subject with the Cas effector(s), and encoding and expressing in vivo the remaining portions of the composition, system, (e.g., RNA, guides), complex or component of the engineered retron. A suitable repair template may also be provided by the engineered retron as described herein elsewhere.

Also provided is a method of treating a subject, e.g., a subject in need thereof, comprising inducing transcriptional activation or repression by transforming the subject with the systems or compositions herein.

Also provided is a method of inducing one or more polynucleotide modifications in a eukaryotic or prokaryotic cell or component thereof (e.g. a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus vector (in viral particle) of any one of the above embodiments or the cell of any one of the above embodiments in ex vivo or in vivo gene or genome editing; or for use in in vitro, ex vivo or in vivo gene therapy.

Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, target polynucleotide modification using the subject engineered retron and the associated composition, vectors, system and methods comprises addition, deletion, or substitution of 1-about 10 k nucleotides at each target sequence of said polynucleotide of said cell(s). The modification can include the addition, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, 75, 100, 200, 250, 300, 500, 1000, 1500, 2000, 2500, 3000, 3500, 4000, 5000, 6000, 7000, 8000, 9000, 10,000 or more nucleotides at each target sequence.

In some embodiments, formation of system or complex results in cleavage, nicking, and/or another modification of one or both strands in or near (e.g., within 1, 2, 3, 4, 5, 6, 7, 8, 9, 10, 20, 50, or more base pairs from) the target sequence.

In some embodiments, a method of modifying a target polynucleotide in a cell to treat or prevent a disease can include allowing a composition, system, or component of the subject engineered retron to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some embodiments, modification can include cleaving or nicking one or two strands at the location of the target sequence by one or more components of the composition, system, or component thereof.

In some embodiments, the engineered retron and the associated compositions, systems, vectors, uses, and methods of use, can be used to treat diseases of the circulatory system. In some embodiments, the treatment can be carried out by using an AAV or a lentiviral vector to deliver the engineered retron, composition, system, and/or vector described herein to modify hematopoietic stem cells (HSCs) or iPSCs in vivo or ex vivo. In some embodiments, the treatment can be carried out by correcting HSCs or iPSCs as to the disease using a composition, system, herein or a component thereof, wherein the composition, system, optionally includes a suitable HDR repair template (e.g., a template in the msDNA of the engineered retron).

In some embodiments, the treatment or prevention for treating a circulatory system or blood disease can include modifying a human cord blood cell. In some embodiments, the treatment or prevention for treating a circulatory system or blood disease can include modifying a granulocyte colony-stimulating factor-mobilized peripheral blood cell (mPB) with any modification described herein. In some embodiments, the human cord blood cell or mPB can be CD34⁺. In some embodiments, the cord blood cells or mPB cells modified are autologous. In some embodiments, the cord blood cells or mPB cells are allogenic. In addition to the modification of the disease genes, allogenic cells can be further modified using the composition, system, described herein to reduce the immunogenicity of the cells when delivered to the recipient. The modified cord blood cells or mPB cells can be optionally expanded in vitro. The modified cord blood cell(s) or mPB cells can be derived to a subject in need thereof using any suitable delivery technique.

The composition and system may be engineered to target genetic locus or loci in HSCs. In some embodiments, the components of the systems can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles, such as the lipid nanoparticle delivery system described herein. The particles may be formed by the components of the systems herein being admixed.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In some embodiments, the HSCs or iPSCs modified are autologous. In some embodiments, the HSCs or iPSCs are allogenic. In addition to the modification of the disease genes, allogenic cells can be further modified using the composition, system, described herein to reduce the immunogenicity of the cells when delivered to the recipient.

In some embodiments, the engineered retron and the associated compositions, systems, vectors, uses, and methods of use, can be used to treat neurological diseases. In some embodiments, the neurological diseases comprise diseases of the brain and CNS.

Delivery options for the diseases in the brain include encapsulation of the systems in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors or vector systems of the invention. In other embodiments, an artificial virus can be generated for CNS and/or brain delivery.

In some embodiments, the engineered retron and the associated compositions, systems, vectors, uses, and methods of use, can be used to treat hearing diseases or hearing loss in one or both ears. Deafness is often caused by lost or damaged hair cells that cannot relay signals to auditory neurons. In some embodiments, the composition, system, or modified cells can be delivered to one or both cars for treating or preventing hearing disease or loss by any suitable method or technique known in the art, such as US20120328580 (e.g., auricular administration), by intratympanic injection (e.g., into the middle ear), and/or injections into the outer, middle, and/or inner ear; administration in situ, via a catheter or pump (U.S. 2006/0030837) and Jacobsen (U.S. Pat. No. 7,206,639). Also see US20120328580. Cells resulting from such methods can then be transplanted or implanted into a patient in need of such treatment.

In some embodiments, the engineered retron and the associated compositions, systems, vectors, uses, and methods of use, can be used to treat diseases in non-dividing cells. Exemplary non-dividing cells include muscle cells or neurons. In such cells, homologous recombination (HR) is generally suppressed in the G1 cell-cycle phase, but can be turned back on using art-recognized methods, such as Orthwein et al. (Nature. 2015 Dec. 17; 528(7582): 422-426).

In some embodiments, the engineered retron and the associated compositions, systems, vectors, uses, and methods of use, can be used to treat diseases of the eye.

In some embodiments, the engineered retron and the associated compositions, systems, vectors, uses, and methods of use, can be used to treat muscle diseases and cardiovascular diseases.

In some embodiments, the engineered retron and the associated compositions, systems, vectors, uses, and methods of use, can be used to treat diseases of the liver and kidney.

In some embodiments, the engineered retron and the associated compositions, systems, vectors, uses, and methods of use, can be used to treat epithelial and lung diseases.

In some embodiments, the engineered retron and the associated compositions, systems, vectors, uses, and methods of use, can be used to treat diseases of the skin.

In some embodiments, the engineered retron and the associated compositions, systems, vectors, uses, and methods of use, can be used to treat cancer.

In some embodiments, the engineered retron and the associated compositions, systems, vectors, uses, and methods of use, can be used in adoptive cell therapy.

In some embodiments, the engineered retron and the associated compositions, systems, vectors, uses, and methods of use, can be used to treat infectious diseases.

In some embodiments, the engineered retron and the associated compositions, systems, vectors, uses, and methods of use, can be used to treat mitochondrial diseases.

All publications, published patent documents, and patent applications cited herein are hereby incorporated by reference to the same extent as though each individual publication, published patent document, or patent application was specifically and individually indicated as being incorporated by reference.

### Ex Vivo Cellular Modification

In certain embodiments, gene transfer may more easily be performed under ex vivo conditions. Ex vivo gene therapy refers to the isolation of cells from a subject, the delivery of a nucleic acid into cells in vitro, and then the return of the modified cells back into the subject. This may involve the collection of a biological sample comprising cells from the subject. For example, blood can be obtained by venipuncture, cells can be obtained by scrapings, and solid tissue samples can be obtained by surgical techniques etc. according to methods available in the art.

Usually, but not always, the subject who receives the cells (i.e., the recipient) is also the subject from whom the cells are harvested or obtained, which provides the advantage that the donated cells are autologous. However, cells can be obtained from another subject (i.e., donor), a culture of cells from a donor, or from established cell culture lines. Cells may be obtained from the same or a different species than the subject to be treated, but preferably are of the same species, and more preferably of the same immunological profile as the subject. Such cells can be obtained, for example, from a biological sample comprising cells from a close relative or matched donor, then transfected with nucleic acids (e.g., comprising an engineered retron), and administered to a subject in need of genome modification, for example, for treatment of a disease or condition.

### Kits

Also provided are kits comprising expression cassettes or expression systems for retron ncRNA, reverse transcriptases, and/or Cas nucleases constructs as described herein. The expression cassettes or expression systems of the kits can include a promoter operably linked to a DNA segment encoding a retron ncRNA, where the ncRNA with instructions for inserting a guide RNA sequence and/or a repair template sequence into the ncRNA. The expression cassettes or expression systems of the kits can include a promoter operably linked to a DNA segment encoding a reverse transcriptase and/or Cas nuclease. Other agents may also be included in the kit such as transfection agents, host cells, suitable media for culturing cells, buffers, and the like.

In the context of a kit, the components can be provided in liquid or sold form in any convenient packaging (e.g., vials, powders pack, dose pack, etc.). The components of a kit can be present in the same or separate containers. The components may also be present in the same container. In addition to the above components, the components may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The following Examples illustrate some of the experiments performed in the develop of the invention.

### EXAMPLES

### Example 1: Materials and Methods

This Example illustrates some of the material and methods used in the development of the invention.

### Constructs and Strains

For bacterial expression, a plasmid encoding the Eco1 ncRNA and reverse transcriptase (RT) in that order were expressed from a T7 promoter (pSLS.436). This plasmid was constructed by amplifying the retron elements from the BL21-A1 genome and using Gibson assembly for integration into a backbone based on pRSFDUETI. The Eco1 RT was cloned separately into the erythromycin-inducible vector pJKR-O-mphR (Rogers et al. Nucleic acids research 43, 7648-7660 (2015)) to generate pSLS.402.

Eco1 ncRNA variants were cloned behind a T7/lac promoter in a vector based on pRSFDUET-1 with BsaI sites removed to facilitate Golden Gate cloning (pSLS.601), described further below. Eco1 reverse transcriptases along with recombineering ncRNAs driven by T7/lac promoters (pSLS.491 and pSLS.492) were synthesized by Twist in pET-21(+).

Bacterial experiments were carried out in BL21-AI cells, or a derivative of BL21-AI cells. These cells harbor a T7 polymerase driven by a ParaB, arabinose-inducible, promoter. A knockout strain for the Eco1 operon (bSLS.114) was constructed from BL21-AI cells, using a strategy based on Datsenko & Wanner (Proc. Nat'l Acad Sci. USA 97, 6640-6645 (2000)) to replace the retron operon with an FRT-flanked chloramphenicol resistance cassette. The replacement cassette was amplified from pKD3, adding homology arms to the Eco1 locus. This amplicon was electroporated into BL21-AI cells expressing lambda Red genes from pKD46 and clones were isolated by selection on 10 μg/ml strength chloramphenicol plates. After genotyping to confirm locus-specific insertion, the chloramphenicol cassette was excised by transient expression of FLP recombinase to leave only a FRT scar.

For yeast expression, four sets of plasmids were generated. The first set of plasmids, designed to express the protein components for yeast genome editing, were based off of pZS.157 (Sharon et al., Cell 175, 544-557 (2018)), a HIS3 yeast integrating plasmid for Galactose inducible Eco1RT and Cas9 expression (Gal1-10 promoter). A first set of variants of pZS.157, designed to compare the effect of wt vs. extended a1/a2 region lengths on genome editing were generated by PCR, and expressed either an empty cassette (pSCL.004), only Cas9 (pSCL.005), only the Eco1RT (pSCL.006), or both (pZS.157). A second set of variants was generated to test single-promoter expression of Cas9-Eco1 RT variants. Six of such plasmids were designed: Eco1RT-linker1-Cas9 (pSCL.71); Cas9-linker1-Eco1RT (pSCL.72); Eco1RT-linker2-Cas9 (pSCL.94); Cas9-linker2-Eco1RT (pSCL.95); Eco1RT-P2A-Cas9 (pSCL.102), and Cas9-P2A-Eco1RT (pSCL.103). The intervening sequences used were: Linker1 (GGTSSGGSGTAGSSGATSGG; SEQ ID NO: 14); Linker2 (SGGSSGGSSGSETPGTSESATPESSGGSSGGSS; SEQ ID NO: 15; Anzalone et al. Nature 576, 149-157 (2019); and P2A (ATNFSLLKQAGDVEENPGP; SEQ ID NO: 16; see Lui et al., Nature 566, 218-223 (2019)).

The second set of plasmids built for the genome editing experiments were based off of pZS.165 (Sharon et al. Cell 175, 544-557 (2018)), a URA3+ centromere plasmid for Galactose (Gal7) inducible expression of a modified Eco1 retron ncRNA, which consists of an Eco1 msr-ADE2-targetting gRNA chimera, flanked by HH-HDV ribozymes. An initial variant of pZS.165 was generated by cloning an IDT-synthesized gBlock consisting of an Eco1 ncRNA (a1/a2 length: 12 bp), which, when reverse transcribed, encodes a 200 bp Ade2 repair template to introduce a stop codon (P272X) into the ADE2 gene (pSCL.002). Two additional plasmids were generated to extend the a1/a2 region of the Eco1 ncRNA to 27 bp, with variations in the a1/a2 sequence (pSCL.039 and pSCL.040).

The third set of plasmids was built to assess the generalizability of the extended a1/a2 modification. The plasmids carrying wt length a1/a2 retrons are based off of pSCL.002, where the Ade2-targeting gRNA and Ade2-editing msd were replaced with analogous sequences to target and insert the following mutations: Can1 G444X (pSCL.106); Lyp1 E27X (pSCL.108); Trp2 E64X (pSCL.110); and Faa1 P233X (pSCL.112). The plasmids carrying extended length a1/a2 retrons are based off of pSCL.039 and were generated similarly to the wt length a1/a2 retron-encoding plasmids: Can1 G444X (pSCL.107); Lyp1 E27X (pSCL.109); Trp2 E64X (pSCL. 111); and Faa1 P233X (pSCL.113).

The last set of plasmids, designed to compare the levels of RT-DNA production by the different retron systems, were derived from pSCL.002. IDT-synthesized gBlocks encoding a mammalian codon-optimized Eco1 RT and ncRNA (wt), a dead Eco1RT and ncRNA (wt), and a human codon-optimized Eco2RT and ncRNA (wt), were cloned into pSCL.002 by Gibson Assembly, thereby generating pSCL.027, pSCL.031 and pSCL.017, respectively. pSCL.027 was used to generate pSCL.028 by PCR, which carries a mammalian codon optimized Eco1RT and ncRNA (extended a1/a2: 27 bp). Similarly, pSC.0L17 was used to generate pSCL.034 by PCR, which carries a mammalian codon optimized Eco2RT and ncRNA (extended a1/a2: 29 bp).

All yeast strains were created by LiAc/SS carrier DNA/PEG transformation (Gietz & Schiestl, Nature protocols 2, 31-34 (2007)) of BY4742 (Brachmann et al. Yeast (Chichester, England) 14, 115-132 (1998)). Strains for evaluating the genome editing efficiency of various retron ncRNAs were created by BY4742 integration of plasmids pZS.157, pSCL.004, pSCL.005 or pSCL.006 using KpnI-linearized plasmids for homologous recombination into the HIS3 locus. Transformants were isolated on SC-HIS plates. To evaluate effect of the length of the Eco1 ncRNA a1/a2 region on genome editing efficiency, these parental strains were transformed with episomal plasmids carrying the different retron ncRNA cassettes (pSCL.002, pSCL.039, or pSCL.040), and double transformants were isolated on SC-HIS-URA plates. The result was a set of control strains which were lacking one or both components of the genome editing machinery (i.e., Eco1RT, Cas9), and three strains which had all components necessary for retron-mediated genome editing but differed in the length of the Eco1 ncRNA a1/a2 region (12 bp vs. 27 bp).

Strains designed to assess the generalizability of the extended a1/a2 modification were created by transformation of a HIS3:pZS.157 yeast strain with plasmids carrying either wt or extended a1/a2 retrons for the editing of the four additional loci. Transformants were isolated on SC-HIS-URA plates. Strains to test single-promoter expression of Cas9-Eco1 RT variants were created by BY4742 integration of plasmids pSCL.71, pSCL.72, pSCL.94, pSCL.95, pSCL.102 or pSCL.103 using KpnI-linearized plasmids for homologous recombination into the HIS3 locus. Transformants were isolated on SC-HIS plates. These strains were then transformed with pSCL.39, and transformants isolated on SC-HIS-URA plates.

Strains designed to compare the levels of RT-DNA production by the different retron constructs were created by transformation of plasmids pSCL.027, pSCL.037 and pSCL.028 for Eco1 (wt, wt dead RT, and extended a1/a2, respectively) into BY4742; pSCL.017 and pSCL.031 for Eco2 (wt, and extended a1/a2, respectively) into BY4742. Transformants were isolated by plating on SC-URA agar plates. Expression of proteins and ncRNAs from all yeast strains was performed in liquid SC-Ura 2% Galactose media for 24 h, unless specified.

For mammalian retron expression and quantification of RT-DNA production, synthesized gBlocks encoding human codon optimized Eco1 and Eco2 were cloned into a PiggyBac-integrating plasmid for doxycycline-inducible human protein expression (TetOn-3G promoter). Eco1 variants are wt retron-Eco1 RT and ncRNA (pKDC.018, with a1/a2 length: 12 bp), extended a1/a2 length ncRNA (pKDC.019, with a1/a2 length: 27 bp), and a dead-Eco1 RT control (pKDC.020, with a1/a2 length: 27 bp). Eco2 variants were wt retron-Eco2 RT and ncRNA (pKDC.015, with a1/a2 length: 13 bp), extended a1/a2 length ncRNA (pKDC.031, with a1/a2 length: 29 bp).

Stable mammalian cell lines for assessing RT-DNA production by wild type (wt) and extended a1/a2 regions were created using the Lipofectamine 3000 transfection protocol (Invitrogen) and a PiggyBac transposase system. T25s of 50-70% confluent HEK293T cells were transfected using 8.3 ug of retron expression plasmids (pKDC.015, pKDC.018, pKDC.019, pKDC.020, or pKDC.031) and 4.2 ug PiggyBac transposase plasmid (pCMV-hyPBase). Stable cell lines were selected with puromycin.

For assessment of retron-mediated precise genome editing in mammalian cells, two sets of plasmids were generated. The first set of plasmids, carrying either the SpCas9 gene or the SpCas9-P2A-Eco1RT construct, was built by restriction cloning of the respective genes, PCR amplified off of the aforementioned yeast vectors, into a PiggyBac-integrating plasmid for doxycycline-inducible human protein expression (TetOn-3G promoter). The second set of plasmids carried the ncRNA/gRNA targeting one of six loci in the human genome: HEK3 (pSCL.175); RNF2 (pSCL.176); EMX1 (pSCL.177); FANCF (pSCL.178); HEK4 (pSCL.179); and AAVS1 (pSCL.180). These were generated by restriction cloning of the ncRNA/gRNA cassette, built by primer assembly (Tian & Das, Bioinformatics (Oxford, England) 33, 1405-1406 (2017)), into an H1 expression plasmid (FHUGW).

The ncRNA/gRNA cassette was designed as follows. The msd contained a repair template-encoding, 120 bp sequence in its loop. The plasmid-encoded repair template was slightly asymmetric (49 bp of genome site homology upstream of Cas9 cut site; 71 bp of genome site homology downstream of cut site) and was complementary to the target strand (which in practice, this means that after reverse transcription). The repair template RT-DNA was complementary to the non-target strand. The repair template carried two distinct mutations: the first introduced a 1 bp SNP at the Cas9 cut site; the second, designed to be at least 2 bp away from the first mutation, recoded the Cas9 PAM (NGG→NHH, where H is any nucleotide beside G). The gRNA is 20 bp.

Stable mammalian cell lines for assessing retron-mediated precise genome editing were created using the Lipofectamine 3000 transfection protocol (Invitrogen) and a PiggyBac transposase system. T25 flasks of 50-70% confluent HEK293T cells were transfected using 8.3 μg of protein expression plasmids (pSCL.139 and pSCL.140) and 4.2 μg PiggyBac transposase plasmid (pCMV-hyPBase). Stable cell lines were selected with puromycin.

Plasmids are listed in Tables 2-4, and strains are listed in Table 5.

TABLE 2
Bacterial Plasmids

| | | | | Data |
|---|---|---|---|---|
| | | Pro- | | Shown in |
| Name | Description | moter | Inducer | Figures |
| pSLS.436 | Eco1: ncRNA(wt) | T7 | L-arabinose | 1D |
| | and RT | | (0.2% w/w) | |
| pSLS.402 | Eco1 RT | mphR | erythromycin | 2B, 2C, |
| | | | (400 uM) | 2H |
| pSLS.601 | Eco1 ncRNA (variants) | T7/lac | L-arabinose | 2B, 2C, |
| | | | (0.2% w/w) + | 2H |
| | | | IPTG (1 mM) | |
| pSLS.491 | Eco1 RT and | T7/lac | L-arabinose | 4B, 4C, |
| | recombineering | | (0.2% w/w) + | 4D |
| | ncRNA, rpoB T1534C, | | IPTG (1 mM) | |
| | a1/a2 length: 12 | | | |
| pSLS.492 | Eco1 RT and | T7/lac | L-arabinose | 4B, 4C, |
| | recombineering | | (0.2% w/w) + | 4D |
| | ncRNA, rpoB T1534C, | | IPTG (1 mM) | |
| | a1/a2 length: 22 | | | |
| pSLS.539 | Eco1 RT and | T7/lac | L-arabinose | 3I |
| | recombineering | | (0.2% w/w) + | |
| | ncRNA, fabH G954A, | | IPTG (1 mM) | |
| | a1/a2 length: 12 | | | |
| pSLS.540 | Eco1 RT and | T7/lac | L-arabinose | 3I |
| | recombineering | | (0.2% w/w) + | |
| | ncRNA, fabH G954A, | | IPTG (1 mM) | |
| | a1/a2 length: 22 | | | |
| pSLS.543 | Eco1 RT and | T7/lac | L-arabinose | 3J |
| | recombineering | | (0.2% w/w) + | |
| | ncRNA, murF | | IPTG (1 mM) | |
| | G1359A, | | | |
| | a1/a2 length: 12 | | | |
| pSLS.544 | Eco1 RT and | T7/lac | L-arabinose | 3J |
| | recombineering | | (0.2% w/w) + | |
| | ncRNA, murF G1359A, | | IPTG (1 mM) | |
| | a1/a2 length: 22 | | | |
| pSLS.545 | Eco1 RT and | T7/lac | L-arabinose | 3K |
| | recombineering | | (0.2% w/w) + | |
| | ncRNA, priB G1069A, | | IPTG (1 mM) | |
| | a1/a2 length: 12 | | | |
| pSLS.546 | Eco1 RT and | T7/lac | L-arabinose | 3K |
| | recombineering | | (0.2% w/w) + | |
| | ncRNA, priB G1069A, | | IPTG (1 mM) | |
| | a1/a2 length: 22 | | | |
| pORTMAGE- | CspRecT and mutL | Pm | m-toluic acid | 4B, 4C, |
| Ec1 | E32K | | (1 mM) | 4D |
| pKD46 | lambda Red genes | ParaB | L-arabinose | — |
| | | | (0.2% w/w) | |
| pKD3 | FRT-cat-FRT (gene | cat | — | — |
| | disruption) | | | |

TABLE 3
Yeast Plasmids

| Name | Description | Promoter | Inducer |
|---|---|---|---|
| pSCL.027 | Eco1: RT and ncRNA(wt), | Gal7 | Galactose (2% w/w) |
| | a1/a2length: 12 | | |
| pSCL.028 | Eco1: RT and | Gal7 | Galactose (2% w/w) |
| | ncRNA(extended), a1/a2 | | |
| | length: 27 | | |
| pSCL.037 | Eco1: RT and ncRNA(wt), | Gal7 | Galactose (2% w/w) |
| | a1/a2length: 12, dead RT | | |
| pSCL.017 | Eco2: RT and ncRNA(wt), | Gal7 | Galactose (2% w/w) |
| | a1/a2 | | |
| | length: 13 | | |
| pSCL.031 | Eco2: RT and | Gal7 | Galactose (2% w/w) |
| | ncRNA(extended), a1/a2 | | |
| | length: 29 | | |
| pSCL.004 | Integrating inducible | Gal1-10 | Galactose (2% w/w) |
| | cassette: empty | | |
| pSCL.006 | Integrating inducible | Gal1-10 | Galactose (2% w/w) |
| | cassette: Eco1 RT | | |
| pSCL.005 | Integrating inducible | Gal1-10 | Galactose (2% w/w) |
| | cassette: Cas9 | | |
| pZS.157 | Integrating inducible | Gal1-10 | Galactose (2% w/w) |
| | cassette: Cas9and Eco1 | | |
| | RT | | |
| pSCL.002 | Eco1 editing ncRNA and | Gal7 | Galactose (2% w/w) |
| | gRNA, ADE2P272X, | | |
| | a1/a2 length: 12 | | |
| pSCL.039 | Eco1 editing ncRNA and | Gal7 | Galactose (2% w/w) |
| | gRNA, ADE2 P272X, | | |
| | a1/a2 length: 27 v1 | | |
| pSCL.040 | Eco1 editing ncRNA and | Gal7 | Galactose (2% w/w) |
| | gRNA, ADE2P272X, | | |
| | a1/a2 length: 27 v2 | | |
| pSCL.106 | Eco1 editing ncRNA and | Gal7 | Galactose (2% w/w) |
| | gRNA, CAN1G444X, | | |
| | a1/a2 length: 12 | | |
| pSCL.107 | Eco1 editing ncRNA and | Gal7 | Galactose (2% w/w) |
| | gRNA, CAN1G444X, | | |
| | a1/a2 length: 27 v1 | | |
| pSCL.108 | Eco1 editing ncRNA and | Gal7 | Galactose (2% w/w) |
| | gRNA, LYP1E27X. | | |
| | a1/a2 length: 12 | | |
| pSCL.109 | Eco1 editing neRNA and | Gal7 | Galactose (2% w/w) |
| | gRNA, LYP1E27X, | | |
| | a1/a2 length: 27 v1 | | |
| pSCL.110 | Eco1 editing ncRNA and | Gal7 | Galactose (2% w/w) |
| | gRNA, TRP2E64X, | | |
| | a1/a2 length: 12 | | |
| pSCL.111 | Eco1 editing ncRNA | Gal7 | Galactose (2% w/w) |
| | and gRNA, TRP2 | | |
| | E64X, a1/a2 length: 27 v1 | | |
| pSCL.112 | Eco1 editing ncRNA and | Gal7 | Galactose (2% w/w) |
| | gRNA, FAA1P233X, | | |
| | a1/a2 length: 12 | | |
| pSCL.113 | Eco1 editing ncRNA and | Gal7 | Galactose (2% w/w) |
| | gRNA, FAA1P233X, | | |
| | a1/a2 length: 27 v1 | | |
| pSCL.71 | Eco1RT-fusion_linker1- | Gal1-10 | Galactose (2% w/w) |
| | Cas9 | | |
| pSCL.72 | Cas9-fusion_linker1- | Gal1-10 | Galactose (2% w/w) |
| | EcoIRT | | |
| pSCL.94 | Eco1RT-fusion_linker2- | Gal1-10 | Galactose (2% w/w) |
| | Cas9 | | |
| pSCL.95 | Cas9-fusion_linker2- | Gal1-10 | Galactose (2% w/w) |
| | Eco1RT | | |
| pSCL.102 | Eco1RT-P2A-Cas9 | Gal1-10 | Galactose (2% w/w) |
| pSCL.103 | Cas9-P2A-Eco1RT | Gal1-10 | Galactose (2% w/w) |

TABLE 4
Mammalian Plasmids

| | | | | Data |
|---|---|---|---|---|
| | | | | Shown |
| | | | | in |
| Name | Description | Promoter | Inducer | Figures |
| pKDC.018 | Eco1: RT and ncRNA | TetOn- | Doxycycline | 3E |
| | (wt), a1/a2length: 12 | 3g | (1 ug/ml) | |
| pKDC.019 | Eco1: RT and | TetOn- | Doxycycline | 3E |
| | ncRNA(extended), | 3g | (1 ug/ml) | |
| | a1/a2 length: 27 | | | |
| pKDC.020 | Eco1: RT and ncRNA | TetOn- | Doxycycline | 2I |
| | (wt), a1/a2length: 12, | 3g | (1 ug/ml) | |
| | dead RT | | | |
| pKDC.015 | Eco2: RT and ncRNA | TetOn- | Doxycycline | 3F |
| | (wt), a1/a2 length: 13 | 3g | (1 ug/ml) | |
| pKDC.031 | Eco2: RT and | TetOn- | Doxycycline | 3F |
| | ncRNA (extended), | 3g | (1 ug/ml) | |
| | a1/a2 length: 29 | | | |
| pSCL.139 | SpCas9-P2A-Eco1RT | TetOn- | Doxycycline | 5B-5H |
| | | 3g | (1 ug/ml) | |
| pSCL.140 | SpCas9 | TetOn- | Doxycycline | 5B-5H |
| | | 3g | (1 ug/ml) | |
| pSCL.175 | Eco1: | H1 | NA | 5B-5H |
| | HEK3 targeting and | | | |
| | editing ncRNA-gRNA | | | |
| | (a1/a2 length: 27 vl), | | | |
| | expressed constitutively | | | |
| | from a pol III (H1) | | | |
| | promoter | | | |
| pSCL.176 | Eco1: RNF2 targeting and | H1 | NA | 5B-5H |
| | editing ncRNA-gRNA | | | |
| | (a1/a2 length: 27 v1), | | | |
| | expressed constitutively | | | |
| | from a polIII (H1) | | | |
| | promoter | | | |
| pSCL.177 | Eco1: EMX1 targeting | H1 | NA | 5B-5H |
| | and editing ncRNA-gRNA | | | |
| | (a1/a2 length: 27 v1), | | | |
| | expressed constitutively | | | |
| | from a polIII (H1) | | | |
| | promoter | | | |
| pSCL.178 | Eco1: FANCF targeting | H1 | NA | 5B-5H |
| | and editing ncRNA-gRNA | | | |
| | (a1/a2 length: 27 v1), | | | |
| | expressed constitutively | | | |
| | from a polIII (H1) | | | |
| | promoter | | | |
| pSCL.179 | Eco1: HEK4 targeting and | H1 | NA | 5B-5H |
| | editing ncRNA-gRNA | | | |
| | (a1/a2 length: 27 v1), | | | |
| | expressed constitutively | | | |
| | from a polIII (H1) | | | |
| | promoter | | | |
| pSCL.180 | Eco1: AAVS1 targeting | H1 | NA | 5B-5H |
| | and editing ncRNA-gRNA | | | |
| | (a1/a2 length: 27 v1), | | | |
| | expressed constitutively | | | |
| | from a polIII (H1) | | | |
| | promoter | | | |

TABLE 5
Strains

| | | Parental | | |
|---|---|---|---|---|
| Name | Species | Line | Genotype | Method |
| bSLS.114 | E. coli | BL21-AI | Eco1 KO | lambda Red recombinase |
| | | | | mediated insertion |
| | | | | of chloramphenicol |
| | | | | resistance, marker |
| | | | | excision by FLP |
| ySCL2 | S. | BY4742 | HIS3: | LiAc mediated plasmid |
| | cerevisiae | | pSCL.004 | transformation and |
| | | | | HR mediated insertion |
| | | | | of empty Galactose |
| | | | | inducible cassette |
| | | | | (pSCL.004) |
| | | | | into the HIS3 locus |
| ySCL3 | S. | BY4742 | HIS3: | LiAc mediated plasmid |
| | cerevisiae | | pSCL.005 | transformation and |
| | | | | HR mediated insertion |
| | | | | of a Galactose inducible |
| | | | | cassette for Cas9 |
| | | | | expression (pSCL.005) |
| | | | | into the HIS3 locus |
| ySCL4 | S. | BY4742 | HIS3: | LiAc mediated plasmid |
| | cerevisiae | | pSCL.006 | transformation and |
| | | | | HR mediated insertion |
| | | | | of a Galactose inducible |
| | | | | cassette for Eco1RT |
| | | | | expression (pSCL.006) |
| | | | | into the HIS3 locus |
| ySCL5 | S. | BY4742 | HIS3: | LiAc mediated plasmid |
| | cerevisiae | | pZS.157 | transformation and HR |
| | | | | mediated insertion |
| | | | | of a Galactose inducible |
| | | | | cassette for Cas9 and |
| | | | | Eco1RT expression |
| | | | | (pZS.157) into the HIS3 |
| | | | | locus |
| ySCL53 | S. | BY4742 | HIS3: | LiAc mediated plasmid |
| | cerevisiae | | pSCL.71 | transformation and |
| | | | | HR mediated insertion |
| | | | | of a Galactose inducible |
| | | | | cassette for Eco1RT- |
| | | | | fusion linker1-Cas9 |
| | | | | expression (pSCL.71) |
| | | | | into the HIS3 locus |
| ySCL54 | S. | BY4742 | HIS3: | LiAc mediated plasmid |
| | cerevisiae | | pSCL.72 | transformation and |
| | | | | HR mediated insertion |
| | | | | of a Galactose inducible |
| | | | | cassette for Cas9-fusion |
| | | | | linker1-Eco1RT |
| | | | | (pSCL.72) into the HIS3 |
| | | | | locus |
| ySCL76 | S. | BY4742 | HIS3: | LiAc mediated plasmid |
| | cerevisiae | | pSCL.94 | transformation and |
| | | | | HR mediated insertion |
| | | | | of a Galactose inducible |
| | | | | cassette for Eco1RT- |
| | | | | fusion linker2-Cas9 |
| | | | | (pSCL.94) into the |
| | | | | HIS3 locus |
| ySCL77 | S. | BY4742 | HIS3: | LiAc mediated plasmid |
| | cerevisiae | | pSCL.95 | transformation and |
| | | | | HR mediated insertion |
| | | | | of a Galactose inducible |
| | | | | cassette for Cas9-fusion |
| | | | | linker2-Eco1RT |
| | | | | (pSCL.95) into the HIS3 |
| | | | | locus |
| ySCL92 | S. | BY4742 | HIS3: | LiAc mediated plasmid |
| | cerevisiae | | pSCL.102 | transformation and |
| | | | | HR mediated insertion |
| | | | | of a Galactose inducible |
| | | | | cassette for Eco1RT-P2A- |
| | | | | Cas9 (pSCL.102) |
| | | | | into the HIS3 locus |
| ySCL86 | S. | BY4742 | HIS3: | LiAc mediated plasmid |
| | cerevisiae | | pSCL.103 | transformation and |
| | | | | HR mediated insertion |
| | | | | of a Galactose inducible |
| | | | | cassette for Cas9-P2A- |
| | | | | Eco1RT (pSCL.103) |
| | | | | into the HIS3 locus |
| pKDC.018 | H. sapiens | HEK293T | Integrated | Lipofectamine 3000 |
| | | | pKDC.018 | mediated plasmid |
| | | | | transfection and |
| | | | | PiggyBac mediated |
| | | | | insertion of doxycycline |
| | | | | inducible cassette |
| | | | | for Eco1RT and wt |
| | | | | ncRNA |
| pKDC.019 | H. sapiens | HEK293T | Integrated | Lipofectamine 3000 |
| | | | pKDC.019 | mediated plasmid |
| | | | | transfection and |
| | | | | PiggyBac mediated |
| | | | | insertion of doxycycline |
| | | | | inducible cassette |
| | | | | for Eco1RT and |
| | | | | extended a1/a2 nCRNA |
| pKDC.020 | H. sapiens | HEK293T | Integrated | Lipofectamine 3000 |
| | | | pKDC.020 | mediated plasmid |
| | | | | transfection and |
| | | | | PiggyBac mediated |
| | | | | insertion of doxycycline |
| | | | | inducible cassette for |
| | | | | Eco1 deadRT and |
| | | | | extended a1/a2 nCRNA |
| pKDC.015 | H. sapiens | HEK293T | Integrated | Lipofectamine 3000 |
| | | | pKDC.015 | mediated plasmid |
| | | | | transfection and |
| | | | | PiggyBac mediated |
| | | | | insertion of doxycycline |
| | | | | inducible cassette |
| | | | | for Eco2RT and wt |
| | | | | nCRNA |
| pKDC.031 | H. sapiens | HEK293T | Integrated | Lipofectamine 3000 |
| | | | pKDC.031 | mediated plasmid |
| | | | | transfection and |
| | | | | PiggyBac mediated |
| | | | | insertion of doxycycline |
| | | | | inducible cassette for |
| | | | | Eco2RT and extended |
| | | | | a1/a2 nCRNA |
| pSCL.139 | H. sapiens | HEK293T | Integrated | Lipofectamine 3000 |
| | | | pSCL.139 | mediated plasmid |
| | | | | transfection and |
| | | | | Piggy Bac mediated |
| | | | | insertion of doxycycline |
| | | | | inducible cassette |
| | | | | for SpCas9 and Eco1RT |
| | | | | expression; proteins |
| | | | | are separated |
| | | | | by a P2A sequence |
| pSCL.140 | H. sapiens | HEK293T | Integrated | Lipofectamine 3000 |
| | | | psCL.140 | mediated plasmid |
| | | | | transfection and |
| | | | | PiggyBac mediated |
| | | | | insertion of doxycycline |
| | | | | inducible cassette |
| | | | | for SpCas9 expression |

### Examples of Sequences Used in the Plasmids are Shown Below.

pSCL.39: Eco1 editing ncRNA and gRNA,
ADE2 P272X, a1/a2 length: 27 v1
HH ribozyme (bold font)-Eco1 ncRNA-
ADE2 P272X donor (italic font)-Eco1 ncRNA-
ADE2 gRNA-SpCas9 Scaffold-HDV ribozyme
(underline)
(SEQ ID NO: 17)
GGGTGCGCATCTGATGAGTCCGTGAGGACGAAACGAGCTAGCTCG
TCATGATAAGATTCCGTATGCGCACCCTTAGCGAGAGGTTTATCA
TTAAGGTCAACCTCTGGATGTTGTTTCGGCATCCTGCATTGAATC
TGAGTTACTGTCTGTTTTCCTTGGAAATGTTCTATTTAGAAACAG
GGGAATTGCTTATTAACGAAATTGCCTGAAGGCCTCACAACTCTG
GACATTATACCATTGATGCTTGCGTCACTTCAGGAAACCCGTTTC
TTCTGACGTAAGGGTGCGCATACGGAATCTTATCAATTAACGAAA
TTGCCCCAGTTTCAGAGCTATGCTGGAAACAGCATAGCAAGTTGA
AATAAGGCTAGTCCGTTATCAACTTGAAAAAGTGGCACCGAGTCG
GTGCTTTTTGATGGCCGGCATGGTCCCAGCCTCCTCGCTGGCGCC
GGCTGGGCAACACCTTCGGGTGGCGAATGGGACTT
pSCL.75: Sen2 RT
SV40 NLS (bold font)-Sen2 RT
(SEQ ID NO: 18)
MPPKKKRKVDILQHISDLLLTKKSEIISFSLTAPYRYKIYKIAKR
NSDKKRTIAHPSKELKFIQREITEYLTDKLPVHECAFAYKKGSSI
KTNAQVHLHTKYLLKMDFENFFPSITPRLFFSKLRLANIDLTADD
KVLLENILFFKSKRNSNLRLSIGAPSSPLISNFVMYFWDIEVQEI
CSKIGVNYTRYADDLTFSTNNKDVLFDIPDMLENVLPKYSLGRIR
INHEKTVFSSKGHNRHVTGITLTNDNKLSIGRERKRKISAMIHHF
INGKLSTDECNKLVGLLAFAKNIEPSFYKSMVIKYGSDNIYKLQK
QKDK
pSCL.76:Eco4RT
SV40 NLS (bold font)-Eco4 RT
(SEQ ID NO: 19)
MPPKKKRKVSIDIETTLQKAYPDFDVLLKSRPATHYKVYKIPKRT
IGYRIIAQPTPRVKAIQRDIIEILKQHTHIHDAATAYVDGKNILD
NAKIHQSSVYLLKLDLVNFENKITPELLFKALARQKVDISDINKN
LLKQFCFWNRTKRKNGALVLSVGAPSSPFISNIVMSSFDEEISSF
CKENKISYSRYADDLTFSTNERDVLGLAHQKVKTTLIRFFGTRII
INNNKIVYSSKAHNRHVTGVTLTNNNKLSLGRERKRYITSLVFKF
KEGKLSNVDINHLRGLIGFAYNIEPAFIERLEKKYGESTIKSIKK
YSEGG
pSCL.80: Eco4 editing ncRNA and gRNA,
ADE2 P272X, a1/a2 length: 27 v1
HH ribozyme (bold font)-Eco4 ncRNA-ADE2
P272X donor (italic font)-Eco4 ncRNA-
ADE2 gRNA-SpCas9 Scaffold-HDV ribozyme
(underline)
(SEQ ID NO: 20)
GGGTGCGCATCTGATGAGTCCGTGAGGACGAAACGAGCTAGCTCG
TCTGATAAGATTCCGTAGCTCTTTAGCGTTTTATGGATTTACCAC
CTGATTGGTCAAATCTAGTTGGGCGTTGCGCCAAACTCTAATTTA
TTGATTACATTTACAGTTGCGGAACAAACTTTTTGAGCCGCAATT
GGAAATGTTCTATTTAGAAACAGGGGAATTGCTTATTAACGAAAT
TGCCTGAAGGCCTCACAACTCTGGACATTATACCATTGATGCTTG
CGTCACTTCGTTGCGGATCAAAAAGTTTGTTCCGCGGCTTCAAGT
AAAGAGCTACGGAATCTTATCAATTAACGAAATTGCCCCAGTTTC
AGAGCTATGCTGGAAACAGCATAGCAAGTTGAAATAAGGCTAGTC
CGTTATCAACTTGAAAAAGTGGCACCGAGTCGGTGCTTTTTGATG
GCCGGCATGGTCCCAGCCTCCTCGCTGGCGCCGGCTGGGCAACAC
CTTCGGGTGGCGAATGGGACTT
pSCL.81: Sen2 editing ncRNA and gRNA,
ADE2 P272X, a1/a2 length: 27 v1
HH ribozyme (bold font)-Sen2 ncRNA-ADE2
P272X donor (italic font)-Sen2 ncRNA-
ADE2 gRNA-SpCas9 Scaffold-HDV ribozyme
(underline)
(SEQ ID NO: 21)
GGGTGCGCATCTGATGAGTCCGTGAGGACGAAACGAGCTAGCTCG
TCTGATAAGATTCCGTAACTCTTTAGCGTTAGGCTTTGATTTATA
GCCTTGTCGAGCGTTTCGCCAGACACTAACTTATTGAGTACTTTT
AGGGTTGCGCTAGAAAGTTTTCTACCGATCCTAGTGGAAATGTTC
TATTTAGAAACAGGGGAATTGCTTATTAACGAAATTGCCTGAAGG
CCTCACAACTCTGGACATTATACCATTGATGCTTGCGTCACTTCC
TAGGATCGGTAGAAAACTTTCTAGCGCCTCCTCTAGTAAAGAGTT
ACGGAATCTTATCAATTAACGAAATTGCCCCAGTTTCAGAGCTAT
GCTGGAAACAGCATAGCAAGTTGAAATAAGGCTAGTCCGTTATCA
ACTTGAAAAAGTGGCACCGAGTCGGTGCTTTTTGATGGCCGGCAT
GGTCCCAGCCTCCTCGCTGGCGCCGGCTGGGCAACACCTTCGGGT
GGCGAATGGGACTT
pSCL.71; Eco1RT-fusion_linker1-SpCas9
SV40 NLS (bold font)-Eco1 RT-fusion
linker 1 (italic font)-SpCas9-SV40 NLS
(underline)
(SEQ ID NO: 22)
MPPKKKRKVKSAEYLNTFRLRNLGLPVMNNLHDMSKATRISVETL
RLLIYTADFRYRIYTVEKKGPEKRMRTIYQPSRELKALQGWVLRN
ILDKLSSSPFSIGFEKHQSILNNATPHIGANFILNIDLEDFFPSL
TANKVFGVFHSLGYNRLISSVLTKICCYKNLLPQGAPSSPKLANL
ICSKLDYRIQGYAGSRGLIYTRYADDLTLSAQSMKKVVKARDFLF
SHIPSEGLVINSKKTCISGPRSQRKVTGLVISQEKVGIGREKYKE
IRAKIHHIFCGKSSEIEHVRGWLSFILSVDSKSHRRLITYISKLE
KKYGKNPLNKAKTGGTSSGGSGPAGSSGATSGGDKKYSIGLDIGT
NSVGWAVITDEYKVPSKKFKVLGNTDRHSIKKNLIGALLFDSGET
AEATRLKRTARRRYTRRKNRICYLQEIFSNEMAKVDDSFFHRLEE
SFLVEEDKKHERHPIFGNIVDEVAYHEKYPTIYHLRKKLVDSTDK
ADLRLIYLALAHMIKFRGHFLIEGDLNPDNSDVDKLFIQLVQTYN
QLFEENPINASGVDAKAILSARLSKSRRLENLIAQLPGEKKNGLF
GNLIALSLGLTPNFKSNFDLAEDAKLQLSKDTYDDDLDNLLAQIG
DQYADLFLAAKNLSDAILLSDILRVNTEITKAPLSASMIKRYDEH
HQDLTLLKALVRQQLPEKYKEIFFDQSKNGYAGYIDGGASQEEFY
KFIKPILEKMDGTEELLVKLNREDLLRKQRTFDNGSIPHQIHLGE
LHAILRRQEDFYPFLKDNREKIEKILTFRIPYYVGPLARGNSRFA
WMTRKSEETITPWNFEEVVDKGASAQSFIERMTNFDKNLPNEKVL
PKHSLLYEYFTVYNELIKVKYVTEGMRKPAFLSGEQKKAIVDLLF
KTNRKVTVKQLKEDYFKKIECFDSVEISGVEDRFNASLGTYHDLL
KIIKDKDFLDNEENEDILEDIVLTLTLFEDREMIEERLKTYAHLF
DDKVMKQLKRRRYTGWGRLSRKLINGIRDKQSGKTILDFLKSDGF
ANRNFMQLIHDDSLTFKEDIQKAQVSGQGDSLHEHIANLAGSPAI
KKGILQTVKVVDELVKVMGRHKPENIVIEMARENQTTQKGQKNSR
ERMKRIEEGIKELGSQILKEHPVENTQLQNEKLYLYYLQNGRDMY
VDQELDINRLSDYDVDHIVPQSFLKDDSIDNKVLTRSDKNRGKSD
NVPSEEVVKKMKNYWRQLLNAKLITQRKFDNLTKAERGGLSELDK
AGFIKRQLVETRQITKHVAQILDSRMNTKYDENDKLIREVKVITL
KSKLVSDFRKDFQFYKVREINNYHHAHDAYLNAVVGTALIKKYPK
LESEFVYGDYKVYDVRKMIAKSEQEIGKATAKYFFYSNIMNFFKT
EITLANGEIRKRPLIETNGETGEIVWDKGRDFATVRKVLSMPQVN
IVKKTEVQTGGFSKESILPKRNSDKLIARKKDWDPKKYGGFDSPT
VAYSVLVVAKVEKGKSKKLKSVKELLGITIMERSSFEKNPIDFLE
AKGYKEVKKDLIIKLPKYSLFELENGRKRMLASAGELQKGNELAL
PSKYVNFLYLASHYEKLKGSPEDNEQKQLFVEQHKHYLDEIIEQI
SEFSKRVILADANLDKVLSAYNKHRDKPIREQAENIIHLFTLTNL
GAPAAFKYFDTTIDRKRYTSTKEVLDATLIHQSITGLYETRIDLS
QLGGDSRADPKKKRKV
pSCL.72: SpCas9-fusion linker1-Eco1RT
SV40 NLS (bold font)-SpCas9-fusion linker 1
(italic font)-Eco1 RT-SV40 NLS
(underline)
(SEQ ID NO: 23)
MSRADPKKKRKVDKKYSIGLDIGTNSVGWAVITDEYKVPSKKFKV
LGNTDRHSIKKNLIGALLFDSGETAEATRLKRTARRRYTRRKNRI
CYLQEIFSNEMAKVDDSFFHRLEESFLVEEDKKHERHPIFGNIVD
EVAYHEKYPTIYHLRKKLVDSTDKADLRLIYLALAHMIKFRGHFL
IEGDLNPDNSDVDKLFIQLVQTYNQLFEENPINASGVDAKAILSA
RLSKSRRLENLIAQLPGEKKNGLFGNLIALSLGLTPNFKSNFDLA
EDAKLQLSKDTYDDDLDNLLAQIGDQYADLFLAAKNLSDAILLSD
ILRVNTEITKAPLSASMIKRYDEHHQDLTLLKALVRQQLPEKYKE
IFFDQSKNGYAGYIDGGASQEEFYKFIKPILEKMDGTEELLVKLN
REDLLRKQRTFDNGSIPHQIHLGELHAILRRQEDFYPFLKDNREK
IEKILTFRIPYYVGPLARGNSRFAWMTRKSEETITPWNFEEVVDK
GASAQSFIERMTNFDKNLPNEKVLPKHSLLYEYFTVYNELTKVKY
VTEGMRKPAFLSGEQKKAIVDLLFKTNRKVTVKQLKEDYFKKIEC
FDSVEISGVEDRFNASLGTYHDLLKIIKDKDFLDNEENEDILEDI
VLTLTLFEDREMIEERLKTYAHLFDDKVMKQLKRRRYTGWGRLSR
KLINGIRDKQSGKTILDFLKSDGFANRNFMQLIHDDSLTFKEDIQ
KAQVSGQGDSLHEHIANLAGSPAIKKGILQTVKVVDELVKVMGRH
KPENIVIEMARENQTTQKGQKNSRERMKRIEEGIKELGSQILKEH
PVENTQLQNEKLYLYYLQNGRDMYVDQELDINRLSDYDVDHIVPQ
SFLKDDSIDNKVLTRSDKNRGKSDNVPSEEVVKKMKNYWRQLLNA
KLITQRKFDNLTKAERGGLSELDKAGFIKRQLVETRQITKHVAQI
LDSRMNTKYDENDKLIREVKVITLKSKLVSDFRKDFQFYKVREIN
NYHHAHDAYLNAVVGTALIKKYPKLESEFVYGDYKVYDVRKMIAK
SEQEIGKATAKYFFYSNIMNFFKTEITLANGEIRKRPLIETNGET
GEIVWDKGRDFATVRKVLSMPQVNIVKKTEVQTGGFSKESILPKR
NSDKLIARKKDWDPKKYGGFDSPTVAYSVLVVAKVEKGKSKKLKS
VKELLGITIMERSSFEKNPIDFLEAKGYKEVKKDLIIKLPKYSLF
ELENGRKRMLASAGELQKGNELALPSKYVNFLYLASHYEKLKGSP
EDNEQKQLFVEQHKHYLDEIIEQISEFSKRVILADANLDKVLSAY
NKHRDKPIREQAENIIHLFTLTNLGAPAAFKYFDTTIDRKRYTST
KEVLDATLIHQSITGLYETRIDLSQLGGDGGTSSGGSGPAGSSGA
TSGGKSAEYLNTFRLRNLGLPVMNNLHDMSKATRISVETLRLLIY
TADFRYRIYTVEKKGPEKRMRTIYQPSRELKALQGWVLRNILDKL
SSSPFSIGFEKHQSILNNATPHIGANFILNIDLEDFFPSLTANKV
FGVFHSLGYNRLISSVLTKICCYKNLLPQGAPSSPKLANLICSKL
DYRIQGYAGSRGLIYTRYADDLTLSAQSMKKVVKARDFLFSIIPS
EGLVINSKKTCISGPRSQRKVTGLVISQEKVGIGREKYKEIRAKI
HHIFCGKSSEIEHVRGWLSFILSVDSKSHRRLITYISKLEKKYGK
NPLNKAKTPPKKKRKV
pSCL.94: Eco1RT-fusion linker2-SpCas9
SV40 NLS (bold font)-Eco1 RT-fusion
linker 2 (italic font)-SpCas9-SV40 NLS
(underline)
(SEQ ID NO: 24)
MPPKKKRKVKSAEYLNTFRLRNLGLPVMNNLHDMSKATRISVETL
RLLIYTADFRYRIYTVEKKGPEKRMRTIYQPSRELKALQGWVLRN
ILDKLSSSPFSIGFEKHQSILNNATPHIGANFILNIDLEDFFPSL
TANKVFGVFHSLGYNRLISSVLTKICCYKNLLPQGAPSSPKLANL
ICSKLDYRIQGYAGSRGLIYTRYADDLTLSAQSMKKVVKARDFLF
SHIPSEGLVINSKKTCISGPRSQRKVTGLVISQEKVGIGREKYKE
IRAKIHHIFCGKSSEIEHVRGWLSFILSVDSKSHRRLITYISKLE
KKYGKNPLNKAKTSGGSSGGSSGSETPGTSESATPESSGGSSGGS
SDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGDSRADPKKKRKV
pSCL.95: SpCas9-fusion_linker2-Eco1RT
SV40 NLS (bold font)-SpCas9-fusion linker
2 (italic font)-Eco1RT-SV40 NLS
(underline)
(SEQ ID NO: 25)
MSRADPKKKRKVDKKYSIGLDIGTNSVGWAVITDEYKVPSKKFKV
LGNTDRHSIKKNLIGALLFDSGETAEATRLKRTARRRYTRRKNRI
CYLQEIFSNEMAKVDDSFFHRLEESFLVEEDKKHERHPIFGNIVD
EVAYHEKYPTIYHLRKKLVDSTDKADLRLIYLALAHMIKFRGHFL
IEGDLNPDNSDVDKLFIQLVQTYNQLFEENPINASGVDAKAILSA
RLSKSRRLENLIAQLPGEKKNGLFGNLIALSLGLTPNFKSNFDLA
EDAKLQLSKDTYDDDLDNLLAQIGDQYADLFLAAKNLSDAILLSD
ILRVNTEITKAPLSASMIKRYDEHHQDLTLLKALVRQQLPEKYKE
IFFDQSKNGYAGYIDGGASQEEFYKFIKPILEKMDGTEELLVKLN
REDLLRKQRTFDNGSIPHQIHLGELHAILRRQEDFYPFLKDNREK
IEKILTFRIPYYVGPLARGNSRFAWMTRKSEETITPWNFEEVVDK
GASAQSFIERMTNFDKNLPNEKVLPKHSLLYBYFTVYNELTKVKY
VTEGMRKPAFLSGEQKKAIVDLLFKTNRKVTVKQLKEDYFKKIEC
FDSVEISGVEDRFNASLGTYHDLLKIIKDKDFLDNEENEDILEDI
VLTLTLFEDREMIEERLKTYAHLFDDKVMKQLKRRRYTGWGRLSR
KLINGIRDKQSGKTILDFLKSDGFANRNFMQLIHDDSLTFKEDIQ
KAQVSGQGDSLHEHIANLAGSPAIKKGILQTVKVVDELVKVMGRH
KPENIVIEMARENQTTQKGQKNSRERMKRIEEGIKELGSQILKEH
PVENTQLQNEKLYLYYLQNGRDMYVDQELDINRLSDYDVDHIVPQ
SFLKDDSIDNKVLTRSDKNRGKSDNVPSEEVVKKMKNYWRQLLNA
KLITQRKFDNLTKAERGGLSELDKAGFIKRQLVETRQITKHVAQI
LDSRMNTKYDENDKLIREVKVITLKSKLVSDFRKDFQFYKVREIN
NYHHAHDAYLNAVVGTALIKKYPKLESEFVYGDYKVYDVRKMIAK
SEQEIGKATAKYFFYSNIMNFFKTEITLANGEIRKRPLIETNGET
GEIVWDKGRDFATVRKVLSMPQVNIVKKTEVQTGGFSKESILPKR
NSDKLIARKKDWDPKKYGGFDSPTVAYSVLVVAKVEKGKSKKLKS
VKELLGITIMERSSFEKNPIDFLEAKGYKEVKKDLIIKLPKYSLF
ELENGRKRMLASAGELQKGNELALPSKYVNFLYLASHYEKLKGSP
EDNEQKQLFVEQHKHYLDEIIEQISEFSKRVILADANLDKVLSAY
NKHRDKPIREQAENIIHLFTLTNLGAPAAFKYFDTTIDRKRYTST
KEVLDATLIHQSITGLYETRIDLSQLGGDSGGSSGGSSGSETPGT
SESATPESSGGSSGGSSKSAEYLNTFRLRNLGLPVMNNLHDMSKA
TRISVETLRLLIYTADFRYRIYTVEKKGPEKRMRTIYQPSRELKA
LQGWVLRNILDKLSSSPFSIGFEKHQSILNNATPHIGANFILNID
LEDFFPSLTANKVFGVFHSLGYNRLISSVLTKICCYKNLLPQGAP
SSPKLANLICSKLDYRIQGYAGSRGLIYTRYADDLTLSAQSMKKV
VKARDFLFSIIPSEGLVINSKKTCISGPRSQRKVTGLVISQEKVG
IGREKYKEIRAKIHHIFCGKSSEIEHVRGWLSFILSVDSKSHRRL
ITYISKLEKKYGKNPLNKAKTPPKKKRKV
pSCL.102: Eco1RT-P2A-SpCas9
SV40 NLS (bold font)-Eco1 RT-P2A
(italic font)-SpCas9-SV40 NLS (underline)
(SEQ ID NO: 26)
MPPKKKRKVKSAEYLNTFRLRNLGLPVMNNLHDMSKATRISVETL
RLLIYTADFRYRIYTVEKKGPEKRMRTIYQPSRELKALQGWVLRN
ILDKLSSSPFSIGFEKHQSILNNATPHIGANFILNIDLEDFFPSL
TANKVFGVFHSLGYNRLISSVLTKICCYKNLLPQGAPSSPKLANL
ICSKLDYRIQGYAGSRGLIYTRYADDLTLSAQSMKKVVKARDFLF
SHIPSEGLVINSKKTCISGPRSQRKVTGLVISQEKVGIGREKYKE
IRAKIHHIFCGKSSEIEHVRGWLSFILSVDSKSHRRLITYISKLE
KKYGKNPLNKAKTATNFSLLKQAGDVEENPGPDKKYSIGLDIGTN
SVGWAVITDEYKVPSKKFKVLGNTDRHSIKKNLIGALLFDSGETA
EATRLKRTARRRYTRRKNRICYLQEIFSNEMAKVDDSFFHRLEES
FLVEEDKKHERHPIFGNIVDEVAYHEKYPTIYHLRKKLVDSTDKA
DLRLIYLALAHMIKFRGHFLIEGDLNPDNSDVDKLFIQLVQTYNQ
LFEENPINASGVDAKAILSARLSKSRRLENLIAQLPGEKKNGLFG
NLIALSLGLTPNFKSNFDLAEDAKLQLSKDTYDDDLDNLLAQIGD
QYADLFLAAKNLSDAILLSDILRVNTEITKAPLSASMIKRYDEHH
QDLTLLKALVRQQLPEKYKEIFFDQSKNGYAGYIDGGASQEEFYK
FIKPILEKMDGTEELLVKLNREDLLRKQRTFDNGSIPHQIHLGEL
HAILRRQEDFYPFLKDNREKIEKILTFRIPYYVGPLARGNSRFAW
MTRKSEETITPWNFEEVVDKGASAQSFIERMTNFDKNLPNEKVLP
KHSLLYEYFTVYNELTKVKYVTEGMRKPAFLSGEQKKAIVDLLFK
TNRKVTVKQLKEDYFKKIECFDSVEISGVEDRFNASLGTYHDLLK
IIKDKDFLDNEENEDILEDIVLTLTLFEDREMIEERLKTYAHLFD
DKVMKQLKRRRYTGWGRLSRKLINGIRDKQSGKTILDFLKSDGFA
NRNFMQLIHDDSLTFKEDIQKAQVSGQGDSLHEHIANLAGSPAIK
KGILQTVKVVDELVKVMGRHKPENIVIEMARENQTTQKGQKNSRE
RMKRIEEGIKELGSQILKEHPVENTQLQNEKLYLYYLQNGRDMYV
DQELDINRLSDYDVDHIVPQSFLKDDSIDNKVLTRSDKNRGKSDN
VPSEEVVKKMKNYWRQLLNAKLITQRKFDNLTKAERGGLSELDKA
GFIKRQLVETRQITKHVAQILDSRMNTKYDENDKLIREVKVITLK
SKLVSDFRKDFQFYKVREINNYHHAHDAYLNAVVGTALIKKYPKL
ESEFVYGDYKVYDVRKMIAKSEQEIGKATAKYFFYSNIMNFFKTE
ITLANGEIRKRPLIETNGETGEIVWDKGRDFATVRKVLSMPQVNI
VKKTEVQTGGFSKESILPKRNSDKLIARKKDWDPKKYGGFDSPTV
AYSVLVVAKVEKGKSKKLKSVKELLGITIMERSSFEKNPIDFLEA
KGYKEVKKDLIIKLPKYSLFELENGRKRMLASAGELQKGNELALP
SKYVNFLYLASHYEKLKGSPEDNEQKQLFVEQHKHYLDEIIEQIS
EFSKRVILADANLDKVLSAYNKHRDKPIREQAENIIHLFTLTNLG
APAAFKYFDTTIDRKRYTSTKEVLDATLIHQSITGLYETRIDLSQ
LGGDSRADPKKKRKV
pSCL.103 and pSCL.139: SpCas9-P2A-Eco1RT
SV40 NLS (bold font)-SpCas9-P2A
(italic font)-Eco1RT-SV40 NLS (underline)
(SEQ ID NO: 27)
MSRADPKKKRKVDKKYSIGLDIGTNSVGWAVITDEYKVPSKKFKV
LGNTDRHSIKKNLIGALLFDSGETAEATRLKRTARRRYTRRKNRI
CYLQEIFSNEMAKVDDSFFHRLEESFLVEEDKKHERHPIFGNIVD
EVAYHEKYPTIYHLRKKLVDSTDKADLRLIYLALAHMIKFRGHFL
IEGDLNPDNSDVDKLFIQLVQTYNQLFEENPINASGVDAKAILSA
RLSKSRRLENLIAQLPGEKKNGLFGNLIALSLGLTPNFKSNFDLA
EDAKLQLSKDTYDDDLDNLLAQIGDQYADLFLAAKNLSDAILLSD
ILRVNTEITKAPLSASMIKRYDEHHQDLTLLKALVRQQLPEKYKE
IFFDQSKNGYAGYIDGGASQEEFYKFIKPILEKMDGTEELLVKLN
REDLLRKQRTFDNGSIPHQIHLGELHAILRRQEDFYPFLKDNREK
IEKILTFRIPYYVGPLARGNSRFAWMTRKSEETITPWNFEEVVDK
GASAQSFIERMTNFDKNLPNEKVLPKHSLLYEYFTVYNELTKVKY
VTEGMRKPAFLSGEQKKAIVDLLFKTNRKVTVKQLKEDYFKKIEC
FDSVEISGVEDRFNASLGTYHDLLKIIKDKDFLDNEENEDILEDI
VLTLTLFEDREMIEERLKTYAHLFDDKVMKQLKRRRYTGWGRLSR
KLINGIRDKQSGKTILDFLKSDGFANRNFMQLIHDDSLTFKEDIQ
KAQVSGQGDSLHEHIANLAGSPAIKKGILQTVKVVDELVKVMGRH
KPENIVIEMARENQTTQKGQKNSRERMKRIEEGIKELGSQILKEH
PVENTQLQNEKLYLYYLQNGRDMYVDQELDINRLSDYDVDHIVPQ
SFLKDDSIDNKVLTRSDKNRGKSDNVPSEEVVKKMKNYWRQLLNA
KLITQRKFDNLTKAERGGLSELDKAGFIKRQLVETRQITKHVAQI
LDSRMNTKYDENDKLIREVKVITLKSKLVSDFRKDFQFYKVREIN
NYHHAHDAYLNAVVGTALIKKYPKLESEFVYGDYKVYDVRKMIAK
SEQEIGKATAKYFFYSNIMNFFKTEITLANGEIRKRPLIETNGET
GEIVWDKGRDFATVRKVLSMPQVNIVKKTEVQTGGFSKESILPKR
NSDKLIARKKDWDPKKYGGFDSPTVAYSVLVVAKVEKGKSKKLKS
VKELLGITIMERSSFEKNPIDFLEAKGYKEVKKDLIIKLPKYSLF
ELENGRKRMLASAGELQKGNELALPSKYVNFLYLASHYEKLKGSP
EDNEQKQLFVEQHKHYLDEIIEQISEFSKRVILADANLDKVLSAY
NKHRDKPIREQAENIIHLFTLTNLGAPAAFKYFDTTIDRKRYTST
KEVLDATLIHQSITGLYETRIDLSQLGGDATNFSLLKQAGDVEEN
PGPKSAEYLNTFRLRNLGLPVMNNLHDMSKATRISVETLRLLIYT
ADFRYRIYTVEKKGPEKRMRTIYQPSRELKALQGWVLRNILDKLS
SSPFSIGFEKHQSILNNATPHIGANFILNIDLEDFFPSLTANKVF
GVFHSLGYNRLISSVLTKICCYKNLLPQGAPSSPKLANLICSKLD
YRIQGYAGSRGLIYTRYADDLTLSAQSMKKVVKARDFLFSIIPSE
GLVINSKKTCISGPRSQRKVTGLVISQEKVGIGREKYKEIRAKIH
HIFCGKSSEIEHVRGWLSFILSVDSKSHRRLITYISKLEKKYGKN
PLNKAKTPPKKKRKV
pSCL.175: HEK3 targeting and editing
Eco1 neRNA-gRNA (a1/a2 length: 27
v1), expressed constitutively from
a pol III (H1) promoter
H1 promoter (bold font)-linker
(bold underlined font)-Eco1 ncRNA-HEK3
donor (italic font)-Eco1 ncRNA-HEK3
gRNA (underline)-SpCas9 Scaffold
(SEQ ID NO: 28)
AATTCGGAACGCTGACGTCATCAACCCGCTCCAAGGAATCGCGGG
CCCAGTGTCACTAGGCGGGAACACCCAGCGCGCGTGCGCCCTGGC
AGGAAGATGGCTGTGAGGGACAGGGGAGTGGCGCCCTGCAATATT
TGCATGTCGCTATGTGTTCTGGGAAATCACCATAAACGTGAAATG
TCTTTGGATTTGGGAATCTTATAAGTTCTGTATGAGACCACCCGA
AACGAGCTAGCTCGTCATGATAAGATTCCGTATGCGCACCCTTAG
CGAGAGGTTTATCATTAAGGTCAACCTCTGGATGTTGTTTCGGCA
TCCTGCATTGAATCTGAGTTACTGTCTGTTTTCCTCTTCTCCAGC
CCTGGCCTGGGTCAATCCTTGGGGCCCAGACTGAGCACGTGCTAA
CAGAGGAAAGGAAGCCCTGCTTCCTCCAGAGGGCGTCGCAGGACA
GCTTTTCCTAGACAGGGGCTAGGAAACCCGTTTCTTCTGACGTAA
GGGTGCGCATACGGAATCTTATCAGGCCCAGACTGAGCACGTGAG
TTTCAGAGCTATGCTGGAAACAGCATAGCAAGTTGAAATAAGGCT
AGTCCGTTATCAACTTGAAAAAGTGGCACCGAGTCGGTGCTTTTT
pSCL.176: RNF2 targeting and editing
Eco1 ncRNA-gRNA (a1/a2 length: 27
v1), expressed constitutively from a
pol III (H1) promoter
H1 promoter (bold font)-linker (bold
underlined font)-Eco1 neRNA-RNF2 donor
(italic font)-Eco1 ncRNA-RNF2 gRNA
(underlined)-SpCas9 Scaffold
(SEQ ID NO: 29)
AATTCGGAACGCTGACGTCATCAACCCGCTCCAAGGAATCGCGGG
CCCAGTGTCACTAGGCGGGAACACCCAGCGCGCGTGCGCCCTGGC
AGGAAGATGGCTGTGAGGGACAGGGGAGTGGCGCCCTGCAATATT
TGCATGTCGCTATGTGTTCTGGGAAATCACCATAAACGTGAAATG
TCTTTGGATTTGGGAATCTTATAAGTTCTGTATGAGACCACCCGA
AACGAGCTAGCTCGTCATGATAAGATTCCGTATGCGCACCCTTAG
CGAGAGGTTTATCATTAAGGTCAACCTCTGGATGTTGTTTCGGCA
TCCTGCATTGAATCTGAGTTACTGTCTGTTTTCCTCCCAGTTTAC
ACGTCTCATATGCCCCTTGGCAGTCATCTTAGTCATTACATGAAA
TGTTCGTTGTAACTCATATAAACTGAGTTCCCATGTTTTGCTTAA
TGGTTGAGTTCCGTTTGTCTAGGAAACCCGTTTCTTCTGACGTAA
GGGTGCGCATACGGAATCTTATCAGTCATCTTAGTCATTACCTGG
TTTCAGAGCTATGCTGGAAACAGCATAGCAAGTTGAAATAAGGCT
AGTCCGTTATCAACTTGAAAAAGTGGCACCGAGTCGGTGCTTTTT
pSCL. 177: EMX1 targeting and
editing Eco1 ncRNA-gRNA (a1/a2 length: 27
v1), expressed constitutively from a
pol III (H1) promoter H1 promoter
(bold font)-linker (bold underlined
font)-Eco1 ncRNA-EMX1 donor (italic
font)-Eco1 ncRNA-EMX1 gRNA (underlined)-
SpCas9 Scaffold
(SEQ ID NO: 30)
AATTCGGAACGCTGACGTCATCAACCCGCTCCAAGGAATCGCGGG
CCCAGTGTCACTAGGCGGGAACACCCAGCGCGCGTGCGCCCTGGC
AGGAAGATGGCTGTGAGGGACAGGGGAGTGGCGCCCTGCAATATT
TGCATGTCGCTATGTGTTCTGGGAAATCACCATAAACGTGAAATG
TCTTTGGATTTGGGAATCTTATAAGTTCTGTATGAGACCACCCGA
AACGAGCTAGCTCGTCATGATAAGATTCCGTATGCGCACCCTTAG
CGAGAGGTTTATCATTAAGGTCAACCTCTGGATGTTGTTTCGGCA
TCCTGCATTGAATCTGAGTTACTGTCTGTTTTCCTACAAACGGCA
GAAGCTGGAGGAGGAAGGGCCTGAGTCCGAGCAGAAGAAAAAGTT
CTCCCATCACATCAACCGGTGGCGCATTGCCACGAAGCAGGCCAA
TGGGGAGGACATCGATGTCAAGGAAACCCGTTTCTTCTGACGTAA
GGGTGCGCATACGGAATCTTATCAGAGTCCGAGCAGAAGAAGAAG
TTTCAGAGCTATGCTGGAAACAGCATAGCAAGTTGAAATAAGGCT
AGTCCGTTATCAACTTGAAAAAGTGGCACCGAGTCGGTGCTTTTT
pSCL.178: FANCF targeting and editing Eco1
ncRNA-gRNA (a1/a2 length: 27 v1), expressed
constitutively from a pol III (H1) promoter
H1 promoter (bold font)-linker (bold
underlined font)-Eco1 ncRNA-FANCF donor
(italic font)-Eco1 ncRNA-FANCF gRNA
(underlined)-SpCas9 Scaffold
(SEQ ID NO: 31)
AATTCGGAACGCTGACGTCATCAACCCGCTCCAAGGAATCGCGGG
CCCAGTGTCACTAGGCGGGAACACCCAGCGCGCGTGCGCCCTGGC
AGGAAGATGGCTGTGAGGGACAGGGGAGTGGCGCCCTGCAATATT
TGCATGTCGCTATGTGTTCTGGGAAATCACCATAAACGTGAAATG
TCTTTGGATTTGGGAATCTTATAAGTTCTGTATGAGACCACCCGA
AACGAGCTAGCTCGTCATGATAAGATTCCGTATGCGCACCCTTAG
CGAGAGGTTTATCATTAAGGTCAACCTCTGGATGTTGTTTCGGCA
TCCTGCATTGAATCTGAGTTACTGTCTGTTTTCCTGAAAGCGGAA
GTAGGGCCTTCGCGCACCTCATGGAATCCCTTCTGCAGCATCTAG
ATCGCTTTTCCGAGCTTCTGGCGGTCTCAAGCACTACCTACGTCA
GCACCTGGGACCCCGCCACCAGGAAACCCGTTTCTTCTGACGTAA
GGGTGCGCATACGGAATCTTATCAGGAATCCCTTCTGCAGCACCG
TTTCAGAGCTATGCTGGAAACAGCATAGCAAGTTGAAATAAGGCT
AGTCCGTTATCAACTTGAAAAAGTGGCACCGAGTCGGTGCTTTTT
pSCL.179: HEK4 targeting and editing Eco1
ncRNA-gRNA (a1/a2 length: 27 v1), expressed
constitutively from a pol III (H1) promoter
H1 promoter (bold font)-linker (bold
underlined font)-Eco1 ncRNA-HEK4 donor
(italic font)-Eco1 ncRNA-HEK4 gRNA
(underline)-SpCas9 Scaffold
(SEQ ID NO: 32)
AATTCGGAACGCTGACGTCATCAACCCGCTCCAAGGAATCGCGGG
CCCAGTGTCACTAGGCGGGAACACCCAGCGCGCGTGCGCCCTGGC
AGGAAGATGGCTGTGAGGGACAGGGGAGTGGCGCCCTGCAATATT
TGCATGTCGCTATGTGTTCTGGGAAATCACCATAAACGTGAAATG
TCTTTGGATTTGGGAATCTTATAAGTTCTGTATGAGACCACCCGA
AACGAGCTAGCTCGTCATGATAAGATTCCGTATGCGCACCCTTAG
CGAGAGGTTTATCATTAAGGTCAACCTCTGGATGTTGTTTCGGCA
TCCTGCATTGAATCTGAGTTACTGTCTGTTTTCCTGATGACAGGC
AGGGGCACCGCGGCGCCCCGGTGGCACTGCGGCTGGAGGCGGGAA
TTAAAGCGGAGACTCTGGTGCTGTGTGACTACAGTGGGGGCCCTG
CCCTCTCTGAGCCCCCGCCTAGGAAACCCGTTTCTTCTGACGTAA
GGGTGCGCATACGGAATCTTATCAGGCACTGCGGCTGGAGGTGGG
TTTCAGAGCTATGCTGGAAACAGCATAGCAAGTTGAAATAAGGCT
AGTCCGTTATCAACTTGAAAAAGTGGCACCGAGTCGGTGCTTTTT
pSCL.180: AAVS1 targeting and editing Eco1
ncRNA-gRNA (a1/a2 length: 27 v1), expressed
constitutively from a pol III (H1) promoter
H1 promoter (bold font)-linker (bold
underlined font)-Eco1 ncRNA-AAVS1 donor
(italic font)-Eco1 ncRNA-AAVS1 gRNA
(underlined)-SpCas9 Scaffold
(SEQ ID NO: 33)
AATTCGGAACGCTGACGTCATCAACCCGCTCCAAGGAATCGCGGG
CCCAGTGTCACTAGGCGGGAACACCCAGCGCGCGTGCGCCCTGGC
AGGAAGATGGCTGTGAGGGACAGGGGAGTGGCGCCCTGCAATATT
TGCATGTCGCTATGTGTTCTGGGAAATCACCATAAACGTGAAATG
TCTTTGGATTTGGGAATCTTATAAGTTCTGTATGAGACCACCCGA
AACGAGCTAGCTCGTCATGATAAGATTCCGTATGCGCACCCTTAG
CGAGAGGTTTATCATTAAGGTCAACCTCTGGATGTTGTTTCGGCA
TCCTGCATTGAATCTGAGTTACTGTCTGTTTTCCTTAATGTGGCT
CTGGTTCTGGGTACTTTTATCTGTCCCCTCCACCCCACAATGGAA
CCACTAGGGACAGGATTGGTGACAGAAAAGCCCCCATCCTTAGGC
CTCCTCCTTCCTAGTCTCCTAGGAAACCCGTTTCTTCTGACGTAA
GGGTGCGCATACGGAATCTTATCAGTCCCCTCCACCCCACAGTGG
TTTCAGAGCTATGCTGGAAACAGCATAGCAAGTTGAAATAAGGCT
AGTCCGTTATCAACTTGAAAAAGTGGCACCGAGTCGGTGCTTTTT

qPCR

qPCR analysis of RT-DNA was carried out by comparing amplification from samples using two sets of primers. One set could only use the plasmid as a template because they bound outside the msd region (outside) and the other set could use either the plasmid or RT-DNA as a template because they bound inside the msd region (inside). Results were analyzed by first taking the difference in cycle threshold (CT) between the inside and outside primer sets for each biological replicate. Next, each biological replicate ΔCT was subtracted from the average ΔCT of the control condition (e.g. uninduced). Fold change was calculated as 2^{-ΔΔCT} for each biological replicate. This fold change represents the difference in abundance of the inside versus outside template, where the presence of RT-DNA leads to fold change values greater than 1.

For the initial analysis of Eco1 RT-DNA when overexpressed in E. coli, the qPCR analysis used just three primers, two of which bound inside the msd and one which bound outside. The inside PCR was generated using both inside primers, while the outside PCR used one inside and one outside primer. For all other experiments, four primers were used. Two bound inside the msd and two bound outside the msd in the RT. qPCR primers are all listed in Table 6.

TABLE 6
Primers

| Name | Sequence | Purpose |
|---|---|---|
| pKD3_Eco1_ | GGATCTATTCATAACTTGATGTAT | Generating a knockout |
| KO_F | AAAGTAGAAAAAAAAGCGGGGAG | template |
| | ATTGTGTAGGCTGGAGCTGCTTC | for the Eco1 |
| | (SEQ ID NO: 34) | locus from pKD3 |
| pKD3_Eco1_ | GAAGTGCTCAATTTTTCAACCTA | |
| KO_R | TGAGCTTTAGTTTTAAAACGAAG | |
| | ACCACATATGAATATCCTCCTTA | |
| | (SEQ ID NO: 35) | |
| Eco1_KO_ | CATGTGCATGAAAACCACTGC | Verifying locus-specific |
| genotyping_F | (SEQ ID NO: 36) | |
| Eco1_KO_ | ATCATGCCGTTTGTGATGG | insertion of CM cassette |
| genotyping_R | (SEQ ID NO: 37) | |
| Eco1_KO_ | GCAATGCGATGAAAAGTGCC | With Eco1_KO_ |
| CM_marker_ | (SEQ ID NO: 38) | genotyping_F, |
| loss_R | | verifying excision |
| | | of CM cassette |
| qPCR_Eco1_ | GTCAGAAAAAACGGGTTT | qPCR quantification of |
| wt_1 | CCTGGTTGG | RT-DNA from Eco1 (wt) |
| | (SEQ ID NO: 39) | |
| qPCR_Eco1_ | TCTGAGTTACTGTCTG | with qPCR_Eco1_wt_1, |
| _wt_2 | TTTTCCTTGTTGG | amplifies the RT-DNA |
| | (SEQ ID NO: 99) | and plasmid |
| qPCR_Eco1_ | CCTCTGGATGTTGTTTCGGCA | with qPCR_Eco1_wt_1, |
| wt_3 | (SEQ ID NO: 40) | amplifies only the plasmid |
| qPCR_Eco1_ | CAAGGATGGGTTCTACGTAACA | qPCR quantification |
| Recombineering_ | (SEQ ID NO: 41) | of RT-DNA from Eco1 |
| RT_F | | |
| qPCR_Eco1_ | GCTAATTTAGGTGATGATGGAGC | Recombineering ncRNA |
| Recombineering | (SEQ ID NO: 42) | amplifies only plasmid (RT) |
| RT_R | | |
| qPCR_Eco1_ | CTGAGTTACTGTCTGT | qPCR quantification |
| Recombineering_ | TTTCCTG | of RT-DNA from Eco1 |
| RTDNA_F | (SEQ ID NO: 43) | Recombineering ncRNA |
| qPCR_Eco1_ | TCAGAAAAAACGGGTTT | amplifies plasmid and |
| Recombineering_ | CCTGAATTC | RT-DNA (RT-DNA) |
| RTDNA_R | (SEQ ID NO: 44) | |
| rpoB_ | CTTTCCCTACACGAC | Genotyping of single base |
| genotyping_F | GCTCTTCCGATCTCTCTGGG | change in rpoB gene |
| | CGATCTGGATACC | by Illumina sequencing |
| | (SEQ ID NO: 45) | |
| rpoB_ | GGAGTTCAGACGTGTGC | |
| genotyping_R | TCTTCCGATCTTTCGATT | |
| | GGACATACGCGAC | |
| | (SEQ ID NO: 46) | |
| fabH_ | CTTTCCCTACACGACG | Genotyping of single base |
| genotyping_F | CTCTTCCGATCTTATCAG | change in fabH gene |
| | CCAGCATTCCAACG | by Illumina sequencing |
| | (SEQ ID NO: 47) | |
| fabH_ | GGAGTTCAGACGTGTG | |
| genotyping_R | CTCTTCCGATCTGGCT | |
| | AACCTGCGTATTATCA | |
| | GTG | |
| | (SEQ ID NO: 48) | |
| murF_ | CTTTCCCTACACGAC | Genotyping of single base |
| genotyping_F | GCTCTTCCGATCTAAT | Change in murF gene |
| | GAGCAATCATACGCGGG | by Illumina sequencing |
| | (SEQ ID NO: 49) | |
| murF_ | GGAGTTCAGACGTGTGCT | |
| genotyping_R | CTTCCGATCTACGATTTT | |
| | AGTTAAGGGTTCACG | |
| | (SEQ ID NO: 50) | |
| priB_ | CTTTCCCTACACGACGCT | Genotyping of single base |
| genotyping_F | CTTCCGATCTGGGACAATC | change in priB gene |
| | TTACCGCTTTCG | by Illumina sequencing |
| | (SEQ ID NO: 51) | |
| priB_ | GGAGTTCAGACGTGTGCT | |
| genotyping_R | CTTCCGATCTACAAGGCAAA | |
| | GAACGGACTG | |
| | (SEQ ID NO: 52) | |
| Vector_BB_ | agctaGGTCTCATTCAATG | Amplifies vector |
| for_stem_and_ | CAGGATGCCGAAACAAC | for insertion |
| loop_library_F | (SEQ ID NO: 53) | of stem and loop |
| | | library parts |
| Vector_BB_ | agctaGGTCTCAGTAAGGGTG | |
| for_stem_and_ | CGCAACTTTCATG | |
| loop_library_R | (SEQ ID NO: 54) | |
| Vector_BB_ | agctaGGTCTCATTCAATGC | Amplifies vector |
| for_a1/a2_ | AGGATGCCGAAACAAC | for insertion |
| library_F | (SEQ ID NO: 55) | of a1/a2 library parts |
| Vector_BB_ | agctaGGTCTCAACTTTCAT | |
| for_a1/a2_ | GAAATCCGCTGCATCAC | |
| library_R | (SEQ ID NO: 56) | |
| Stem_Library_ | AGTGACCCGTCCCTG | Amplifies stem library |
| part_F | (SEQ ID NO: 57) | variant parts |
| Stem_Library_ | AGTCGACCTCTGCCC | |
| part_R | (SEQ ID NO: 58) | |
| a1/a2_ | ACTGGTGCGTCGTCT | Amplifies a1/a2 library |
| Library_ | (SEQ ID NO: 59) | variant parts |
| part_F | | |
| a1/a2_ | CGGGAAGTGTTCGCC | |
| Library_ | (SEQ ID NO: 60) | |
| part_R | | |
| Eco1_Variant_ | CTTTCCCTACACGACGCTCTTCC | Amplifies ncRNA |
| Plasmids_for_ | GATCTNNNNNTTATGCTAGGT | from variant |
| Sequencing_F | GATGCAGCGGATTTCATGAAAG | |
| | (SEQ ID NO: 61) | |
| Eco1_Variant_ | GGAGTTCAGACGTGTGCTCTT | library plasmids |
| Plasmids_for_ | CCGATCTCATTTAACTATGATAA | for Illumina sequencing |
| Sequencing_R | GATTCCGTATGCGCACC | |
| | (SEQ ID NO: 62) | |
| ssExt_pG6_ | GGAGTTCAGACGTGTGCTCTT | Creates complementary |
| anchor | CCGATCTGGGGGGH | strand from |
| | (SEQ ID NO: 63) | extended RT-DNA |
| Eco1_ | CTTTCCCTACACGACGCTCTT | Amplifies barcodes |
| msdloop_for_ | CCGATCTTCAGAAAAAACGGG | in the msd loop |
| Sequencing_F | TTGTCGCC | |
| | (SEQ ID NO: 64) | |
| Eco1_ | GGAGTTCAGACGTGTGCTCTT | from purified RT-DNA |
| msdloop_for | CCGATCTGTTACAAGCTG | for Illumina sequencing |
| _Sequencing_R | TTTGTCGCCAG | |
| | (SEQ ID NO: 65) | |
| SCL325 | GTCGAAAAGAAGGGGCCTGA | qPCR primers to amplify |
| | (SEQ ID NO: 66) | the Eco1RT |
| | | (yeast version) |
| SCL326 | CCGCGTACCCTTGTATTCGA | |
| | (SEQ ID NO: 67) | |
| SCL287 | TCCTTGTTGGAACGGAGAGC | qPCR primers to amplify |
| | (SEQ ID NO: 100) | the Eco1 RT-DNA |
| | | (yeast version) |
| SCL288 | TAACGGGTTTCCTGGTTGGC | |
| | (SEQ ID NO: 68) | |
| SCL196 | ACCTTAAAGCTGCCCTOCAT | qPCR primers to amplify |
| | (SEQ ID NO: 69) | the Eco2RT |
| | | (yeast and human version) |
| SCL197 | GGTACGCTGCCGTAATAGGA | |
| | (SEQ ID NO: 70) | |
| SCL34 | GAATCGCCTCCCTAAAATCC | qPCR primers to amplify |
| | (SEQ ID NO: 71) | the Eco2 RT-DNA |
| | | (yeast and human version) |
| SCL37 | GCACACCTGCCGTATAGCTC | |
| | (SEQ ID NO: 72) | |
| SCL378 | CTTTCCCTACACGACGCTC | Amplify the yeast |
| | TTCCGATCTTATGCGCCTGC | ADE2 locus |
| | TAGAGTTCC | for Illumina sequencing |
| | (SEQ ID NO: 73) | |
| SCL193 | GGAGTTCAGACGTGTG | |
| | CTCTTCCGATCTGCGTT | |
| | CGTTGTAATGGTGGAG | |
| | (SEQ ID NO: 74) | |
| SCL654 | CTTTCCCTACACGACG | Amplify the yeast |
| | CTCTTCCGATCTTGTCAA | CAN1 locus |
| | GGACCACCAAAGGT | for Illumina sequencing |
| | (SEQ ID NO: 75) | |
| SCL655 | GGAGTTCAGACGTGTG | |
| | CTCTTCCGATCTGCCGCAT | |
| | AATAAGCCAAGCC | |
| | (SEQ ID NO: 76) | |
| SCL656 | CTTTCCCTACACGACG | Amplify the yeast |
| | CTCTTCCGATCTGACGGC | LYP1 locus |
| | GAGGAGAACTGAAA | for Illumina sequencing |
| | (SEQ ID NO: 77) | |
| SCL657 | GGAGTTCAGACGTGTG | |
| | CTCTTCCGATCTCACCTTG | |
| | CAATGACCCATGC | |
| | (SEQ ID NO: 78) | |
| SCL658 | CTTTCCCTACACGACG | Amplify the yeast |
| | CTCTTCCGATCTCCCGTG | TRP2 locus |
| | TATGCGTATTTGCC | for Illumina sequencing |
| | (SEQ ID NO: 79) | |
| SCL659 | GGAGTTCAGACGTG | |
| | TGCTCTTCCGATCTGCCT | |
| | TCTTGTTTTTGGCTCGA | |
| | (SEQ ID NO: 80) | |
| SCL660 | CTTTCCCTACACGACGC | Amplify the yeast |
| | TCTTCCGATCTGATCAAA | FAA1 locus |
| | CCAGTGCAAGCCG | for Illumina sequencing |
| | (SEQ ID NO: 81) | |
| SCL661 | GGAGTTCAGACGTGTG | |
| | CTCTTCCGATCTTAACA | |
| | CGGTCGGTATTGCCC | |
| | (SEQ ID NO: 82) | |
| SCL815 | CTTTCCCTACACGACG | Amplify the human |
| | CTCTTCCGATCTAGACAG | HEK3 locus |
| | GGATCCCAGGGAAA | for Illumina sequencing |
| | (SEQ ID NO: 83) | |
| SCL816 | GGAGTTCAGACGTGT | |
| | GCTCTTCCGATCTGCCC | |
| | AGCCAAACTTGTCAAC | |
| | (SEQ ID NO: 84) | |
| SCL817 | CTTTCCCTACACGAC | Amplify the human |
| | GCTCTTCCGATCTCTCTT | RNF2 locus |
| | CTTTATTTCCAGCAATGTCT | for Illumina sequencing |
| | (SEQ ID NO: 85) | |
| SCL818 | GGAGTTCAGACGTG | |
| | TGCTCTTCCGATCTAGCC | |
| | AACATACAGAAGTCAGGA | |
| | (SEQ ID NO: 101) | |
| SCL819 | CTTTCCCTACACGACG | Amplify the human |
| | CTCTTCCGATCTAGGT | EMX1 locus |
| | GAAGGTGTGGTTCCAG | for Illumina sequencing |
| | (SEQ ID NO: 86) | |
| SCL820 | GGAGTTCAGACGTGT | |
| | GCTCTTCCGATCTGCCA | |
| | GAGTCCAGCTTGGG | |
| | (SEQ ID NO: 87) | |
| SCL821 | CTTTCCCTACACGACG | Amplify the human |
| | CTCTTCCGATCTTCGC | FANCF locus |
| | GGATGTTCCAATCAGT | for Illumina sequencing |
| | (SEQ ID NO: 102) | |
| SCL822 | GGAGTTCAGACGTGTG | |
| | CTCTTCCGATCTGATGGA | |
| | TGTGGCGCAGGTAG | |
| | (SEQ ID NO: 88) | |
| SCL823 | CTTTCCCTACACGAC | Amplify the human |
| | GCTCTTCCGATCTCCCT | HEK4 locus |
| | CCCTTCAAGATGGCTG | for Illumina sequencing |
| | (SEQ ID NO: 89) | |
| SCL824 | GGAGTTCAGACGTGT | |
| | GCTCTTCCGATCTTCCTT | |
| | TCAACCCGAACGGAG | |
| | (SEQ ID NO: 90) | |
| SCL650 | CTTTCCCTACACGA | Amplify the human |
| | CGCTCTTCCGATCTG | AAVS1 locus |
| | CTCAGCTAGTCTTCTT | for Illumina sequencing |
| | CCTCC (SEQ ID NO: 91) | |
| SCL651 | GGAGTTCAGACGTGTG | |
| | CTCTTCCGATCTGGG | |
| | GGTGTGTCACCAGATAA | |
| | (SEQ ID NO: 92) | |
| SCL | GAATCTGAGTTACTGTCTGTTT | Cloned into pZS.165 |
| gblock_001 | TCCTTGGAAATGTTCTATTTAG | to make pSCL.002- |
| | AAACAGGGGAATTGCTTATTAA | ade2hdr-sgAde2 oligo: |
| | CGAAATTGCCTGAAGGCCTCAC | edits the ade2 locus |
| | AACTCTGGACATTATACCATT | |
| | GATGCTTGCGTCACTTCAGG | |
| | AAACCCGTTTCTTCTGACGT | |
| | AAGGGTGCGCAATTAACG | |
| | AAATTGCCCCAGTTTCAGAG | |
| | CTATGCTGGAAACAGCATAG | |
| | CAAGTTGAAATAAGGCTAGT | |
| | CCGTTATCAACTTGAAAAAGT | |
| | GGCACCGAGTCGGTGCTTTTT | |
| | GATGG (SEQ ID NO: 93) | |
| Eco1_RT_ | ACGTTCCGCCTTAGGAATTT | qPCR primers to amplify |
| fow | (SEQ ID NO: 94) | the Eco1RT |
| | | (human version) |
| Eco1_RT_ | TTTCTCAGGCCCCTTCTTTT | |
| rev | (SEQ ID NO: 95) | |
| Eco1_ | AAATAACGGGTTTCCTGGTTG | qPCR primers to amplify |
| RTDNA_fow | (SEQ ID NO: 96) | the Eco1 RT-DNA |
| | | (human version) |
| Eco1_ | TCTGTTTTCCTTGTTGGAACG | |
| RTDNA_rev | (SEQ ID NO: 97) | |

For bacterial experiments, constructs were expressed in liquid culture, shaking at 37° C. for 6-16 hours after which a volume of 25 μl of culture was harvested, mixed with 25 μl H₂O, and incubated at 95° C. for 5 minutes. A volume of 0.3 ul of this boiled culture was used as a template in 30 μl reactions using a KAPA SYBR FAST qPCR mix.

For yeast experiments, single colonies were inoculated into SC-URA 2% Glucose and grown shaking overnight at 30° C. To express the constructs, the overnight cultures were spun down, washed and resuspended in 1 mL of water and passaged at a 1:30 dilution into SC-URA 2% Galactose, grown shaking for 24 h at 30° C. 250 μl aliquots of the uninduced and induced cultures were collected for qPCR analysis. For qPCR sample preparation, the aliquots were spun down, resuspended in 50 μl of water, and incubated at 100° C. for 15 minutes. The samples were then briefly spun down, placed on ice to cool, and 50 μl of the supernatant was treated with Proteinase K by combining it with 29 μl of water, 9 μl of CutSmart buffer and 2 μl of Proteinase K (NEB), followed by incubation at 56° C. for 30 minutes. The Proteinase K was inactivated by incubation at 95° C. for 10 minutes, followed by a 1.5-minute centrifugation at maximum speed (about 21,000 g). The supernatant was collected and used as a template for qPCR reactions, consisting of 2.5 μl of template in 10 μl KAPA SYBR FAST qPCR reactions.

For mammalian experiments, retron expression in stable HEK293T cell lines was induced using 1 μg/mL doxycycline for 24 h at 37° C. in 6-well plates. One ml aliquots of induced and uninduced cell lines were collected for qPCR analysis. qPCR sample preparation and reaction mix followed the yeast experimental protocol.

Primers used to generate and verify strains are listed in Table 5

### RT-DNA Purification and PAGE Analysis

To analyze RT-DNA on a PAGE gel after expression in E. coli, 2 ml of culture were pelleted and nucleotides were prepared using a Qiagen mini prep protocol, substituting Epoch mini spin columns and buffers MX2 and MX3 for Qiagen components. Purified DNA was then treated with additional RNaseA/T1 mix (NEB) for 30 minutes at 37° C. and then single stranded DNA was isolated from the prep using an ssDNA/RNA Clean & Concentrator kit from Zymo Research. The purified RT-DNA was then analyzed on 10% Novex TBE-Urea Gels (Invitrogen), with a 1×TBE running buffer that was heated to greater than 80° C. before loading. Gels were stained with Sybr Gold (Thermo Fisher) and imaged on a Gel Doc imager (Bio-Rad).

To analyze RT-DNA on a PAGE gel after expression in S. cerevisiae, 5 ml of overnight culture in SC-URA 2% Galactose was pelleted and RT-DNA was isolated by RNAse A/T1 treatment of the aqueous (RNA) phase after TRIzol extraction (Invitrogen), following the manufacturer's recommendations with few modifications, as noted here. Cell pellets were resuspended in 500 ul of RNA lysis buffer (100 mM EDTA pH8, 50 mM Tris-HCl pH8, 2% SDS) and incubated for 20 minutes at 85° C., prior to the addition of the TRizol reagent. The aqueous phase was chloroform-extracted twice. Following isopropanol precipitation, the RNA+RT-DNA pellet was resuspended in 265 ul of TE and treated with 5 ul of RNAse A/T1+30 ul NEB2 buffer. The mixture was incubated for 25 minutes at 37° C., after which the RT-DNA was re-precipitated by addition of equal volumes of isopropanol. The resulting RT-DNA was analyzed on Novex 10% TBE-Urea gels as described above.

### Variant Library Cloning

Eco1 ncRNA variant parts were synthesized by Agilent. Variant parts were flanked by BsaI type IIS cut sites and specific primers that allowed amplification of the sublibraries from a larger synthesis run. Random nucleotides were appended to the 3′ end of synthesized parts so that all sequences were the same length (150 bases). The vector to accept these parts (pSLS.601) was amplified with primers that also added BsaI sites, so that the ncRNA variant amplicons and amplified vector backbone could be combined into a Golden Gate reaction using BsaI-HFv2 and T4 ligase to generate a pool of variant plasmids at high efficiency when electroporated into a cloning strain. Variant libraries were miniprepped from the cloning strain and electroporated into the expression strain. Primers for library construction are listed in Table 6.

### Variant Library Expression and Analysis

Eco1 ncRNA variant libraries were grown overnight and then diluted 1:500 for expression. A sample of the culture pre-expression was taken to quantify the variant plasmid library, mixed 1:1 with H2O and incubated at 95° C. for 5 minutes and then frozen at −20° C. Constructs were expressed (arabinose and IPTG for the ncRNA, erythromycin for the RT) as the cells grew shaking at 37° C. for 5 hours, after which time two samples were collected. One was collected to quantify the variant plasmid library. That sample was mixed 1:1 with H2O and incubated at 95° C. for 5 minutes and then frozen at −20° C., identically to the pre-expression sample. The other sample was collected to sequence the RT-DNA. That sample was prepared as described above for RT-DNA purification.

The two variant plasmid library samples (boiled cultures) taken before and after expression were amplified by PCR using primers flanking the ncRNA region that also contained adapters for Illumina sequencing preparation. The purified RT-DNA was prepared for sequencing by first treating with DBR1 (OriGene) to remove the branched RNA, then extending the 3′ end with a single nucleotide, dCTP, in a reaction with terminal deoxynucleotidyl transferase (TdT). This reaction was carried out in the absence of cobalt for 120 seconds at room temperature with the aim of adding only 5-10 cytosines before inactivating the TdT at 70° C. A second complementary strand was then created from that extended product using Klenow Fragment (3′→5′ exo-) with a primer containing an Illumina adapter sequence, six guanines, and a non-guanine (H) anchor. Finally, Illumina adapters were ligated on at the 3′ end of the complementary strand using T4 ligase. In one variation, the loop of the RT-DNA for the a1/a2 library was amplified using Illumina adapter-containing primers in the RT-DNA, but outside the variable region from the purified RT-DNA directly. All products were indexed and sequenced on an Illumina MiSeq. Primers used for sequencing are listed in Table 6.

Python software was custom written to extract variant counts from each plasmid and RT-DNA sample. In each case, these counts were then converted to a percentage of each library, or relative abundance (e.g., raw count for a variant over total counts for all variants). The relative abundance of a given variant in the RT-DNA sample was then divided by the relative abundance of that same variant in the plasmid library, using the average of the pre-induction and post-induction values, to control for differences in the abundance of each variant plasmid in the expression strain. Finally, these corrected abundance values were normalized to the average corrected abundance of the wt variant (set to 100%) or the loop length of 5 (set to 100%).

### Recombineering Expression and Analysis

In experiments using the retron ncRNA to edit bacterial genomes, the retron cassette was co-expressed with CspRecT and mutL E32K from the plasmid pORTMAGE-Ec1 for 16 hours, shaking at 37° C. After expression, a volume of 25 ul of culture was harvested, mixed with 25 ul H₂O, and incubated at 95° C. for 5 minutes. A volume of 0.3 μl of this boiled culture was used as a template in 30 μl reactions with primers flanking the edit site, which additionally contained adapters for Illumina sequencing preparation. These amplicons were indexed and sequenced on an Illumina MiSeq instrument and processed with custom Python software to quantify the percent precisely edited genomes.

### Yeast Editing Expression and Analysis

For yeast genome editing experiments, single colonies from strains containing variants of the Eco1 ncRNA-gRNA cassette (wt or extended a1/a2 length for wt vs. extended a1/a2 region experiments; extended a1/a2 length v1 to test single-promoter expression of Cas9-Eco1RT variants) and editing machinery (−/+Cas9, −/+Eco1RT for wt vs. extended a1/a2 region experiments; Eco1RT-linker1-Cas9, Cas9-linker1-Eco1 RT, Eco1RT-linker2-Cas9, Cas9-linker2-Eco1RT, Eco1RT-P2A-Cas9. Cas9-P2A-Eco1 RT to test single-promoter expression of Cas9-Eco1RT variants) were grown in SC-HIS-URA 2% Raffinose for 24 hours, shaking at 30 C. Cultures were passaged twice into SC-URA 2% Galactose (1:30 dilutions) for 24 hours, for a total of 48 hours of editing. At each timepoint (after 24 h Raffinose, 24 h Galactose. 48 h Galactose), an aliquot of the cultures was harvested, diluted and plated on SC-URA low-ADE plates. Plates were incubated at 30C for 2-3 days, until visible and countable pink (ADE2 KO) and white (ADE2 WT) colonies grew. Editing efficiency was calculated in two ways. The first was by calculating the ratio of pink to total colonies on each plate for each timepoint. This counting was performed by an experimenter blind to the condition. The second was by deep sequencing of the target ADE2 locus. For this, we harvested cells from 250 ul aliquots of the culture for each timepoint in PCR strips, and performed a genomic prep as follows. The pellets were resuspended in 120 μl lysis buffer (see above), heated at 100 C for 15 minutes and cooled on ice. 60 μl of protein precipitation buffer (7.5M Ammonium Acetate) was added and the samples were gently inverted and placed at −20° C. for 10 minutes. The samples were then centrifuged at maximum speed for 2 minutes, and the supernatant was collected in new Eppendorf tubes. Nucleic acids were precipitated by adding equal parts ice-cold isopropanol and incubating the samples at −20° C. for 10 minutes, followed by pelleting by centrifugation at maximum speed for 2 minutes. The pellets were washed twice with 200 ul ice-cold 70% ethanol and dissolved in 40 ul of water. 0.5 μl of the gDNA was used as template in 10 ul reactions with primers flanking the edit site in ADE2, which additionally contained adapters for Illumina sequencing preparation (see Table 6 for primer and oligo sequences). Importantly, the primers do not bind to the ncRNA/gRNA plasmids. These amplicons were indexed and sequenced on an Illumina MiSeq instrument and processed with custom Python software to quantify the percent of P272X edits, caused by Cas9 cleavage of the target site on the ADE2 locus and repair using the Eco1 ncRNA-derived RT-DNA template.

The editing experiments at additional loci were carried out as described above, with the difference that editing was quantified by amplifying 0.5 ul of the gDNA with loci-specific primers, adapters for Illumina sequencing preparation. These primers are listed in Table 6. Custom Python software was used to quantify the percent of precise edits, caused by Cas9 cleavage of the target site on the ADE2 locus and repair using the Eco1 ncRNA-derived RT-DNA template.

### Human Editing Expression and Analysis

For human genome editing experiments, Cas9 or Cas9-P2A-Eco1RT expression in stable HEK293T cell lines was induced using 1 μg/mL doxycycline for 24 h at 37° C. in T12.5 flasks. Then, cultures were transiently transfected with a plasmid constitutively expressing ncRNA/gRNA at a concentration of 5 μg plasmid per T12.5 flask using Lipofectamine 3000 (plasmid list in Tables 2-6). Cultures were passaged and doxycycline refreshed the following day for 48 more hours. Three days post-transfection, cells were harvested for sequencing analysis.

To prepare samples for sequencing, cell pellets were processed and gDNA extracted using a QIAamp DNA mini kit, according to the manufacturer's instructions. DNA was eluted in 200 μL of ultra-pure, nuclease free water. Then. 0.5 μl of the gDNA was used as template in 12.5 μl PCR reactions with primer pairs to amplify the locus of interest, which additionally contained adapters for Illumina sequencing preparation (see Table 6 for primer and oligo sequences). Importantly, the primers do not bind to the ncRNA/gRNA plasmids. The amplicons were purified using a QIAquick PCR purification kit according to the manufacturer's instructions, and the amplicons eluted in 12 uL of ultra-pure, nuclease free water. Lastly, the amplicons were indexed and sequenced on an Illumina MiSeq instrument and processed with custom Python software to quantify the percent of on target precise and imprecise genomic edits.

Throughout the methods employed, biological replicates were collected from distinct samples and not the same sample measured repeatedly.

### Example 2: Modifications to the Retron ncRNA that Affect RT-DNA Production

A typical retron operon consists of a reverse transcriptase (RT), a non-coding RNA (ncRNA) that is both the primer and template for the reverse transcriptase, and one or more accessory proteins (FIG. 1A). The RT partially reverse transcribes the ncRNA to produce a single-stranded reverse transcribed DNA (RT-DNA) with a characteristic hairpin structure, which generally varies in length from 48-163 bases. The ncRNA can be sub-divided into a region that is reverse transcribed (msd) and a region that remains RNA in the final molecule (msr), which are partially overlapping.

One of the first described retrons was found in E. coli, called Eco1 (previously ec86). In BL21 cells, this retron is both present and active, producing RT-DNA that can be detected at the population level, which is eliminated by removing the retron operon from the genome (FIG. 1B). In the absence of this native operon, the ncRNA and RT can be expressed from a plasmid lacking the accessory protein, which is a minimal system for RT-DNA production. Because the accessory protein is a core component of the phage-defense conferred by retrons, retrons produced from this non-accessory protein plasmid have reduced phage defense capacity, yet this plasmid continues to produce abundant RT-DNA, due to expression of the ncRNA and reverse transcription of each ncRNA to generate multiple copies of reverse transcribed msd DNA (RT-DNA).

The inventors quantified the RT-DNA from Eco1 retrons using a relative qPCR assay that compared amplification by primers that bind different sections of the msd region. Such msd-primers can use both the RT-DNA and the ncRNA-reverse transcriptase encoding plasmids as a template (blue-black primers shown in FIG. 1C). A second set of primers was used to amplify only a portion of the plasmid, without amplifying the RT-DNA (red-black primers shown in FIG. 1C). In E. coli lacking an endogenous retron, overexpression of the ncRNA and RT from a plasmid yielded an approximate 8-10 fold enrichment of the reverse transcribed DNA/plasmid region over the plasmid alone, which is evidence of substantial reverse transcription (FIG. 1D).

Retrons are particularly useful for biotechnology at least in part because they have the potential to produce increased RT-DNA abundance in cells well above what can be achieved with delivery of a synthetic template.

The inventors evaluated how to modify various features of the ncRNA to produce even more abundant RT-DNA. To do this, variants of the Eco1 ncRNA were synthesized and cloned into expression vectors, with a retron reverse transcriptase expressed from a separate vector. The initial modified-retron library contained variants that extended or reduced the length of the hairpin stem of the RT-DNA. This variant cloning took place in single-pot, golden gate reactions and the resulting libraries were purified and then cloned into an expression strain. Cells harboring these library ncRNA-encoding vector sets were grown overnight and then diluted and ncRNA expression was induced during growth for 5 hours (FIG. 1E).

RT-DNA production was analyzed compared to plasmid concentration (FIG. 1E). The relative abundance of each variant plasmid in the expression strain was quantified by multiplexed Illumina sequencing before and after expression. After expression, purified RT-DNA was additionally analyzed from pools of cells harboring different retron variants by isolating cellular nucleic acids, treating that population with an RNase mixture (A/T1), and then isolating single-stranded DNA from double-stranded DNA using a commercial column-based kit. The RT-DNAs were then sequenced and their relative abundance was compared to that of their plasmid of origin to quantify the influence of different ncRNA parameters on RT-DNA production. To sequence the RT-DNA variants in this library, a custom sequencing pipeline was used to prepare each RT-DNA without bias toward any variant. This involved tailing purified RT-DNA with a string of polynucleotides using a template-independent polymerase (TdT), and then generating a complementary strand via an adapter-containing, inverse anchored primer. Finally, a second adapter was ligated to this double-stranded DNA and proceed to indexing and multiplexed sequencing (FIG. 1K-L).

In this first library, the msd stem length was modified from 0-31 base pairs. As illustrated in FIG. 1F, the msd stem length can have a large impact on RT-DNA production. The reverse transcriptase tolerated modifications of the msd stem length that deviate by a small amount from the wild-type (wt) length of 25 base pairs. However, variants with stem lengths less than 12 and greater than 30 produced less than half as much RT-DNA compared to the wild type. Therefore, stem length of between 12 and 30 base pairs was used going forward.

In a second library, the effect of increasing the loop length at the top of what becomes the RT-DNA stem was investigated. To do this, five random sequences of 70 bases each were created. Variant ncRNAs were then synthesized by incorporating 5-70 of these bases into the msd top loop. Five versions of each loop length were tested, each with different base content. Each variant's RT-DNA production, at every loop length, was then averaged. The wild type loop was not included in this library, so RT-DNA production was normalized to the five base loops that are closest in size to the wild type length of four bases.

As illustrated in FIG. 1G, substantial declines in RT-DNA production were observed as the loop length increased from 5 to about 14 bases, but almost no further decline in RT-DNA production was observed beyond that loop length, other than at loop length 28 bases, which inexplicably produced more RT-DNA than loops of neighboring lengths. While synthesis and sequencing parameters were limited to 70 bases in these studies, the data indicate that loops shorter than 14 bases are ideal for RT-DNA production. However, use of loops that extend beyond 14 bases still allow significant RT-DNA production.

Another investigated parameter was the length of a1/a2 complementarity, a region of the ncRNA structure where the 5′ and 3′ ends of the ncRNA fold back upon themselves, and which the inventors hypothesized plays a role in initiating reverse transcription (FIG. 1H). Because this region of the ncRNA is not reverse transcribed, variants in this region within the RT-DNA population could not be directly sequenced. Instead, a nine base barcode was introduced in an extended loop of each msd that could be sequenced as a proxy for the modification (FIG. 1I). These barcodes were amplified directly from the purified RT-DNA for sequencing (FIG. 1J). Alternatively, the RT-DNA was prepared using the terminal deoxynucleotidyl transferase (TdT) method described above (FIG. 1L).

In both cases, a similar result was obtained: reducing the length of a1/a2 complementarity in this region below seven base pairs substantially impaired RT-DNA production (FIG. 1J). These findings are consistent with a critical role of the a1/a2 complementarity region in reverse transcription. However, extending the a1/a2 length resulted in increased production of RT-DNA relative to the wild type length (FIG. 1J). Importantly, this is the first modification to a retron ncRNA that has been shown to increase RT-DNA production.

### Example 3: RT-DNA Production in Eukaryotic Cells

This Example describes experiments designed to evaluate whether increased production by the extended a1/a2 region is a useful modification of retrons expressed and reverse transcribed in eukaryotic cells.

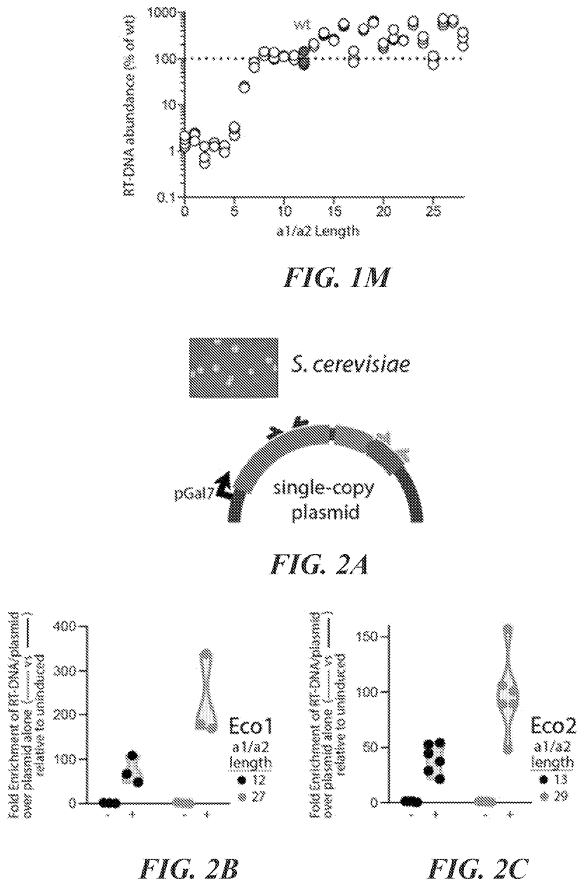

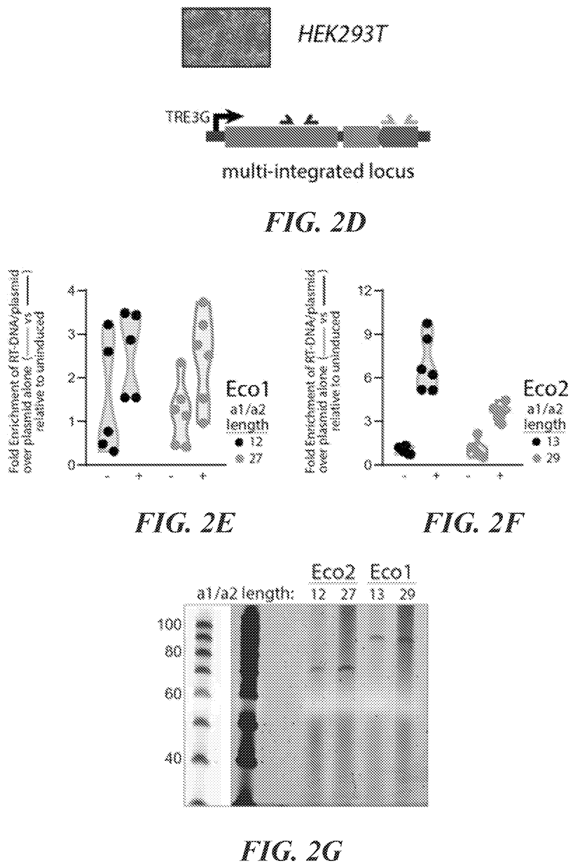

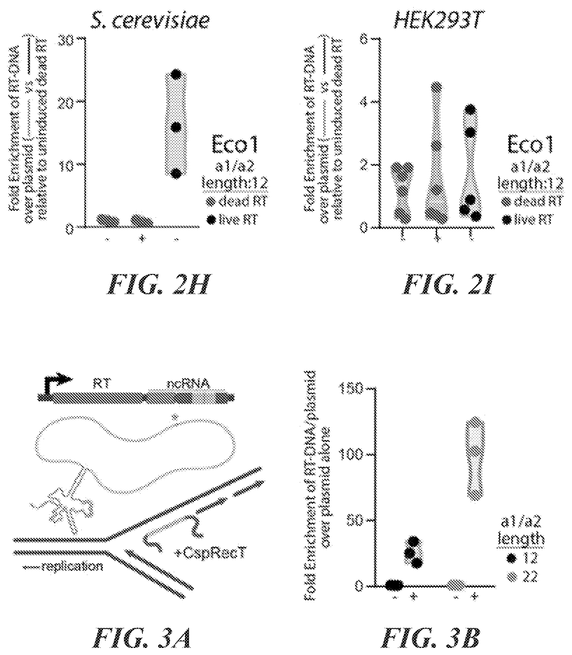

To facilitate expression of Eco1 in eukaryotic cells, the operon was inverted from its native configuration. In the endogenous arrangement, the ncRNA is in the 5′ UTR of the reverse transcriptase transcript, requiring internal ribosome entry for the reverse transcriptase from a ribosomal binding site (RBS) that is in or near the a2 region of the ncRNA. In eukaryotic cells, this arrangement puts the entire ncRNA between the 5′ mRNA cap and the initiation codon for the reverse transcriptase. This increased distance between the cap and initiation codon, as well as the ncRNA structure and out-of-frame ATG codons, can negatively affect reverse transcriptase translation. Moreover, altering the a1/a2 region in the native arrangement could have unintended effects on reverse transcriptase translation. In the inverted architecture designed by the inventors, expression of the reverse transcriptase (gray-shaded in FIG. 2A) is driven by a pol II promoter directly with its initiation codon near the 5′ end of the transcript and the ncRNA in the 3′ UTR, where variations are unlikely to influence reverse transcriptase translation. In FIG. 2A, the segment encoding the reverse transcriptase is shaded grey, while the msd region is blue, and the msr region is pink.

This inverted arrangement was first tested for Eco1 retrons in S. cerevisiae, by placing the RT-ncRNA cassette under the expression of Galactose inducible promoter, on a single-copy plasmid. RT-DNA production was detected using a qPCR assay analogous to that described for E. coli above, comparing amplification from primers that could use the plasmid or RT-DNA as a template to amplification from primers that could anneal only to the plasmid.

As illustrated in FIG. 2B, increasing the length of the Eco1 a1/a2 region from 12 to 27 base pairs resulted in more abundant RT-DNA production in yeast (FIGS. 2B, 2G). This analysis was extended to another retron, Eco2. Similar results were obtained: though the wild type ncRNA produced detectable RT-DNA, modified ncRNAs with extended a1/a2 regions ranging from 13 to 29 base pairs produced significantly more RT-DNA in yeast (FIGS. 2C, 2G). In each case, induced yeast cells were compared to uninduced cells, which likely under-reports the total RT-DNA abundance if there is any transcriptional ‘leak’ from the plasmid in the absence of inducers. For example, RT-DNA production was detected in the uninduced condition relative to a control expressing a catalytically dead RT, indicating some transcriptional ‘leak’ occurred in uninduced yeast cells (FIG. 2I).

Cultured human cells, HEK293T, were then evaluated for RT-DNA production. The gene architecture used for HEK293T cells was similar to that used for yeast, but with a genome-integrating cassette (FIG. 2D).

As shown in FIG. 2E and FIG. 2I, regardless of a1/a2 length and even when using a tightly regulated promoter, Eco1 did not produce significant abundance of RT-DNA in human cells that was detectable by qPCR. In contrast, Eco2 produced detectable RT-DNA in human cells, with both a wild type and an extended a1/a2 region (FIG. 2F). In contrast to the results in yeast and bacteria, however, the introduction of an extended a1/a2 region diminishes, rather than enhances, production of RT-DNA in human cells. However, RT-DNA production by a retron (e.g., Eco2) can be achieved in human cells when using non-extended (or only slightly extended) a1/a2 regions.

### Example 4: Improvements Extend to Applications in Genome Editing

In prokaryotes, retron-derived RT-DNA can be used as a template for recombineering (Farzadfard & Lu, Science 346, 1256272 (2014); Schubert, M. G. et al. High throughput functional variant screens via in-vivo production of single-stranded DNA. bioRxiv. 2020.2003.2005.975441 (2020)). As illustrated in FIG. 3A, retron ncRNA was modified to include a long loop in the msd region that contains homology to a bacterial genomic locus along with one or more nucleotide modifications. When RT-DNA from this modified ncRNA was produced along with a single stranded annealing protein (SSAP; e.g., lambda Reds), the RT-DNA was incorporated into the lagging strand during bacterial genome replication, thereby editing the genome of half of the bacterial cell progeny. This process is typically carried out in modified bacterial strains with numerous nucleases and repair proteins knocked out, because editing occurs at a low rate in wild type cells (Shubert et al. (2020)).

The inventors evaluated whether increased RT-DNA abundance using retrons with extended a1/a2 regions could increase the rate of editing in relatively unmodified strains. The RT-DNA was designed to edit a single nucleotide in the rpoB gene and the retron had the same flexible architecture that was used for the yeast and mammalian expression, with the ncRNA in the 3′ UTR of the reverse transcriptase. A 12 base stem was used for the msd, which retains near-wt RT-DNA production.

Two versions of the editing retron were constructed, one with the wild type 12-base a1/a2 region and another with an extended 22-base a1/a2 length. PAGE and qPCR analysis confirmed that the extended 22-base a1/a2 version produced more abundant RT-DNA (FIG. 3B-3C). Each version of the ncRNA was expressed in BL21-AI cells along with CspRecT (a high-efficiency single stranded annealing protein (SSAP); Wannier et al. Proc. Nat'l Acad. Sci. USA 117, 13689-13698 (2020)), as well as mutL E32K (a dominant-negative mutL that eliminates mismatch repair at sites of single-base mismatch (Aronshtam & Marinus, Nuc. Acids Res. 24, 2498-2504 (1996); Nyerges et al. Proc. Nat'l Acad. Sci. USA 113, 2502-2507 (2016)). The BL21-AI cells were unmodified, other than the removal of the endogenous Eco1 retron operon.

As shown in FIG. 3D, both ncRNAs resulted in appreciable editing after a single 16 h overnight expression, but the extended version was significantly more effective.

To test whether the effect of the a1/a2 extension was locus-specific or generalized across prokaryotic-eukaryotic genomic sites, an additional three loci were tested for precise editing. The engineered retron mediated editing at each additional loci, and the efficiency of editing was improved by the a1/a2 extension at all three additional sites (FIG. 3I-3K). These data show that the abundance of the RT-DNA template for recombineering is a limiting factor for editing, and that modified ncRNA can be used to introduce edits at a higher rate than other expression systems.

Retron-derived RT-DNA can also be used to edit eukaryotic cells (Sharon et al., Cell 175, 544-557 (2018)). Specifically, in yeast, the ncRNA is modified in the region that will become a loop of a msd to contain have a region homologous to a genomic locus but with one or more nucleotide modifications. This step is similar to the process of making a retron for modifying prokaryotes. However, in this yeast version, the ncRNA is on a transcript that also includes an SpCas9 guide RNA (gRNA) and scaffold. When these components are expressed along with RT and SpCas9, the genomic site is cut and repaired precisely using the RT-DNA as a template (FIG. 3E). The modified ncRNAs were tested using methods similar to those described by Sharon et al. (Cell 175, 544-557 (2018)).

The ncRNA/gRNA transcript was expressed from a Galactose inducible promoter on a single-copy plasmid, flanked by ribozymes. Along with the plasmid-encoded ncRNA/gRNA, the following were expressed: either Eco1 RT, Cas9, both the RT and Cas9, or neither, from Galactose inducible cassettes integrated into the genome. The ncRNA/gRNA was designed to target and edit the ADE2 locus in yeast, providing a two-nucleotide modification and producing a cellular phenotype (pink colonies).

As illustrated in FIG. 3F-3G, when using the ncRNA with a 12-base a1/a2 length, the expression of both the RT and Cas9 was necessary for editing, as detected by pink yeast colony counts, with only a small amount of background editing when Cas9 was expressed alone. This is consistent with the reverse transcription of the ncRNA being required, rather than having the editing arising from the plasmid. In other words, the plasmid was not the immediate donor of the repair nucleic acids.

To test the effect of extending the a1/a2 region on genome editing efficiency, two versions of a1/a2 extended constructs were designed, both of which had a length of 27 base pairs, but the constructs differed in their a1/a2 sequence. As shown in FIG. 3F-3G, both versions outperformed the standard 12 base form for precise genome editing in yeast. Consistent with the results in E. coli, these data indicate that RT-DNA production is a limiting factor for precise genome editing, and that an extended a1/a2 length is a modification that can be used in yeast as a feature that enhances retron-based genome engineering. As shown in FIG. 3H, these phenotypic results were further confirmed by sequencing the ADE2 locus from batch cultures of yeast cells. Precise modifications of the site, resulting from edits that use the RT-DNA as a template, follow the same pattern as the phenotypic results, showing that editing depends on both the Cas9 nuclease and reverse transcriptase and precise editing is increased by extension of the a1/a2 region.

The rates of precise editing as determined by nucleic acid sequencing of batch culture extracts were consistently lower than those estimated from counting colonies. This is likely due to additional editing that continues to occur on the plate before counting, and the method used for counting colonies as pink even if they were only partially pink. Another source of pink colonies could be any imprecise edits to the site that resulted in a non-functional ADE2 gene. For example, some ADE2 loci were observed that matched neither the wild type nor the precisely edited sequence. These occurred at a low rate (˜1-3%) under all conditions, which was slightly elevated by Cas9 expression, but unaffected by RT expression/RT-DNA production (FIG. 4I). This, as well as the pattern of insertions, deletions, transitions, and transversions is consistent with a combination of sequencing errors and Cas9-produced indels (FIG. 4J).

As in the bacterial experiments, constructs with an extended a1/a2 region in yeast were tested to determine if this modification was an improvement allowing additional loci to be targeted across the yeast genome. To this end, wild type and extended a1/a2 retrons were generated to edit four additional loci in yeast (TRP2. FAA1, CAN1, and LYP1). We found that for three out of four additional loci, the extended a1/a2 retrons yielded higher rates of precise editing, whereas one site showed lower, but still substantial rates of editing with the extended version (FIG. 3L-3S). Overall, across the nine sites tested in bacteria and yeast, the a1/a2 extension improved editing rates at eight sites.

Different retrons were also tested to check whether the type of retron affected editing. Retron Eco1, Eco4 and Sen2 ncRNAs were engineered for genome editing to introduce a 2 bp mutation in the ADE2 gene as described in Example 1. These ncRNAs were then co-expressed in yeast with retron reverse transcriptases and SpCas9, and the precise edit rates were determined by deep sequencing of the ADE2 gene.

As illustrated in FIG. 3T, the Eco1 retron, the Eco4 retron, and the Sen2 retron all mediated high rates of precise editing.

### Example 5: Precise Editing by Retrons Extends to Human Cells

This Example described experiments designed to evaluate whether retron-produced RT-DNA could be used to for precise editing of human cells. Such editing can provide therapy for a variety of diseases and conditions.

Adapting the editing machinery to cultured human cells required some modifications to the constructs and methods. In yeast, the Cas9 and the retron RT were produced from separate promoters. In human cells, expressing both of these proteins from a single promoter simplifies the system and increases its portability. To identify an optimal single promoter architecture, six constructs were tested in yeast: four fusion proteins using two different linker sequences with both orientations of Cas9 and Eco1 RT; and two versions where Cas9 and Eco1 RT were separated by a P2A sequence (Liu et al., Nature 566, 218-223 (2019)) in both possible orientations. P2A sequences are between two coding regions and cause ribosomal “skipping” during translation, which results in a missing peptide bond and effectively separates the two encoded proteins. One advantage of P2A is its size, about 18-22 amino acids (e.g., P2A can have the sequence ATNFSLLKQAGDVEENPGP; SEQ ID NO: 98).

These constructs were co-expressed in yeast with the best performing ADE2 editing ncRNA/gRNA construct described above (extended v1, a1/a2 length 27 bp). As illustrated in FIG. 4A, expression of these constructs resulted in a range of precise editing rates, with the Cas9-P2A-RT version yielding editing rates comparable to our previous versions based on two promoters.

Two human (HEK293T) cell lines were then created that each harbored one of two integrating cassettes: Cas9 alone or Cas9-P2A-Eco1 reverse transcriptase (FIG. 4B). Initially, precise genome editing was tested using a pol II driven ncRNA/gRNA flanked by ribozymes, as had been done in yeast. However, no evidence was found of either precise editing or indels. This may be due to inefficient ribozyme-mediated gRNA release in human cells (Knapp et al. Nature communications 10, 1490 (2019)).

The expression of the ncRNA/gRNA retron was changed to be driven by a pol III H1 promoter, using a transiently transfected plasmid (FIG. 4B). Six genomic loci (HEK3, RNF2, EMX1, FANCF, HEK4, and AAVS1) were selected for editing, and ncRNA/gRNA plasmids aiming to target and edit the sites were generated.

The repair template was designed to introduce two distinct mutations, separated by at least 2 bp: the first introduced a single nucleotide change near the cut-site; the second recoded the PAM nucleotides (NGG→NHH, H: non-G nucleotide). The reasoning for this was two-fold: first, the multiple changes should both eliminate Cas9 cutting of the ncRNA/RT plasmid and re-cutting of the precisely recoded site; and second, these multiple, separated changes make it much less likely to mistakenly assign a Cas9-induced indel as a precise edit. As a technical aside, the inventors recommend against using single-base modifications to benchmark Cas9-induced precise editing applications as they are a common outcome of imprecise repair and can easily lead to inaccurate estimates of editing rate. Expression of the protein(s) was induced for 24 hours, then the ncRNA/gRNA plasmids were transfected into the cells. The cells were harvested cells three days after transfection.

As illustrated in FIG. 4C-4H, precise editing of each site in the presence of the reverse transcriptase was detected using targeted Illumina sequencing. The percentage of cells with precise editing was well above the background rate of editing in the absence of the reverse transcriptase. The small percentage of precise edits in the absence of the reverse transcriptase likely represents use of the plasmid as a repair template, and the gain in the editing rate in the presence of the reverse transcriptase indicates edits using RT-DNA as the template. Interestingly, the rates of imprecise edits (indels) decline in the presence of the reverse transcriptase by roughly the same magnitude as the precise edits themselves, indicating that the RT-DNA is being used to precisely edit sites that would have otherwise been edited imprecisely (FIG. 4K-4P).

### DISCUSSION

The bacterial retron is a molecular component that can be exploited to produce designer DNA sequences in vivo. The results yield a generalizable framework for retron RT-DNA production. Specifically, it is shown that a minimal stem length must be maintained in the msd to yield abundant RT-DNA and that the msd loop length affects RT-DNA production. It is also shown that there is a minimum length for the a1/a2 complementary region. Further, it is demonstrated that the a1/a2 region can be extended beyond its wt length to produce more abundant RT-DNA, and that increasing template abundance in both bacteria and yeast increases editing efficiency.

These modifications are portable, both across retrons and across species. The extended a1/a2 region produces more RT-DNA using Eco1 in bacteria and both Eco1 and Eco2 in yeast. Oddly, the extended a1/a2 region did not increase RT-DNA production in cultured human cells. Nonetheless, we provide a clear demonstration of retron-produced RT-DNA in human cells.

Retrons have been used to produce DNA templates for genome engineering (6,8,9), driven by the rationale that an intracellularly produced template eliminates the issues related to exogenous template delivery and availability. However, there have been no investigations of whether the RT-DNA templates are abundant enough to saturate the editing, or if even more template would lead to higher rates of editing. The results establish that editing template abundance is limiting for genome editing in both bacteria and yeast, because extension of the a1/a2 region, which increases the abundance of the RT-DNA, also increases editing efficiency.

Additionally, the inverted arrangement of the retron operon, with the ncRNA in the 3′ UTR of the RT transcript, was found to produce RT-DNA in bacteria, yeast, and mammalian cells. This is the first time that a single, unifying retron architecture has been shown to be compatible with all of these host systems, simplifying comparisons and portability across kingdoms.

It is also shown, consistent with contemporaneous studies (31), that the retron RT-DNA can be used as a template to precisely edit human cells. Further, the repair template design allows one to confidently call the precise editing rates. The same analysis has also been applied to the Cas9 only conditions and reported the precise editing rates therein. This allows for estimations of the proportion of precise editing attributable to nuclease-only activity, and ultimately aids in obtaining more realistic estimates of the precise editing rates attributable to the genome engineering tool of interest.

One major difference between the two eukaryotic systems (yeast/human) is the ratio of precise to imprecise editing. Yeast RT-DNA-based editing occurs at a ratio of ˜74:1 precise edits to imprecise edits, while human editing inverted at a ratio of ˜1:15 precise edits to imprecise edits. In summary, this work represents an advance in the versatile use of retron in vivo DNA synthesis and RT-DNA for genome editing across kingdoms.

### BIBLIOGRAPHY

1 Luo, D. & Saltzman, W. M. Synthetic DNA delivery systems. Nat Biotechnol 18, 33-37, doi:10.1038/71889 (2000).
2 Lin, S., Staahl, B. T., Alla. R. K. & Doudna, J. A. Enhanced homology-directed human genome engineering by controlled timing of CRISPR/Cas9 delivery. eLife 3, e04766, doi:10.7554/eLife.04766 (2014).
3 Paquet, D. et al. Efficient introduction of specific homozygous and heterozygous mutations using CRISPR/Cas9. Nature 533, 125-129, doi:10.1038/nature17664 (2016).
4 Kosicki, M., Tomberg, K. & Bradley. A. Repair of double-strand breaks induced by CRISPR-Cas9 leads to large deletions and complex rearrangements. Nat Biotechnol 36, 765-771, doi:10.1038/nbt.4192 (2018).
Devkota, S. The road less traveled: strategies to enhance the frequency of homology-directed repair (HDR) for increased efficiency of CRISPR/Cas-mediated transgenesis. BMB reports 51, 437-443 (2018).
6 Farzadfard, F. & Lu, T. K. Synthetic biology. Genomically encoded analog memory with precise in vivo DNA writing in living cell populations. Science 346, 1256272, doi:10.1126/science.1256272 (2014).
7 Anzalone, A. V. et al. Search-and-replace genome editing without double-strand breaks or donor DNA. Nature 576, 149-157, doi:10.1038/s41586-019-1711-4 (2019).
8 Sharon, E. et al. Functional Genetic Variants Revealed by Massively Parallel Precise Genome Editing. Cell 175, 544-557.e516, doi:10.1016/j.cell.2018.08.057 (2018).
9 Schubert, M. G. et al. High throughput functional variant screens via in-vivo production of single-stranded DNA. bioRxiv, 2020.2003.2005.975441, doi:10.1101/2020.03.05.975441 (2020).
10 Millman, A. et al. Bacterial Retrons Function In Anti-Phage Defense. Cell 183, 1551-1561.e1512, doi:10.1016/j.cell.2020.09.065 (2020).
11 Bobonis, J. et al. Bacterial retrons encode tripartite toxin/antitoxin systems. bioRxiv, 2020.2006.2022.160168, doi:10.1101/2020.06.22.160168 (2020).
12 Bobonis, J. et al. Phage proteins block and trigger retron toxin/antitoxin systems. bioRxiv, 2020.2006.2022.160242, doi:10.1101/2020.06.22.160242 (2020).
13 Gao, L. et al. Diverse enzymatic activities mediate antiviral immunity in prokaryotes. Science 369, 1077-1084, doi:10.1126/science.aba0372 (2020).
14 Mirochnitchenko, O., Inouye, S. & Inouye, M. Production of single-stranded DNA in mammalian cells by means of a bacterial retron. J Biol Chem 269, 2380-2383 (1994).
15 Lampson, B. C., Inouye, M. & Inouye, S. Retrons, msDNA, and the bacterial genome. Cytogenetic and genome research 110, 491-499, doi:10.1159/000084982 (2005).
16 Simon, A. J., Ellington, A. D. & Finkelstein, I. J. Retrons and their applications in genome engineering. Nucleic acids research 47, 11007-11019, doi:10.1093/nar/gkz865 (2019).
17 Inouye, S., Hsu, M. Y., Eagle, S. & Inouye, M. Reverse transcriptase associated with the biosynthesis of the branched RNA-linked msDNA in Myxococcus xanthus. Cell 56, 709-717, doi:10.1016/0092-8674(89)90593-x (1989).
18 Lampson, B. C., Inouye, M. & Inouye, S. Reverse transcriptase with concomitant ribonuclease H activity in the cell-free synthesis of branched RNA-linked msDNA of Myxococcus xanthus. Cell 56, 701-707, doi:10.1016/0092-8674(89)90592-8 (1989).
19 Lampson, B. C. et al. Reverse transcriptase in a clinical strain of Escherichia coli: production of branched RNA-linked msDNA. Science 243, 1033-1038 (1989).
20 Lim, D. & Maas, W. K. Reverse transcriptase-dependent synthesis of a covalently linked, branched DNA-RNA compound in E. coli B. Cell 56, 891-904 (1989).
21 Miyata, S., Ohshima, A., Inouye, S. & Inouye, M. In vivo production of a stable single-stranded cDNA in Saccharomyces cerevisiae by means of a bacterial retron. Proceedings of the National Academy of Sciences of the United States of America 89, 5735-5739, doi:10.1073/pnas.89.13.5735 (1992).
22 Chappell, S. A., Edelman, G. M. & Mauro, V. P. Ribosomal tethering and clustering as mechanisms for translation initiation. Proceedings of the National Academy of Sciences of the United States of America 103, 18077-18082, doi:10.1073/pnas.0608212103 (2006).
23 Wannier, T. M. et al. Improved bacterial recombineering by parallelized protein discovery. Proceedings of the National Academy of Sciences of the United States of America 117, 13689-13698, doi:10.1073/pnas.2001588117 (2020).
24 Aronshtam, A. & Marinus, M. G. Dominant negative mutator mutations in the mutL gene of Escherichia coli. Nucleic acids research 24, 2498-2504, doi:10.1093/nar/24.13.2498 (1996).
25 Nyerges, Á. et al. A highly precise and portable genome engineering method allows comparison of mutational effects across bacterial species. Proceedings of the National Academy of Sciences of the United States of America 113, 2502-2507, doi:10.1073/pnas.1520040113 (2016).
26 Wang, H. H. et al. Programming cells by multiplex genome engineering and accelerated evolution. Nature 460, 894-898, doi:10.1038/nature08187 (2009).
27 Zhang, Y. et al. A gRNA-tRNA array for CRISPR-Cas9 based rapid multiplexed genome editing in Saccharomyces cerevisiae. Nature communications 10, 1053, doi:10.1038/s41467-019-09005-3 (2019).
28 Liu, J. J. et al. CasX enzymes comprise a distinct family of RNA-guided genome editors. Nature 566, 218-223, doi:10.1038/s41586-019-0908-x (2019).
29 Knapp, D. et al. Decoupling tRNA promoter and processing activities enables specific Pol-II Cas9 guide RNA expression. Nature communications 10, 1490, doi:10.1038/s41467-019-09148-3 (2019).
30 Mali, P. et al. RNA-guided human genome engineering via Cas9. Science 339, 823-826, doi:10.1126/science.1232033 (2013).
31 Kong, X. et al. Precise genome editing without exogenous donor DNA via retron editing system in human cells. Protein & cell, doi:10.1007/s13238-021-00862-7 (2021).
32 Rogers, J. K. et al. Synthetic biosensors for precise gene control and real-time monitoring of metabolites. Nucleic acids research 43, 7648-7660, doi:10.1093/nar/gkv616 (2015).
33 Datsenko, K. A. & Wanner, B. L. One-step inactivation of chromosomal genes in Escherichia coli K-12 using PCR products. Proceedings of the National Academy of Sciences of the United States of America 97, 6640-6645, doi:10.1073/pnas. 120163297 (2000).
34 Gietz, R. D. & Schiestl, R. H. High-efficiency yeast transformation using the LiAc/SS carrier DNA/PEG method. Nature protocols 2, 31-34, doi:10.1038/nprot.2007.13 (2007).
35 Brachmann, C. B. et al. Designer deletion strains derived from Saccharomyces cerevisiae S288C: a useful set of strains and plasmids for PCR-mediated gene disruption and other applications. Yeast (Chichester, England) 14, 115-132, doi:10.1002/(sici)1097-0061(19980130)14:2<115::Aid-yea204>3.0.Co; 2-2 (1998).
36 Tian, S. & Das, R. Primerize-2D: automated primer design for RNA multidimensional chemical mapping. Bioinformatics (Oxford, England) 33, 1405-1406, doi:10.1093/bioinformatics/btw814 (2017).
37 Richardson, C. D., Ray, G. J., DeWitt, M. A., Curie. G. L. & Corn, J. E. Enhancing homology-directed genome editing by catalytically active and inactive CRISPR-Cas9 using asymmetric donor DNA. Nat Biotechnol 34, 339-344, doi:10.1038/nbt.3481 (2016).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements of Exemplary Embodiments:

1. An engineered retron ncRNA comprising (1) a guide RNA linked or inserted into an al or a2 complementary region of the ncRNA; and (2) a repair template inserted into the ncRNA msd-encoding loop.
2. The engineered retron ncRNA of statement 1, wherein the retron al complementary region and the a2 complementary region has at least 7 nucleotides of complementarity.
3. The engineered retron ncRNA of statement 1 or 2, wherein the guide RNA is linked or inserted into the retron al or a2 complementary region increases the length of al or a2 complementary region by at least 15 nucleotides, or by at least 16 nucleotides, or by at least 17 nucleotides, or by at least 18 nucleotides, or by at least 19 nucleotides, or by at least 20 nucleotides, or by at least 22 nucleotides, or by at least 25 nucleotides, or by at least 30 nucleotides.
4. The engineered retron ncRNA of any of statements 1-3, wherein the guide RNA binds to a target genomic DNA.
5. The engineered retron ncRNA of any of statements 1-4, wherein the guide RNA binds to a target genomic DNA in a bacterial, yeast, or mammalian cell.
6. The engineered retron ncRNA of any of statements 1-5, wherein the guide RNA binds to a target genomic DNA in a mammalian cell and the guide RNA increases the length of the al or a2 complementary region by at least 20 nucleotides.
7. The engineered retron ncRNA of any of statements 5 or 6, wherein the mammalian cell is a human cell.
8. The engineered retron ncRNA of any of statements 1-7, wherein the repair template binds to a target genomic DNA.
9. The engineered retron ncRNA of any of statements 1-8, wherein the repair template binds to a target genomic DNA in a bacterial, yeast, or mammalian cell.
10. The engineered retron ncRNA of any of statements 1-9, wherein the repair template binds to a target genomic DNA having at least one allele with a mutation or polymorphism.
11. The engineered retron ncRNA of any of statements 1-10, wherein the repair template comprises one or more non-complementary nucleotides compared to the target genomic DNA.
12. The engineered retron ncRNA of any of statements 1-11, wherein the repair template comprises two or more, or three or more non-complementary nucleotides compared to the target genomic DNA.
13. The engineered retron ncRNA of any of statements 11 or 12, wherein the non-complementary nucleotides are ‘repair’ nucleotides that can substitute for mutant, variant, or polymorphism nucleotides in the target genomic DNA.
14. The engineered retron ncRNA of any of statements 1-13, wherein the ncRNA msd-encoding loop is a fold of a double-stranded stem, and the stem is at least 12 nucleotides in length.
15. The engineered retron ncRNA of any of statements 1-14, wherein the ncRNA msd-encoding loop is a fold of a double-stranded stem, and the stem is 30 or fewer nucleotides in length.
16. A composition comprising a carrier and the engineered retron ncRNA of any of statements 1-14.
17. A method comprising administering the engineered retron ncRNA of any of statements 1-14, or the composition of statement 16 to a subject or to cell(s) from the subject.
18. The method of statement 17, wherein the subject has, or is suspected of having or developing a disease or condition.
19. The method of statement 18, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis. Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.
20. An expression cassette comprising a promoter operably linked to a DNA segment encoding the engineered ncRNA of any one of statements 1-15.
21. The expression cassette of statement 20, wherein the promoter is an RNA polymerase III promoter.
22. The expression cassette of statement 20 or 21, wherein the promoter is a 7SK, U6, or H1 RNA polymerase III promoter.
23. The expression cassette of statement 20, wherein the promoter is an RNA polymerase II promoter.
24. The expression cassette of any one of statements 20-23, wherein the promoter is also operably linked to a DNA segment encoding a retron reverse transcriptase.
25. The expression cassette of any one of statements 20-24, which does not comprise a DNA segment encoding a retron reverse transcriptase.
26. The expression cassette of any one of statements 20-25, which is within an expression vector.
27. A composition comprising a carrier and the expression cassette of any one of statements 20-26.
28. A method comprising administering the expression cassette of any one of statements 20-26, or the composition of statement 27 to a subject or to cell(s) from the subject.
29. The method of statement 28, wherein the subject has, or is suspected of having or developing a disease or condition.
30. The method of statement 29, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.
31. An expression system comprising at least one expression cassette comprising a promoter operably linked to a DNA segment encoding a retron reverse transcriptase, a DNA segment encoding a Cas nuclease, or a DNA segment encoding both a retron reverse transcriptase and a Cas nuclease.
32. The expression system of statement 31, wherein the DNA segment encodes a fusion of the retron reverse transcriptase and the Cas nuclease.
33. The expression system of statement 31 or 33, wherein the DNA segment encodes a fusion of the retron reverse transcriptase and the Cas nuclease, separated by a ribosomal skipping sequence.
34. The expression system of statement 33, wherein the skipping sequence comprises DxExNPGP (SEQ ID NO: 9), and each x is independently an amino acid.
35. The expression system of statement 33 or 34, wherein the skipping sequence comprises one of the following sequences:

T2A
(SEQ ID NO: 10))
(GSG) EGRGSLL TCGDVEENPGP
P2A
(SEQ ID NO: 11)
(GSG) ATNFSLLKQAGDVEENPGP
E2A
(SEQ ID NO: 12)
(GSG) QCTNYALLKLAGDVESNPGP
F2A
(SEQ ID NO: 13)
(GSG) VKQTLNFDLLKLAGDVESNPGP

36. The expression system of any one of statements 31-36, further comprising at least one expression cassette comprising a promoter operably linked to a DNA segment encoding a retron ncRNA.
37. The expression system of statement 36, wherein the retron ncRNA is an engineered retron ncRNA comprising (1) a guide RNA linked or inserted into an al or a2 complementary region of the ncRNA; and (2) a repair template inserted into the ncRNA msd-encoding loop.
38. The expression system of any one of statements 36-37, wherein the retron al complementary region and the a2 complementary region has at least 7 nucleotides of complementarity.
39. The expression system of any one of statements 36-38, wherein the guide RNA is linked or inserted into the retron al or a2 complementary region increases the length of al or a2 complementary region by at least 15 nucleotides, or by at least 16 nucleotides, or by at least 17 nucleotides, or by at least 18 nucleotides, or by at least 19 nucleotides, or by at least 20 nucleotides, or by at least 22 nucleotides, or by at least 25 nucleotides, or by at least 30 nucleotides.
40. The expression system of any one of statements 36-39, wherein the guide RNA binds to a target genomic DNA.
41. The expression system of any one of statements 36-40, wherein the guide RNA binds to a target genomic DNA in a bacterial, yeast, or mammalian cell.
42. The expression system of any one of statements 36-41, wherein the guide RNA binds to a target genomic DNA in a mammalian cell and the guide RNA increases the length of the al or a2 complementary region by at least 20 nucleotides.
43. The expression system of any one of statements 3642, wherein the mammalian cell is a human cell.
44. The expression system of any one of statements 37-43, wherein the repair template binds to a target genomic DNA.
45. The expression system of any one of statements 37-44, wherein the repair template binds to a target genomic DNA in a bacterial, yeast, or mammalian cell.
46. The expression system of any one of statements 3745, wherein the repair template binds to a target genomic DNA having at least one allele with a mutation or polymorphism.
47. The expression system of any one of statements 37-46, wherein the repair template comprises one or more non-complementary nucleotides compared to the target genomic DNA.
48. The expression system of any one of statements 37-47, wherein the repair template comprises two or more, or three or more non-complementary nucleotides compared to the target genomic DNA.
49. The expression system of any one of statements 47-48, wherein the non-complementary nucleotides are ‘repair’ nucleotides that can substitute for mutant, variant, or polymorphism nucleotides in the target genomic DNA.
50. The expression system of any one of statements 31-49, wherein at least one promoter is an RNA polymerase III promoter.
51. The expression system of statement 50, wherein the RNA polymerase III promoter is a 7SK, U6, or H1 RNA polymerase III promoter.
52. The expression system of any one of statements 31-51, wherein at least one promoter is an RNA polymerase II promoter.
53. The expression system of any one of statements 37-52, wherein the expression cassette comprising a retron ncRNA is separate from the expression cassette comprising a promoter operably linked to a DNA segment encoding a retron reverse transcriptase, a DNA segment encoding a Cas nuclease, or a DNA segment encoding both a retron reverse transcriptase and a Cas nuclease.
54. A composition comprising a carrier and the expression system of any one of statements 31-53.
55. A method comprising administering the expression system of any one of statements 31-53, or the composition of statement 54 to a subject or to cell(s) from the subject.
56. The method of statement 55, wherein the subject has, or is suspected of having or developing a disease or condition.
57. The method of statement 56, wherein the disease or condition is cystic fibrosis, thalassemia, sickle cell anemia, Huntington's disease, diabetes, Duchenne's Muscular Dystrophy, Tay-Sachs Disease. Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.
58. A method of genetically editing one or more cells, comprising:
   (a) transfecting a population of cells with the expression cassette of any one of statements 20-26, or the expression system of any one of statements 31-53 to generate a population of transfected cells; and
   (b) selecting one or more cells from the population of transfected cells as genetically edited cells.
59. The method of statement 58, wherein selecting one or more cells comprises generating colonies from individual transfected cells to provide isogenic individual colonies and selecting one or more precisely edited cells from at least one isogenic colony.
60. The method of statement 58 or 59, further comprising sequencing one or more genomic target sites in cells from one or more isogenic individual colonies to confirm that the genomic target sites in at least one of the isogenic individual colonies are precisely edited, thereby generating precisely edited cells.
61. The method of statement 60, further comprising administering a population of the precisely edited cells to a subject.
62. The method of statement 61, wherein the subject has, or is suspected of having or developing a disease or condition.
63. The method of statement 62, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis. Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A.” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. An engineered retron ncRNA comprising: an msr region, an msd region having an msd stem and msd loop, and an a1/a2 duplex region, wherein a1/a2 duplex region comprises at least 7 nucleotide base pairs, wherein the a1/a2 duplex further comprises a guide RNA, and wherein the msd loop comprises a repair template.

2. The engineered retron ncRNA of claim 1, wherein the msd stem is between 12 and 30 nucleotide base pairs in length.

3. The engineered retron ncRNA of claim 1, wherein the msd loop is between 5-14 nucleotides in length or alternately is at least 12 nucleotides in length and optionally may comprise the repair template.

4. The engineered retron ncRNA of claim 1, wherein the a1/a2 duplex is modified by increasing its length by at least 15 nucleotides, or by at least 16 nucleotides, or by at least 17 nucleotides, or by at least 18 nucleotides, or by at least 19 nucleotides, or by at least 20 nucleotides, or by at least 22 nucleotides, or by at least 25 nucleotides, or by at least 30 nucleotides.

5. The engineered retron ncRNA of claim 1, wherein the guide RNA binds to a target genomic DNA.

6. The engineered retron ncRNA of claim 1, wherein the guide RNA binds to a target genomic DNA in a bacterial, yeast, or mammalian cell.

7. The engineered retron ncRNA of claim 1, wherein the guide RNA is fused to the end of either strand of the a1/a2 duplex.

8. The engineered retron ncRNA of claim 5, wherein the mammalian cell is a human cell.

9. The engineered retron ncRNA of claim 1, wherein the repair template binds to a target genomic DNA.

10. The engineered retron ncRNA of claim 1, wherein the repair template binds to a target genomic DNA in a bacterial, yeast, or mammalian cell.

11. The engineered retron ncRNA of claim 1, wherein the repair template binds to a target genomic DNA having at least one allele with a mutation or polymorphism.

12. The engineered retron ncRNA of claim 1, wherein the repair template comprises one or more non-complementary nucleotides compared to the target genomic DNA.

13. The engineered retron ncRNA of claim 1, wherein the repair template comprises two or more, or three or more non-complementary nucleotides compared to the target genomic DNA.

14. The engineered retron ncRNA of claim 11, wherein the non-complementary nucleotides are ‘repair’ nucleotides that can substitute for mutant, variant, or polymorphism nucleotides in the target genomic DNA.

15. The engineered retron ncRNA of claim 1, wherein the msd stem is at least 12 nucleotides in length.

16. The engineered retron ncRNA of claim 1, wherein the msd stem is 30 or fewer nucleotides in length.

17. A composition comprising a carrier and the engineered retron ncRNA of any one of claims 1-16.

18. A method comprising administering the engineered retron ncRNA of any one of claims 1-16, or the composition of claim 17 to a subject or to cell(s) from the subject.

19. The method of claim 18, wherein the subject has, or is suspected of having or developing a disease or condition.

20. The method of claim 19, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

21. An expression cassette comprising a nucleotide sequence encoding the engineered ncRNA of any one of claims 1-16, and optionally a nucleotide sequence encoding a retron reverse transcriptase.

22. The expression cassette of claim 21, wherein the nucleotide sequence encoding the engineered ncRNA further comprises a first promoter, wherein the first promoter is optionally an RNA polymerase III promoter.

23. The expression cassette of claim 22, wherein the first promoter is a 7SK, U6, or H1 RNA polymerase III promoter.

24. The expression cassette of claim 22, wherein the first promoter is an RNA polymerase II promoter.

25. The expression cassette of claim 22, wherein the nucleotide sequence encoding the retron reverse transcriptase further comprises a second promoter.

26. The expression cassette of claim 25, wherein the second promoter is the same or different as the first promoter.

27. A vector comprising the expression cassette of any one of claims 21-26.

28. A composition comprising a carrier and the expression cassette of one of claims 21-26 or the vector of claim 27.

29. A method comprising administering the expression cassette of any one of claims 21-26 or the vector of claim 27, or the composition of claim 28 to a subject or to cell(s) from the subject.

30. The method of claim 29, wherein the subject has, or is suspected of having or developing a disease or condition.

31. The method of claim 30, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

32. A gene editing system comprising: one or more vectors comprising one or more nucleotide sequences encoding an engineered retron ncRNA of any one of claims 1-16, a retron reverse transcriptase, and a Cas nuclease.

33. The gene editing system of claim 32, wherein the retron reverse transcriptase and Cas nuclease are encoded as a fusion protein.

34. The gene editing system of claim 33, wherein the nucleotide sequence encoding the fusion protein comprising the retron reverse transcriptase and the Cas nuclease further comprises a ribosomal skipping sequence.

35. The gene editing system of claim 34, wherein the skipping sequence comprises DxExNPGP (SEQ ID NO: 9), and each x is independently an amino acid.

36. The gene editing system of claim 34 or 35, wherein the skipping sequence comprises one of the following sequences:
T2A
(SEQ ID NO: 10))
(GSG) EGRGSLL TCGDVEENPGP
P2A
(SEQ ID NO: 11)
(GSG) ATNFSLLKQAGDVEENPGP
E2A
(SEQ ID NO: 12)
(GSG) QCTNYALLKLAGDVESNPGP
F2A
(SEQ ID NO: 13)
(GSG) VKQTLNFDLLKLAGDVESNPGP

37. The gene editing system of claim 32, wherein the one or more vectors comprising one or more promoters.

38. The gene editing system of claim 32, wherein the guide RNA of the ncRNA binds to a target genomic DNA.

39. The gene editing system of claim 32, wherein the guide RNA of the ncRNA binds to a target genomic DNA in a bacterial, yeast, or mammalian cell.

40. The gene editing system of claim 32, wherein the guide RNA of the ncRNA binds to a target genomic DNA in a mammalian cell.

41. The gene editing system of claim 40, wherein the mammalian cell is a human cell.

42. The gene editing system of claim 32, wherein the repair template of the ncRNA binds to a target genomic DNA.

43. The gene editing system of claim 32, wherein the repair template of the ncRNA binds to a target genomic DNA in a bacterial, yeast, or mammalian cell.

44. The gene editing system of claim 32, wherein the repair template of the ncRNA binds to a target genomic DNA having at least one allele with a mutation or polymorphism.

45. The gene editing system of claim 32, wherein the repair template of the ncRNA comprises one or more non-complementary nucleotides compared to the target genomic DNA.

46. The gene editing system of claim 32, wherein the repair template of the ncRNA comprises two or more, or three or more non-complementary nucleotides compared to the target genomic DNA.

47. The gene editing system of claim 45, wherein the non-complementary nucleotides are ‘repair’ nucleotides that can substitute for mutant, variant, or polymorphism nucleotides in the target genomic DNA.

48. The gene editing system of claim 37, wherein at least one promoter is an RNA polymerase III promoter.

49. The gene editing system of claim 48, wherein the RNA polymerase III promoter is a 7SK, U6, or H1 RNA polymerase III promoter.

50. The gene editing system of claim 37, wherein at least one promoter is an RNA polymerase 11 promoter.

51. The gene editing system of claim 32, comprising a first vector encoding the ncRNA and a second vector encoding the retron reverse transcriptase and Cas nuclease.

52. A composition comprising a carrier and the gene editing system of any one of claims 32-51.

53. A method comprising administering the gene editing system of any one of claims 32-51, or the composition of claim 52 to a subject or to cell(s) from the subject.

54. The method of claim 53, wherein the subject has, or is suspected of having or developing a disease or condition.

55. The method of claim 54, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.

56. A method of genetically editing one or more cells, comprising:
(a) transfecting a population of cells with the expression cassette of any one of claims 21-26, or the gene editing system of any one of claims 32-51 to generate a population of transfected cells; and
(b) selecting one or more cells from the population of transfected cells as genetically edited cells.

57. The method of claim 56, wherein selecting one or more cells comprises generating colonies from individual transfected cells to provide isogenic individual colonies and selecting one or more precisely edited cells from at least one isogenic colony.

58. The method of claim 56, further comprising sequencing one or more genomic target sites in cells from one or more isogenic individual colonies to confirm that the genomic target sites in at least one of the isogenic individual colonies are precisely edited, thereby generating precisely edited cells.

59. The method of claim 58, further comprising administering a population of the precisely edited cells to a subject.

60. The method of claim 59, wherein the subject has, or is suspected of having or developing a disease or condition.

61. The method of claim 60, wherein the disease or condition is cystic fibrosis, thalassemia, sickle cell anemia, Huntington's disease, diabetes, Duchenne's Muscular Dystrophy, Tay-Sachs Disease, Marfan syndrome, Alzheimer's disease, Leber's hereditary optic atrophy (LHON), myoclonic epilepsy with ragged red fibers (MERRF), mitochondrial encephalopathy, lactic acidosis, and stroke-like episodes (MELAS; a type of dementia), obesity, cancers, brain ischemia, coronary disease, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), and combinations thereof.


SEQUENCE LISTING (102 sequences)

SEQ ID NO: 1 - DNA, 170 bp, synthetic construct
  source 1..170: mol_type = other DNA
atgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttatttctgacgtaagggtgcgca

SEQ ID NO: 2 - DNA, 963 bp, synthetic construct
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 3 - DNA, 1761 bp, synthetic construct
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 4 - protein, 319 aa, synthetic construct
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 5 - DNA, 132 bp, synthetic construct
  source 1..132: mol_type = other DNA
cacgcatgtaggcagatttgttggttgtgaatcgcaaccagtggccttaatggcaggaggaatcgcctccctaaaatccttgattcagagctatacggcaggtgtgctgtgcgaaggagtgcctgcatgcgt

SEQ ID NO: 6 - protein, 312 aa, synthetic construct
MSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 7 - protein, 311 aa, synthetic construct
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 8 - DNA, 221 bp, synthetic construct
  source 1..221: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 9 - protein, 8 aa, synthetic construct
DXEXNPGP

SEQ ID NO: 10 - protein, 21 aa, synthetic construct
GSGEGRGSLLTCGDVEENPGP

SEQ ID NO: 11 - protein, 22 aa, synthetic construct
GSGATNFSLLKQAGDVEENPGP

SEQ ID NO: 12 - protein, 23 aa, synthetic construct
GSGQCTNYALLKLAGDVESNPGP

SEQ ID NO: 13 - protein, 25 aa, synthetic construct
GSGVKQTLNFDLLKLAGDVESNPGP

SEQ ID NO: 14 - protein, 20 aa, synthetic construct
GGTSSGGSGTAGSSGATSGG

SEQ ID NO: 15 - protein, 33 aa, synthetic construct
SGGSSGGSSGSETPGTSESATPESSGGSSGGSS

SEQ ID NO: 16 - protein, 19 aa, synthetic construct
ATNFSLLKQAGDVEENPGP

SEQ ID NO: 17 - DNA, 485 bp, synthetic construct
  source 1..485: mol_type = other DNA
gggtgcgcatctgatgagtccgtgaggacgaaacgagctagctcgtcatgataagattccgtatgcgcacccttagcgagaggtttatcattaaggtcaacctctggatgttgtttcggcatcctgcattgaatctgagttactgtctgttttccttggaaatgttctatttagaaacaggggaattgcttattaacgaaattgcctgaaggcctcacaactctggacattataccattgatgcttgcgtcacttcaggaaacccgtttcttctgacgtaagggtgcgcatacggaatcttatcaattaacgaaattgccccagtttcagagctatgctggaaacagcatagcaagttgaaataaggctagtccgttatcaacttgaaaaagtggcaccgagtcggtgctttttgatggccggcatggtcccagcctcctcgctggcgccggctgggcaacaccttcgggtggcgaatgggactt

SEQ ID NO: 18 - protein, 319 aa, synthetic construct
MPPKKKRKVDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 19 - protein, 320 aa, synthetic construct
MPPKKKRKVSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 20 - DNA, 517 bp, synthetic construct
  source 1..517: mol_type = other DNA
gggtgcgcatctgatgagtccgtgaggacgaaacgagctagctcgtctgataagattccgtagctctttagcgttttatggatttaccacctgattggtcaaatctagttgggcgttgcgccaaactctaatttattgattacatttacagttgcggaacaaactttttgagccgcaattggaaatgttctatttagaaacaggggaattgcttattaacgaaattgcctgaaggcctcacaactctggacattataccattgatgcttgcgtcacttcgttgcggatcaaaaagtttgttccgcggcttcaagtaaagagctacggaatcttatcaattaacgaaattgccccagtttcagagctatgctggaaacagcatagcaagttgaaataaggctagtccgttatcaacttgaaaaagtggcaccgagtcggtgctttttgatggccggcatggtcccagcctcctcgctggcgccggctgggcaacaccttcgggtggcgaatgggactt

SEQ ID NO: 21 - DNA, 509 bp, synthetic construct
  source 1..509: mol_type = other DNA
gggtgcgcatctgatgagtccgtgaggacgaaacgagctagctcgtctgataagattccgtaactctttagcgttaggctttgatttatagccttgtcgagcgtttcgccagacactaacttattgagtacttttagggttgcgctagaaagttttctaccgatcctagtggaaatgttctatttagaaacaggggaattgcttattaacgaaattgcctgaaggcctcacaactctggacattataccattgatgcttgcgtcacttcctaggatcggtagaaaactttctagcgcctcctctagtaaagagttacggaatcttatcaattaacgaaattgccccagtttcagagctatgctggaaacagcatagcaagttgaaataaggctagtccgttatcaacttgaaaaagtggcaccgagtcggtgctttttgatggccggcatggtcccagcctcctcgctggcgccggctgggcaacaccttcgggtggcgaatgggactt

SEQ ID NO: 22 - protein, 1726 aa, synthetic construct
MPPKKKRKVKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTGGTSSGGSGPAGSSGATSGGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 23 - protein, 1726 aa, synthetic construct
MSRADPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGTSSGGSGPAGSSGATSGGKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTPPKKKRKV

SEQ ID NO: 24 - protein, 1739 aa, synthetic construct
MPPKKKRKVKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 25 - protein, 1739 aa, synthetic construct
MSRADPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTPPKKKRKV

SEQ ID NO: 26 - protein, 1725 aa, synthetic construct
MPPKKKRKVKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTATNFSLLKQAGDVEENPGPDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 27 - protein, 1725 aa, synthetic construct
MSRADPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDATNFSLLKQAGDVEENPGPKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKTPPKKKRKV

SEQ ID NO: 28 - DNA, 630 bp, synthetic construct
  source 1..630: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacccgaaacgagctagctcgtcatgataagattccgtatgcgcacccttagcgagaggtttatcattaaggtcaacctctggatgttgtttcggcatcctgcattgaatctgagttactgtctgttttcctcttctccagccctggcctgggtcaatccttggggcccagactgagcacgtgctaacagaggaaaggaagccctgcttcctccagagggcgtcgcaggacagcttttcctagacaggggctaggaaacccgtttcttctgacgtaagggtgcgcatacggaatcttatcaggcccagactgagcacgtgagtttcagagctatgctggaaacagcatagcaagttgaaataaggctagtccgttatcaacttgaaaaagtggcaccgagtcggtgcttttt

SEQ ID NO: 29 - DNA, 630 bp, synthetic construct
  source 1..630: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacccgaaacgagctagctcgtcatgataagattccgtatgcgcacccttagcgagaggtttatcattaaggtcaacctctggatgttgtttcggcatcctgcattgaatctgagttactgtctgttttcctcccagtttacacgtctcatatgccccttggcagtcatcttagtcattacatgaaatgttcgttgtaactcatataaactgagttcccatgttttgcttaatggttgagttccgtttgtctaggaaacccgtttcttctgacgtaagggtgcgcatacggaatcttatcagtcatcttagtcattacctggtttcagagctatgctggaaacagcatagcaagttgaaataaggctagtccgttatcaacttgaaaaagtggcaccgagtcggtgcttttt

SEQ ID NO: 30 - DNA, 630 bp, synthetic construct
  source 1..630: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacccgaaacgagctagctcgtcatgataagattccgtatgcgcacccttagcgagaggtttatcattaaggtcaacctctggatgttgtttcggcatcctgcattgaatctgagttactgtctgttttcctacaaacggcagaagctggaggaggaagggcctgagtccgagcagaagaaaaagttctcccatcacatcaaccggtggcgcattgccacgaagcaggccaatggggaggacatcgatgtcaaggaaacccgtttcttctgacgtaagggtgcgcatacggaatcttatcagagtccgagcagaagaagaagtttcagagctatgctggaaacagcatagcaagttgaaataaggctagtccgttatcaacttgaaaaagtggcaccgagtcggtgcttttt

SEQ ID NO: 31 - DNA, 630 bp, synthetic construct
  source 1..630: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacccgaaacgagctagctcgtcatgataagattccgtatgcgcacccttagcgagaggtttatcattaaggtcaacctctggatgttgtttcggcatcctgcattgaatctgagttactgtctgttttcctgaaagcggaagtagggccttcgcgcacctcatggaatcccttctgcagcatctagatcgcttttccgagcttctggcggtctcaagcactacctacgtcagcacctgggaccccgccaccaggaaacccgtttcttctgacgtaagggtgcgcatacggaatcttatcaggaatcccttctgcagcaccgtttcagagctatgctggaaacagcatagcaagttgaaataaggctagtccgttatcaacttgaaaaagtggcaccgagtcggtgcttttt

SEQ ID NO: 32 - DNA, 630 bp, synthetic construct
  source 1..630: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacccgaaacgagctagctcgtcatgataagattccgtatgcgcacccttagcgagaggtttatcattaaggtcaacctctggatgttgtttcggcatcctgcattgaatctgagttactgtctgttttcctgatgacaggcaggggcaccgcggcgccccggtggcactgcggctggaggcgggaattaaagcggagactctggtgctgtgtgactacagtgggggccctgccctctctgagcccccgcctaggaaacccgtttcttctgacgtaagggtgcgcatacggaatcttatcaggcactgcggctggaggtgggtttcagagctatgctggaaacagcatagcaagttgaaataaggctagtccgttatcaacttgaaaaagtggcaccgagtcggtgcttttt

SEQ ID NO: 33 - DNA, 630 bp, synthetic construct
  source 1..630: mol_type = other DNA
aattcggaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacccgaaacgagctagctcgtcatgataagattccgtatgcgcacccttagcgagaggtttatcattaaggtcaacctctggatgttgtttcggcatcctgcattgaatctgagttactgtctgttttccttaatgtggctctggttctgggtacttttatctgtcccctccaccccacaatggaaccactagggacaggattggtgacagaaaagcccccatccttaggcctcctccttcctagtctcctaggaaacccgtttcttctgacgtaagggtgcgcatacggaatcttatcagtcccctccaccccacagtggtttcagagctatgctggaaacagcatagcaagttgaaataaggctagtccgttatcaacttgaaaaagtggcaccgagtcggtgcttttt

SEQ ID NO: 34 - DNA, 70 bp, synthetic construct
  source 1..70: mol_type = other DNA
ggatctattcataacttgatgtataaagtagaaaaaaaagcggggagattgtgtaggctggagctgcttc

SEQ ID NO: 35 - DNA, 69 bp, synthetic construct
  source 1..69: mol_type = other DNA
gaagtgctcaatttttcaacctatgagctttagttttaaaacgaagaccacatatgaatatcctcctta

SEQ ID NO: 36 - DNA, 21 bp, synthetic construct
  source 1..21: mol_type = other DNA
catgtgcatgaaaaccactgc

SEQ ID NO: 37 - DNA, 19 bp, synthetic construct
  source 1..19: mol_type = other DNA
atcatgccgtttgtgatgg

SEQ ID NO: 38 - DNA, 20 bp, synthetic construct
  source 1..20: mol_type = other DNA
gcaatgcgatgaaaagtgcc

SEQ ID NO: 39 - DNA, 27 bp, synthetic construct
  source 1..27: mol_type = other DNA
gtcagaaaaaacgggtttcctggttgg

SEQ ID NO: 40 - DNA, 21 bp, synthetic construct
  source 1..21: mol_type = other DNA
cctctggatgttgtttcggca

SEQ ID NO: 41 - DNA, 22 bp, synthetic construct
  source 1..22: mol_type = other DNA
caaggatgggttctacgtaaca

SEQ ID NO: 42 - DNA, 23 bp, synthetic construct
  source 1..23: mol_type = other DNA
gctaatttaggtgatgatggagc

SEQ ID NO: 43 - DNA, 23 bp, synthetic construct
  source 1..23: mol_type = other DNA
ctgagttactgtctgttttcctg

SEQ ID NO: 44 - DNA, 26 bp, synthetic construct
  source 1..26: mol_type = other DNA
tcagaaaaaacgggtttcctgaattc

SEQ ID NO: 45 - DNA, 48 bp, synthetic construct
  source 1..48: mol_type = other DNA
ctttccctacacgacgctcttccgatctctctgggcgatctggatacc

SEQ ID NO: 46 - DNA, 48 bp, synthetic construct
  source 1..48: mol_type = other DNA
ggagttcagacgtgtgctcttccgatctttcgattggacatacgcgac

SEQ ID NO: 47 - DNA, 48 bp, synthetic construct
  source 1..48: mol_type = other DNA
ctttccctacacgacgctcttccgatcttatcagccagcattccaacg

SEQ ID NO: 48 - DNA, 51 bp, synthetic construct
  source 1..51: mol_type = other DNA
ggagttcagacgtgtgctcttccgatctggctaacctgcgtattatcagtg

SEQ ID NO: 49 - DNA, 48 bp, synthetic construct
  source 1..48: mol_type = other DNA
ctttccctacacgacgctcttccgatctaatgagcaatcatacgcggg

SEQ ID NO: 50 - DNA, 51 bp, synthetic construct
  source 1..51: mol_type = other DNA
ggagttcagacgtgtgctcttccgatctacgattttagttaagggttcacg

SEQ ID NO: 51 - DNA, 49 bp, synthetic construct
  source 1..49: mol_type = other DNA
ctttccctacacgacgctcttccgatctgggacaatcttaccgctttcg

SEQ ID NO: 52 - DNA, 48 bp, synthetic construct
  source 1..48: mol_type = other DNA
ggagttcagacgtgtgctcttccgatctacaaggcaaagaacggactg

SEQ ID NO: 53 - DNA, 36 bp, synthetic construct
  source 1..36: mol_type = other DNA
agctaggtctcattcaatgcaggatgccgaaacaac

SEQ ID NO: 54 - DNA, 34 bp, synthetic construct
  source 1..34: mol_type = other DNA
agctaggtctcagtaagggtgcgcaactttcatg

SEQ ID NO: 55 - DNA, 36 bp, synthetic construct; identical to SEQ ID NO: 53
  source 1..36: mol_type = other DNA

SEQ ID NO: 56 - DNA, 37 bp, synthetic construct
  source 1..37: mol_type = other DNA
agctaggtctcaactttcatgaaatccgctgcatcac

SEQ ID NO: 57 - DNA, 15 bp, synthetic construct
  source 1..15: mol_type = other DNA
agtgacccgtccctg

SEQ ID NO: 58 - DNA, 15 bp, synthetic construct
  source 1..15: mol_type = other DNA
agtcgacctctgccc

SEQ ID NO: 59 - DNA, 15 bp, synthetic construct
  source 1..15: mol_type = other DNA
actggtgcgtcgtct

SEQ ID NO: 60 - DNA, 15 bp, synthetic construct
  source 1..15: mol_type = other DNA
cgggaagtgttcgcc

SEQ ID NO: 61 - DNA, 66 bp, synthetic construct
  source 1..66: mol_type = other DNA
ctttccctacacgacgctcttccgatctnnnnnttatgctaggtgatgcagcggatttcatgaaag

SEQ ID NO: 62 - DNA, 61 bp, synthetic construct
  source 1..61: mol_type = other DNA
ggagttcagacgtgtgctcttccgatctcatttaactatgataagattccgtatgcgcacc

SEQ ID NO: 63 - DNA, 35 bp, synthetic construct
  source 1..35: mol_type = other DNA
ggagttcagacgtgtgctcttccgatctggggggh

SEQ ID NO: 64 - DNA, 50 bp, synthetic construct
  source 1..50: mol_type = other DNA
ctttccctacacgacgctcttccgatcttcagaaaaaacgggttgtcgcc

SEQ ID NO: 65 - DNA, 50 bp, synthetic construct
  source 1..50: mol_type = other DNA
ggagttcagacgtgtgctcttccgatctgttacaagctgtttgtcgccag

SEQ ID NO: 66 - DNA, 20 bp, synthetic construct
  source 1..20: mol_type = other DNA
gtcgaaaagaaggggcctga

SEQ ID NO: 67 - DNA, 20 bp, synthetic construct
  source 1..20: mol_type = other DNA
ccgcgtacccttgtattcga

SEQ ID NO: 68 - DNA, 20 bp, synthetic construct
  source 1..20: mol_type = other DNA
taacgggtttcctggttggc

SEQ ID NO: 69 - DNA, 20 bp, synthetic construct
  source 1..20: mol_type = other DNA
accttaaagctgccctccat

SEQ ID NO: 70 - DNA, 20 bp, synthetic construct
  source 1..20: mol_type = other DNA
ggtacgctgccgtaatagga

SEQ ID NO: 71 - DNA, 20 bp, synthetic construct
  source 1..20: mol_type = other DNA
gaatcgcctccctaaaatcc

SEQ ID NO: 72 - DNA, 20 bp, synthetic construct
  source 1..20: mol_type = other DNA
gcacacctgccgtatagctc

SEQ ID NO: 73 - DNA, 48 bp, synthetic construct
  source 1..48: mol_type = other DNA
ctttccctacacgacgctcttccgatcttatgcgcctgctagagttcc

SEQ ID NO: 74 - DNA, 49 bp, synthetic construct
  source 1..49: mol_type = other DNA
ggagttcagacgtgtgctcttccgatctgcgttcgttgtaatggtggag

SEQ ID NO: 75 - DNA, 48 bp, synthetic construct
  source 1..48: mol_type = other DNA
ctttccctacacgacgctcttccgatcttgtcaaggaccaccaaaggt

SEQ ID NO: 76 - DNA, 48 bp, synthetic construct
  source 1..48: mol_type = other DNA
ggagttcagacgtgtgctcttccgatctgccgcataataagccaagcc

SEQ ID NO: 77 - DNA, 48 bp, synthetic construct
  source 1..48: mol_type = other DNA
ctttccctacacgacgctcttccgatctgacggcgaggagaactgaaa

SEQ ID NO: 78 - DNA, 48 bp, synthetic construct
  source 1..48: mol_type = other DNA
ggagttcagacgtgtgctcttccgatctcaccttgcaatgacccatgc

SEQ ID NO: 79 - DNA, 48 bp, synthetic construct
  source 1..48: mol_type = other DNA
ctttccctacacgacgctcttccgatctcccgtgtatgcgtatttgcc

SEQ ID NO: 80 - DNA, 49 bp, synthetic construct
  source 1..49: mol_type = other DNA
ggagttcagacgtgtgctcttccgatctgccttcttgtttttggctcga

SEQ ID NO: 81 - DNA, 48 bp, synthetic construct
  source 1..48: mol_type = other DNA
ctttccctacacgacgctcttccgatctgatcaaaccagtgcaagccg

SEQ ID NO: 82 - RNA, 48 nt, synthetic construct
  source 1..48: mol_type = other RNA
ggagttcagacgtgtgctcttccgatcttaacacggtcggtattgccc

SEQ ID NO: 83 - DNA, 48 bp, synthetic construct
  source 1..48: mol_type = other DNA
ctttccctacacgacgctcttccgatctagacagggatcccagggaaa

SEQ ID NO: 84 - DNA, 48 bp, synthetic construct
  source 1..48: mol_type = other DNA
ggagttcagacgtgtgctcttccgatctgcccagccaaacttgtcaac

SEQ ID NO: 85 - DNA, 53 bp, synthetic construct
  source 1..53: mol_type = other DNA
ctttccctacacgacgctcttccgatctctcttctttatttccagcaatgtct

SEQ ID NO: 86 - DNA, 48 bp, synthetic construct
  source 1..48: mol_type = other DNA
ctttccctacacgacgctcttccgatctaggtgaaggtgtggttccag

SEQ ID NO: 87 - DNA, 46 bp, synthetic construct
  source 1..46: mol_type = other DNA
ggagttcagacgtgtgctcttccgatctgccagagtccagcttggg

SEQ ID NO: 88 - DNA, 48 bp, synthetic construct
  source 1..48: mol_type = other DNA
ggagttcagacgtgtgctcttccgatctgatggatgtggcgcaggtag

SEQ ID NO: 89 - DNA, 48 bp, synthetic construct
  source 1..48: mol_type = other DNA
ctttccctacacgacgctcttccgatctccctcccttcaagatggctg

SEQ ID NO: 90 - DNA, 48 bp, synthetic construct
  source 1..48: mol_type = other DNA
ggagttcagacgtgtgctcttccgatcttcctttcaacccgaacggag

SEQ ID NO: 91 - DNA, 50 bp, synthetic construct
  source 1..50: mol_type = other DNA
ctttccctacacgacgctcttccgatctgctcagctagtcttcttcctcc

SEQ ID NO: 92 - DNA, 48 bp, synthetic construct
  source 1..48: mol_type = other DNA
ggagttcagacgtgtgctcttccgatctgggggtgtgtcaccagataa

SEQ ID NO: 93 - DNA, 274 bp, synthetic construct
  source 1..274: mol_type = other DNA
gaatctgagttactgtctgttttccttggaaatgttctatttagaaacaggggaattgcttattaacgaaattgcctgaaggcctcacaactctggacattataccattgatgcttgcgtcacttcaggaaacccgtttcttctgacgtaagggtgcgcaattaacgaaattgccccagtttcagagctatgctggaaacagcatagcaagttgaaataaggctagtccgttatcaacttgaaaaagtggcaccgagtcggtgctttttgatgg

SEQ ID NO: 94 - DNA, 20 bp, synthetic construct
  source 1..20: mol_type = other DNA
acgttccgccttaggaattt

SEQ ID NO: 95 - DNA, 20 bp, synthetic construct
  source 1..20: mol_type = other DNA
tttctcaggccccttctttt

SEQ ID NO: 96 - DNA, 21 bp, synthetic construct
  source 1..21: mol_type = other DNA
aaataacgggtttcctggttg

SEQ ID NO: 97 - DNA, 21 bp, synthetic construct
  source 1..21: mol_type = other DNA
tctgttttccttgttggaacg

SEQ ID NO: 98 - protein, 19 aa, synthetic construct
ATNFSLLKQAGDVEENPGP

SEQ ID NO: 99 - DNA, 29 bp, synthetic construct
  source 1..29: mol_type = other DNA
tctgagttactgtctgttttccttgttgg

SEQ ID NO: 100 - DNA, 20 bp, synthetic construct
  source 1..20: mol_type = other DNA
tccttgttggaacggagagc

SEQ ID NO: 101 - DNA, 50 bp, synthetic construct
  source 1..50: mol_type = other DNA
ggagttcagacgtgtgctcttccgatctagccaacatacagaagtcagga

SEQ ID NO: 102 - DNA, 48 bp, synthetic construct
  source 1..48: mol_type = other DNA
ctttccctacacgacgctcttccgatcttcgcggatgttccaatcagt